(19) United States
(12) Patent Application Publication
(10) Pub. No.: US 2014/0066324 A1
(43) Pub. Date: Mar. 6, 2014

(54) GENE EXPRESSION SIGNATURE IN SKIN PREDICTS RESPONSE TO MYCOPHENOLATE MOFETIL

(71) Applicants: Michael L. Whitfield, Etna, NH (US); Monique Hinchcliff, Chicago, IL (US); Chiang-Ching Huang, Chicago, IL (US)

(72) Inventors: Michael L. Whitfield, Etna, NH (US); Monique Hinchcliff, Chicago, IL (US); Chiang-Ching Huang, Chicago, IL (US)

(73) Assignees: NORTHWESTERN UNIVERSITY, Evanston, IL (US); TRUSTEES OF DARTMOUTH COLLEGE, Hanover, NH (US)

(21) Appl. No.: 14/001,979

(22) PCT Filed: Feb. 28, 2012
(86) PCT No.: PCT/US2012/027020

§ 371 Date: Nov. 5, 2013

Related U.S. Application Data
(60) Provisional application No. 61/447,545, filed on Feb. 28, 2011.

(51) Int. Cl. C12Q 1/68

(52) U.S. Cl. CPC C12Q 1/6883
USPC 506/9; 435/6.11

(57) ABSTRACT

Described herein are methods and compositions for identifying patients with SSc or scleroderma who are responders to MMF treatment.

### RELATED APPLICATION

This application claims the benefit of the filing date of U.S. provisional application U.S. Ser. No. 61/447,545, filed Feb. 28, 2011. The referenced application is incorporated herein by reference in its entirety.

### GOVERNMENT FUNDING

This application was made with government support under Grant No. HD055884, awarded by National Institutes of Health (NIH) Eunice Kennedy Shriver National Institute of Child Health & Human Development, and under Grant No. AR048098, awarded by NIH. The government has certain rights in the invention

### FIELD

The present invention is directed generally to methods for identifying mycophenolate mofetil (MMF)-responsive patients diagnosed with systemic sclerosis (SSc)/scleroderma. In particular, the invention relates to the identification of gene expression signatures or molecular profiles of skin, which correlate with a SSc/scleroderma patient's responsiveness to treatment with MMF.

### BACKGROUND

Systemic sclerosis (SSc)/scleroderma is a phenotypically diverse autoimmune disease of unknown etiology with a clinical hallmark of skin and internal organ fibrosis. Skin tightening and internal organ dysfunction cause considerable morbidity and mortality in those with SSc, respectively. Patients are prescribed various experimental treatments, including mycophenolate mofetil (MMF) (e.g., CELLCEPT®, Roche), an immunosuppressant, to halt and/or reverse skin and lung disease. Response to treatment is variable.

MMF inhibits inosine monophosphate dehydrogenase, the enzyme that controls the rate of synthesis of guanine monophosphate in the de novo pathway of purine synthesis used in the proliferation of B and T lymphocytes. Inhibition of the enzyme results in alteration of cell-mediated immunity and antibody formation [Ransom, J. T., Therapeutic Drug Monitoring, 1995. 17(6): p. 681-4]. MMF is a well-tolerated, safe, and FDA-approved medication for the prevention and treatment of solid organ transplant rejection. Its off-label indications include treatment for lupus nephritis, idiopathic thrombocytopenic purpura, Wegener's granulomatosis, myasthenia gravis and psoriasis, which are diseases that are thought to result in part from immune system activation. Because allograft vasculopathy resembles the vasculopathy that occurs in SSc, and because allograft vasculopathy is thought to play a major role in allograft rejection, MMF has been used in transplant recipients and SSc patients. Further evidence that supports a role for MMF in the treatment of SSc includes the ability of MMF to inhibit neointimal hyperplasia and prevent collagen production [Roos, N., et al., J Pharmacol Exp Ther, 2007. 321(2): p. 583-9; Schwarze, M. L., et al., J Heart Lung Transplant, 2001. 20(2): p. 160].

The modified Rodnan Skin Score (mRSS) is a validated marker of skin disease in SSc [Clements, P. J., et al., J Rheumatol, 1993. 20(11): p. 1892-6]. Improvement in the score is associated with improved survival. It appears to improve in some patients taking MMF. Lung function and radiographic findings improve in some patients prescribed MMF. Case reports and retrospective studies suggest that MMF may effectively treat SSc skin and lung disease in some patients, but the response is variable and there is no biomarker capable of predicting response to MMF treatment [Plastiras, S. C. et al, Rheumatology, 2006. 45(12): p. 1572; Derk, C. T., et al., Rheumatology (Oxford), 2009. 48(12): p. 1595-9; Gerbino, A. J., et al., Chest, 2008. 133(2): p. 455-60; Herrick, A. L., et al., Journal of Rheumatology, 2010. 37(1): p. 116-24; Stratton, R. J., et al., Rheumatology (Oxford), 2001. 40(1): p. 84-8; Vanthuyne, M., et al., Clin Exp Rheumatol, 2007. 25(2): p. 287-92; Miniati, I., et al., Clinical and Experimental Rheumatology, 2007. 25(2): p. 169-71].

### SUMMARY

Described herein are methods and compositions for identifying patients with SSc who are likely to respond to MMF treatment.

In one embodiment, the method is a method of determining if a patient having SSc or scleroderma is a responder to MMF treatment or a non-responder to MMF treatment, comprising obtaining a gene expression signature (patient gene expression signature) from skin obtained from a patient (referred to as a patient to be assessed); and determining if the patient gene expression signature comprises increased expression of one or more (at least one) genes selected from A2M, AIF1, ALOX5AP, APOL2, APOL3, BATF, BCL3, BIRC1, BTN3A2, C10orf10, C1orf38, C6orf80, CCL2, CCL4, CCR5, CD8A, CDW52, COL6A3, COTL1, CPA3, CPVL, CTAG1B, DDX58, EBI2, EVI2B, F13A1, FAM20A, FAP, FCGR3A, FLJ11259, FLJ22573, FLJ23221, FLJ25200, FYB, GBP1, GBP3, GEM, GIMAP6, GMFG, GZMH, GZMK, HAVCR2, HCLS1, HLA-DMA, HLA-DOA, HLA-DPA1, HLA-DPB1, HLA-DQA1, HLA-DQA2, HLA-DQB1, HLA-DRB1, HLA-DRB5, ICAM2, IFI16, IFIT1, IFIT2, IFITM1, IFITM2, IFITM3, IL10RA, INDO, ITGB2, KIAA0063, LAMB1, LCP1, LGALS2, LGALS9, LILRB2, LOC387763, LOC400759, LUM, LYZ, MARCKS, MFNG, MGC24133, MPEG1, MRC1, MRCL3, MS4A6A, MX1, NNMT, NUP62, PAG, PLAU, PPIC, PTPRC, RAC2, RGS10, RGS16, RSAFD1, SAT, SCGB2A1, SLC20A1, SLCO2B1, SPARC, SULF1, TAP1, TCTEL1, TIMP1, TNFSF4, UBD, VSIG4, and ZFYVE26 compared to expression of the gene in a control sample, wherein increased expression of one or more of the genes in the patient gene signature classifies the patient as a MMF treatment responder. A patient skin sample used in the method can be referred to as a test genetic sample. If the patient gene signature does not comprise increased expression of one or more (at least one) genes from the group above (does not have a gene expression signature indicative of being a MMF treatment responder, as described herein), the patient is classified as a MMF treatment non-responder. Alternatively, the patient gene expression signature can be assessed to determine if it comprises increased expression of one or more (at least one) genes herein as genes within Group III (see below).

In an alternative embodiment, the method of determining if a patient having SSc or scleroderma is a responder to MMF treatment or a non-responder to MMF treatment, comprises obtaining a gene expression signature (patient gene expression signature) from skin obtained from a patient (referred to as a patient to be assessed); and determining if the patient gene expression signature comprises increased expression of one or more (at least one) genes (referred to herein as genes within Group II) selected from the following: genes, identified by name: AADAC, ADAM17, ADH1A, ADH1C, AHNAK, ALG1, ALG5, AMOT, AOX1, AP2A2, ARK5, ARL6IP5, ARMCX1, BECN1, BECN1, BMP8A, BNIP3L, C10orf119, C1orf24, C1orf37, C20orf10, C20orf22, C5orf14, C6orf64, C9orf61, CAPS, CASP4, CASP5, CAST, CAV2, CCDC6, CCNG2, CDC26, CDK2AP1, CDR1, CFHL1, CNTN3, CPNE5, CRTAP, CTNNA1, CTSC, CUTL1, CXCL5, CYBRD1, CYP2R1, DBN1, DCAMKL1, DCL-1, DIAPH2, DKK2, ECHDC3, ECM2, EIF3S7, EMB, EMCN, EMILIN2, ENPP2, EPB41L2, FBLN1, FBLN2, FEM1A, FGL2, FHL5, FKBP7, FLI1, FLJ10986, FLJ20032, FLJ20701, FLJ23861, FLJ34969, FLJ36748, FLJ36888, FLJ43339, FZR1, GABPB2, GARNL4, GHITM, GHR, GIT2, GLYAT, GPM6B, GTPBP5, HELB, HOXB4, IFNA6, IGFBP5, IL13RA1, IL15, KAZALD1, KCNK4, KCNS3, KCTD10, KIAA0232, KIAA0494, KIAA0562, KIAA0870, KIAA1190, KIF25, KLHL18, KLK2, LAMP2, LEPROTL1, LHFP, LMO2, LOC114990, LOC255458, LOC387680, LOC400027, LOC493869, LOC87769, LRBA, MAFB, MAGEH1, MAN2B2, MCCC2, MEGF10, MFAP5, MGC11308, MGC15523, MGC3200, MGC35048, MGC45780, MOGAT3, MPPE1, MPZ, MYO1B, MYOC, NFYC, NIPSNAP3B, OPTN, OSR2, PAM, PBXIP1, PCOLCE2, PDGFC, PDGFRA, PDGFRL, PEX19, PHAX, PIP, PKM2, PKP2, PMP22, POU2F1, PPAP2B, PRAC, PSMA5, PSORS1C1, PTGIS, RECK, RGS11, RGS5, RIMS3, RIPK2, RNASE4, RNF125, RNF13, RNF146, RNF19, ROBO1, ROBO3, RPL7A, SARA1, SAV1, SCGB1D1, SDK1, SECP43, SECTM1, SERPINB2, SGCA, SH3BGRL, SH3GLB1, SH3RF2, SLC10A3, SLC12A2, SLC14A1, SLC39A14, SLC7A7, SLC9A9, SLPI, SMAD1, SMAP1, SMARCE1, SMP1, SNTG2, SNX7, SOCS5, SSPN, STX7, SUMF1, TAS2R10, TDE2, TFAP2B, TGFBR2, THSD2, TM4SF3, TMEM25, TMEM34, TNA, TNKS2, TRAD, TRAF3IP1, TREM4, TRIM35, TRIM9, TTYH2, TUBB1, UBL3, ULK2, URB, USP54, UST, UTRN, UTX, WIF1, WWOX, XG, YPEL5, and ZFHX1B and genes, each identified by GenBank accession number only: A_{—}32_BS 169243, A_{—}32_BS200773, A_{—}32_BS53976, AC025463, AF124368, AF161364, AF318337, AF372624, AK001565, AK022793, AK055621, AK056856, AL050042, AL137761, BC035102, BC038761, BC039664, BG252130, BI014689, D80006, ENST00000298643, ENST00000300068, ENST00000305402, ENST00000307901, ENST00000321656, ENST00000322803, ENST00000329246, ENST00000331640, ENST00000332271, ENST00000333784, H16080, I_{—}1861543, I_{—}1882608, I_{—}1985061, I_{—}3335767, I_{—}3551568, I_{—}3588329, I_{—}932413, I_{—}962800, I_{—}966091, NM_{—}001008528, NM_{—}001009555, NM_{—}001013632, NM_{—}001014975, NM_{—}001018006, NM_{—}001018076, NM_{—}001025077, NM_{—}003671, NM_{—}014758, NM_{—}015262, NM_{—}138411, NM_{—}153030, NM_{—}173709, NM_{—}213595, NR_{—}002184, S62210, THC1419743, THC1429821, THC1457118, THC1459712, THC1461073, THC1506312, THC1511927, THC1515028, THC1525318, THC1531579, THC1544941, THC1551463, THC1559236, THC1560798, THC1563147, THC1572906, THC1574967, THC1591470, XM_{—}165930, and XM_{—}209429, compared to expression of the gene in a control sample, wherein increased expression of one or more of the genes in the patient gene signature classifies the patient as a MMF treatment responder. If the patient gene signature does not comprise increased expression of one or more (at least one) genes from the group above (does not have a gene expression signature indicative of being a MMF treatment responder, as described herein), the patient is classified as a MMF treatment non-responder.

In a further embodiment, the method of determining if a patient having SSc or scleroderma is a responder to MMF treatment or a non-responder to MMF treatment, comprises obtaining a gene expression signature (patient gene expression signature) from skin obtained from a patient (referred to as a patient to be assessed); and determining if the patient gene expression signature comprises decreased expression of one or more genes (referred to herein as genes within Group I) selected from the following: genes, each identified by name: ANP32A, APOH, ATAD2, B3GALT6, B3GAT3, C12orf14, C14orf131, CACNG6, CBLL1, CBX8, CDC7, CDT1, CENPE, CGI-90, CLDN6, CREB3L3, CROC4, DDX3Y, DERP6, DJ971N18.2, EHD2, ESPL1, FGF5, FLJ10902, FLJ12438, FLJ12443, FLJ12484, FLJ12572, FLJ20245, FLJ32009, FLJ35757, FXYD2, GABRA2, GATA2, GK, GSG2, HPS3, IKBKG, IL23A, INSIG1, KIAA1509, KIAA1609, KIAA1666, LDLR, LGALS8, LILRB5, LOC123876, LOC128977, LOC153561, LOC283464, LRRIQ2, LY6K, MAC30, ME2, MGC13186, MGC16044, MGC16075, MGC29784, MGC33839, MGC35212, MGC4293, MICB, MLL5, MTRF1L, MUC20, NICN1, NPTX1, OAS3, OGDHL, OPRK1, PCNT2, PDZK1, PITPNC1, PPFIA4, PREB, PRKY, PSMD11, PSPH, PSPHL, PTP4A3, PXMP2, RAB15, RAD51AP1, RIP, RNF121, RPL41, RPS18, RPS4Y1, RPS4Y2, S100P, SORD, SP1, SYMPK, SYT6, TM9SF4, TMOD3, TNFRSF12A, TPRA40, TRIP, TRPM7, TTR, TUBB4, VARS2L, ZNF572, and ZSCAN2 and genes, each identified by GenBank accession number: A_{—}24_BS934268, AB065507, AC007051, AI791206, AK022745, AK022893, AK022997, AK094044, AL391244, AL731541, AL928970, BC010544, BC020847, BM925639, BM928667, ENST00000328708, ENST00000333517, I_{—}1891291, I_{—}3580313, NM_{—}001009569, NM_{—}001024808, NM_{—}172020, NM_{—}173705, NM_{—}178467, NR_{—}001544, THC1434038, THC1484458, THC1504780, U62539, XM_{—}210579, XM_{—}303638, and XM_{—}371684, compared to expression of the gene in a control sample, wherein decreased expression of one or more of the genes in the patient gene signature classifies the patient as a MMF treatment responder. If the patient gene signature does not comprise decreased expression of one or more (at least one) genes from the group above (does not have a gene expression signature indicative of being a MMF treatment responder), the patient is classified as a MMF treatment non-responder.

Alternatively, the method of determining if a patient having SSc or scleroderma is a responder to MMF treatment or a non-responder to MMF treatment, comprises obtaining a gene expression signature (patient gene expression signature) from skin obtained from a patient (referred to as a patient to be assessed); and determining if the patient gene expression signature comprises: (a) increased expression of any one or more genes in Group III and decreased expression of any one or more genes in Group I; (b) increased expression of any one or more genes in Group III and increased expression of any one or more genes in Group II; or increased expression of any one or more genes in Group III, decreased expression of any one or more genes in Group I and increased expression of any one or more genes in Group II.

In a further embodiment of a method of determining if a patient having Systemic Sclerosis (SSc) or scleroderma (referred to as a patient to be assessed) is a responder to mycophenolate mofetil (MMF) treatment or a non-responder to MMF treatment, the method comprises: (a) comparing a gene expression signature obtained from skin of a patient having SSc or scleroderma (patient gene expression signature) to a gene expression signature that is characteristic of a responder to MMF treatment (to a responder gene expression signature) and (b) based on results of comparing in (a), determining if the patient is a responder or a non-responder to MMF treatment. If the patient gene expression signature is sufficiently similar to the responder gene expression signature, the patient to be assessed is a responder to MMF treatment. For example, a patient gene expression signature is compared with a responder gene expression signature that comprises increased expression of at least one of the following: A2M, AIF1, ALOX5AP, APOL2, APOL3, BATF, BCL3, BIRC1, BTN3A2, C10orf10, C1orf38, C6orf80, CCL2, CCL4, CCR5, CD8A, CDW52, COL6A3, COTL1, CPA3, CPVL, CTAG1B, DDX58, EBI2, EVI2B, F13A1, FAM20A, FAP, FCGR3A, FLJ11259, FLJ22573, FLJ23221, FLJ25200, FYB, GBP1, GBP3, GEM, GIMAP6, GMFG, GZMH, GZMK, HAVCR2, HCLS1, HLA-DMA, HLA-DOA, HLA-DPA1, HLA-DPB1, HLA-DQA1, HLA-DQA2, HLA-DQB1, HLA-DRB1, HLA-DRB5, ICAM2, IFI16, IFIT1, IFIT2, IFITM2, IFITM3, IL10RA, INDO, ITGB2, KIAA0063, LAMB1, LCP1, LGALS2, LGALS9, LILRB2, LOC387763, LOC400759, LUM, LYZ, MARCKS, MFNG, MGC24133, MPEG1, MRC1, MRCL3, MS4A6A, MX1, NNMT, NUP62, PAG, PLAU, PPIC, PTPRC, RAC2, RGS10, RGS16, RSAFD1, SAT, SCGB2A1, SLC20A1, SLCO2B1, SPARC, SULF1, TAP1, TCTEL1, TIMP1, TNFSF4, UBD, VSIG4, and ZFYVE26 and, if expression of at least one (one or more) gene(s) in the sample obtained from the patient (in the patient gene expression signature) is substantially the same as expression in the responder gene expression signature (is increased to the same extent or greater extent, compared to expression in a control sample, as the increased expression in the responder gene expression signature), the patient is classified as a MMF responder.

Alternatively, the patient gene expression signature is compared with a responder gene expression signature that comprises increased expression of one or more (at least one) genes (referred to herein as genes within Group II) selected from the following: genes, identified by name: AADAC, ADAM17, ADH1A, ADH1C, AHNAK, ALG1, ALG5, AMOT, AOX1, AP2A2, ARK5, ARL6IP5, ARMCX1, BECN1, BECN1, BMP8A, BNIP3L, C10orf119, C1orf24, C1orf37, C20orf10, C20orf22, C5orf14, C6orf64, C9orf61, CAPS, CASP4, CASP5, CAST, CAV2, CCDC6, CCNG2, CDC26, CDK2AP1, CDR1, CFHL1, CNTN3, CPNE5, CRTAP, CTNNA1, CTSC, CUTL1, CXCL5, CYBRD1, CYP2R1, DBN1, DCAMKL1, DCL-1, DIAPH2, DKK2, ECHDC3, ECM2, EIF3S7, EMB, EMCN, EMILIN2, ENPP2, EPB41L2, FBLN1, FBLN2, FEM1A, FGL2, FHL5, FKBP7, FLI1, FLJ10986, FLJ20032, FLJ20701, FLJ23861, FLJ34969, FLJ36748, FLJ36888, FLJ43339, FZR1, GABPB2, GARNL4, GHITM, GHR, GIT2, GLYAT, GPM6B, GTPBP5, HELB, HOXB4, IFNA6, IGFBP5, IL13RA1, IL15, KAZALD1, KCNK4, KCNS3, KCTD10, KIAA0232, KIAA0494, KIAA0562, KIAA0870, KIAA1190, KIF25, KLHL18, KLK2, LAMP2, LEPROTL1, LHFP, LMO2, LOC114990, LOC255458, LOC387680, LOC400027, LOC493869, LOC87769, LRBA, MAFB, MAGEH1, MAN2B2, MCCC2, MEGF10, MFAP5, MGC11308, MGC15523, MGC3200, MGC35048, MGC45780, MOGAT3, MPPE1, MPZ, MYO1B, MYOC, NFYC, NIPSNAP3B, OPTN, OSR2, PAM, PBXIP1, PCOLCE2, PDGFC, PDGFRA, PDGFRL, PEX19, PHAX, PIP, PKM2, PKP2, PMP22, POU2F1, PPAP2B, PRAC, PSMA5, PSORS1C1, PTGIS, RECK, RGS11, RGS5, RIMS3, RIPK2, RNASE4, RNF125, RNF13, RNF146, RNF19, ROBO1, ROBO3, RPL7A, SARA1, SAV1, SCGB1D1, SDK1, SECP43, SECTM1, SERPINB2, SGCA, SH3BGRL, SH3GLB1, SH3RF2, SLC10A3, SLC12A2, SLC14A1, SLC39A14, SLC7A7, SLC9A9, SLPI, SMAD1, SMAP1, SMARCE1, SMP1, SNTG2, SNX7, SOCS5, SSPN, STX7, SUMF1, TAS2R10, TDE2, TFAP2B, TGFBR2, THSD2, TM4SF3, TMEM25, TMEM34, TNA, TNKS2, TRAD, TRAF3IP1, TREM4, TRIM35, TRIM9, TTYH2, TUBB1, UBL3, ULK2, URB, USP54, UST, UTRN, UTX, WIF1, WWOX, XG, YPEL5, and ZFHX1B and genes, each identified by GenBank accession number: A_{—}32_BS 169243, A_{—}32_BS200773, A_{—}32_BS53976, AC025463, AF124368, AF161364, AF318337, AF372624, AK001565, AK022793, AK055621, AK056856, AL050042, AL137761, BC035102, BC038761, BC039664, BG252130, BI014689, D80006, ENST00000298643, ENST00000300068, ENST00000305402, ENST00000307901, ENST00000321656, ENST00000322803, ENST00000329246, ENST00000331640, ENST00000332271, ENST00000333784, H16080, I_{—}1861543, I_{—}1882608, I_{—}1985061, I_{—}3335767, I_{—}3551568, I_{—}3588329, I_{—}932413, I_{—}962800, I_{—}966091, NM_{—}001008528, NM_{—}001009555, NM_{—}001013632, NM_{—}001014975, NM_{—}001018006, NM_{—}001.018076, NM_{—}001025077, NM_{—}003671, NM_{—}014758, NM_{—}015262, NM_{—}138411, NM_{—}153030, NM_{—}173709, NM_{—}213595, NR_{—}002184, S62210, THC1419743, THC1429821, THC1457118, THC1459712, THC1461073, THC1506312, THC1511927, THC1515028, THC1525318, THC1531579, THC1544941, THC1551463, THC1559236, THC1560798, THC1563147, THC1572906, THC1574967, THC1591470, XM_{—}165930, and XM_{—}209429, compared to expression of the gene in a control sample, wherein increased expression of one or more of the genes in the patient gene signature classifies the patient as a MMF treatment responder. If the patient gene signature does not comprise increased expression of one or more (at least one) genes from the group above (does not have a gene expression signature indicative of being a MMF treatment responder, as described herein), the patient is classified as a MMF treatment non-responder.

Alternatively, the patient gene expression signature is compared with a responder gene expression signature that comprises decreased expression of one or more (at least one) genes (referred to herein as genes within Group I) selected from the following: genes, each identified by name: ANP32A, APOH, ATAD2, B3GALT6, B3GAT3, C12orf14, C14orf131, CACNG6, CBLL1, CBX8, CDC7, CDT1, CENPE, CGI-90, CLDN6, CREB3L3, CROC4, DDX3Y, DERP6, DJ971N18.2, EHD2, ESPL1, FGF5, FLJ10902, FLJ12438, FLJ12443, FLJ12484, FLJ12572, FLJ20245, FLJ32009, FLJ35757, FXYD2, GABRA2, GATA2, GK, GSG2, HPS3, IKBKG, IL23A, INSIG1, KIAA1509, KIAA1609, KIAA1666, LDLR, LGALS8, LILRB5, LOC123876, LOC128977, LOC153561, LOC283464, LRRIQ2, LY6K, MAC30, ME2, MGC13186, MGC16044, MGC16075, MGC29784, MGC33839, MGC35212, MGC4293, MICB, MLL5, MTRF1L, MUC20, NICN1, NPTX1, OAS3, OGDHL, OPRK1, PCNT2, PDZK1, PITPNC1, PPFIA4, PREB, PRKY, PSMD11, PSPH, PSPHL, PTP4A3, PXMP2, RAB15, RAD51AP1, RIP, RNF121, RPL41, RPS18, RPS4Y1, RPS4Y2, S100P, SORD, SP1, SYMPK, SYT6, TM9SF4, TMOD3, TNFRSF12A, TPRA40, TRIP, TRPM7, TTR, TUBB4, VARS2L, ZNF572, and ZSCAN2 and genes, each identified by GenBank accession number: A_{—}24_BS934268, AB065507, AC007051, AI791206, AK022745, AK022893, AK022997, AK094044, AL391244, AL731541, AL928970, BC010544, BC020847, BM925639, BM928667, ENST00000328708, ENST00000333517, I_{—}1891291, I_{—}3580313, NM_{—}001009569, NM_{—}001024808, NM_{—}172020, NM_{—}173705, NM_{—}178467, NR_{—}001544, THC1434038, THC1484458, THC1504780, U62539, XM_{—}210579, XM_{—}303638, and XM_{—}371684, compared to expression of the gene in a control sample, wherein decreased expression of one or more of the genes in the patient gene signature classifies the patient as a MMF treatment responder. If the patient gene signature does not comprise decreased expression of one or more (at least one) genes from the group above (does not have a gene expression signature indicative of being a MMF treatment responder, as described herein), the patient is classified as a MMF treatment non-responder.

In all embodiments of the method described herein, a gene expression signature is obtained from a sample, such as a skin sample, obtained from an individual and assessed using methods known to those of skill in the art, as discussed further herein. The skin sample can be obtained from a variety of locations and in some embodiments, is obtained from the arm of the back.

Also described herein are nucleic acid (DNA, RNA) microarrays, such as a nucleic acid micorarray comprising nucleic acids that hybridize to at least one gene selected from Group III or to a complement of at least one gene selected from Group III, a gene selected from Group II or to a complement thereof, and a gene selected from Group I or to a complement thereof.

### BRIEF DESCRIPTION OF THE DRAWINGS

The patent or patent application contains at least one drawing executed in color. Copies of this patent or patent application publication with color drawing(s) will be provided by the Office upon request and payment of the necessary fee.

FIG. 1 shows unsupervised hierarchical clustering analysis of the inflammatory genes that identify the inflammatory intrinsic subset of SSc.

FIG. 2A shows differences in gene expression in skin samples excised from the arm between responders and non-responders at baseline as demonstrated by SAM and cluster analysis.

FIG. 2B shows differences in gene expression in skin samples excised from the back between responders and non-responders at baseline as demonstrated by SAM and cluster analysis

### DETAILED DESCRIPTION

Gene expression analysis by DNA microarrays is a useful approach for identifying novel SSc biomarkers that are capable of meaningfully classifying patients into distinct molecularly defined subsets. A growing body of evidence suggests that DNA microarray analyses of skin may identify useful SSc biomarkers. Published studies have identified SSc gene expression signatures in skin that reliably distinguish patients from healthy individuals and correlate with mRSS [Gardner, H., et al., Arthritis Rheum, 2006. 54(6): p. 1961-73; Whitfield, M. L., et al., Proc Natl Acad Sci USA, 2003. 100(21): p. 12319-24; Milano, A., et al., PLoS One, 2008. 3(7): p. e2696; Sargent, J. L., et al., Curr Rheumatol Rep, 2008. 10(3): p. 205-11] Pendergrass, S. A. et al., J. Invest. Derm. 2012 Feb. 9. Gene expression in skin of patients with SSc has been used by Applicant to identify five molecular subsets of the condition (Diffuse 1, Diffuse 2, Inflammatory, Normal-like and Limited) that help to explain the observed heterogeneity in disease course and response to experimental treatments [Milano, A., et al., PLoS One, 2008. 3(7): p. e2696]. A recent study that assessed clinical response to rituximab in SSc patients found no clinical benefit and a lack of significant changes in gene expression in skin biopsies. Pendergrass, S. A. et al., J. Invest. Derm. 2012 Feb. 9. These findings contrast with the results of a small study that reported clinical improvement in skin and lung disease in two patients treated with 6 months of imatinib mesylate (e.g., GLEEVEC®, Novartis). Another experimental treatment for SSc identified biologically relevant changes in skin gene expression that coincided with improvement in lung and skin disease [Chung, L., et al., Arthritis Rheum, 2009. 60(2): p. 584-91].

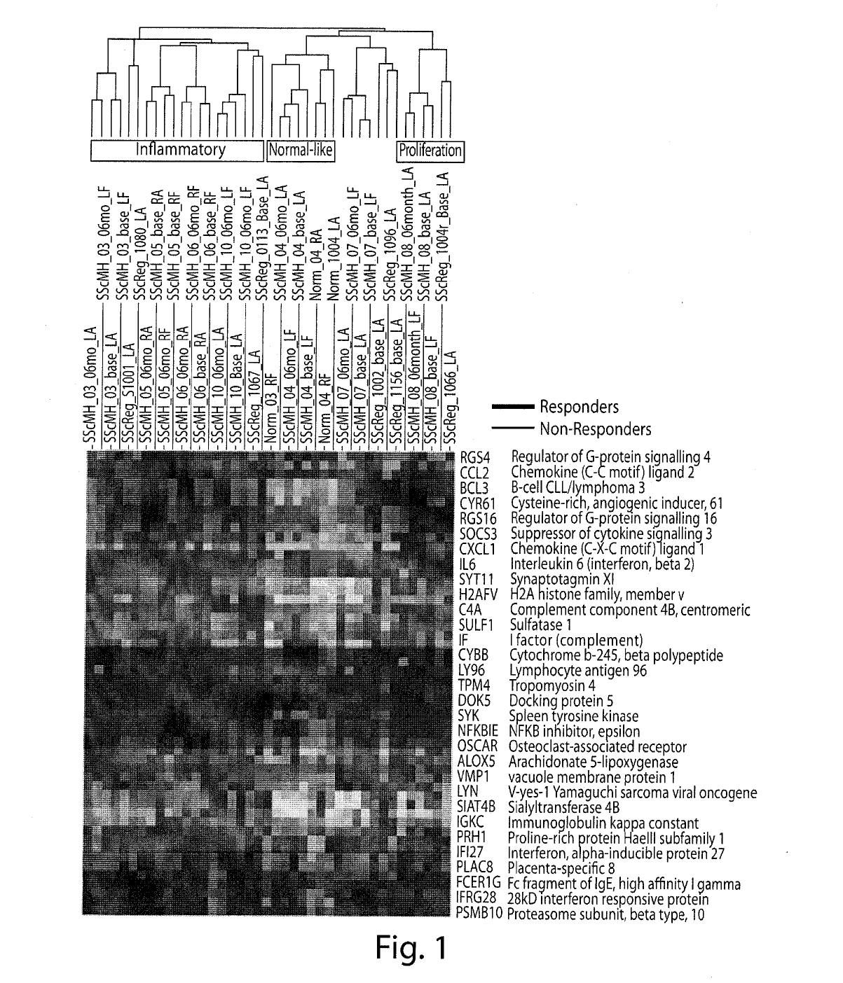

Described herein is a method of predicting whether (determining if) a patient with SSc will respond to treatment with MMF, based on assessment of the individual's gene expression signature. As described herein, Applicant has shown that patients with SSc who are responders to MMF treatment (MMF responders) have a gene expression signature that distinguishes them from patients with SSc who do not respond to MMF treatment (MMF nonresponders). The gene expression signature of a patient identified as a MMF treatment responder comprises one or more genes listed in tables and figures included herein. The method comprises comparing a gene expression signature obtained from a sample, such as a skin sample, obtained from a patient having SSc or scleroderma to a gene expression signature standard or reference, and identifying the patient as a responder or non-responder to MMF treatment. In specific embodiments, the gene expression signature obtained from a sample, such as a skin sample, is compared with a gene expression signature standard or reference that is one or more (at least one, a) gene (such as a gene set) that differentiates (distinguishes) MMF treatment responders from MMF treatment non-responders. For example, one or more genes whose presence or level or expression in patients with SSc who are known MMF treatment responders can be such a standard. For example, the gene expression signature of a patient with SSc can be compared with the genes listed in Table 1, Table 1A, Table 2, Table 3, Table 4 and/or Table 5. For example, the gene expression signature of a patient identified as a responder comprises one or more genes listed in Table 1, Table 1A, Table 2, Table 3, Table 4 and/or Table 5 and the difference in expression of the one or more genes between the patient and the standard is statistically significant. In a further embodiment, the gene expression signature of a patient identified as a responder comprises one or more genes listed in FIG. 1 and the difference in expression of the one or more genes between the patient and the standard is statistically significant.

In another embodiment, the standard genome-wide expression signature is obtained from skin of one or more SSc or scleroderma patient who is not responsive to MMF treatment.

Alternatively, the gene expression signature of a patient to be assessed for responsiveness to MMF treatment can be compared with expression of genes in Group I, Group II and/or Group III, as described herein. For example, the gene expression signature of the patient can be assessed for increased expression of one or more genes in Group III, increased expression of one or more genes in Group II, decreased expression of one or more genes in Group I or any combination of the three (e.g., increased expression of one or more genes in Group III and increased expression of one or more genes in Group II).

In specific embodiments, the gene expression signature of a MMF treatment responder comprises at least one gene that categorizes a patient with SSc as in the Inflammatory subtype, as defined by Milano, A. et al., PLoS One, 2008. 3(7); p. e2696 and in co-pending U.S. patent application Ser. No. 13/054,244. These include at least one gene named in any of the following: Table 1, Table 1A, Table 2, Table 3, Table 4 and Table 5 or otherwise presented herein (Example 4: IL-13 and IL-4 Gene Signatures Identify the Inflammatory Subset and the tables included therein)

In specific embodiments, a MMF treatment responder can be identified by the increased expression in a sample obtained from a patient with SSc of one or more genes (referred to in U.S. patent application Ser. No. 13/054,244 and herein as genes within Group III) selected from the following: genes identified by name: A2M, AIF1, ALOX5AP, APOL2, APOL3, BATF, BCL3, BIRC1, BTN3A2, C10orf10, C1orf38, C6orf80, CCL2, CCL4, CCR5, CD8A, CDW52, COL6A3, COTL1, CPA3, CPVL, CTAG1B, DDX58, EBI2, EVI2B, F13A1, FAM20A, FAP, FCGR3A, FLJ11259, FLJ22573, FLJ23221, FLJ25200, FYB, GBP1, GBP3, GEM, GIMAP6, GMFG, GZMH, GZMK, HAVCR2, HCLS1, HLA-DMA, HLA-DOA, HLA-DPA1, HLA-DPB1, HLA-DQA1, HLA-DQA2, HLA-DQB1, HLA-DRB1, HLA-DRB5, ICAM2, IFI16, IFI16, IFIT1, IFIT2, IFITM1, IFITM2, IFITM3, IL10RA, INDO, ITGB2, KIAA0063, LAMB I, LCP1, LGALS2, LGALS9, LILRB2, LOC387763, LOC400759, LUM, LYZ, MARCKS, MFNG, MGC24133, MPEG1, MRC1, MRCL3, MS4A6A, MX1, NNMT, NUP62, PAG, PLAU, PPIC, PPIC, PTPRC, RAC2, RGS10, RGS16, RSAFD1, SAT, SCGB2A1, SLC20A1, SLCO2B1, SPARC, SULF1, TAP1, TCTEL1, TIMP1, TNFSF4, UBD, VSIG4, and ZFYVE26 and genes, each identified by GenBank accession number: AF533936, BQ049338, ENST00000310210, ENST00000313904, ENST00000329660, I_{—}1000437, I_{—}966691, M 15073, NM_{—}001010919, NM_{—}001025201, NM_{—}001033569, THC1543691, and XM_{—}291496.

In further specific embodiments, a MMF treatment responder can be identified by the increased expression in a sample obtained from a patient with SSc of one or more genes (referred to in U.S. patent application Ser. No. 13/054,244 and herein as genes within Group II) selected from the following: genes, identified by name: AADAC, ADAM17, ADH1A, ADH1C, AHNAK, ALG1, ALG5, AMOT, AOX1, AP2A2, ARK5, ARL6IP5, ARMCX1, BECN1, BECN1, BMP8A, BNIP3L, C10orf119, C1orf24, C1orf37, C20orf10, C20orf22, C5orf14, C6orf64, C9orf61, CAPS, CASP4, CASP5, CAST, CAV2, CCDC6, CCNG2, CDC26, CDK2AP1, CDR1, CFHL1, CNTN3, CPNE5, CRTAP, CTNNA1, CTSC, CUTL1, CXCL5, CYBRD1, CYP2R1, DBN1, DCAMKL1, DCL-1, DIAPH2, DKK2, ECHDC3, ECM2, EIF3S7, EMB, EMCN, EMILIN2, ENPP2, EPB41L2, FBLN1, FBLN2, FEM1A, FGL2, FHL5, FKBP7, FLI1, FLJ10986, FLJ20032, FLJ20701, FLJ23861, FLJ34969, FLJ36748, FLJ36888, FLJ43339, FZR1, GABPB2, GARNL4, GHITM, GHR, GIT2, GLYAT, GPM6B, GTPBP5, HELB, HOXB4, IFNA6, IGFBP5, IL13RA1, IL15, KAZALD1, KCNK4, KCNS3, KCTD10, KIAA0232, KIAA0494, KIAA0562, KIAA0870, KIAA1190, KIF25, KLHL18, KLK2, LAMP2, LEPROTL1, LHFP, LMO2, LOC114990, LOC255458, LOC387680, LOC400027, LOC493869, LOC87769, LRBA, MAFB, MAGEH1, MAN2B2, MCCC2, MEGF10, MFAP5, MGC11308, MGC15523, MGC3200, MGC35048, MGC45780, MOGAT3, MPPE1, MPZ, MYO1B, MYOC, NFYC, NIPSNAP3B, OPTN, OSR2, PAM, PBXIP1, PCOLCE2, PDGFC, PDGFRA, PDGFRL, PEX19, PHAX, PIP, PKM2, PKP2, PMP22, POU2F1, PPAP2B, PRAC, PSMA5, PSORS1C1, PTGIS, RECK, RGS11, RGS5, RIMS3, RIPK2, RNASE4, RNF125, RNF13, RNF146, RNF19, ROBO1, ROBO3, RPL7A, SARA1, SAV1, SCGB1D1, SDK1, SECP43, SECTM1, SERPINB2, SGCA, SH3BGRL, SH3GLB1, SH3RF2, SLC10A3, SLC12A2, SLC14A1, SLC39A14, SLC7A7, SLC9A9, SLPI, SMAD1, SMAP1, SMARCE1, SMP1, SNTG2, SNX7, SOCS5, SSPN, STX7, SUMF1, TAS2R10, TDE2, TFAP2B, TGFBR2, THSD2, TM4SF3, TMEM25, TMEM34, TNA, TNKS2, TRAD, TRAF3IP1, TREM4, TRIM35, TRIM9, TTYH2, TUBB1, UBL3, ULK2, URB, USP54, UST, UTRN, UTX, WIF1, WWOX, XG, YPEL5, and ZFHX1B and genes, each identified by GenBank accession number only: A_{—}32_BS 169243, A_{—}32_BS200773, A_{—}32_BS53976, AC025463, AF124368, AF161364, AF318337, AF372624, AK001565, AK022793, AK055621, AK056856, AL050042, AL137761, BC035102, BC038761, BC039664, BG252130, BI014689, D80006, ENST00000298643, ENST00000300068, ENST00000305402, ENST00000307901, ENST00000321656, ENST00000322803, ENST00000329246, ENST00000331640, ENST00000332271, ENST00000333784, H16080, L1861543, I_{—}1882608, I_{—}1985061, L3335767, I_{—}3551568, I_{—}3588329, I_{—}932413, I_{—}962800, I_{—}966091, NM_{—}001008528, NM_{—}001009555, NM_{—}001013632, NM_{—}001014975, NM_{—}001018006, NM_{—}001018076, NM_{—}001025077, NM_{—}003671, NM_{—}014758, NM_{—}015262, NM_{—}138411, NM_{—}153030, NM_{—}173709, NM_{—}213595, NR_{—}002184, S62210, THC1419743, THC1429821, THC1457118, THC1459712, THC1461073, THC1506312, THC1511927, THC1515028, THC1525318, THC1531579, THC1544941, THC1551463, THC1559236, THC1560798, THC1563147, THC1572906, THC1574967, THC1591470, XM_{—}165930, and XM_{—}209429.

In further specific embodiments, a MMF treatment responder can be identified by the decreased expression in a sample obtained from a patient with SSc of one or more genes (referred to in U.S. patent application Ser. No. 13/054,244 and herein as genes within Group I) selected from the following: genes, each identified by name: ANP32A, APOH, ATAD2, B3GALT6, B3GAT3, C12orf14, C14orf131, CACNG6, CBLL1, CBX8, CDC7, CDT1, CENPE, CGI-90, CLDN6, CREB3L3, CROC4, DDX3Y, DERP6, DJ971N18.2, EHD2, ESPL1, FGF5, FLJ10902, FLJ12438, FLJ12443, FLJ12484, FLJ12572, FLJ20245, FLJ32009, FLJ35757, FXYD2, GABRA2, GATA2, GK, GSG2, HPS3, IKBKG, IL23A, INSIG1, KIAA1509, KIAA1609, KIAA1666, LDLR, LGALS8, LILRB5, LOC123876, LOC128977, LOC153561, LOC283464, LRRIQ2, LY6K, MAC30, ME2, MGC13186, MGC16044, MGC16075, MGC29784, MGC33839, MGC35212, MGC4293, MICB, MLL5, MTRF1L, MUC20, NICN1, NPTX1, OAS3, OGDHL, OPRK1, PCNT2, PDZK1, PITPNC1, PPFIA4, PREB, PRKY, PSMD11, PSPH, PSPHL, PTP4A3, PXMP2, RAB15, RAD51AP1, RIP, RNF121, RPL41, RPS18, RPS4Y1, RPS4Y2, S100P, SORD, SP1, SYMPK, SYT6, TM9SF4, TMOD3, TNFRSF12A, TPRA40, TRIP, TRPM7, TTR, TUBB4, VARS2L, ZNF572, and ZSCAN2 and genes, each identified by GenBank accession number: A_{—}24_BS934268, AB065507, AC007051, AI791206, AK022745, AK022893, AK022997, AK094044, AL391244, AL731541, AL928970, BC010544, BC020847, BM925639, BM928667, ENST00000328708, ENST00000333517, I_{—}1891291, I_{—}3580313, NM_{—}001009569, NM_{—}001024808, NM_{—}172020, NM_{—}173705, NM_{—}178467, NR_{—}001544, THC1434038, THC1484458, THC1504780, U62539, XM_{—}210579, XM_{—}303638, and XM_{—}371684.

In one embodiment, increased expression of one or more genes selected from A2M, AIF1, ALOX5AP, APOL2, APOL3, BATF, BCL3, BIRC1, BTN3A2, C10orf10, C1orf38, C6orf80, CCL2, CCL4, CCR5, CD8A, CDW52, COL6A3, COTL1, CPA3, CPVL, CTAG1B, DDX58, EBI2, EVI2B, F13A1, FAM20A, FAP, FCGR3A, FLJ11259, FLJ22573, FLJ23221, FLJ25200, FYB, GBP1, GBP3, GEM, GIMAP6, GMFG, GZMH, GZMK, HAVCR2, HCLS1, HLA-DMA, HLA-DOA, HLA-DPA1, HLA-DPB1, HLA-DQA1, HLA-DQA2, HLA-DQB1, HLA-DRB1, HLA-DRB5, ICAM2, IFI16, IFIT1, IFIT2, IFITM1, IFITM2, IFITM3, IL10RA, INDO, ITGB2, KIAA0063, LAMB 1, LCP1, LGALS2, LGALS9, LILRB2, LOC387763, LOC400759, LUM, LYZ, MARCKS, MFNG, MGC24133, MPEG1, MRC1, MRCL3, MS4A6A, MX1, NNMT, NUP62, PAG, PLAU, PPIC, PTPRC, RAC2, RGS10, RGS16, RSAFD1, SAT, SCGB2A1, SLC20A1, SLCO2B1, SPARC, SULF1, TAP1, TCTEL1, TIMP1, TNFSF4, UBD, VSIG4, and ZFYVE26 in the test genetic sample compared to the expression in a control sample classifies the patient as a MMF responder.

In further embodiments, a MMF responder can be identified by the increased expression of any one or more genes in Group III and the decreased expression of any one or more genes in Group I in a sample obtained from a patient with SSc; by the increased expression of any one or more genes in Group III and the increased expression of any one or more genes in Group II in a sample obtained from a patient with SSc; by the decreased expression of any one or more genes in Group I and the increased expression of any one or more genes in Group II in a sample obtained from a patient with SSc; or by the increased expression of any one or more genes in Group III, the decreased expression of any one or more genes in Group I and the increased expression of any one or more genes in Group II in a sample obtained from a patient with SSc.

Changes in gene expression in skin biopsies during treatment appear to occur only in patients who demonstrate clinical improvement, as judged by an improvement in mRSS or by other indicators of organ involvement. Described herein are results of microarray gene expression studies of skin biopsies that support use of molecular signatures in skin as SSc biomarker for clinical response to treatment with MMF. Clinical assessments and skin biopsies were performed on a cohort of SSc patients who were receiving MMF treatment for skin disease before the initiation of therapy and at 6 months and 12 months during (after initiation of) MMF treatment. The study identified a gene expression signature in skin in MMF treatment responders and non-responders at baseline and revealed differences in changes in gene expression that occurred in skin during treatment in responders and non-responders.

The terms “gene expression signature” and “molecular signature,” may be used interchangeably and refer to the expression profile of one or more genes obtained from a skin sample. A “gene” may be a single or double-stranded nucleic acid. A gene is known to one of skill in the art to be a locatable region of genomic sequence, corresponding to a unit of inheritance, which is associated with regulatory regions, transcribed regions, and or other functional sequence regions [Pearson, H., Nature, 2006. 441(7092): p. 398-401].

A “responder” is a patient diagnosed with SSc or scleroderma who responds positively to treatment with MMF. A patient experiencing a positive response to treatment with MMF is less symptomatic of SSc or scleroderma as compared to a patient who does not respond positively to treatment with MMF (i.e., a non-responder).

Skin biopsies or samples used herein may be obtained from any location on a patient's body. Preferably, the skin sample is obtained from the arm or back of the patient diagnosed with SSc or scleroderma.

A gene expression signature can be obtained using a variety of known approaches. For example, DNA microarray or gene chip (e.g., AFFYMETRIX®) analyses may be used to obtain a gene expression signature of a patient. Other genetic profiling techniques known to those of skill in the art may also be used, for example, serial analysis of gene expression (SAGE) or other lab-on-a-chip (LOC) technology.

To obtain a gene expression signature, a test sample containing at least one cell from clinically involved (i.e., diseased) tissue is needed (to obtain a sample of nucleic acid, also referred to as a genetic test sample, to be analyzed). Clinically involved tissue can include skin, esophagus, heart, lungs, kidneys, or synovium, but it is not so limited. The test sample may be obtained using any technique known in the art, including biopsy, blood sample, sample of bodily fluid (e.g., urine, lymph, ascites, sputum, stool, tears, sweat, pus, etc.), surgical excisions needle biopsy, scraping, etc. In particular embodiments, the test sample is clinically involved skin. A genetic sample or protein sample is obtained from the test sample, using known methods. The genetic sample contains a nucleic acid, such as RNA (e.g., mRNA) and/or DNA. For example, in determining gene expression, one can obtain mRNA from the test sample and the mRNA may be reverse transcribed into cDNA for further analysis. In another embodiment, mRNA is used in determining the expression of genes of interest. In some embodiments, the expression level of a particular gene can be determined by determining the level or presence of the protein encoded by the mRNA.

The test sample is preferably a sample representative of the scleroderma tissue as a whole. Multiple samples can be taken from the same tissue in order to obtain a representative sampling of the tissue.

A sample containing genetic material (RNA, DNA) can be obtained from the test sample using any suitable technique known in the art. See, e.g., Ausubel et al. (1999) Current Protocols in Molecular Biology (John Wiley & Sons, Inc., New York); Molecular Cloning: A Laboratory Manual (1989) 2nd Ed., ed. by Sambrook, Fritsch, and Maniatis (Cold Spring Harbor Laboratory Press); Nucleic Acid Hybridization (1984) B. D. Hames & S. J. Higgins eds. The nucleic acid can be purified from whole cells using DNA or RNA purification techniques. The genetic sample can also be amplified using PCR or in vivo techniques requiring subcloning. In a particular embodiment, the genetic sample is obtained by isolating mRNA from the cells of the test sample and creating cRNA as described herein.

Genetic samples are typically obtained from an individual (patient) having or suspected of having scleroderma. The individual is a mammal, e.g., a mouse, rat, hamster, rabbit, goat, sheep, cat, dog, pig, horse, cow, non-human primate, or human. In specific embodiments, the individual is a human.

As used herein, a “patient having scleroderma” has at least one recognized clinical manifestation of scleroderma. In one embodiment, a patient having scleroderma has been diagnosed as having scleroderma. Clinical diagnosis of scleroderma is well known in the medical arts. In one embodiment a patient having scleroderma has been diagnosed as having scleroderma on the basis, at least in part, of histological (optionally immunohistological) examination.

As used herein, a “patient suspected of having scleroderma” has at least one clinical sign or symptom that may suggest that the individual has scleroderma. In one embodiment a patient suspected of having scleroderma is suspected to have scleroderma but has not been diagnosed as having scleroderma. In one embodiment a patient suspected of having scleroderma is suspected to have scleroderma, but has not been diagnosed as having scleroderma on the basis, at least in part, of histological (optionally immunohistological) examination.

Raynaud's phenomenon is the presenting symptom in 75 percent of human subjects with scleroderma. This well-described phenomenon is characterized by episodic digital ischemia, clinically manifested by the sequential development of digital blanching, cyanosis, and rubor (redness) of the fingers or toes following cold exposure and subsequent rewarming. In one embodiment, a patient suspected of having scleroderma has Raynaud's phenomenon.

Once a test sample (also referred to here as a genetic sample) has been obtained, it can be analyzed for the presence, absence, or level of expression of one or more marker genes, e.g., intrinsic genes as disclosed herein. The analysis can be performed using any techniques known in the art including, but not limited to, sequencing, PCR, RT-PCR, quantitative PCR, hybridization techniques, northern blot analysis, microarray technology, DNA microarray technology, etc. In determining the expression level of a biomarker gene or genes in a genetic sample, the level of expression can be normalized by comparison to the expression of another gene such as a well-known, well-characterized gene or a housekeeping gene.

In particular embodiments, expression of a marker gene of interest is determined using microarray technology. Generally, an array is a solid support with peptide or nucleic acid probes attached to the support. Arrays typically include a plurality of different nucleic acid or peptide probes that are coupled to a surface of a substrate in different, known locations. These arrays, also described as microarrays or colloquially “chips”, have been generally described in the art, (e.g., U.S. Pat. Nos. 5,143,854, 5,445,934, 5,744,305, 5,677,195, 6,040,193, 5,424,186 and Fodor, et al. (1991) Science 251:767-777). Arrays can be peptides or nucleic acids on beads, gels, polymeric surfaces, fibers such as fiber optics, glass or any other appropriate substrate, see U.S. Pat. Nos. 5,770,358, 5,789,162, 5,708,153, 6,040,193 and 5,800,992. Arrays can be packaged in such a manner as to allow for diagnostics or other manipulation of sample in an all inclusive device, see for example, U.S. Pat. Nos. 5,856,174 and 5,922,591. The use and analysis of arrays is routinely practiced in the art and any conventional scanner and software can be employed.

The expression data from a particular marker gene or group of marker genes can be analyzed using statistical methods, such as those described herein, to classify or determine the clinical endpoints of scleroderma patients. In this analysis, the expression of one or more marker genes in the patient skin sample is compared to the expression of the one or more marker genes in a control or reference sample. A control or reference sample can be a sample taken from the same patient, such as clinically uninvolved tissue or normal tissue, a sample from a MMF treatment responder, a sample from a MMF treatment nonresponder or a sample from a healthy individual. In addition, a control sample can be expression, such as the average expression of a gene of interest from a cohort of healthy individuals. The control or reference can be a predetermined value, such as values obtained for each gene to be assessed (e.g., each intrinsic gene included in Group III, Group II or Group I or presented elsewhere herein) from a population of patients with SSc or scleroderma who are MMF treatment responders or non-responders.

In one embodiment, a control or reference sample includes a composite of data derived from a plurality of nucleic acid microarray hybridizations representative of the subtype. In this case, the control can serve as a baseline (pretreatment) indicator, useful to identify a patient as a MMF treatment responder or a MMF treatment nonresponder and also as values toward which a non-responder patient must move (perhaps through treatment with other medications) in order to become a MMF treatment responder.

In one embodiment, a control or reference sample includes a composite of data derived from a plurality of nucleic acid microarray hybridizations used to assess gene expression signatures of patients known to respond to MMF treatment, such as microarray hybridizations analyzing MMF treatment responders described herein.

Based on data and principles set forth herein, a subject having or suspected of having scleroderma can be identified as MMF treatment responsive (or likely to respond to MMF treatment) or MMF treatment non-responsive. That a patient or subject is an MMF treatment responder can be confirmed using additional known methods. For example, the patient can be treated with MMF and the effect on mRSS or other indicators of organ involvement assessed. For example, an improvement in mRSS is an indicator of positive response to MMF treatment. In one embodiment, sample classification is performed by Pearson correlations to the average centroid of the genes shown to be up- or down-regulated in each group. Both up- and down-regulated genes can be important. As explained herein, this profile can be measured in skin biopsies of patients with scleroderma using either a gene expression microarray or, especially for small subsets of genes, by a method such as quantitative PCR.

### Example 1

### Identification of Gene Expression Signatures in SSc Patients Who are MMF Treatment Responders or Non-Responders

A prospective, open-label, observational study was conducted in a clinically well-characterized cohort of SSc patients who were receiving MMF treatment for progressive skin disease in the opinion of the treating physician. Detailed clinical assessments and skin biopsies were performed before the initiation of therapy and at 6 and 12 months during MMF treatment. The purpose of the study was to identify a gene expression signature in skin in MMF treatment responders (responders) and those who do not respond to MMF treatment (non-responders) at baseline (before MMF treatment) and to analyze the differences in the changes in gene expression that occurred in skin during treatment in responders and non-responders. The hypothesis was that the pattern of gene expression in skin at baseline would differ between MMF responders and non-responders, and that there would be alterations in gene expression during MMF treatment in responders, but not in non-responders. The differences between responders and nonresponders can be used to predict or aid in predicting whether an SSc patient will respond to MMF treatment and to assess the effectiveness of MMF treatment.

Patients seen at the Northwestern Scleroderma Program who met American College of Rheumatology criteria for SSc with a mRSS≧16 and would be receiving mycophenolate mofetil for the indication of progressive skin tightening were assessed. All patients provided written informed consent in accordance with the Institutional Review Board Guidelines of Northwestern University. Patients underwent a standardized physical exam, including mRSS performed by one clinician at study entry (prior to treatment) and at 6 months and 12 months post MMF initiation. A pair of skin biopsies was removed from clinically involved skin (forearm) and clinically uninvolved skin (back) from the non-dominant side of the body. Two biopsies (one from forearm and back) were placed in RNAlater® (Applied Biosystems, Ambion®, Carlsbad, Calif., U.S.A.) and used for DNA microarray analysis and the other biopsy was placed in 10% neutral buffered formalin and used for histological analyses.

Response to MMF was defined as an improvement in mRSS which is the minimal clinically important difference [Amjadi, S., et al., Arthritis Rheum, 2009. 60(8): p. 2490-8] or a ≧20% improvement in skin score from baseline (which ever was greater) that published studies suggest is a treatment response rather than the natural history of SSc [Khanna, D., et al., Arthritis Rheum, 2007. 56(5): p. 1676-84].

To examine genome-wide changes in gene expression induced by MMF on a cellular level, 200 ng total RNA was amplified and labeled using the Agilent Technologies (Santa Clara, Calif., U.S.A.) low input fluorescent linear amplification for hybridization as described previously [Milano, A., et al., PLoS One, 2008. 3(7): p. e2696]. Briefly, subject RNA was labeled with Cy3 dye, and Universal Human Reference RNA (UHR) (Stratagene) (Agilent Technologies) was labeled with fluorescent Cy5 dye. After amplification and labeling, the cRNA was purified and co-hybridized with UHR RNA to whole human genome microarrays, representing 41,000 unique probes. The hybridization was carried out for 17 hours at 65° C. A total of 89 arrays were hybridized, which included technical replicates.

Arrays were washed and scanned and Gene Pix Pro 5.0 software. Data were Log 2 LOWESS normalized for the Cy5/Cy3 ratio, and data was filtered to select array spots with an intensity 2 fold or greater than the local background in either the Cy3 or Cy5 channel. Any probes missing more than 20% of the data across all arrays were omitted from further analysis.

Intrinsic gene analysis was conducted as previously described to identify the genes that are most similar between forearm and back samples for a given individual, but most different between individuals [Milano, A., et al., PLoS One, 2008. 3(7): p. e2696]. For the gene expression of each probe, the algorithm calculated the ratio of the variability within each group of arrays to the variability between each group of arrays. The weighted average of the variance of each group of arrays formed the value of the numerator (A). The weighted average of the gene expression for arrays in each group was calculated and the variance of the average gene expression across all groups of arrays formed the denominator (B). The within group variance divided by the between group variance resulted in an “intrinsic score” for each probe. To determine an FDR value, the gene expression data was permuted multiple times, and the intrinsic gene algorithm was used with each of the permuted gene expression datasets. The average number of probes that were less than a given intrinsic score cutoff in the permuted data was divided by the number of probes that obtained the intrinsic score cutoff in the non-permuted data. This value was multiplied by 100 to provide a percentage FDR rate.

To determine if the baseline gene expression in skin was different between MMF responders and non-responders, and whether MMF responders cluster in the same molecularly defined subset that have been previously described (Diffuse 1, Diffuse 2, Inflammatory, Normal-like and Limited) [Milano, A., et al., PLoS One, 2008. 3(7): p. e2696], a gene expression analysis was conducted. This analysis identified genes having expression patterns most similar between arm and back biopsies for each subject having the greatest variance among all samples, which has been termed ‘intrinsic gene analysis’ [Milano, A., et al., PLoS One, 2008. 3(7): p. e2696]. The hypothesis was that MMF responders would be members of the inflammatory molecularly defined subset because MMF inhibits lymphocyte proliferation [Milano, A., et al., PLoS One, 2008. 3(7): p. e2696]. Using data obtained from the gene expression study, an unsupervised hierarchical clustering analysis was performed to characterize inherent data-driven groupings (FIG. 1). Included in the experiment were previously analyzed samples from SSc controls (SSc registry), with known molecularly defined signatures. Also included were biopsies from normal healthy controls (Cntrl). The biopsies from the SSc registry and healthy controls were employed to enable identification of the molecularly defined clusters. Findings revealed that expression signatures from pre-treatment (arm and back) biopsies amongst MMF responders (R) and non-responders (NR) were distinctly separate. Further, MMF treatment responders clustered within the inflammatory molecularly defined subset apart from the MMF-non-responders [Milano, A., et al., PLoS One, 2008. 3(7): p. e2696] (FIG. 1).

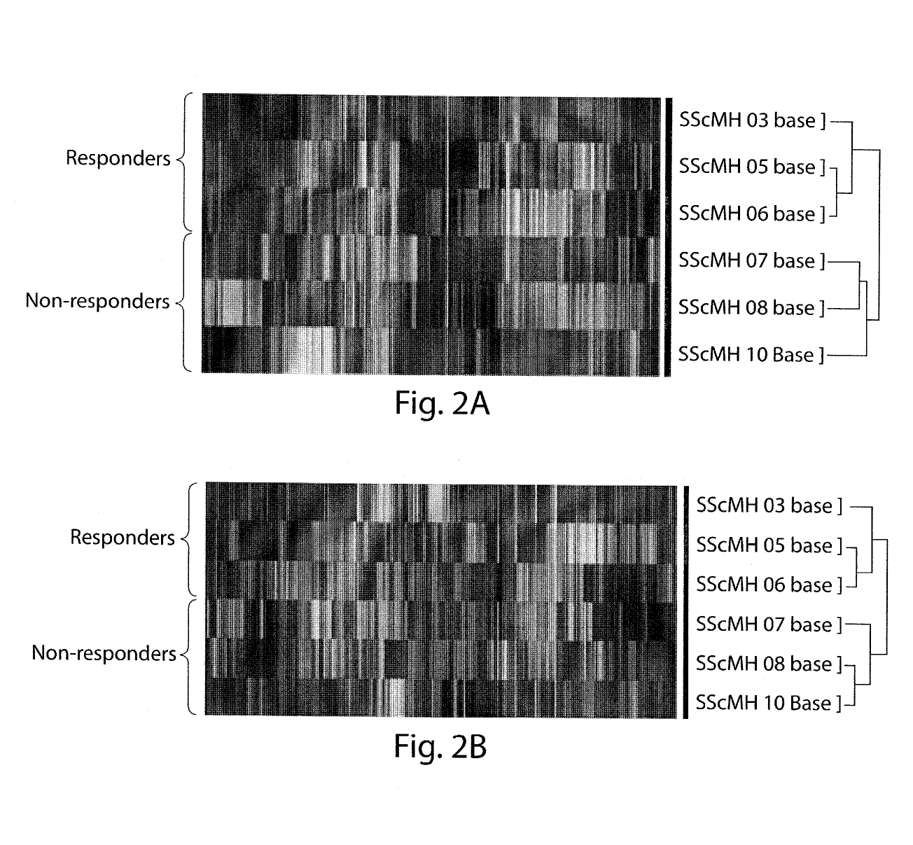

To identify an MMF responsive gene expression signature in skin at baseline, MMF-responders' baseline gene expression patterns was compared to 6 or 12 month post-MMF treatment levels. Genes having expression that changed concordantly between pre- and post-treatment in arm and back samples were analyzed further. In arm or back samples, 572 genes that had a p<0.002 (FDR 10%) were identified (Table 1). See also Table 1A. There were dramatic differences in gene expression in arm samples between responders and non-responders at baseline as demonstrated by SAM and cluster analysis (FIG. 2A). The difference in back samples was not as clear, suggesting that skin biopsies from the forearm may be more useful in predicting response to MMF in patients with SSc (FIG. 2B).

To confirm the physical exam finding of softened skin in MMF-responders, the morphology of skin biopsy specimens was examined using routine H&E staining of paraffin embedded sections. There are clear differences in the extent of fibrosis between pre- and post-treatment biopsies in responders. The MMF-nonresponder demonstrated a greater degree in skin fibrosis and a further loss of the normal dermal appendages.

TABLE 1

| Entrez ID | Gene Symbol | Gene Name | Accession |
|---|---|---|---|
| 21 | ABCA3 | ATP-binding cassette, sub-family A | NM_001089 |
| | | (ABC1), member 3 | |
| 25 | ABL1 | c-abl oncogene 1, non-receptor | NM_005157 |
| | | tyrosine kinase | |
| 47 | ACLY | ATP citrate lyase | NM_001096 |
| 115 | ADCY9 | adenylate cyclase 9 | NM_001116 |
| 118 | ADD1 | adducin 1 (alpha) | NM_001119 |
| 132 | ADK | adenosine kinase | NM_001123 |
| 164 | AP1G1 | adaptor-related protein complex 1, | NM_001030007 |
| | | gamma 1 subunit | |
| 185 | AGTR1 | angiotensin II receptor, type 1 | NM_000685 |
| 351 | APP | amyloid beta (A4) precursor protein | NM_000484 |
| 367 | AR | androgen receptor | NM_000044 |
| 375 | ARF1 | ADP-ribosylation factor 1 | NM_001024226 |
| 488 | ATP2A2 | ATPase, Ca++ transporting, cardiac | NM_001135765 |
| | | muscle, slow twitch 2 | |
| 534 | ATP6V1G2 | ATPase, H+ transporting, lysosomal | NM_001204078 |
| | | 13 kDa, V1 subunit G2 | |
| 710 | SERPING1 | serpin peptidase inhibitor, clade G (C1 | NM_000062 |
| | | inhibitor), member 1 | |
| 975 | CD81 | CD81 molecule | NM_004356 |
| 983 | CDK1 | cyclin-dependent kinase 1 | NM_001170406 |
| 1022 | CDK7 | cyclin-dependent kinase 7 | NM_001799 |
| 1033 | CDKN3 | cyclin-dependent kinase inhibitor 3 | NM_001130851 |
| 1039 | CDR2 | cerebellar degeneration-related protein | NM_001802 |
| | | 2, 62 kDa | |
| 1063 | CENPF | centromere protein F, 350/400 kDa | NM_016343 |
| | | (mitosin) | |
| 1277 | COL1A1 | collagen, type I, alpha 1 | NM_000088 |
| 1305 | COL13A1 | collagen, type XIII, alpha 1 collagen, | NM_001130103 |
| | | type XIII, alpha 1 | |
| 1349 | COX7B | cytochrome c oxidase subunit VIIb | NM_001866 |
| 1387 | CREBBP | CREB binding protein | NM_001079846 |
| 1452 | CSNK1A1 | casein kinase 1, alpha 1 casein kinase | NM_001025105 |
| | | 1, alpha 1 | |
| 1593 | CYP27A1 | cytochrome P450, family 27, | NM_000784 |
| | | subfamily A, polypeptide 1 | |
| 1627 | DBN1 | drebrin 1 | NM_004395 |
| 1727 | CYB5R3 | cytochrome b5 reductase 3 | NM_000398 |
| 1876 | E2F6 | E2F transcription factor 6 | NM_198256 |
| 1933 | EEF1B2 | eukaryotic translation elongation factor | NM_001037663 |
| | | 1 beta 2 | |
| 1964 | EIF1AX | eukaryotic translation initiation factor | NM_001412 |
| | | 1A, X-linked | |
| 2026 | ENO2 | enolase 2 (gamma, neuronal) | NM_001975 |
| 2030 | SLC29A1 | solute carrier family 29 (nucleoside | NM_001078174 |
| | | transporters), member 1 | |
| 2057 | EPOR | erythropoietin receptor | NM_000121 |
| 2108 | ETFA | electron-transfer-flavoprotein, alpha | NM_000126 |
| | | polypeptide | |
| 2140 | EYA3 | Eyes absent homolog 3 (Drosophila) | NM_001990 |
| 2149 | F2R | coagulation factor II (thrombin) | NM_001992 |
| | | receptor | |
| 2155 | F7 | coagulation factor VII (serum | NM_000131 |
| | | prothrombin conversion accelerator) | |
| 2159 | F10 | coagulation factor X | NM_000504 |
| 2186 | BPTF | bromodomain PHD finger transcription | NM_004459 |
| | | factor | |
| 2192 | FBLN1 | fibulin 1 | NM_001996 |
| 2199 | FBLN2 | fibulin 2 | NM_001004019 |
| 2350 | FOLR2 | folate receptor 2 (fetal) | NM_000803 |
| 2662 | GDF10 | growth differentiation factor 10 | NM_004962 |
| 2720 | GLB1 | galactosidase, beta 1 | NM_000404 |
| 2743 | GLRB | glycine receptor, beta | NM_000824 |
| 2781 | GNAZ | guanine nucleotide binding protein (G | NM_002073 |
| | | protein), alpha z polypeptide | |
| 2821 | GPI | glucose-6-phosphate isomerase | NM_000175 |
| 2950 | GSTP1 | glutathione S-transferase pi 1 | NM_000852 |
| 2963 | GTF2F2 | general transcription factor IIF, | NM_004128 |
| | | polypeptide 2, 30 kDa | |
| 2969 | GTF2I | general transcription factor IIi | NM_001163636 |
| 2990 | GUSB | glucuronidase, beta | NM_000181 |
| 3015 | H2AFZ | H2A histone family, member Z H2A | NM_002106 |
| | | histone family, member Z H2A histone | |
| | | family, member Z | |
| 3069 | HDLBP | High density lipoprotein binding | NM_005336 |
| | | protein | |
| 3094 | HINT1 | histidine triad nucleotide binding | NM_005340 |
| | | protein 1 histidine triad nucleotide | |
| | | binding protein 1 | |
| 3148 | HMGB2 | High-mobility group box 2 | NM_001130688 |
| 3399 | ID3 | inhibitor of DNA binding 3, dominant | NM_002167 |
| | | negative helix-loop-helix protein | |
| 3454 | IFNAR1 | interferon (alpha, beta and omega) | NM_000629 |
| | | receptor 1 | |
| 3482 | IGF2R | insulin-like growth factor 2 receptor | NM_000876 |
| 3667 | IRS1 | insulin receptor substrate 1 | NM_005544 |
| 3671 | ISLR | immunoglobulin superfamily | NM_005545 |
| | | containing leucine-rich repeat | |
| 3680 | ITGA9 | integrin, alpha 9 | NM_002207 |
| 3693 | ITGB5 | integrin, beta 5 | NM_002213 |
| 3913 | LAMB2 | laminin, beta 2 (laminin S) | NM_002292 |
| 3916 | LAMP1 | lysosomal-associated membrane | NM_005561 |
| | | protein 1 | |
| 3931 | LCAT | lecithin-cholesterol acyltransferase | NM_000229 |
| 3984 | LIMK1 | LIM domain kinase 1 | NM_002314 |
| 4043 | LRPAP1 | low density lipoprotein receptor-related | NM_002337 |
| | | protein associated protein 1 | |
| 4054 | LTBP3 | latent transforming growth factor beta | NM_001130144 |
| | | binding protein 3 | |
| 4066 | LYL1 | lymphoblastic leukemia derived | NM_005583 |
| | | sequence 1 | |
| 4085 | MAD2L1 | MAD2 mitotic arrest deficient-like 1 | NM_002358 |
| | | (yeast) | |
| 4111 | MAGEA12 | melanoma antigen family A, 12 | NM_001166386 |
| 4116 | MAGOH | mago-nashi homolog, proliferation- | NM_002370 |
| | | associated (Drosophila) | |
| 4194 | MDM4 | Mdm4 p53 binding protein homolog | NM_002393 |
| | | (mouse) | |
| 4245 | MGAT1 | mannosyl (alpha-1,3-)-glycoprotein | NM_001114617 |
| | | beta-1,2-N- | |
| | | acetylglucosaminyltransferase | |
| 4324 | MMP15 | matrix metallopeptidase 15 | NM_002428 |
| | | (membrane-inserted) | |
| 4515 | MTCP1 | mature T-cell proliferation 1 | NM_001018025 |
| 4528 | MTIF2 | mitochondrial translational initiation | NM_001005369 |
| | | factor 2 | |
| 4628 | MYH10 | myosin, heavy chain 10, non-muscle | NM_005964 |
| 4692 | NDN | necdin homolog (mouse) | NM_002487 |
| 4712 | NDUFB6 | NADH dehydrogenase (ubiquinone) 1 | NM_001199987 |
| | | beta subcomplex, 6, 17 kDa | |
| 4729 | NDUFV2 | NADH dehydrogenase (ubiquinone) | NM_021074 |
| | | flavoprotein 2, 24 kDa | |
| 4733 | DRG1 | developmentally regulated GTP | NM_004147 |
| | | binding protein 1 | |
| 4758 | NEU1 | sialidase 1 (lysosomal sialidase) | NM_000434 |
| 4771 | NF2 | neurofibromin 2 (merlin) | NM_000268 |
| 4779 | NFE2L1 | nuclear factor (erythroid-derived 2)- | NM_003204 |
| | | like 1 | |
| 4836 | NMT1 | N-myristoyltransferase 1 | NM_021079 |
| 4853 | NOTCH2 | notch 2 | NM_001200001 |
| 4856 | NOV | nephroblastoma overexpressed gene | NM_002514 |
| | | nephroblastoma overexpressed gene | |
| 4869 | NPM1 | nucleophosmin (nucleolar | NM_001037738 |
| | | phosphoprotein B23, numatrin) | |
| 4947 | OAZ2 | ornithine decarboxylase antizyme 2 | NM_002537 |
| 4952 | OCRL | oculocerebrorenal syndrome of Lowe | NM_000276 |
| 4976 | OPA1 | optic atrophy 1 (autosomal dominant) | NM_015560 |
| 5104 | SERPINA5 | serpin peptidase inhibitor, clade A | NM_000624 |
| | | (alpha-1 antiproteinase, antitrypsin), | |
| | | member 5 | |
| 5111 | PCNA | proliferating cell nuclear antigen | NM_002592 |
| 5159 | PDGFRB | platelet-derived growth factor receptor, | NM_002609 |
| | | beta polypeptide | |
| 5163 | PDK1 | pyruvate dehydrogenase kinase, | NM_002610 |
| | | isozyme 1 | |
| 5253 | PHF2 | PHD finger protein 2 | NM_005392 |
| 5351 | PLOD1 | procollagen-lysine 1, 2-oxoglutarate 5- | NM_000302 |
| | | dioxygenase 1 | |
| 5393 | EXOSC9 | exosome component 9 | NM_001034194 |
| 5420 | PODXL | podocalyxin-like | NM_001018111 |
| 5514 | PPP1R10 | protein phosphatase 1, regulatory | NM_002714 |
| | | (inhibitor) subunit 10 | |
| 5557 | PRIM1 | primase, DNA, polypeptide 1 (49 kDa) | NM_000946 |
| 5565 | PRKAB2 | protein kinase, AMP-activated, beta 2 | NM_005399 |
| | | non-catalytic subunit | |
| 5648 | MASP1 | mannan-binding lectin serine peptidase | NM_001031849 |
| | | 1 (C4/C2 activating component of Ra- | |
| | | reactive factor) | |
| 5664 | PSEN2 | presenilin 2 (Alzheimer disease 4) | NM_000447 |
| 5681 | PSKH1 | protein serine kinase H1 | NM_006742 |
| 5684 | PSMA3 | proteasome (prosome, macropain) | NM_002788 |
| | | subunit, alpha type, 3 | |
| 5757 | PTMA | prothymosin, alpha prothymosin, alpha | NM_001099285 |
| 5771 | PTPN2 | protein tyrosine phosphatase, non- | NM_002828 |
| | | receptor type 2 | |
| 5828 | PEX2 | peroxisomal biogenesis factor 2 | NM_000318 |
| 5868 | RAB5A | RAB5A, member RAS oncogene | NM_004162 |
| | | family | |
| 5884 | RAD17 | RAD17 homolog (S. pombe) | NM_002873 |
| 5914 | RARA | retinoic acid receptor, alpha | NM_000964 |
| 5976 | UPF1 | UPF1 regulator of nonsense transcripts | NM_002911 |
| | | homolog (yeast) | |
| 6046 | BRD2 | bromodomain containing 2 | NM_001113182 |
| 6050 | RNH1 | ribonuclease/angiogenin inhibitor 1 | NM_002939 |
| 6144 | RPL21 | ribosomal protein L21 | NM_000982 |
| 6280 | S100A9 | S100 calcium binding protein A9 | NM_002965 |
| 6297 | SALL2 | sal-like 2 (Drosophila) | NM_005407 |
| 6330 | SCN4B | sodium channel, voltage-gated, type | NM_001142348 |
| | | IV, beta | |
| 6347 | CCL2 | chemokine (C-C motif) ligand 2 | NM_002982 |
| 6387 | CXCL12 | chemokine (C-X-C motif) ligand 12 | NM_000609 |
| 6389 | SDHA | succinate dehydrogenase complex, | NM_004168 |
| | | subunit A, flavoprotein (Fp) | |
| 6430 | SRSF5 | serine/arginine-rich splicing factor 5 | NM_001039465 |
| 6433 | SFSWAP | splicing factor, suppressor of white- | NM_004592 |
| | | apricot homolog (Drosophila) | |
| 6483 | ST3GAL2 | ST3 beta-galactoside alpha-2,3- | NM_006927 |
| | | sialyltransferase 2 | |
| 6586 | SLIT3 | slit homolog 3 (Drosophila) | NM_003062 |
| 6617 | SNAPC1 | small nuclear RNA activating complex, | NM_003082 |
| | | polypeptide 1, 43 kDa | |
| 6632 | SNRPD1 | small nuclear ribonucleoprotein D1 | NM_006938 |
| | | polypeptide 16 kDa | |
| 6636 | SNRPF | small nuclear ribonucleoprotein | NM_003095 |
| | | polypeptide F | |
| 6648 | SOD2 | superoxide dismutase 2, mitochondrial | NM_000636 |
| 6659 | SOX4 | SRY (sex determining region Y)-box 4 | NM_003107 |
| 6728 | SRP19 | signal recognition particle 19 kDa | NM_003135 |
| 6734 | SRPR | signal recognition particle receptor | NM_001177842 |
| | | (docking protein) | |
| 6744 | SSFA2 | sperm specific antigen 2 | NM_001130445 |
| 6815 | STYX | serine/threonine/tyrosine interacting | NM_001130701 |
| | | protein | |
| 6821 | SUOX | sulfite oxidase | NM_000456 |
| 6836 | SURF4 | surfeit 4 | NM_033161 |
| 6885 | MAP3K7 | mitogen-activated protein kinase | NM_003188 |
| | | kinase kinase 7 | |
| 6886 | TAL1 | T-cell acute lymphocytic leukemia 1 | NM_003189 |
| 6917 | TCEA1 | transcription elongation factor [symbol] (SII), | NM_006756 |
| | | 1 | |
| 6929 | TCF3 | transcription factor 3 (E2A | NM_001136139 |
| | | immunoglobulin enhancer binding | |
| | | factors E12/E47) | |
| 6942 | TCF20 | transcription factor 20 (AR1) | NM_005650 |
| 7024 | TFCP2 | transcription factor CP2 | NM_001173452 |
| 7031 | TFF1 | trefoil factor 1 | NM_003225 |
| 7067 | THRA | thyroid hormone receptor, alpha | NM_001190918 |
| | | (erythroblastic leukemia viral (v-erb-a) | |
| | | oncogene homolog, avian) | |
| 7148 | TNXB | tenascin XB | NM_019105 |
| 7153 | TOP2A | topoisomerase (DNA) II alpha 170 kDa | NM_001067 |
| 7187 | TRAF3 | TNF receptor-associated factor 3 | NM_001199427 |
| 7204 | TRIO | triple functional domain (PTPRF | NM_007118 |
| | | interacting) | |
| 7272 | TTK | TTK protein kinase | NM_001166691 |
| 7327 | UBE2G2 | ubiquitin-conjugating enzyme E2G 2 | NM_001202489 |
| | | (UBC7 homolog, yeast) | |
| 7357 | UGCG | UDP-glucose ceramide | NM_003358 |
| | | glucosyltransferase | |
| 7373 | COL14A1 | collagen, type XIV, alpha 1 | NM_021110 |
| 7596 | ZNF45 | zinc finger protein 45 | NM_003425 |
| 7625 | ZNF74 | zinc finger protein 74 | NM_003426 |
| 7633 | ZNF79 | zinc finger protein 79 | NM_007135 |
| 7743 | ZNF189 | zinc finger protein 189 | NM_003452 |
| 7753 | ZNF202 | zinc finger protein 202 | NM_003455 |
| 7775 | ZNF232 | zinc finger protein 232 | NM_014519 |
| 7798 | LUZP1 | leucine zipper protein 1 | NM_001142546 |
| 7841 | MOGS | mannosyl-oligosaccharide glucosidase | NM_001146158 |
| | | mannosyl-oligosaccharide glucosidase | |
| 7869 | SEMA3B | sema domain, immunoglobulin domain | NM_001005914 |
| | | (Ig), short basic domain, secreted, | |
| | | (semaphorin) 3B sema domain, | |
| | | immunoglobulin domain (Ig), short | |
| | | basic domain, secreted, (semaphorin) | |
| | | 3B | |
| 8085 | MLL2 | myeloid/lymphoid or mixed-lineage | NM_003482 |
| | | leukemia 2 | |
| 8106 | PABPN1 | Poly(A) binding protein, nuclear 1 | NM_004643 |
| 8325 | FZD8 | frizzled homolog 8 (Drosophila) | NM_031866 |
| 8350 | HIST1H3A | histone cluster 1, H3a | NM_003529 |
| 8353 | HIST1H3E | histone cluster 1, H3e | NM_003532 |
| 8357 | HIST1H3H | histone cluster 1, H3h | NM_003536 |
| 8409 | UXT | ubiquitously-expressed transcript | NM_004182 |
| 8425 | LTBP4 | latent transforming growth factor beta | NM_001042544 |
| | | binding protein 4 | |
| 8554 | PIAS1 | protein inhibitor of activated STAT, 1 | NM_016166 |
| 8559 | PRPF18 | PRP18 pre-mRNA processing factor | NM_003675 |
| | | 18 homolog (S. cerevisiae) | |
| 8574 | AKR7A2 | aldo-keto reductase family 7, member | NM_003689 |
| | | A2 (aflatoxin aldehyde reductase) | |
| 8663 | EIF3C | eukaryotic translation initiation factor | NM_001037808 |
| | | 3, subunit C | |
| 8664 | EIF3D | eukaryotic translation initiation factor | NM_003753 |
| | | 3, subunit D | |
| 8678 | BECN1 | beclin 1, autophagy related | NM_003766 |
| 8722 | CTSF | cathepsin F | NM_003793 |
| 8760 | CDS2 | CDP-diacylglycerol synthase | NM_003818 |
| | | (phosphatidate cytidylyltransferase) 2 | |
| 8767 | RIPK2 | receptor-interacting serine-threonine | NM_003821 |
| | | kinase 2 | |
| 8780 | RIOK3 | RIO kinase 3 (yeast) | NM_003831 |
| 8848 | TSC22D1 | TSC22 domain family, member 1 | NM_006022 |
| 8974 | P4HA2 | prolyl 4-hydroxylase, alpha | NM_001017973 |
| | | polypeptide II | |
| 8991 | SELENBP1 | selenium binding protein 1 | NM_003944 |
| 9131 | AIFM1 | apoptosis-inducing factor, | NM_001130846 |
| | | mitochondrion-associated, 1 | |
| 9139 | CBFA2T2 | core-binding factor, runt domain, alpha | NM_001032999 |
| | | subunit 2; translocated to, 2 | |
| 9141 | PDCD5 | programmed cell death 5 | NM_004708 |
| 9188 | DDX21 | DEAD (Asp-Glu-Ala-Asp) box | NM_004728 |
| | | polypeptide 21 | |
| 9262 | STK17B | serine/threonine kinase 17b | NM_004226 |
| 9320 | TRIP12 | thyroid hormone receptor interactor 12 | NM_004238 |
| 9326 | ZNHIT3 | zinc finger, HIT-type containing 3 | NM_004773 |
| 9440 | MED17 | mediator complex subunit 17 | NM_004268 |
| 9476 | NAPSA | napsin A aspartic peptidase | NM_004851 |
| 9493 | KIF23 | kinesin family member 23 | NM_004856 |
| 9541 | CIR1 | corepressor interacting with RBPJ, 1 | NM_004882 |
| 9551 | ATP5J2 | ATP synthase, H+ transporting, | NM_001003713 |
| | | mitochondrial Fo complex, subunit F2 | |
| 9570 | GOSR2 | golgi SNAP receptor complex member 2 | NM_001012511 |
| 9590 | AKAP12 | A kinase (PRKA) anchor protein 12 | NM_005100 |
| 9701 | PPP6R2 | protein phosphatase 6, regulatory | NM_014678 |
| | | subunit 2 | |
| 9751 | SNPH | syntaphilin | NM_014723 |
| 9768 | KIAA0101 | KIAA0101 | NM_001029989 |
| 9794 | MAML1 | mastermind-like 1 (Drosophila) | NM_014757 |
| 9798 | KIAA0174 | KIAA0174 | NM_014761 |
| 9805 | SCRN1 | secernin 1 | NM_001145513 |
| 9815 | GIT2 | G protein-coupled receptor kinase | NM_001135213 |
| | | interacting ArfGAP 2 | |
| 9843 | HEPH | hephaestin | NM_001130860 |
| 9895 | TECPR2 | tectonin beta-propeller repeat | NM_001172631 |
| | | containing 2 | |
| 9915 | ARNT2 | aryl-hydrocarbon receptor nuclear | NM_014862 |
| | | translocator 2 | |
| 9962 | SLC23A2 | solute carrier family 23 (nucleobase | NM_005116 |
| | | transporters), member 2 | |
| 9978 | RBX1 | ring-box 1, E3 ubiquitin protein ligase | NM_014248 |
| 9991 | ROD1 | ROD1 regulator of differentiation 1 (S. | NM_001163788 |
| | | pombe) ROD1 regulator of | |
| | | differentiation 1 (S. pombe) | |
| 10018 | BCL2L11 | BCL2-like 11 (apoptosis facilitator) | NM_001204106 |
| 10040 | TOM1L1 | target of myb1 (chicken)-like 1 | NM_005486 |
| 10049 | DNAJB6 | DnaJ (Hsp40) homolog, subfamily B, | NM_005494 |
| | | member 6 DnaJ (Hsp40) homolog, | |
| | | subfamily B, member 6 DnaJ (Hsp40) | |
| | | homolog, subfamily B, member 6 | |
| 10106 | CTDSP2 | CTD (carboxy-terminal domain, RNA | NM_005730 |
| | | polymerase II, polypeptide A) small | |
| | | phosphatase 2 | |
| 10127 | ZNF263 | zinc finger protein 263 | NM_005741 |
| 10135 | NAMPT | nicotinamide | NM_005746 |
| | | phosphoribosyltransferase | |
| | | nicotinamide | |
| | | phosphoribosyltransferase | |
| | | nicotinamide | |
| | | phosphoribosyltransferase | |
| 10161 | LPAR6 | lysophosphatidic acid receptor 6 | NM_001162497 |
| 10168 | ZNF197 | zinc finger protein 197 | NM_001024855 |
| 10200 | MPHOSPH6 | M-phase phosphoprotein 6 | NM_005792 |
| 10209 | EIF1 | eukaryotic translation initiation factor | NM_005801 |
| | | 1 eukaryotic translation initiation | |
| | | factor 1 | |
| 10231 | RCAN2 | regulator of calcineurin 2 | NM_005822 |
| 10391 | CORO2B | coronin, actin binding protein, 2B | NM_001190456 |
| 10403 | NDC80 | NDC80 homolog, kinetochore complex | NM_006101 |
| | | component (S. cerevisiae) | |
| 10417 | SPON2 | spondin 2, extracellular matrix protein | NM_001128325 |
| 10421 | CD2BP2 | CD2 (cytoplasmic tail) binding protein | NM_006110 |
| | | 2 | |
| 10438 | C1D | C1D nuclear receptor corepressor C1D | NM_001190263 |
| | | nuclear receptor corepressor | |
| 10481 | HOXB13 | homeobox B13 | NM_006361 |
| 10493 | VAT1 | vesicle amine transport protein 1 | NM_006373 |
| | | homolog (T. californica) | |
| 10541 | ANP32B | acidic (leucine-rich) nuclear | NM_006401 |
| | | phosphoprotein 32 family, member B | |
| 10606 | PAICS | phosphoribosylaminoimidazole | NM_001079524 |
| | | carboxylase, | |
| | | phosphoribosylaminoimidazole | |
| | | succinocarboxamide synthetase | |
| 10609 | LEPREL4 | leprecan-like 4 | NM_006455 |
| 10614 | HEXIM1 | hexamethylene bis-acetamide inducible | NM_006460 |
| | | 1 | |
| 10618 | TGOLN2 | Trans-golgi network protein 2 | NM_006464 |
| 10670 | RRAGA | Ras-related GTP binding A | NM_006570 |
| 10671 | DCTN6 | dynactin 6 | NM_006571 |
| 10742 | RAI2 | retinoic acid induced 2 | NM_001172732 |
| 10746 | MAP3K2 | mitogen-activated protein kinase | NM_006609 |
| | | kinase kinase 2 | |
| 10766 | TOB2 | transducer of ERBB2, 2 | NM_016272 |
| 10775 | POP4 | processing of precursor 4, ribonuclease | NM_006627 |
| | | P/MRP subunit (S. cerevisiae) | |
| 10910 | SUGT1 | SGT1, suppressor of G2 allele of SKP1 | NM_001130912 |
| | | (S. cerevisiae) | |
| 10916 | MAGED2 | melanoma antigen family D, 2 | NM_014599 |
| 10919 | EHMT2 | euchromatic histone-lysine N- | NM_006709 |
| | | methyltransferase 2 euchromatic | |
| | | histone-lysine N-methyltransferase 2 | |
| 10923 | SUB1 | SUB1 homolog (S. cerevisiae) | NM_006713 |
| 10940 | POP1 | processing of precursor 1, ribonuclease | NM_001145860 |
| | | P/MRP subunit (S. cerevisiae) | |
| 11011 | TLK2 | tousled-like kinase 2 | NM_001112707 |
| 11162 | NUDT6 | nudix (nucleoside diphosphate linked | NM_007083 |
| | | moiety X)-type motif 6 | |
| 11177 | BAZ1A | bromodomain adjacent to zinc finger | NM_013448 |
| | | domain, 1A | |
| 11193 | WBP4 | WW domain binding protein 4 (formin | NM_007187 |
| | | binding protein 21) | |
| 11328 | FKBP9 | FK506 binding protein 9, 63 kDa | NM_007270 |
| 22822 | PHLDA1 | pleckstrin homology-like domain, | NM_007350 |
| | | family A, member 1 | |
| 22880 | MORC2 | MORC family CW-type zinc finger 2 | NM_014941 |
| | | MORC family CW-type zinc finger 2 | |
| 22882 | ZHX2 | zinc fingers and homeoboxes 2 | NM_014943 |
| 22927 | HABP4 | hyaluronan binding protein 4 | NM_014282 |
| 22931 | RAB18 | RAB18, member RAS oncogene | NM_021252 |
| | | family | |
| 22950 | SLC4A1AP | solute carrier family 4 (anion | NM_018158 |
| | | exchanger), member 1, adaptor protein | |
| 22954 | TRIM32 | tripartite motif containing 32 tripartite | NM_001099679 |
| | | motif containing 32 | |
| 23002 | DAAM1 | dishevelled associated activator of | NM_014992 |
| | | morphogenesis 1 dishevelled | |
| | | associated activator of morphogenesis | |
| | | 1 | |
| 23077 | MYCBP2 | MYC binding protein 2 | NM_015057 |
| 23160 | WDR43 | WD repeat domain 43 | NM_015131 |
| 23176 | 8-Sep | septin 8 | NM_001098811 |
| 23192 | ATG4B | ATG4 autophagy related 4 homolog B | NM_013325 |
| | | (S. cerevisiae) | |
| 23216 | TBC1D1 | TBC1 (tre-2/USP6, BUB2, cdc16) | NM_015173 |
| | | domain family, member 1 | |
| 23229 | ARHGEF9 | Cdc42 guanine nucleotide exchange | NM_001173479 |
| | | factor (GEF) 9 | |
| 23245 | ASTN2 | astrotactin 2 | NM_001184734 |
| 23253 | ANKRD12 | ankyrin repeat domain 12 | NM_001083625 |
| 23417 | MLYCD | malonyl-CoA decarboxylase malonyl- | NM_012213 |
| | | CoA decarboxylase | |
| 23428 | SLC7A8 | solute carrier family 7 (amino acid | NM_012244 |
| | | transporter, L-type), member 8 | |
| 23462 | HEY1 | hairy/enhancer-of-split related with | NM_001040708 |
| | | YRPW motif 1 | |
| 23466 | CBX6 | chromobox homolog 6 | NM_014292 |
| 23468 | CBX5 | chromobox homolog 5 | NM_001127321 |
| 23500 | DAAM2 | dishevelled associated activator of | NM_001201427 |
| | | morphogenesis 2 | |
| 23598 | PATZ1 | POZ (BTB) and AT hook containing | NM_014323 |
| | | zinc finger 1 | |
| 23636 | NUP62 | nucleoporin 62 kDa | NM_001193357 |
| 25758 | C11orf41 | chromosome 11 open reading frame 41 | NM_012194 |
| 25849 | PARM1 | prostate androgen-regulated mucin-like | NM_015393 |
| | | protein 1 | |
| 25888 | ZNF473 | zinc finger protein 473 | NM_001006656 |
| 25903 | OLFML2B | olfactomedin-like 2B | NM_015441 |
| 25917 | THUMPD3 | THUMP domain containing 3 | NM_001114092 |
| 25960 | GPR124 | G protein-coupled receptor 124 | NM_032777 |
| 25977 | NECAP1 | NECAP endocytosis associated 1 | NM_015509 |
| | | NECAP endocytosis associated 1 | |
| 25978 | CHMP2B | chromatin modifying protein 2B | NM_014043 |
| 26137 | ZBTB20 | zinc finger and BTB domain | NM_001164342 |
| | | containing 20 | |
| 26275 | HIBCH | 3-hydroxyisobutyryl-CoA hydrolase | NM_014362 |
| 26289 | AK5 | adenylate kinase 5 | NM_012093 |
| 26508 | HEYL | hairy/enhancer-of-split related with | NM_014571 |
| | | YRPW motif-like | |
| 26608 | TBL2 | transducin (beta)-like 2 | NM_012453 |
| 27042 | C1orf107 | chromosome 1 open reading frame 107 | NM_014388 |
| 27109 | ATP5S | ATP synthase, H+ transporting, | NM_001003803 |
| | | mitochondrial Fo complex, subunit s | |
| | | (factor B) ATP synthase, H+ | |
| | | transporting, mitochondrial Fo | |
| | | complex, subunit s (factor B) | |
| 27122 | DKK3 | dickkopf homolog 3 (Xenopus laevis) | NM_001018057 |
| | | dickkopf homolog 3 (Xenopus laevis) | |
| | | dickkopf homolog 3 (Xenopus laevis) | |
| 27249 | MMADHC | methylmalonic aciduria (cobalamin | NM_015702 |
| | | deficiency) cblD type, with | |
| | | homocystinuria | |
| 27258 | LSM3 | LSM3 homolog, U6 small nuclear | NM_014463 |
| | | RNA associated (S. cerevisiae) | |
| 28955 | DEXI | Dexi homolog (mouse) Dexi homolog | NM_014015 |
| | | (mouse) | |
| 29015 | SLC43A3 | solute carrier family 43, member 3 | NM_014096 |
| 29035 | C16orf72 | chromosome 16 open reading frame 72 | NM_014117 |
| 29080 | CCDC59 | coiled-coil domain containing 59 | NM_014167 |
| 29088 | MRPL15 | mitochondrial ribosomal protein L15 | NM_014175 |
| 29103 | DNAJC15 | DnaJ (Hsp40) homolog, subfamily C, | NM_013238 |
| | | member 15 | |
| 29920 | PYCR2 | pyrroline-5-carboxylate reductase | NM_013328 |
| | | family, member 2 | |
| 30850 | CDR2L | cerebellar degeneration-related protein | NM_014603 |
| | | 2-like | |
| 50801 | KCNK4 | potassium channel, subfamily K, | NM_033310 |
| | | member 4 | |
| 50861 | STMN3 | stathmin-like 3 | NM_015894 |
| 50865 | HEBP1 | heme binding protein 1 | NM_015987 |
| 51000 | SLC35B3 | solute carrier family 35, member B3 | NM_001142540 |
| 51003 | MED31 | mediator complex subunit 31 | NM_016060 |
| 51063 | CALHM2 | calcium homeostasis modulator 2 | NM_015916 |
| 51115 | FAM82B | family with sequence similarity 82, | NM_016033 |
| | | member B family with sequence | |
| | | similarity 82, member B | |
| 51118 | UTP11L | UTP11-like, U3 small nucleolar | NM_016037 |
| | | ribonucleoprotein, (yeast) | |
| 51150 | SDF4 | stromal cell derived factor 4 | NM_016176 |
| 51167 | CYB5R4 | cytochrome b5 reductase 4 | NM_016230 |
| 51182 | HSPA14 | Heat shock 70 kDa protein 14 | NM_016299 |
| 51185 | CRBN | cereblon | NM_001173482 |
| 51199 | NIN | ninein (GSK3B interacting protein) | NM_016350 |
| 51246 | SHISA5 | Shisa homolog 5 (Xenopus laevis) | NM_016479 |
| 51251 | NT5C3 | 5′-nucleotidase, cytosolic III | NM_001002010 |
| 51263 | MRPL30 | mitochondrial ribosomal protein L30 | NM_145212 |
| 51292 | GMPR2 | guanosine monophosphate reductase 2 | NM_001002000 |
| 51535 | PPHLN1 | periphilin 1 | NM_001143787 |
| 51540 | SCLY | selenocysteine lyase | NM_016510 |
| 51604 | PIGT | phosphatidylinositol glycan anchor | NM_001184728 |
| | | biosynthesis, class T | |
| | | phosphatidylinositol glycan anchor | |
| | | biosynthesis, class T | |
| 51619 | UBE2D4 | ubiquitin-conjugating enzyme E2D 4 | NM_015983 |
| | | (putative) | |
| 51754 | TMEM8B | transmembrane protein 8B | NM_001042589 |
| | | transmembrane protein 8B | |
| 53342 | IL17D | interleukin 17D | NM_138284 |
| 53947 | A4GALT | alpha 1,4-galactosyltransferase | NM_017436 |
| 54149 | C21orf91 | chromosome 21 open reading frame 91 | NM_001100420 |
| 54206 | ERRFI1 | ERBB receptor feedback inhibitor 1 | NM_018948 |
| 54468 | MIOS | missing oocyte, meiosis regulator, | NM_019005 |
| | | homolog (Drosophila) | |
| 54503 | ZDHHC13 | zinc finger, DHHC-type containing 13 | NM_001001483 |
| 54662 | TBC1D13 | TBC1 domain family, member 13 | NM_018201 |
| 54681 | P4HTM | prolyl 4-hydroxylase, transmembrane | NM_177938 |
| | | (endoplasmic reticulum) prolyl 4- | |
| | | hydroxylase, transmembrane | |
| | | (endoplasmic reticulum) | |
| 54834 | GDAP2 | ganglioside induced differentiation | NM_001135589 |
| | | associated protein 2 | |
| 54980 | C2orf42 | chromosome 2 open reading frame 42 | NM_017880 |
| 54985 | HCFC1R1 | host cell factor C1 regulator 1 (XPO1 | NM_001002017 |
| | | dependent) | |
| 55055 | ZWILCH | Zwilch, kinetochore associated, | NM_017975 |
| | | homolog (Drosophila) | |
| 55069 | C7orf42 | chromosome 7 open reading frame 42 | NM_017994 |
| 55070 | DET1 | de-etiolated homolog 1 (Arabidopsis) | NM_001144074 |
| 55071 | C9orf40 | chromosome 9 open reading frame 40 | NM_017998 |
| 55100 | WDR70 | WD repeat domain 70 | NM_018034 |
| 55142 | HAUS2 | HAUS augmin-like complex, subunit 2 | NM_001130447 |
| 55204 | GOLPH3L | golgi phosphoprotein 3-like | NM_018178 |
| 55215 | FANCI | Fanconi anemia, complementation | NM_001113378 |
| | | group I | |
| 55239 | OGFOD1 | 2-oxoglutarate and iron-dependent | NM_018233 |
| | | oxygenase domain containing 1 | |
| 55246 | CCDC25 | coiled-coil domain containing 25 | NM_018246 |
| 55652 | SLC48A1 | solute carrier family 48 (heme | NM_017842 |
| | | transporter), member 1 solute carrier | |
| | | family 48 (heme transporter), member 1 | |
| 55698 | RADIL | Ras association and DIL domains | NM_018059 |
| 55742 | PARVA | parvin, alpha | NM_018222 |
| 55751 | TMEM184C | transmembrane protein 184C | NM_018241 |
| 55818 | KDM3A | lysine (K)-specific demethylase 3A | NM_001146688 |
| | | lysine (K)-specific demethylase 3A | |
| | | lysine (K)-specific demethylase 3A | |
| 55839 | CENPN | centromere protein N | NM_001100624 |
| 55846 | ITFG2 | integrin alpha FG-GAP repeat | NM_018463 |
| | | containing 2 | |
| 55872 | PBK | PDZ binding kinase | NM_018492 |
| 55892 | MYNN | myoneurin | NM_001185118 |
| 56052 | ALG1 | asparagine-linked glycosylation 1, | NM_019109 |
| | | beta-1,4-mannosyltransferase homolog | |
| | | (S. cerevisiae) | |
| 56257 | MEPCE | methylphosphate capping enzyme | NM_001194990 |
| 56658 | TRIM39 | tripartite motif containing 39 | NM_021253 |
| 56683 | C21orf59 | chromosome 21 open reading frame 59 | NM_021254 |
| 56731 | SLC2A4RG | SLC2A4 regulator | NM_020062 |
| 56925 | LXN | latexin | NM_020169 |
| 56938 | ARNTL2 | aryl hydrocarbon receptor nuclear | NM_020183 |
| | | translocator-like 2 | |
| 56963 | RGMA | RGM domain family, member A | NM_001166283 |
| 56974 | DKFZp547K054 | hypothetical protein DKFZp547K054 | AL390175.1 |
| 56981 | PRDM11 | PR domain containing 11 | NM_020229 |
| 56992 | KIF15 | kinesin family member 15 | NM_020242 |
| 57082 | CASC5 | cancer susceptibility candidate 5 | NM_144508 |
| 57128 | LYRM4 | LYR motif containing 4 | NM_001164840 |
| 57134 | MAN1C1 | mannosidase, alpha, class 1C, member | NM_020379 |
| | | 1 | |
| 57178 | ZM1Z1 | zinc finger, MIZ-type containing 1 | NM_020338 |
| 57212 | KIAA0495 | KIAA0495 | NR_033708.1 |
| 57226 | LYRM2 | LYR motif containing 2 LYR motif | NM_020466 |
| | | containing 2 | |
| 57405 | SPC25 | SPC25, NDC80 kinetochore complex | NM_020675 |
| | | component, homolog (S. cerevisiae) | |
| 57446 | NDRG3 | NDRG family member 3 | NM_022477 |
| 57456 | KIAA1143 | KIAA1143 | NM_020696 |
| 57617 | VPS18 | vacuolar protein sorting 18 homolog | NM_020857 |
| | | (S. cerevisiae) | |
| 57664 | PLEKHA4 | pleckstrin homology domain | NM_001161354 |
| | | containing, family A (phosphoinositide | |
| | | binding specific) member 4 | |
| 57692 | MAGEE1 | melanoma antigen family E, 1 | NM_020932 |
| 57701 | NCKAP5L | NCK-associated protein 5-like | NM_001037806 |
| 58190 | CTDSP1 | CTD (carboxy-terminal domain, RNA | NM_021198 |
| | | polymerase II, polypeptide A) small | |
| | | phosphatase 1 | |
| 58476 | TP53INP2 | tumor protein p53 inducible nuclear | NM_021202 |
| | | protein 2 | |
| 58491 | ZNF71 | zinc finger protein 71 | NM_021216 |
| 59345 | GNB4 | guanine nucleotide binding protein (G | NM_021629 |
| | | protein), beta polypeptide 4 | |
| 60386 | SLC25A19 | solute carrier family 25 (mitochondrial | NM_001126121 |
| | | thiamine pyrophosphate carrier), | |
| | | member 19 | |
| 60492 | CCDC90B | coiled-coil domain containing 90B | NM_021825 |
| 60626 | RIC8A | resistance to inhibitors of | NM_021932 |
| | | cholinesterase 8 homolog A (C. | |
| | | elegans) | |
| 63894 | VIPAR | VPS33B interacting protein, apical- | NM_001193314 |
| | | basolateral polarity regulator | |
| 64219 | PJA1 | Praja ring finger 1 | NM_001032396 |
| 64326 | RFWD2 | ring finger and WD repeat domain 2 | NM_001001740 |
| 64766 | S100PBP | S100P binding protein | NM_001017406 |
| 65265 | C8orf33 | chromosome 8 open reading frame 33 | NM_023080 |
| 79041 | TMEM38A | transmembrane protein 38A | NM_024074 |
| 79072 | FASTKD3 | FAST kinase domains 3 | NM_024091 |
| 79083 | MLPH | melanophilin melanophilin | NM_001042467 |
| 79084 | WDR77 | WD repeat domain 77 | NM_024102 |
| 79178 | THTPA | thiamine triphosphatase | NM_001126339 |
| 79447 | C16orf53 | chromosome 16 open reading frame 53 | NM_024516 |
| 79600 | TCTN1 | tectonic family member 1 | NM_001082537 |
| 79634 | SCRN3 | secernin 3 | NM_001193528 |
| 79717 | PPCS | phosphopantothenoylcysteine | NM_001077447 |
| | | synthetase | |
| 79723 | SUV39H2 | suppressor of variegation 3-9 homolog | NM_001193424 |
| | | 2 (Drosophila) | |
| 79768 | C15orf29 | chromosome 15 open reading frame 29 | NM_024713 |
| 79812 | MMRN2 | multimerin 2 | NM_024756 |
| 79825 | CCDC48 | coiled-coil domain containing 48 | NM_024768 |
| 79842 | ZBTB3 | zinc finger and BTB domain | NM_024784 |
| | | containing 3 | |
| 79877 | DCAKD | dephospho-CoA kinase domain | NM_001128631 |
| | | containing | |
| 79992 | NCRNA00241 | non-protein coding RNA 241 | AK026765.1 |
| 80079 | FLJ13769 | hypothetical protein FLJ13769 | XR_040824.1 |
| 80258 | EFHC2 | EF-hand domain (C-terminal) | NM_025184 |
| | | containing 2 | |
| 80303 | EFHD1 | EF-hand domain family, member D1 | NM_025202 |
| 80745 | THUMPD2 | THUMP domain containing 2 | NM_025264 |
| 80760 | ITIH5 | Inter-alpha (globulin) inhibitor H5 | NM_001001851 |
| 80818 | ZNF436 | zinc finger protein 436 | NM_001077195 |
| 80824 | DUSP16 | Dual specificity phosphatase 16 | NM_030640 |
| 81562 | LMAN2L | lectin, mannose-binding 2-like | NM_001142292 |
| 81614 | NIPA2 | non imprinted in Prader- | NM_001008860 |
| | | Willi/Angelman syndrome 2 | |
| 81618 | ITM2C | integral membrane protein 2C | NM_001012514 |
| 81689 | ISCA1 | iron-sulfur cluster assembly 1 homolog | NM_030940 |
| | | (S. cerevisiae) | |
| 81892 | C14orf156 | chromosome 14 open reading frame | NM_031210 |
| | | 156 | |
| 81930 | KIF18A | kinesin family member 18A | NM_031217 |
| 83716 | CRISPLD2 | cysteine-rich secretory protein LCCL | NM_031476 |
| | | domain containing 2 | |
| 83742 | MARVELD1 | MARVEL domain containing 1 | NM_031484 |
| 83759 | RBM4B | RNA binding motif protein 4B | NM_031492 |
| 83858 | ATAD3B | ATPase family, AAA domain | NM_031921 |
| | | containing 3B | |
| 83931 | STK40 | serine/threonine kinase 40 | NM_032017 |
| 83932 | C1orf124 | chromosome 1 open reading frame 124 | NM_001010984 |
| 84079 | ANKRD27 | ankyrin repeat domain 27 (VPS9 | NM_032139 |
| | | domain) | |
| 84135 | UTP15 | UTP15, U3 small nucleolar | NM_032175 |
| | | ribonucleoprotein, homolog (S. | |
| | | cerevisiae) | |
| 84138 | SLC7A6OS | solute carrier family 7, member 6 | NM_032178 |
| | | opposite strand | |
| 84154 | RPF2 | ribosome production factor 2 homolog | NM_032194 |
| | | (S. cerevisiae) | |
| 84168 | ANTXR1 | anthrax toxin receptor 1 | NM_018153 |
| 84172 | POLR1B | polymerase (RNA) I polypeptide B, | NM_001137604 |
| | | 128 kDa | |
| 84236 | RHBDD1 | rhomboid domain containing 1 | NM_001167608 |
| 84246 | MED10 | mediator complex subunit 10 | NM_032286 |
| 84247 | LDOC1L | leucine zipper, down-regulated in | NM_032287 |
| | | cancer 1-like | |
| 84268 | RPAIN | RPA interacting protein | NM_001033002 |
| 84298 | LLPH | LLP homolog, long-term synaptic | NM_032338 |
| | | facilitation (Aplysia) | |
| 84464 | SLX4 | SLX4 structure-specific endonuclease | NM_032444 |
| | | subunit homolog (S. cerevisiae) | |
| 84498 | FAM120B | family with sequence similarity 120B | NM_032448 |
| 84515 | MCM8 | minichromosome maintenance | NM_032485 |
| | | complex component 8 | |
| 84661 | DPY30 | dpy-30 homolog (C. elegans) | NM_032574 |
| 84701 | COX4I2 | cytochrome c oxidase subunit IV | NM_032609 |
| | | isoform 2 (lung) | |
| 84750 | FUT10 | fucosyltransferase 10 (alpha (1,3) | NM_032664 |
| | | fucosyltransferase) | |
| 84752 | B3GNT9 | UDP-GlcNAc: betaGal beta-1,3-N- | NM_033309 |
| | | acetylglucosaminyltransferase 9 | |
| 84775 | ZNF607 | zinc finger protein 607 | NM_001172677 |
| 84814 | PPAPDC3 | phosphatidic acid phosphatase type 2 | NM_032728 |
| | | domain containing 3 | |
| 84826 | SFT2D3 | SFT2 domain containing 3 | NM_032740 |
| 84865 | CCDC142 | coiled-coil domain containing 142 | NM_032779 |
| 84866 | TMEM25 | transmembrane protein 25 | NM_001144034 |
| 84872 | ZC3H10 | zinc finger CCCH-type containing 10 | NM_032786 |
| 84886 | C1orf198 | chromosome 1 open reading frame 198 | NM_001136494 |
| 84894 | LINGO1 | leucine rich repeat and Ig domain | NM_032808 |
| | | containing 1 | |
| 84930 | MASTL | microtubule associated | NM_001172303 |
| | | serine/threonine kinase-like | |
| 84939 | MUM1 | melanoma associated antigen (mutated) | NM_032853 |
| | | 1 | |
| 84952 | CGNL1 | cingulin-like 1 | NM_032866 |
| 85004 | RERG | RAS-like, estrogen-regulated, growth | NM_001190726 |
| | | inhibitor | |
| 85315 | PAQR8 | progestin and adipoQ receptor family | NM_133367 |
| | | member VIII | |
| 85376 | RIMBP3 | RIMS binding protein 3 | NM_015672 |
| 90293 | KLHL13 | kelch-like 13 (Drosophila) | NM_001168299 |
| 90324 | CCDC97 | coiled-coil domain containing 97 | NM_052848 |
| 90780 | PYGO2 | pygopus homolog 2 (Drosophila) | NM_138300 |
| 91373 | UAP1L1 | UDP-N-acteylglucosamine | NM_207309 |
| | | pyrophosphorylase 1-like 1 | |
| 91408 | BTF3L4 | basic transcription factor 3-like 4 | NM_001136497 |
| 92140 | MTDH | metadherin | NM_178812 |
| 92181 | UBTD2 | ubiquitin domain containing 2 | NM_152277 |
| 92595 | ZNF764 | zinc finger protein 764 | NM_001172679 |
| 92806 | CENPBD1 | CENPB DNA-binding domains | NM_145039 |
| | | containing 1 | |
| 92856 | IMP4 | IMP4, U3 small nucleolar | NM_033416 |
| | | ribonucleoprotein, homolog (yeast) | |
| 92999 | ZBTB47 | zinc finger and BTB domain | NM_145166 |
| | | containing 47 zinc finger and BTB | |
| | | domain containing 47 | |
| 94039 | ZNF101 | zinc finger protein 101 | NM_033204 |
| 94121 | SYTL4 | synaptotagmin-like 4 | NM_001129896 |
| 113115 | FAM54A | family with sequence similarity 54, | NM_001099286 |
| | | member A | |
| 113189 | CHST14 | carbohydrate (N-acetylgalactosamine | NM_130468 |
| | | 4-0) sulfotransferase 14 | |
| 113612 | CYP2U1 | cytochrome P450, family 2, subfamily | NM_183075 |
| | | U, polypeptide 1 | |
| 114824 | PNMA5 | paraneoplastic antigen like 5 | NM_001103150 |
| 116039 | OSR2 | odd-skipped related 2 (Drosophila) | NM_001142462 |
| | | odd-skipped related 2 (Drosophila) | |
| 117581 | TWIST2 | twist homolog 2 (Drosophila) | NM_057179 |
| 118487 | CHCHD1 | coiled-coil-helix-coiled-coil-helix | NM_203298 |
| | | domain containing 1 | |
| 122011 | CSNK1A1L | casein kinase 1, alpha 1-like | NM_145203 |
| 123096 | SLC25A29 | solute carrier family 25, member 29 | NM_001039355 |
| 123207 | C15orf40 | chromosome 15 open reading frame 40 | NM_00160113 |
| 124790 | HEXIM2 | hexamthylene bis-acetamide inducible | NM_144608 |
| | | 2 | |
| 125058 | TBC1D16 | TBC1 domain family, member 16 | NM_019020 |
| 126006 | PCP2 | Purkinje cell protein 2 | NM_174895 |
| 127703 | C1orf216 | chromosome 1 open reading frame 216 | NM_152374 |
| 128637 | TBC1D20 | TBC1 domain family, member 20 | NM_144628 |
| 129804 | FBLN7 | fibulin 7 | NM_001128165 |
| 129807 | NEU4 | sialidase 4 sialidase 4 | NM_001167599 |
| 131965 | METTL6 | methyltransferase like 6 | NM_152396 |
| 133584 | EGFLAM | EGF-like, fibronectin type III and | NM_152403 |
| | | laminin G domains | |
| 140706 | C20orf160 | chromosome 20 open reading frame | NM_080625 |
| | | 160 | |
| 140831 | ZSWIM3 | zinc finger, SWIM-type containing 3 | NM_080752 |
| 140862 | ISM1 | isthmin 1 homolog (zebrafish) | NM_080826 |
| 146346 | LOC146346 | hypothetical protein LOC146346 | AK057359.1, |
| | | | BX648396.1 |
| 148223 | C19orf25 | chromosome 19 open reading frame 25 | NM_152482 |
| 148932 | MOBKL2C | MOB1, Mps One Binder kinase | NM_145279 |
| | | activator-like 2C (yeast) | |
| 149111 | CNIH3 | cornichon homolog 3 (Drosophila) | NM_152495 |
| 154141 | MBOAT1 | membrane bound O-acyltransferase | NM_001080480 |
| | | domain containing 1 | |
| 154796 | AMOT | angiomotin | NM_001113490 |
| 155061 | ZNF746 | zinc finger protein 746 | NM_001163474 |
| 157247 | MGC27345 | hypothetical protein MGC27345 | XR_108807.1 |
| 158293 | FAM120AOS | family with sequence similarity 120A | NM_198841 |
| | | opposite strand | |
| 163049 | ZNF791 | zinc finger protein 791 | NM_153358 |
| 170506 | DHX36 | DEAH (Asp-Glu-Ala-His) box | NM_001114397 |
| | | polypeptide 36 | |
| 199745 | THAP8 | THAP domain containing 8 | NM_152658 |
| 199857 | ALG14 | asparagine-linked glycosylation 14 | NM_144988 |
| | | homolog (S. cerevisiae) | |
| 200728 | TMEM17 | transmembrane protein 17 | NM_198276 |
| 201163 | FLCN | folliculin | NM_144606 |
| 219539 | YPEL4 | yippee-like 4 (Drosophila) | NM_145008 |
| 220988 | HNRNPA3 | heterogeneous nuclear | NM_194247 |
| | | ribonucleoprotein A3 | |
| 221362 | LOC221362 | hypothetical protein LOC221362 | XP_001134277.1 |
| 221442 | LOC221442 | adenylate cyclase 10 pseudogene | NR_026938.2 |
| 223082 | ZNRF2 | zinc and ring finger 2 | NM_147128 |
| 245812 | CNPY4 | canopy 4 homolog (zebrafish) | NM_152755 |
| 255326 | LOC255326 | hypothetical protein LOC255326 | NR_003611.2 |
| 255458 | LOC255458 | hypothetical protein LOC255458 | NR_027183.1 |
| 256691 | MAMDC2 | MAM domain containing 2 | NM_153267 |
| 259266 | ASPM | asp (abnormal spindle) homolog, | NM_018136 |
| | | microcephaly associated (Drosophila) | |
| 283130 | SLC25A45 | solute carrier family 25, member 45 | NM_001077241 |
| 283635 | FAM177A1 | family with sequence similarity 177, | NM_001079519 |
| | | member A1 | |
| 284542 | LOC284542 | hypothetical protein LOC284542 | NM_001194986.1 |
| 285313 | IGSF10 | immunoglobulin superfamily, member | NM_001178145 |
| | | 10 | |
| 285958 | C7orf40 | chromosome 7 open reading frame 40 | NR_003697.1 |
| 339487 | ZBTB8OS | zinc finger and BTB domain | NM_178547 |
| | | containing 8 opposite strand | |
| 342892 | ZNF850 | zinc finger protein 850 | NM_001193552 |
| 360132 | FKBP9L | FK506 binding protein 9-like | NR_003949.1 |
| 375096 | FLJ44670 | FLJ44670 protein | NM_080627.2 |
| 376497 | SLC27A1 | solute carrier family 27 (fatty acid | NM_198580 |
| | | transporter), member 1 | |
| 389033 | LOC389033 | placenta-specific 9 pseudogene | NR_026740.1 |
| 390299 | LOC390299 | similar to peptidylprolyl isomerase A | XM_372452.4 |
| | | isoform 1 | |
| 400604 | LOC400604 | hypothetical LOC400604 | XR_109405.1 |
| 400629 | TEX19 | testis expressed 19 | NM_207459 |
| 440498 | HSBP1L1 | heat shock factor binding protein 1-like | NM_001136180 |
| | | 1 | |
| 440796 | LOC440796 | similar to Gamma- | NR_003267.1 |
| | | glutamyltranspeptidase 1 precursor | |
| | | (Gamma-glutamyltransferase 1) | |
| | | (CD224 antigen) | |

### Example 2

### Classification of SSc into Subtypes

As described previously (U.S. Ser. No. 13/054,244), Applicant has shown that five distinct SSc subtypes can be identified by different gene expression profiles of a set of intrinsic genes. As used herein, an “intrinsic gene” is a gene that shows little variance within repeated samplings of tissue from an individual subject having scleroderma, but which shows high variance across the same tissue in multiple subjects, wherein the multiple subjects include both subjects having scleroderma and subjects not having scleroderma. For example, an intrinsic gene can be a gene that shows little variance within repeated samplings of forearm-back skin pairs in a subject having scleroderma, but which shows high variance across forearm-back skin pairs of other subjects, wherein the other subjects include both subjects having scleroderma and subjects not having scleroderma.

An intrinsic gene set is a group of genes including one or more intrinsic genes. A minimal intrinsic gene set is defined herein as being derived from an intrinsic gene set, and is comprised of the smallest number of intrinsic genes that can be used to classify a sample.

As described previously, intrinsic gene sets were used to classify scleroderma into a Diffuse-Proliferation group or subtype thereof, Inflammatory group, Limited group or Normal-Like group. The Diffuse-Proliferation group is composed solely of patients with a diagnosis of dSSc. The Inflammatory group includes patients with dSSc, lSSc and morphea. The Limited group is composed solely of patients with lSSc. The Normal-Like group includes healthy controls along with dSSc and lSSc patients. These intrinsic groups or subsets create a more refined division of the disease than current clinical diagnoses and allow for the assessment of patients in different subsets and their likelihood of responding to therapy. For example, it has been shown that patients in the Diffuse-Proliferation group are likely to respond to the drug imatinib mesylate, marketed under the trade name of GLEEVEC® (Novartis Pharmaceuticals, East Hanover, N.J.). Furthermore, selected genes from this gene expression signature provide a basis for identifying patients having, or at risk of having, ILD or digital ulcer involvement.

Based on analysis of the ca. 1000 identified intrinsic genes as disclosed, it was possible to categorize non-overlapping sets of genes from within these approximately 1000 intrinsic genes that differentiate the Diffuse-Proliferation group, the Inflammatory group, the Limited group, and the Normal-Like group.

Genes that differentiate the Diffuse-Proliferation group. There are two major sets of genes that differentiate the Diffuse-Proliferation group. One set (Group I) shows higher expression in the Diffuse-Proliferation group and the other set (Group II) shows lower expression in the Diffuse-Proliferation group. The Diffuse-Proliferation group is also defined in part by the general absence of an Inflammatory signature, although there can be some overlap between the Inflammatory and Diffuse-Proliferation signatures.

Group I genes include 138 genes, the increased expression of which is indicative of the Diffuse-Proliferation group. Expression of these genes is decreased in the Inflammatory, Limited, and Normal-Like groups. Referring to Table 3 below, included in the genes of Group I are the following genes, each identified by name: ANP32A, APOH, ATAD2, B3GALT6, B3GAT3, C12orf14, C14orf131, CACNG6, CBLL1, CBX8, CDC7, CDT1, CENPE, CGI-90, CLDN6, CREB3L3, CROC4, DDX3Y, DERP6, DJ971N18.2, EHD2, ESPL1, FGF5, FLJ10902, FLJ12438, FLJ12443, FLJ12484, FLJ12572, FLJ20245, FLJ32009, FLJ35757, FXYD2, GABRA2, GATA2, GK, GSG2, HPS3, IKBKG, IL23A, INSIG1, KIAA1509, KIAA1609, KIAA1666, LDLR, LGALS8, LILRB5, LOC123876, LOC128977, LOC153561, LOC283464, LRRIQ2, LY6K, MAC30, ME2, MGC13186, MGC16044, MGC16075, MGC29784, MGC33839, MGC35212, MGC4293, MICB, MLL5, MTRF1L, MUC20, NICN1, NPTX1, OAS3, OGDHL, OPRK1, PCNT2, PDZK1, PITPNC1, PPFIA4, PREB, PRKY, PSMD11, PSPH, PSPHL, PTP4A3, PXMP2, RAB15, RAD51AP1, RIP, RNF121, RPL41, RPS18, RPS4Y1, RPS4Y2, S100P, SORD, SP1, SYMPK, SYT6, TM9SF4, TMOD3, TNFRSF12A, TPRA40, TRIP, TRPM7, TTR, TUBB4, VARS2L, ZNF572, and ZSCAN2. Also included in the genes of Group I are the following genes, each identified by GenBank accession number only: A_{—}24_BS934268, AB065507, AC007051, AI791206, AK022745, AK022893, AK022997, AK094044, AL391244, AL731541, AL928970, BC010544, BC020847, BM925639, BM928667, ENST00000328708, ENST00000333517, I_{—}1891291, L3580313, NM_{—}001009569, NM_{—}001024808, NM_{—}172020, NM_{—}173705, NM_{—}178467, NR_{—}001544, THC1434038, THC1484458, THC1504780, U62539, XM_{—}210579, XM_{—}303638, and XM_{—}371684.

Group II genes include 298 genes, the decreased expression of which is also indicative of the Diffuse-Proliferation group. Expression of these genes is increased in the Inflammatory, Limited, and Normal-Like groups. Referring to Table 3 below, included in the genes of Group II are the following genes, each identified by name: AADAC, ADAM17, ADH1A, ADH1C, AHNAK, ALG1, ALG5, AMOT, AOX1, AP2A2, ARK5, ARMCX1, BECN1, BECN1, BMP8A, BNIP3L, C10orf119, C1orf24, C1orf37, C20orf10, C20orf22, C5orf14, C6orf64, C9orf61, CAPS, CASP4, CASP5, CAST, CAV2, CCDC6, CCNG2, CDC26, CDK2AP1, CDR1, CFHL1, CNTN3, CPNE5, CRTAP, CTNNA1, CTSC, CUTL1, CXCL5, CYBRD1, CYP2R1, DBN1, DCAMKL1, DCL-1, DIAPH2, DKK2, ECHDC3, ECM2, E1F3S7, EMB, EMCN, EMILIN2, ENPP2, EPB41L2, FBLN1, FBLN2, FEM1A, FGL2, FHL5, FKBP7, FLI1, FLJ10986, FLJ20032, FLJ20701, FLJ23861, FLJ34969, FLJ36748, FLJ36888, FLJ43339, FZR1, GABPB2, GARNL4, GHITM, GHR, GIT2, GLYAT, GPM6B, GTPBP5, HELB, HOXB4, IFNA6, IGFBP5, IL13RA1, IL15, KAZALD1, KCNK4, KCNS3, KCTD10, KIAA0232, KIAA0494, KIAA0562, KIAA0870, KIAA1190, KIF25, KLHL18, KLK2, LAMP2, LEPROTL1, LHFP, LMO2, LOC114990, LOC255458, LOC387680, LOC400027, LOC493869, LOC87769, LRBA, MAFB, MAGEH1, MAN2B2, MCCC2, MEGF10, MFAP5, MGC11308, MGC15523, MGC3200, MGC35048, MGC45780, MOGAT3, MPPE1, MPZ, MYO1B, MYOC, NFYC, NIPSNAP3B, OPTN, OSR2, PAM, PBXIP1, PCOLCE2, PDGFC, PDGFRA, PDGFRL, PEX19, PHAX, PIP, PKM2, PKP2, PMP22, POU2F1, PPAP2B, PRAC, PSMA5, PSORS1C1, PTGIS, RECK, RGS11, RGS5, RIMS3, RIPK2, RNASE4, RNF125, RNF13, RNF146, RNF19, ROBO1, ROBO3, RPL7A, SARA1, SAV1, SCGB1D1, SDK1, SECP43, SECTM1, SERPINB2, SGCA, SH3BGRL, SH3GLB1, SH3RF2, SLC10A3, SLC12A2, SLC14A1, SLC39A14, SLC7A7, SLC9A9, SLPI, SMAD1, SMAP1, SMARCE1, SMP1, SNTG2, SNX7, SOCS5, SSPN, STX7, SUMF1, TAS2R10, TDE2, TFAP2B, TGFBR2, THSD2, TM4SF3, TMEM25, TMEM34, TNA, TNKS2, TRAD, TRAF3IP1, TREM4, TRIM35, TRIM9, TTYH2, TUBB1, UBL3, ULK2, URB, USP54, UST, UTRN, UTX, WIF1, WWOX, XG, YPEL5, and ZFHX1B. Also included in the genes of Group II are the following genes, each identified by GenBank accession number only: A_{—}32_BS 169243, A_{—}32_BS200773, A_{—}32_BS53976, AC025463, AF124368, AF161364, AF318337, AF372624, AK001565, AK022793, AK055621, AK056856, AL050042, AL137761, BC035102, BC038761, BC039664, BG252130, BI014689, D80006, ENST00000298643, ENST00000300068, ENST00000305402, ENST00000307901, ENST00000321656, ENST00000322803, ENST00000329246, ENST00000331640, ENST00000332271, ENST00000333784, H16080, L1861543, I_{—}1882608, L1985061, L3335767, L3551568, L3588329, L932413, L962800, I_{—}966091, NM_{—}001008528, NM_{—}001009555, NM_{—}001013632, NM_{—}001014975, NM_{—}001018006, NM_{—}001018076, NM_{—}001025077, NM_{—}003671, NM_{—}014758, NM_{—}015262, NM_{—}138411, NM_{—}153030, NM_{—}173709, NM_{—}213595, NR_{—}002184, S62210, THC1419743, THC1429821, THC1457118, THC1459712, THC1461073, THC1506312, THC1511927, THC1515028, THC1525318, THC1531579, THC1544941, THC1551463, THC1559236, THC1560798, THC1563147, THC1572906, THC1574967, THC1591470, XM_{—}165930, and XM_{—}209429.

Genes that differentiate the Inflammatory group. The Inflammatory group is identified by increased expression of a group of 119 genes in Group III. These genes show low expression in the Diffuse-Proliferation, Limited, and Normal-Like groups. Referring to Table 3 below, included in the genes of Group III are the following genes, each identified by name: A2M, AIF1, ALOX5AP, APOL2, APOL3, BATF, BCL3, BIRC1, BTN3A2, C10orf10, C1orf38, C6orf80, CCL2, CCL4, CCR5, CD8A, CDW52, COL6A3, COTL1, CPA3, CPVL, CTAG1B, DDX58, EBI2, EVI2B, F13A1, FAM20A, FAP, FCGR3A, FLJ11259, FLJ22573, FLJ23221, FLJ25200, FYB, GBP1, GBP3, GEM, GIMAP6, GMFG, GZMH, GZMK, HAVCR2, HCLS1, HLA-DMA, HLA-DOA, HLA-DPA1, HLA-DPB1, HLA-DQA1, HLA-DQA2, HLA-DQB1, HLA-DRB1, HLA-DRB5, ICAM2, IFI16, IFI16, IFIT1, IFIT2, IFITM1, IFITM2, IFITM3, IL10RA, INDO, ITGB2, KIAA0063, LAMB 1, LCP1, LGALS2, LGALS9, LILRB2, LOC387763, LOC400759, LUM, LYZ, MARCKS, MFNG, MGC24133, MPEG1, MRC1, MRCL3, MS4A6A, MX1, NNMT, NUP62, PAG, PLAU, PPIC, PPIC, PTPRC, RAC2, RGS10, RGS16, RSAFD1, SAT, SCGB2A1, SLC20A1, SLCO2B1, SPARC, SULF1, TAP1, TCTEL1, TIMP1, TNFSF4, UBD, VSIG4, and ZFYVE26. Also included in the genes of Group III are the following genes, each identified by GenBank accession number only: AF533936, BQ049338, ENST00000310210, ENST00000313904, ENST00000329660, I_{—}1000437, I_{—}966691, M15073, NM_{—}001010919, NM_{—}001025201, NM_{—}001033569, THC1543691, and XM_{—}291496.

In one embodiment the Inflammatory group, and likewise a subject that can be categorized as falling within the Inflammatory group, can be identified by the increased expression of any one or more genes within Group III.

In one embodiment the Inflammatory group, and likewise a subject that can be categorized as falling within the Inflammatory group, can be identified by the increased expression of any one or more genes within Group III and the decreased expression of any one or more genes in Group I.

In one embodiment the Inflammatory group, and likewise a subject that can be categorized as falling within the Inflammatory group, can be identified by the increased expression of any one or more genes within Group III and the increased expression of any one or more genes within Group II.

In one embodiment the Inflammatory group, and likewise a subject that can be categorized as falling within the Inflammatory group, can be identified by the increased expression of any one or more genes within Group III, the decreased expression of any one or more genes in Group I, and the increased expression of any one or more genes within Group II.

TABLE 3

| Gene Symbol | Gene Name | Accession |
|---|---|---|
| A2M | Alpha-2-macroglobulin | M36501 |
| AADAC | Arylacetamide deacetylase (esterase) | NM_001086 |
| ACTB | Actin, beta | NM_001101 |
| ADAM17 | A disintegrin and metalloproteinase domain 17 | NM_003183 |
| | (tumor necrosis factor, alpha, converting enzyme) | |
| ADH1A | Alcohol dehydrogenase 1A (class I), alpha | NM_000667 |
| | polypeptide | |
| ADH1C | Alcohol dehydrogenase 1C (class I), gamma | NM_000669 |
| | polypeptide | |
| AHNAK | AHNAK nucleoprotein (desmoyokin) | NM_024060 |
| AIF1 | Allograft inflammatory factor 1 | NM_004847 |
| AKAP13 | A kinase (PRKA) anchor protein 13 | AF406992 |
| ALG1 | Asparagine-linked glycosylation 1 homolog | NM_019109 |
| | (yeast, beta-1,4-mannosyltransferase) | |
| ALG2 | Asparagine-linked glycosylation 2 homolog | NM_033087 |
| | (yeast, alpha-1,3-mannosyltransferase) | |
| ALG5 | Asparagine-linked glycosylation 5 homolog | NM_013338 |
| | (yeast, dolichyl-phosphate beta- | |
| | glucosyltransferase) | |
| ALOX5AP | Arachidonate 5-lipoxygenase-activating protein | NM_001629 |
| ALS2CR13 | Amyotrophic lateral sclerosis 2 (juvenile) | NM_004703 |
| | chromosome region, candidate 13 | |
| ALX3 | Aristaless-like homeobox 3 | NM_006492 |
| AMFR | Autocrine motility factor receptor | NM_138958 |
| AMOT | Angiomotin | NM_133265 |
| ANP32A | Acidic (leucine-rich) nuclear phosphoprotein 32 | AK021784 |
| | family, member A | |
| AOX1 | Aldehyde oxidase 1 | NM_001159 |
| AP2A2 | Adaptor-related protein complex 2, alpha 2 | NM_012305 |
| | subunit | |
| APOH | Apolipoprotein H (beta-2-glycoprotein I) | NM_000042 |
| APOL2 | Apolipoprotein L, 2 | NM_030882 |
| APOL3 | Apolipoprotein L, 3 | NM_145640 |
| ARHGEF10 | Rho guanine nucleotide exchange factor (GEF) | NM_014629 |
| | 10 | |
| ARK5 | AMP-activated protein kinase family member 5 | NM_014840 |
| ARL6IP5 | ADP-ribosylation-like factor 6 interacting protein 5 | NM_006407 |
| ARMCX1 | Armadillo repeat containing, X-linked 1 | NM_016608 |
| ARX | Aristaless related homeobox | NM_139058 |
| ASCL3 | Achaete-scute complex (Drosophila) homolog- | NM_020646 |
| | like 3 | |
| ATAD2 | ATPase family, AAA domain containing 2 | NM_014109 |
| ATP1A4 | ATPase, Na+/K+ transporting, alpha 4 | NM_144699 |
| | polypeptide | |
| ATP6V1B2 | ATPase, H+ transporting, lysosomal 56/58 kDa, | NM_001693 |
| | V1 subunit B, isoform 2 | |
| AVPI1 | Arginine vasopressin-induced 1 | NM_021732 |
| AXL | AXL receptor tyrosine kinase | NM_001699 |
| B3GALT6 | UDP-Gal:betaGal beta 1,3-galactosyltransferase | NM_080605 |
| | polypeptide 6 | |
| B3GAT3 | Beta-1,3-glucuronyltransferase 3 | NM_012200 |
| | (glucuronosyltransferase I) | |
| B3GTL | Beta 3-glycosyltransferase-like | BC032021 |
| BAALC | Brain and acute leukemia, cytoplasmic | NM_024812 |
| BATF | Basic leucine zipper transcription factor, ATF- | NM_006399 |
| | like | |
| BCAR1 | Breast cancer anti-estrogen resistance 1 | NM_014567 |
| BCKDHB | Branched chain keto acid dehydrogenase E1, beta | NM_183050 |
| | polypeptide (maple syrup urine disease) | |
| BCL3 | B-cell CLL/lymphoma 3 | NM_005178 |
| BECN1 | Beclin 1 (coiled-coil, myosin-like BCL2 | NM_003766 |
| | interacting protein) | |
| BECN1 | Beclin 1 (coiled-coil, myosin-like BCL2 | NM_003766 |
| | interacting protein) | |
| BEXL1 | Brain expressed X-linked-like 1 | XM_043653 |
| BIRC1 | Baculoviral IAP repeat-containing 1 | NM_004536 |
| Bles03 | Basophilic leukemia expressed protein BLES03 | NM_031450 |
| BMP8A | Bone morphogenetic protein 8a | AK093659 |
| BNIP3L | BCL2/adenovirus E1B 19 kDa interacting protein | AF067396 |
| | 3-like | |
| BNIP3L | BCL2/adenovirus E1B 19 kDa interacting protein | NM_004331 |
| | 3-like | |
| BTN3A2 | Butyrophilin, subfamily 3, member A2 | NM_007047 |
| C10orf10 | Chromosome 10 open reading frame 10 | NM_007021 |
| C10orf119 | Chromosome 10 open reading frame 119 | NM_024834 |
| C10orf9 | Chromosome 10 open reading frame 9 | NM_145012 |
| C12orf14 | Chromosome 12 open reading frame 14 | NM_021238 |
| C14orf131 | Chromosome 14 open reading frame 131 | NM_018335 |
| C1orf24 | Chromosome 1 open reading frame 24 | NM_052966 |
| C1orf37 | Chromosome 1 open reading frame 37 | CR591805 |
| C1orf38 | Chromosome 1 open reading frame 38 | NM_004848 |
| C1orf42 | Chromosome 1 open reading frame 42 | NM_019060 |
| C20orf10 | Chromosome 20 open reading frame 10 | NM_014477 |
| C20orf22 | Chromosome 20 open reading frame 22 | NM_015600 |
| C4.4A | GPI-anchored metastasis-associated protein | NM_014400 |
| | homolog | |
| C5orf14 | Chromosome 5 open reading frame 14 | NM_024715 |
| C6orf27 | Chromosome 6 open reading frame 27 | NM_025258 |
| C6orf64 | Chromosome 6 open reading frame 64 | NM_018322 |
| C6orf80 | Chromosome 6 open reading frame 80 | NM_015439 |
| C7orf19 | Chromosome 7 open reading frame 19 | NM_032831 |
| C9orf61 | Chromosome 9 open reading frame 61 | NM_004816 |
| CABP7 | Calcium binding protein 7 | NM_182527 |
| CACNA2D1 | Calcium channel, voltage-dependent, alpha | NM_000722 |
| | 2/delta subunit 1 | |
| CACNG6 | Calcium channel, voltage-dependent, gamma | NM_145814 |
| | subunit 6 | |
| CAPN10 | Calpain 10 | NM_021251 |
| CAPS | Calcyphosine | NM_004058 |
| CASP4 | Caspase 4, apoptosis-related cysteine protease | NM_033307 |
| CASP5 | Caspase 5, apoptosis-related cysteine protease | NM_004347 |
| CAST | Calpastatin | NM_173060 |
| CAV2 | Caveolin 2 | NM_001233 |
| CBLL1 | Cas-Br-M (murine) ecotropic retroviral | NM_024814 |
| | transforming sequence-like 1 | |
| CBX8 | Chromobox homolog 8 (Pc class homolog, | NM_020649 |
| | Drosophila) | |
| CCDC6 | Coiled-coil domain containing 6 | S72869 |
| CCL2 | Chemokine (C-C motif) ligand 2 | NM_002982 |
| CCL4 | Chemokine (C-C motif) ligand 4 | NM_002984 |
| CCNG2 | Cyclin G2 | NM_004354 |
| CCNG2 | Cyclin G2 | NM_004354 |
| CCNT2 | Cyclin T2 | NM_058241 |
| CCR5 | Chemokine (C-C motif) receptor 5 | NM_000579 |
| CCT5 | Chaperonin containing TCP1, subunit 5 (epsilon) | NM_012073 |
| CD33 | CD33 antigen (gp67) | NM_001772 |
| CD86 | CD86 antigen (CD28 antigen ligand 2, B7-2 | NM_006889 |
| | antigen) | |
| CD8A | CD8 antigen, alpha polypeptide (p32) | NM_001768 |
| CDC26 | Cell division cycle 26 | NM_139286 |
| CDC37 | CDC37 cell division cycle 37 homolog (S. cerevisiae) | NM_007065 |
| CDC7 | CDC7 cell division cycle 7 (S. cerevisiae) | NM_003503 |
| CDK2AP1 | CDK2-associated protein 1 | NM_004642 |
| CDR1 | Cerebellar degeneration-related protein 1, 34 kDa | NM_004065 |
| CDT1 | DNA replication factor | NM_030928 |
| CDW52 | CDW52 antigen (CAMPATH-1 antigen) | NM_001803 |
| CEBPD | CCAAT/enhancer binding protein (C/EBP), delta | NM_005195 |
| CENPE | Centromere protein E, 312 kDa | NM_001813 |
| CFHL1 | Complement factor H-related 1 | NM_002113 |
| CGI-111 | CGI-111 protein | NM_016048 |
| CGI-90 | CGI-90 protein | NM_016033 |
| CISH | Cytokine inducible SH2-containing protein | NM_145071 |
| CKLFSF1 | Chemokine-like factor super family 1 | NM_181294 |
| CLDN6 | Claudin 6 | NM_021195 |
| CLIPR-59 | CLIP-170-related protein | BC013116 |
| CLYBL | Citrate lyase beta like | NM_138280 |
| CNFN | Cornifelin | NM_032488 |
| CNTN3 | Contactin 3 (plasmacytoma associated) | AB040929 |
| COL1A2 | Collagen, type I, alpha 2 | NM_000089 |
| COL6A2 | Collagen, type VI, alpha 2 | NM_001849 |
| COL6A3 | Collagen, type VI, alpha 3 | NM_057165 |
| COMMD2 | COMM domain containing 2 | NM_016094 |
| COTL1 | Coactosin-like 1 (Dictyostelium) | NM_021149 |
| COX5A | Cytochrome c oxidase subunit Va | AA129107 |
| CPA3 | Carboxypeptidase A3 (mast cell) | NM_001870 |
| CPNE5 | Copine V | NM_020939 |
| CPVL | Carboxypeptidase, vitellogenic-like | NM_019029 |
| CRBN | Cereblon | AF130117 |
| CREB3L3 | CAMP responsive element binding protein 3-like 3 | NM_032607 |
| CRLF1 | Cytokine receptor-like factor 1 | NM_004750 |
| CROC4 | Transcriptional activator of the c-fos promoter | NM_006365 |
| CRTAP | Cartilage associated protein | NM_006371 |
| CTAG1B | Cancer/testis antigen 1B | NM_139250 |
| CTAGE4 | CTAGE family, member 4 | XM_496933 |
| CTNNA1 | Catenin (cadherin-associated protein), alpha 1, | NM_001903 |
| | 102 kDa | |
| CTSC | Cathepsin C | NM_001814 |
| CTSH | Cathepsin H | NM_148979 |
| CUTL1 | Cut-like 1, CCAAT displacement protein | NM_181500 |
| | (Drosophila) | |
| CXCL5 | Chemokine (C—X—C motif) ligand 5 | NM_002994 |
| CYBRD1 | Cytochrome b reductase 1 | NM_024843 |
| CYP2R1 | Cytochrome P450, family 2, subfamily R, | NM_024514 |
| | polypeptide 1 | |
| CYP4V2 | Cytochrome P450, family 4, subfamily V, | NM_207352 |
| | polypeptide 2 | |
| DBN1 | Drebrin 1 | NM_004395 |
| DCAMKL1 | Doublecortin and CaM kinase-like 1 | NM_004734 |
| DCL-1 | Type I transmembrane C-type lectin receptor | NM_014880 |
| | DCL-1 | |
| DDX3Y | DEAD (Asp-Glu-Ala-Asp) box polypeptide 3, Y- | NM_004660 |
| | linked | |
| DDX58 | DEAD (Asp-Glu-Ala-Asp) box polypeptide 58 | NM_014314 |
| DDX6 | DEAD (Asp-Glu-Ala-Asp) box polypeptide 6 | AK021715 |
| DERP6 | S-phase 2 protein | NM_015362 |
| DIAPH2 | Diaphanous homolog 2 (Drosophila) | NM_006729 |
| DICER1 | Dicer1, Dcr-1 homolog (Drosophila) | NM_177438 |
| DIRC1 | Disrupted in renal carcinoma 1 | NM_052952 |
| DJ971N18.2 | Hypothetical protein DJ971N18.2 | NM_021156 |
| DJ971N18.2 | Hypothetical protein DJ971N18.2 | NM_021156 |
| DKFZp761C169 | Vasculin | CR621588 |
| DKK2 | Dickkopf homolog 2 (Xenopus laevis) | NM_014421 |
| DNCL1 | Dynein, cytoplasmic, light polypeptide 1 | NM_003746 |
| DPCD | Deleted in a mouse model of primary ciliary | AF264625 |
| | dyskinesia | |
| DPP3 | Dipeptidylpeptidase 3 | NM_005700 |
| DREV1 | DORA reverse strand protein 1 | NM_016025 |
| EBI2 | Epstein-Barr virus induced gene 2 (lymphocyte- | NM_004951 |
| | specific G protein-coupled receptor) | |
| ECHDC3 | Enoyl Coenzyme A hydratase domain containing 3 | NM_024693 |
| ECM2 | Extracellular matrix protein 2, female organ and | NM_001393 |
| | adipocyte specific | |
| EDG4 | Endothelial differentiation, lysophosphatidic acid | NM_004720 |
| | G-protein-coupled receptor, 4 | |
| EGFL3 | EGF-like-domain, multiple 3 | NM_001409 |
| EHD2 | EH-domain containing 2 | BC062554 |
| EIF3S3 | Eukaryotic translation initiation factor 3, subunit | NM_003756 |
| | 3 gamma, 40 kDa | |
| EIF3S7 | Eukaryotic translation initiation factor 3, subunit | NM_003753 |
| | 7 zeta, 66/67 kDa | |
| EIF3S8 | Eukaryotic translation initiation factor 3, subunit | NM_003752 |
| | 8, 110 kDa | |
| EIF4B | Eukaryotic translation initiation factor 4B | NM_001417 |
| ELA1 | Elastase 1, pancreatic | NM_001971 |
| EMB | Embigin homolog (mouse) | U52054 |
| EMCN | Endomucin | AL133118 |
| EMILIN2 | Elastin microfibril interfacer 2 | NM_032048 |
| EMR2 | Egf-like module containing, mucin-like, hormone | NM_152918 |
| | receptor-like 2 | |
| ENPP2 | Ectonucleotide | NM_006209 |
| | pyrophosphatase/phosphodiesterase 2 (autotaxin) | |
| EPB41L2 | Erythrocyte membrane protein band 4.1-like 2 | NM_001431 |
| ESM1 | Endothelial cell-specific molecule 1 | NM_007036 |
| ESPL1 | Extra spindle poles like 1 (S. cerevisiae) | NM_012291 |
| ESRRB | Estrogen-related receptor beta | NM_004452 |
| ET | Hypothetical protein ET | NM_024311 |
| EVI2B | Ecotropic viral integration site 2B | NM_006495 |
| EXOSC6 | Exosome component 6 | NM_058219 |
| F13A1 | Coagulation factor XIII, A1 polypeptide | NM_000129 |
| F7 | Coagulation factor VII (serum prothrombin | AF272774 |
| | conversion accelerator) | |
| FABP7 | Fatty acid binding protein 7, brain | NM_001446 |
| FAM12A | Family with sequence similarity 12, member A | NM_006683 |
| FAM20A | Family with sequence similarity 20, member A | NM_017565 |
| FAP | Fibroblast activation protein, alpha | NM_004460 |
| FBLN1 | Fibulin 1 | NM_006486 |
| FBLN2 | Fibulin 2 | NM_001998 |
| FCGR3A | Fc fragment of IgG, low affinity IIIb, receptor for | NM_000569 |
| | (CD16) | |
| FEM1A | Fem-1 homolog a (C. elegans) | NM_018708 |
| FER1L3 | Fer-1-like 3, myoferlin (C. elegans) | NM_133337 |
| FGF19 | Fibroblast growth factor 19 | NM_005117 |
| FGF5 | Fibroblast growth factor 5 | NM_004464 |
| FGL2 | Fibrinogen-like 2 | NM_006682 |
| FHL5 | Four and a half LIM domains 5 | NM_020482 |
| FKBP5 | FK506 binding protein 5 | NM_004117 |
| FKBP7 | FK506 binding protein 7 | NM_181342 |
| FKSG2 | Apoptosis inhibitor | NM_021631 |
| FLI1 | Friend leukemia virus integration 1 | NM_002017 |
| FLJ10647 | Hypothetical protein FLJ10647 | NM_018166 |
| FLJ10781 | Hypothetical protein FLJ10781 | NM_018215 |
| FLJ10902 | Hypothetical protein FLJ10902 | BC021277 |
| FLJ10986 | Hypothetical protein FLJ10986 | NM_018291 |
| FLJ11259 | Hypothetical protein FLJ11259 | NM_018370 |
| FLJ12363 | Hypothetical protein FLJ12363 | NM_032167 |
| FLJ12438 | Hypothetical protein FLJ12438 | NM_021933 |
| FLJ12443 | Hypothetical protein FLJ12443 | NM_024830 |
| FLJ12484 | Hypothetical protein FLJ12484 | NM_022767 |
| FLJ12572 | Hypothetical protein FLJ12572 | AF411456 |
| FLJ12748 | Hypothetical protein FLJ12748 | NM_024871 |
| FLJ20032 | Hypothetical protein FLJ20032 | AK000039 |
| FLJ20245 | Hypothetical protein FLJ20245 | NM_017723 |
| FLJ20701 | Hypothetical protein FLJ20701 | NM_017933 |
| FLJ21616 | Hypothetical protein FLJ21616 | NM_024567 |
| FLJ22573 | Hypothetical protein FLJ22573 | NM_024660 |
| FLJ23221 | Hypothetical protein FLJ23221 | NM_024579 |
| FLJ23861 | Hypothetical protein FLJ23861 | NM_152519 |
| FLJ25200 | Hypothetical protein FLJ25200 | NM_144715 |
| FLJ25222 | CXYorf1-related protein | NM_199163 |
| FLJ31882 | Hypothetical protein FLJ31882 | NM_152460 |
| FLJ32009 | Hypothetical protein FLJ32009 | NM_152718 |
| FLJ34969 | Hypothetical protein FLJ34969 | NM_152678 |
| FLJ35390 | Hypothetical protein FLJ35390 | XM_379820 |
| FLJ35757 | Hypothetical protein FLJ35757 | NM_152598 |
| FLJ35775 | Hypothetical protein FLJ35775 | NM_152418 |
| FLJ36748 | Hypothetical protein FLJ36748 | NM_152406 |
| FLJ36888 | Hypothetical protein FLJ36888 | NM_178830 |
| FLJ38379 | Hypothetical protein FLJ38379 | NM_178530 |
| FLJ39441 | Hypothetical protein FLJ39441 | NM_194285 |
| FLJ43339 | FLJ43339 protein | CR749408 |
| FLJ44896 | FLJ44896 protein | BQ189189 |
| FLJ90661 | Hypothetical protein FLJ90661 | NM_173502 |
| FN3KRP | Fructosamine-3-kinase-related protein | NM_024619 |
| FXN | Frataxin | NM_000144 |
| FXYD2 | FXYD domain containing ion transport regulator 2 | NM_021603 |
| FYB | FYN binding protein (FYB-120/130) | NM_001465 |
| FZR1 | Fizzy/cell division cycle 20 related 1 | NM_016263 |
| | (Drosophila) | |
| G1P2 | Interferon, alpha-inducible protein (clone IFI- | NM_005101 |
| | 15K) | |
| G1P3 | Interferon, alpha-inducible protein (clone IFI-6- | NM_022873 |
| | 16) | |
| GABPB2 | GA binding protein transcription factor, beta | BC009935 |
| | subunit 2, 47 kDa | |
| GABRA2 | Gamma-aminobutyric acid (GABA) A receptor, | NM_000807 |
| | alpha 2 | |
| GARNL4 | GTPase activating Rap/RanGAP domain-like 4 | NM_015085 |
| GATA2 | GATA binding protein 2 | NM_032638 |
| GBP1 | Guanylate binding protein 1, interferon-inducible, | NM_002053 |
| | 67 kDa | |
| GBP3 | Guanylate binding protein 3 | NM_018284 |
| GEM | GTP binding protein overexpressed in skeletal | NM_005261 |
| | muscle | |
| GFAP | Glial fibrillary acidic protein | NM_002055 |
| GH1 | Growth hormone 1 | NM_000515 |
| GHITM | Growth hormone inducible transmembrane | NM_014394 |
| | protein | |
| GHR | Growth hormone receptor | NM_000163 |
| GIMAP6 | GTPase, IMAP family member 6 | NM_024711 |
| GIT2 | G protein-coupled receptor kinase interactor 2 | NM_057170 |
| GK | Glycerol kinase | NM_203391 |
| GLIPR1 | GLI pathogenesis-related 1 (glioma) | NM_006851 |
| GLYAT | Glycine-N-acyltransferase | NM_005838 |
| GMFG | Glia maturation factor, gamma | NM_004877 |
| GPM6B | Glycoprotein M6B | NM_005278 |
| GPSM1 | G-protein signalling modulator 1 (AGS3-like, C. elegans) | AL117478 |
| GPT | Glutamic-pyruvate transaminase (alanine | NM_005309 |
| | aminotransferase) | |
| GPX7 | Glutathione peroxidase 7 | NM_015696 |
| GRINL1A | Glutamate receptor, ionotropic, N-methyl D- | AK074767 |
| | aspartate-like 1A | |
| GRIPAP1 | GRIP1 associated protein 1 | AB032993 |
| GSG2 | Haspin | AK056691 |
| GSPT2 | G1 to S phase transition 2 | NM_018094 |
| GSTM1 | Glutathione S-transferase M1 | NM_000561 |
| GSTM3 | Glutathione S-transferase M3 (brain) | NM_000849 |
| GSTT1 | Glutathione S-transferase theta 1 | NM_000853 |
| GSTT1 | Glutathione S-transferase theta 1 | NM_000853 |
| GSTT2 | Glutathione S-transferase theta 2 | NM_000854 |
| GSTT2 | Glutathione S-transferase theta 2 | NM_000854 |
| GTF3C5 | General transcription factor IIIC, polypeptide 5, | NM_012087 |
| | 63 kDa | |
| GTPBP5 | GTP binding protein 5 (putative) | NM_015666 |
| GTPBP6 | GTP binding protein 6 (putative) | NM_012227 |
| GZMH | Granzyme H (cathepsin G-like 2, protein h- | NM_033423 |
| | CCPX) | |
| GZMK | Granzyme K (serine protease, granzyme 3; | NM_002104 |
| | tryptase II) | |
| H1F0 | H1 histone family, member 0 | NM_005318 |
| HARS | Histidyl-tRNA synthetase | NM_002109 |
| HAVCR2 | Hepatitis A virus cellular receptor 2 | NM_032782 |
| HCLS1 | Hematopoietic cell-specific Lyn substrate 1 | NM_005335 |
| HELB | Helicase (DNA) B | NM_033647 |
| HEPH | Hephaestin | NM_138737 |
| HERPUD1 | Homocysteine-inducible, endoplasmic reticulum | NM_014685 |
| | stress-inducible, ubiquitin-like domain member 1 | |
| HLA-A | Major histocompatibility complex, class I, A | BC020891 |
| HLA-B | Major histocompatibility complex, class I, B | NM_005514 |
| HLA-DMA | Major histocompatibility complex, class II, DM | NM_006120 |
| | alpha | |
| HLA-DOA | Major histocompatibility complex, class II, DO | M38054 |
| | alpha | |
| HLA-DOA | Major histocompatibility complex, class II, DO | NM_002119 |
| | alpha | |
| HLA-DPA1 | Major histocompatibility complex, class II, DP | NM_033554 |
| | alpha 1 | |
| HLA-DPB1 | Major histocompatibility complex, class II, DP | NM_002121 |
| | beta 1 | |
| HLA-DQA1 | Major histocompatibility complex, class II, DQ | NM_002122 |
| | alpha 1 | |
| HLA-DQA2 | Major histocompatibility complex, class II, DQ | NM_020056 |
| | alpha 2 | |
| HLA-DQB1 | Major histocompatibility complex, class II, DQ | M20432 |
| | beta 1 | |
| HLA-DRB1 | Major histocompatibility complex, class II, DR | NM_002124 |
| | beta 4 | |
| HLA-DRB5 | Major histocompatibility complex, class II, DR | NM_002125 |
| | beta 4 | |
| HLA-E | Major histocompatibility complex, class I, E | NM_005516 |
| HLA-E | Major histocompatibility complex, class I, E | NM_005516 |
| HLA-E | Major histocompatibility complex, class I, E | NM_005516 |
| HLA-F | Major histocompatibility complex, class I, F | NM_018950 |
| HLA-G | HLA-G histocompatibility antigen, class I, G | NM_002127 |
| HOXB4 | Homeo box B4 | NM_024015 |
| HPS3 | Hermansky-Pudlak syndrome 3 | NM_032383 |
| HRAS | V-Ha-ras Harvey rat sarcoma viral oncogene | NM_176795 |
| | homolog | |
| HSPBP1 | Hsp70-interacting protein | NM_012267 |
| ICAM2 | Intercellular adhesion molecule 2 | NM_000873 |
| IFI16 | Interferon, gamma-inducible protein 16 | BC017059 |
| IFI16 | Interferon, gamma-inducible protein 16 | NM_005531 |
| IFIT1 | Interferon-induced protein with tetratricopeptide | NM_001001887 |
| | repeats 1 | |
| IFIT2 | Interferon-induced protein with tetratricopeptide | NM_001547 |
| | repeats 2 | |
| IFITM1 | Interferon induced transmembrane protein 1 (9- | NM_003641 |
| | 27) | |
| IFITM2 | Interferon induced transmembrane protein 2 (1- | NM_006435 |
| | 8D) | |
| IFITM3 | Interferon induced transmembrane protein 3 (1- | NM_021034 |
| | 8U) | |
| IFNA6 | Interferon, alpha 6 | NM_021002 |
| IGFBP5 | Insulin-like growth factor binding protein 5 | NM_000599 |
| IGH@ | Immunoglobulin heavy locus | BC040042 |
| IGHG4 | Immunoglobulin heavy constant gamma 4 (G4m | BC025985 |
| | marker) | |
| IGKC | Immunoglobulin kappa constant | AJ399872 |
| IGKC | Immunoglobulin kappa constant | BC030813 |
| IGLL1 | Immunoglobulin lambda-like polypeptide 1 | NM_152855 |
| IGLL1 | Immunoglobulin lambda-like polypeptide 1 | NM_152855 |
| IKBKG | Inhibitor of kappa light polypeptide gene | NM_003639 |
| | enhancer in B-cells, kinase gamma | |
| IL10RA | Interleukin 10 receptor, alpha | NM_001558 |
| IL13RA1 | Interleukin 13 receptor, alpha 1 | NM_001560 |
| IL15 | Interleukin 15 | NM_172175 |
| IL23A | Interleukin 23, alpha subunit p19 | NM_016584 |
| IL27 | Interleukin 27 | NM_145659 |
| INDO | Indoleamine-pyrrole 2,3 dioxygenase | NM_002164 |
| INSIG1 | Insulin induced gene 1 | NM_005542 |
| IQCF2 | IQ motif containing F2 | NM_203424 |
| IRF5 | Interferon regulatory factor 5 | NM_032643 |
| IRF7 | Interferon regulatory factor 7 | NM_004030 |
| IRX3 | Iroquois homeobox protein 3 | NM_024336 |
| IRX5 | Iroquois homeobox protein 5 | NM_005853 |
| ITGAL | Integrin, alpha L (antigen CD11A (p180), | NM_002209 |
| | lymphocyte function-associated antigen 1; alpha | |
| | polypeptide) | |
| ITGB1 | Integrin, beta 1 (fibronectin receptor, beta | NM_033666 |
| | polypeptide, antigen CD29 includes MDF2, | |
| | MSK12) | |
| ITGB1BP1 | Integrin beta 1 binding protein 1 | NM_004763 |
| ITGB2 | Integrin, beta 2 (antigen CD18 (p95), lymphocyte | NM_000211 |
| | function-associated antigen 1; macrophage | |
| | antigen 1 (mac-1) beta subunit) | |
| ITLN1 | Intelectin 1 (galactofuranose binding) | NM_017625 |
| KAZALD1 | Kazal-type serine protease inhibitor domain 1 | NM_030929 |
| KCNK4 | Potassium channel, subfamily K, member 4 | NM_016611 |
| KCNS3 | Potassium voltage-gated channel, delayed- | NM_002252 |
| | rectifier, subfamily S, member 3 | |
| KCTD10 | Potassium channel tetramerisation domain | NM_031954 |
| | containing 10 | |
| KCTD15 | Potassium channel tetramerisation domain | NM_024076 |
| | containing 15 | |
| KEL | Kell blood group | NM_000420 |
| KIAA0063 | KIAA0063 gene product | NM_014876 |
| KIAA0232 | KIAA0232 gene product | NM_014743 |
| KIAA0467 | KIAA0467 protein | NM_015284 |
| KIAA0494 | KIAA0494 gene product | NM_014774 |
| KIAA0562 | Glycine-, glutamate-, | NM_014704 |
| | thienylcyclohexylpiperidine-binding protein | |
| KIAA0664 | KIAA0664 protein | NM_015229 |
| KIAA0676 | KIAA0676 protein | NM_015043 |
| KIAA0870 | KIAA0870 protein | NM_014957 |
| KIAA1190 | Hypothetical protein KIAA1190 | NM_145166 |
| KIAA1463 | KIAA1463 protein | NM_173602 |
| KIAA1509 | KIAA1509 | XM_029353 |
| KIAA1609 | KIAA1609 protein | NM_020947 |
| KIAA1666 | KIAA1666 protein | XM_371429 |
| KIAA1683 | KIAA1683 | NM_025249 |
| KIF25 | Kinesin family member 25 | NM_005355 |
| KLF9 | Kruppel-like factor 9 | NM_001206 |
| KLHL18 | Kelch-like 18 (Drosophila) | AB018338 |
| KLK2 | Kallikrein 2, prostatic | NM_005551 |
| KRT20 | Keratin 20 | NM_019010 |
| LAMB1 | Laminin, beta 1 | NM_002291 |
| LAMP2 | Lysosomal-associated membrane protein 2 | NM_013995 |
| LAMR1P15 | Laminin receptor 1 pseudogene 15 | AF284768 |
| LCP1 | Lymphocyte cytosolic protein 1 (L-plastin) | NM_002298 |
| LDLR | Low density lipoprotein receptor (familial | M28219 |
| | hypercholesterolemia) | |
| LEPR | Leptin receptor | NM_017526 |
| LEPROTL1 | Leptin receptor overlapping transcript-like 1 | AF359269 |
| LGALS2 | Lectin, galactoside-binding, soluble, 2 (galectin | NM_006498 |
| | 2) | |
| LGALS8 | Lectin, galactoside-binding, soluble, 8 (galectin | NM_201543 |
| | 8) | |
| LGALS9 | Lectin, galactoside-binding, soluble, 9 (galectin | NM_002308 |
| | 9) | |
| LHFP | Lipoma HMGIC fusion partner | NM_005780 |
| LILRB2 | Leukocyte immunoglobulin-like receptor, | NM_005874 |
| | subfamily B (with TM and ITIM domains), | |
| | member 2 | |
| LILRB5 | Leukocyte immunoglobulin-like receptor, | NM_006840 |
| | subfamily B (with TM and ITIM domains), | |
| | member 5 | |
| LMO2 | LIM domain only 2 (rhombotin-like 1) | NM_005574 |
| LMOD1 | Leiomodin 1 (smooth muscle) | AW939148 |
| LMOD1 | Leiomodin 1 (smooth muscle) | NM_012134 |
| LOC114990 | Vasorin | NM_138440 |
| LOC123876 | Xenobiotic/medium-chain fatty acid:CoA ligase | NM_182617 |
| LOC128977 | Hypothetical protein LOC128977 | NM_173793 |
| LOC142678 | Skeletrophin | NM_080875 |
| LOC147645 | Hypothetical protein LOC147645 | XM_085831 |
| LOC153561 | Hypothetical LOC389295 | NM_207331 |
| LOC255458 | Hypothetical protein LOC255458 | BC009038 |
| LOC283464 | Hypothetical protein LOC283464 | XM_290597 |
| LOC284323 | Hypothetical protein LOC284323 | AK091274 |
| LOC339834 | Hypothetical protein LOC339834 | NM_178173 |
| LOC387680 | Similar to KIAA0592 protein | NM_001005751 |
| LOC387763 | Hypothetical LOC387763 | XM_373497 |
| LOC400027 | Hypothetical gene supported by BC047417 | XM_378350 |
| LOC400581 | GRB2-related adaptor protein-like | BC026233 |
| LOC400759 | Similar to Interferon-induced guanylate-binding | XM_375747 |
| | protein 1 (GTP-binding protein 1) (Guanine | |
| | nucleotide-binding protein 1) (HuGBP-1) | |
| LOC401565 | Similar to 4931415M17 protein | NM_001001710 |
| LOC441245 | Hypothetical LOC441245 | XM_496889 |
| LOC493869 | Similar to RIKEN cDNA 2310016C16 | AK022110 |
| LOC51035 | ORF | NM_015853 |
| LOC87769 | Hypothetical protein BC004360 | XM_373431 |
| LOC91689 | Hypothetical gene supported by AL449243 | NM_033318 |
| LPXN | Leupaxin | NM_004811 |
| LRAP | Leukocyte-derived arginine aminopeptidase | NM_022350 |
| LRBA | LPS-responsive vesicle trafficking, beach and | NM_006726 |
| | anchor containing | |
| LRRC14 | Leucine rich repeat containing 14 | NM_014665 |
| LRRC2 | Leucine rich repeat containing 2 | NM_024512 |
| LRRIQ2 | Leucine-rich repeats and IQ motif containing 2 | NM_024548 |
| LTBP4 | Latent transforming growth factor beta binding | AF051344 |
| | protein 4 | |
| LTBP4 | Latent transforming growth factor beta binding | NM_003573 |
| | protein 4 | |
| LUM | Lumican | NM_002345 |
| LY6K | Lymphocyte antigen 6 complex, locus K | NM_017527 |
| LY6K | Lymphocyte antigen 6 complex, locus K | NM_017527 |
| LYZ | Lysozyme (renal amyloidosis) | NM_000239 |
| MAB21L2 | Mab-21-like 2 (C. elegans) | NM_006439 |
| MAC30 | Hypothetical protein MAC30 | NM_014573 |
| MAFB | V-maf musculoaponeurotic fibrosarcoma | NM_005461 |
| | oncogene homolog B (avian) | |
| MAGEH1 | Melanoma antigen, family H, 1 | NM_014061 |
| MAN2B2 | Mannosidase, alpha, class 2B, member 2 | NM_015274 |
| MARCH-II | Membrane-associated RING-CH protein II | NM_016496 |
| MARCKS | Myristoylated alanine-rich protein kinase C | NM_002356 |
| | substrate | |
| MCCC1 | Methylcrotonoyl-Coenzyme A carboxylase 1 | NM_020166 |
| | (alpha) | |
| MCCC2 | Methylcrotonoyl-Coenzyme A carboxylase 2 | AK001948 |
| | (beta) | |
| ME2 | Malic enzyme 2, NAD(+)-dependent, | BC000147 |
| | mitochondrial | |
| MED19 | Mediator of RNA polymerase II transcription, | NM_153450 |
| | subunit 19 homolog (yeast) | |
| MEGF10 | MEGF10 protein | BC020198 |
| MERTK | C-mer proto-oncogene tyrosine kinase | U08023 |
| MFAP5 | Microfibrillar associated protein 5 | NM_003480 |
| MFNG | Manic fringe homolog (Drosophila) | NM_002405 |
| MGC10772 | Hypothetical protein MGC10772 | NM_030567 |
| MGC11308 | Hypothetical protein MGC11308 | NM_032889 |
| MGC13186 | Hypothetical protein MGC13186 | NM_032324 |
| MGC15523 | Hypothetical protein MGC15523 | BC020925 |
| MGC15875 | Hypothetical protein MGC15875 | AK090397 |
| MGC16044 | Hypothetical protein MGC16044 | NM_138371 |
| MGC16075 | Hypothetical protein MGC16075 | XM_498434 |
| MGC21654 | Unknown MGC21654 product | NM_145647 |
| MGC23918 | Hypothetical protein MGC23918 | NM_144716 |
| MGC24133 | Hypothetical protein MGC24133 | NM_174896 |
| MGC27165 | Hypothetical protein MGC27165 | AK027379 |
| MGC29784 | Hypothetical protein MGC29784 | NM_173659 |
| MGC29937 | Hypothetical protein MGC29937 | NM_144597 |
| MGC3169 | Hypothetical protein MGC3169 | NM_024074 |
| MGC3200 | Hypothetical protein LOC284615 | NM_032305 |
| MGC33839 | Hypothetical protein MGC33839 | NM_152353 |
| MGC35045 | Chromosome 19 open reading frame 16 | AL834316 |
| MGC35048 | Hypothetical protein MGC35048 | NM_153208 |
| MGC35212 | Hypothetical protein MGC35212 | NM_152764 |
| MGC39584 | Hypothetical gene supported by BC029568 | BC029568 |
| MGC42157 | Hypothetical locus MGC42157 | XM_499573 |
| MGC4293 | Hypothetical protein MGC4293 | NM_031304 |
| MGC45428 | Hypothetical protein MGC45428 | NM_152619 |
| MGC45780 | Hypothetical protein MGC45780 | NM_173833 |
| MGC8721 | Hypothetical protein MGC8721 | NM_016127 |
| MGC9515 | Hypothetical protein MGC9515 | BC036263 |
| MICB | MHC class I polypeptide-related sequence B | NM_005931 |
| MIS12 | MIS12 homolog (yeast) | NM_024039 |
| MKRN1 | Makorin, ring finger protein, 1 | NM_013446 |
| MLL5 | Myeloid/lymphoid or mixed-lineage leukemia 5 | NM_182931 |
| | (trithorax homolog, Drosophila) | |
| MNS1 | Meiosis-specific nuclear structural protein 1 | NM_018365 |
| MOBKL2A | MOB1, Mps One Binder kinase activator-like 2A | AK024373 |
| | (yeast) | |
| MOGAT3 | Monoacylglycerol O-acyltransferase 3 | NM_178176 |
| MPEG1 | Macrophage expressed gene 1 | AK074166 |
| MPP1 | Membrane protein, palmitoylated 1, 55 kDa | NM_002436 |
| MPP2 | Membrane protein, palmitoylated 2 (MAGUK | NM_005374 |
| | p55 subfamily member 2) | |
| MPPE1 | Metallophosphoesterase 1 | NM_138608 |
| MPZ | Myelin protein zero (Charcot-Marie-Tooth | NM_000530 |
| | neuropathy 1B) | |
| MRC1 | Mannose receptor, C type 1 | NM_002438 |
| MRCL3 | Myosin regulatory light chain MRCL3 | NM_006471 |
| MRPL43 | Mitochondrial ribosomal protein L43 | NM_176794 |
| MRPL46 | Mitochondrial ribosomal protein L46 | NM_022163 |
| MS4A6A | Membrane-spanning 4-domains, subfamily A, | NM_022349 |
| | member 6A | |
| MSN | Moesin | NM_002444 |
| MT | Malonyl-CoA:acyl carrier protein transacylase, | NM_014507 |
| | mitochondrial | |
| MT1A | Metallothionein 1A (functional) | NM_005946 |
| MT1E | Metallothionein 1E (functional) | NM_175617 |
| MT1H | Metallothionein 1H | NM_005951 |
| MT1J | Metallothionein 1J | NM_175622 |
| MT1K | Metallothionein 1K | NM_176870 |
| MT1L | Metallothionein 1L | X97261 |
| MT1X | Metallothionein 1X | BC032338 |
| MT1X | Metallothionein 1X | NM_005952 |
| MT1X | Metallothionein 1X | NM_005952 |
| MT2A | Metallothionein 2A | BC007034 |
| MT2A | Metallothionein 2A | NM_005953 |
| MT2A | Metallothionein 2A | NM_005953 |
| MTCBP-1 | Membrane-type 1 matrix metalloproteinase | NM_018269 |
| | cytoplasmic tail binding protein-1 | |
| MTCH2 | Mitochondrial carrier homolog 2 (C. elegans) | NM_014342 |
| MTRF1L | Mitochondrial translational release factor 1-like | NM_019041 |
| MUC20 | Mucin 20 | NM_152673 |
| MUC3A | Mucin 3A, intestinal | M55405 |
| MX1 | Myxovirus (influenza virus) resistance 1, | NM_002462 |
| | interferon-inducible protein p78 (mouse) | |
| MYO1B | Myosin IB | NM_012223 |
| MYOC | Myocilin, trabecular meshwork inducible | NM_000261 |
| | glucocorticoid response | |
| NAP1L4 | Nucleosome assembly protein 1-like 4 | NM_005969 |
| NCKAP1 | NCK-associated protein 1 | NM_205842 |
| NFE2L3 | Nuclear factor (erythroid-derived 2)-like 3 | NM_004289 |
| NFYC | Nuclear transcription factor Y, gamma | NM_014223 |
| NICN1 | Nicolin 1 | NM_032316 |
| NINJ1 | Ninjurin 1 | NM_004148 |
| NIPSNAP3B | Nipsnap homolog 3B (C. elegans) | NM_018376 |
| NISCH | Nischarin | NM_007184 |
| NNMT | Nicotinamide N-methyltransferase | NM_006169 |
| NOL6 | Nucleolar protein family 6 (RNA-associated) | NM_130793 |
| NOSIP | Nitric oxide synthase interacting protein | NM_015953 |
| NPTX1 | Neuronal pentraxin I | NM_002522 |
| NUDT2 | Nudix (nucleoside diphosphate linked moiety X)- | NM_001161 |
| | type motif 2 | |
| NUP62 | Nucleoporin 62 kDa | NM_172374 |
| NXPH4 | Neurexophilin 4 | NM_007224 |
| NYREN18 | NEDD8 ultimate buster-1 | BC034716 |
| OAS3 | 2′-5′-oligoadenylate synthetase 3, 100 kDa | NM_006187 |
| OAS3 | 2′-5′-oligoadenylate synthetase 3, 100 kDa | NM_006187 |
| OCA2 | Oculocutaneous albinism II (pink-eye dilution | NM_000275 |
| | homolog, mouse) | |
| OGDHL | Oxoglutarate dehydrogenase-like | NM_018245 |
| OPLAH | 5-oxoprolinase(ATP-hydrolysing) | NM_017570 |
| OPRK1 | Opioid receptor, kappa 1 | NM_000912 |
| OPTN | Optineurin | NM_021980 |
| OSR2 | Odd-skipped related 2 (Drosophila) | NM_053001 |
| OSTbeta | Organic solute transporter beta | NM_178859 |
| P8 | P8 protein (candidate of metastasis 1) | NM_012385 |
| PAG | Phosphoprotein associated with | NM_018440 |
| | glycosphingolipid-enriched microdomains | |
| PAM | Peptidylglycine alpha-amidating monooxygenase | NM_000919 |
| PAX8 | Paired box gene 8 | AK056052 |
| PBXIP1 | Pre-B-cell leukemia transcription factor | NM_020524 |
| | interacting protein 1 | |
| PCNT2 | Pericentrin 2 (kendrin) | NM_006031 |
| PCOLCE2 | Procollagen C-endopeptidase enhancer 2 | NM_013363 |
| PDGFC | Platelet derived growth factor C | NM_016205 |
| PDGFRA | Platelet-derived growth factor receptor, alpha | NM_006206 |
| | polypeptide | |
| PDGFRL | Platelet-derived growth factor receptor-like | NM_006207 |
| PDK4 | Pyruvate dehydrogenase kinase, isoenzyme 4 | NM_002612 |
| PDZK1 | PDZ domain containing 1 | NM_002614 |
| PERLD1 | Per1-like domain containing 1 | NM_033419 |
| PEX19 | Peroxisomal biogenesis factor 19 | NM_002857 |
| PGM1 | Phosphoglucomutase 1 | NM_002633 |
| PGRMC1 | Progesterone receptor membrane component 1 | NM_006667 |
| PHAX | RNA U, small nuclear RNA export adaptor | AF086448 |
| | (phosphorylation regulated) | |
| PHCA | Phytoceramidase, alkaline | NM_018367 |
| PIP | Prolactin-induced protein | NM_002652 |
| PITPNC1 | Phosphatidylinositol transfer protein, cytoplasmic 1 | NM_012417 |
| PKM2 | Pyruvate kinase, muscle | NM_182471 |
| PKP2 | Plakophilin 2 | X97675 |
| PLAU | Plasminogen activator, urokinase | NM_002658 |
| PMP22 | Peripheral myelin protein 22 | NM_000304 |
| PNPLA4 | Patatin-like phospholipase domain containing 4 | NM_004650 |
| POLD4 | Polymerase (DNA-directed), delta 4 | NM_021173 |
| POLR2L | Polymerase (RNA) II (DNA directed) | NM_021128 |
| | polypeptide L, 7.6 kDa | |
| POU2F1 | POU domain, class 2, transcription factor 1 | S66901 |
| PP3856 | Similar to CG3714 gene product | NM_145201 |
| PPAP2B | Phosphatidic acid phosphatase type 2B | NM_003713 |
| PPFIA4 | Protein tyrosine phosphatase, receptor type, f | NM_015053 |
| | polypeptide (PTPRF), interacting protein (liprin), | |
| | alpha 4 | |
| PPIC | Peptidylprolyl isomerase C (cyclophilin C) | NM_000943 |
| PPIC | Peptidylprolyl isomerase C (cyclophilin C) | NM_000943 |
| PPIL3 | Peptidylprolyl isomerase (cyclophilin)-like 3 | NM_131916 |
| PPM1F | Protein phosphatase 1F (PP2C domain | NM_014634 |
| | containing) | |
| PRAC | Small nuclear protein PRAC | NM_032391 |
| PREB | Prolactin regulatory element binding | BE395450 |
| PRIC285 | Peroxisomal proliferator-activated receptor A | NM_033405 |
| | interacting complex 285 | |
| PRKD2 | Protein kinase D2 | NM_016457 |
| PRKY | Protein kinase, Y-linked | NM_002760 |
| PRSS15 | Protease, serine, 15 | NM_004793 |
| PSMA5 | Proteasome (prosome, macropain) subunit, alpha | NM_002790 |
| | type, 5 | |
| PSMB9 | Proteasome (prosome, macropain) subunit, beta | NM_148954 |
| | type, 9 (large multifunctional protease 2) | |
| PSMD11 | Proteasome (prosome, macropain) 26S subunit, | NM_002815 |
| | non-ATPase, 11 | |
| PSORS1C1 | Psoriasis susceptibility 1 candidate 1 | NM_014068 |
| PSPH | Phosphoserine phosphatase | NM_004577 |
| PSPHL | Phosphoserine phosphatase-like | AJ001612 |
| PTAFR | Platelet-activating factor receptor | S52624 |
| PTGIS | Prostaglandin I2 (prostacyclin) synthase | NM_000961 |
| PTOV1 | Prostate tumor overexpressed gene 1 | NM_017432 |
| PTP4A3 | Protein tyrosine phosphatase type IVA, member 3 | NM_007079 |
| PTPRC | Protein tyrosine phosphatase, receptor type, C | NM_080922 |
| PVRL2 | Poliovirus receptor-related 2 (herpesvirus entry | NM_002856 |
| | mediator B) | |
| PXMP2 | Peroxisomal membrane protein 2, 22 kDa | NM_018663 |
| R30953_1 | Interferon inducible GTPase 5 | NM_019612 |
| RAB15 | RAB15, member RAS onocogene family | NM_198686 |
| RABEP1 | Rabaptin, RAB GTPase binding effector protein 1 | NM_004703 |
| RAC2 | Ras-related C3 botulinum toxin substrate 2 (rho | NM_002872 |
| | family, small GTP binding protein Rac2) | |
| RAC2 | Ras-related C3 botulinum toxin substrate 2 (rho | NM_002872 |
| | family, small GTP binding protein Rac2) | |
| RAD51AP1 | RAD51 associated protein 1 | NM_006479 |
| RAI16 | Retinoic acid induced 16 | NM_022749 |
| RAPH1 | Ras association (RalGDS/AF-6) and pleckstrin | NM_213589 |
| | homology domains 1 | |
| RECK | Reversion-inducing-cysteine-rich protein with | NM_021111 |
| | kazal motifs | |
| RGL2 | Ral guanine nucleotide dissociation stimulator- | NM_004761 |
| | like 2 | |
| RGS10 | Regulator of G-protein signalling 10 | NM_001005339 |
| RGS11 | Regulator of G-protein signalling 11 | BC040504 |
| RGS16 | Regulator of G-protein signalling 16 | NM_002928 |
| RGS5 | Regulator of G-protein signalling 5 | NM_003617 |
| RHBDF1 | Rhomboid family 1 (Drosophila) | NM_022450 |
| RHOT2 | Ras homolog gene family, member T2 | NM_138769 |
| RIMS3 | Regulating synaptic membrane exocytosis 3 | NM_014747 |
| RIP | RPA interacting protein | NM_032308 |
| RIPK2 | Receptor-interacting serine-threonine kinase 2 | NM_003821 |
| RLN3 | Relaxin 3 | NM_080864 |
| RNASE4 | Angiogenin, ribonuclease, RNase A family, 5 | NM_001145 |
| RNASE4 | Angiogenin, ribonuclease, RNase A family, 5 | NM_194431 |
| RNF121 | Ring finger protein 121 | AK023139 |
| RNF125 | Ring finger protein 125 | NM_017831 |
| RNF13 | Ring finger protein 13 | NM_007282 |
| RNF138P1 | Ring finger protein 138 pseudogene 1 | AW975013 |
| RNF146 | Ring finger protein 146 | NM_030963 |
| RNF19 | Ring finger protein 19 | NM_183419 |
| ROBO1 | Roundabout, axon guidance receptor, homolog 1 | NM_002941 |
| | (Drosophila) | |
| ROBO3 | Roundabout, axon guidance receptor, homolog 3 | NM_022370 |
| | (Drosophila) | |
| RPL10A | Ribosomal protein L10a | NM_007104 |
| RPL41 | Ribosomal protein L41 | NM_021104 |
| RPL7A | Ribosomal protein L7a | NM_000972 |
| RPS10 | Ribosomal protein S10 | NM_001014 |
| RPS16 | Ribosomal protein S16 | NM_001020 |
| RPS18 | Ribosomal protein S18 | NM_022551 |
| RPS4X | Ribosomal protein S4, X-linked | NM_001007 |
| RPS4Y1 | Ribosomal protein S4, Y-linked 1 | NM_001008 |
| RPS4Y2 | Ribosomal protein S4, Y-linked 2 | NM_138963 |
| RRAGD | Ras-related GTP binding D | NM_021244 |
| RSAFD1 | Radical S-adenosyl methionine and flavodoxin | NM_018264 |
| | domains 1 | |
| RTN4 | Reticulon 4 | NM_153828 |
| RUTBC3 | RUN and TBC1 domain containing 3 | NM_015705 |
| S100P | S100 calcium binding protein P | NM_005980 |
| SAMD10 | Sterile alpha motif domain containing 10 | NM_080621 |
| SARA1 | SAR1a gene homolog 1 (S. cerevisiae) | NM_020150 |
| SARA1 | SAR1a gene homolog 1 (S. cerevisiae) | NM_020150 |
| SAT | Spermidine/spermine N1-acetyltransferase | NM_002970 |
| SAV1 | Salvador homolog 1 (Drosophila) | NM_021818 |
| SCAP | SREBP CLEAVAGE-ACTIVATING PROTEIN | NM_012235 |
| SCGB1D1 | Secretoglobin, family 1D, member 1 | NM_006552 |
| SCGB2A1 | Secretoglobin, family 2A, member 1 | NM_002407 |
| SCUBE3 | Signal peptide, CUB domain, EGF-like 3 | NM_152753 |
| SDK1 | Sidekick homolog 1 (chicken) | AF052150 |
| SECP43 | TRNA selenocysteine associated protein | NM_017846 |
| SECTM1 | Secreted and transmembrane 1 | NM_003004 |
| SEMA3B | Sema domain, immunoglobulin domain (Ig), | NM_004636 |
| | short basic domain, secreted, (semaphorin) 3B | |
| SERPINB2 | Serine (or cysteine) proteinase inhibitor, clade B | BC012609 |
| | (ovalbumin), member 2 | |
| SESN1 | Sestrin 1 | NM_014454 |
| SESN2 | Sestrin 2 | NM_031459 |
| SF4 | Splicing factor 4 | NM_172231 |
| SGCA | Sarcoglycan, alpha (50 kDa dystrophin-associated | NM_000023 |
| | glycoprotein) | |
| SH3BGRL | SH3 domain binding glutamic acid-rich protein | NM_003022 |
| | like | |
| SH3GLB1 | SH3-domain GRB2-like endophilin B1 | NM_016009 |
| SH3GLB2 | SH3-domain GRB2-like endophilin B2 | NM_020145 |
| SH3RF2 | SH3 domain containing ring finger 2 | NM_152550 |
| ShrmL | Shroom-related protein | NM_020859 |
| SIRPB2 | Signal-regulatory protein beta 2 | NM_018556 |
| SLAMF9 | SLAM family member 9 | NM_033438 |
| SLC10A3 | Solute carrier family 10 (sodium/bile acid | NM_019848 |
| | cotransporter family), member 3 | |
| SLC12A2 | Solute carrier family 12 | NM_001046 |
| | (sodium/potassium/chloride transporters), | |
| | member 2 | |
| SLC12A9 | Solute carrier family 12 (potassium/chloride | NM_020246 |
| | transporters), member 9 | |
| SLC14A1 | Solute carrier family 14 (urea transporter), | L36121 |
| | member 1 (Kidd blood group) | |
| SLC20A1 | Solute carrier family 20 (phosphate transporter), | NM_005415 |
| | member 1 | |
| SLC39A14 | Solute carrier family 39 (zinc transporter), | BC000068 |
| | member 14 | |
| SLC6A15 | Solute carrier family 6 (neurotransmitter | NM_018057 |
| | transporter), member 15 | |
| SLC7A1 | Solute carrier family 7 (cationic amino acid | NM_003045 |
| | transporter, y+ system), member 1 | |
| SLC7A7 | Solute carrier family 7 (cationic amino acid | NM_003982 |
| | transporter, y+ system), member 7 | |
| SLC9A3R2 | Solute carrier family 9 (sodium/hydrogen | NM_004785 |
| | exchanger), isoform 3 regulator 2 | |
| SLC9A9 | Solute carrier family 9 (sodium/hydrogen | NM_173653 |
| | exchanger), isoform 9 | |
| SLCO2B1 | Solute carrier organic anion transporter family, | NM_007256 |
| | member 2B1 | |
| SLPI | Secretory leukocyte protease inhibitor | NM_003064 |
| | (antileukoproteinase) | |
| SLPI | Secretory leukocyte protease inhibitor | NM_003064 |
| | (antileukoproteinase) | |
| SMAD1 | SMAD, mothers against DPP homolog 1 | NM_005900 |
| | (Drosophila) | |
| SMAP1 | Stromal membrane-associated protein 1 | NM_021940 |
| SMARCA4 | SWI/SNF related, matrix associated, actin | NM_003072 |
| | dependent regulator of chromatin, subfamily a, | |
| | member 4 | |
| SMARCE1 | SWI/SNF related, matrix associated, actin | NM_003079 |
| | dependent regulator of chromatin, subfamily e, | |
| | member 1 | |
| SMC5L1 | SMC5 structural maintenance of chromosomes 5- | NM_015110 |
| | like 1 (yeast) | |
| SMN2 | Survival of motor neuron 1, telomeric | NM_022877 |
| SMP1 | NPD014 protein | NM_014313 |
| SMTN | Smoothelin | NM_134269 |
| SNTG2 | Syntrophin, gamma 2 | NM_018968 |
| SNX7 | Sorting nexin 7 | NM_015976 |
| SOCS5 | Suppressor of cytokine signaling 5 | NM_014011 |
| SORD | Sorbitol dehydrogenase | NM_003104 |
| SP1 | Sp1 transcription factor | NM_138473 |
| SPARC | Secreted protein, acidic, cysteine-rich | NM_003118 |
| | (osteonectin) | |
| SRD5A2L | Steroid 5 alpha-reductase 2-like | NM_024592 |
| SRGAP3 | SLIT-ROBO Rho GTPase activating protein 3 | AF086321 |
| SRPK2 | SFRS protein kinase 2 | NM_182691 |
| SSB3 | SPRY domain-containing SOCS box protein | NM_080861 |
| | SSB-3 | |
| SSPN | Sarcospan (Kras oncogene-associated gene) | NM_005086 |
| STAT6 | Signal transducer and activator of transcription 6, | NM_003153 |
| | interleukin-4 induced | |
| STX7 | Syntaxin 7 | NM_003569 |
| SULF1 | Sulfatase 1 | NM_015170 |
| SUMF1 | Sulfatase modifying factor 1 | NM_182760 |
| SYAP1 | Synapse associated protein 1, SAP47 homolog | NM_032796 |
| | (Drosophila) | |
| SYMPK | Symplekin | NM_004819 |
| SYNGR2 | Synaptogyrin 2 | NM_004710 |
| SYT6 | Synaptotagmin VI | NM_205848 |
| TAP1 | Transporter 1, ATP-binding cassette, sub-family | NM_000593 |
| | B (MDR/TAP) | |
| TAS2R10 | Taste receptor, type 2, member 10 | NM_023921 |
| TCTEL1 | T-complex-associated-testis-expressed 1-like 1 | NM_006519 |
| TDE2 | Tumor differentially expressed 2 | NM_020755 |
| TETRAN | Tetracycline transporter-like protein | NM_001120 |
| TFAP2B | Transcription factor AP-2 beta (activating | NM_003221 |
| | enhancer binding protein 2 beta) | |
| TFCP2L3 | Transcription factor CP2-like 3 | NM_024915 |
| TGFB1I1 | Transforming growth factor beta 1 induced | NM_015927 |
| | transcript 1 | |
| TGFBR2 | Transforming growth factor, beta receptor II | NM_003242 |
| | (70/80 kDa) | |
| TGM4 | Transglutaminase 4 (prostate) | U79008 |
| THSD2 | Thrombospondin, type I, domain containing 2 | NM_032784 |
| TIFA | TRAF-interacting protein with a forkhead- | NM_052864 |
| | associated domain | |
| TIMP1 | Tissue inhibitor of metalloproteinase 1 (erythroid | NM_003254 |
| | potentiating activity, collagenase inhibitor) | |
| TLR1 | Toll-like receptor 1 | NM_003263 |
| TM4SF3 | Transmembrane 4 superfamily member 3 | NM_004616 |
| TM9SF4 | Transmembrane 9 superfamily protein member 4 | NM_014742 |
| TMEM25 | Transmembrane protein 25 | NM_032780 |
| TMEM34 | Transmembrane protein 34 | NM_018241 |
| TMOD3 | Tropomodulin 3 (ubiquitous) | NM_014547 |
| Tmp21-II | Tmp21-II, transcribed pseudogene | AJ004914 |
| TNA | Tetranectin (plasminogen binding protein) | NM_003278 |
| TNFRSF12A | Tumor necrosis factor receptor superfamily, | NM_016639 |
| | member 12A | |
| TNFRSF18 | Tumor necrosis factor receptor superfamily, | NM_148902 |
| | member 18 | |
| TNFSF4 | Tumor necrosis factor (ligand) superfamily, | NM_003326 |
| | member 4 (tax-transcriptionally activated | |
| | glycoprotein 1, 34 kDa) | |
| TNKS2 | Tankyrase, TRF1-interacting ankyrin-related | NM_025235 |
| | ADP-ribose polymerase 2 | |
| TPRA40 | Seven transmembrane domain orphan receptor | NM_016372 |
| TRAD | Serine/threonine kinase with Dbl- and pleckstrin | AL137629 |
| | homology domains | |
| TRAF3IP1 | TNF receptor-associated factor 3 interacting | BC059174 |
| | protein 1 | |
| TREM4 | Triggering receptor expressed on myeloid cells 4 | NM_145273 |
| TRIM35 | Tripartite motif-containing 35 | NM_015066 |
| TRIM9 | Tripartite motif-containing 9 | NM_015163 |
| TRIP | TRAF interacting protein | NM_005879 |
| TRPM5 | Transient receptor potential cation channel, | NM_014555 |
| | subfamily M, member 5 | |
| TRPM7 | Transient receptor potential cation channel, | NM_017672 |
| | subfamily M, member 7 | |
| TTC19 | Tetratricopeptide repeat domain 19 | BC066344 |
| TTR | Transthyretin (prealbumin, amyloidosis type I) | NM_000371 |
| TTYH2 | Tweety homolog 2 (Drosophila) | NM_032646 |
| TUBA1 | Tubulin, alpha 1 (testis specific) | NM_006000 |
| TUBB1 | Tubulin, beta 1 | NM_030773 |
| TUBB4 | Tubulin, beta 4 | NM_006087 |
| TXNIP | Thioredoxin interacting protein | NM_006472 |
| UBD | Ubiquitin D | NM_006398 |
| UBE2V1 | Ubiquitin-conjugating enzyme E2 variant 1 | NM_199144 |
| UBE3A | Ubiquitin protein ligase E3A (human papilloma | AF037219 |
| | virus E6-associated protein, Angelman | |
| | syndrome) | |
| UBL3 | Ubiquitin-like 3 | NM_007106 |
| UHSKerB | Keratin, ultrahigh sulfur, B | NM_021046 |
| ULK2 | Unc-51-like kinase 2 (C. elegans) | NM_014683 |
| URB | Steroid sensitive gene 1 | NM_199511 |
| USP54 | Ubiquitin specific protease 54 | NM_152586 |
| UST | Uronyl-2-sulfotransferase | NM_005715 |
| UTRN | Utrophin (homologous to dystrophin) | AK023675 |
| UTX | Ubiquitously transcribed tetratricopeptide repeat, | NM_021140 |
| | X chromosome | |
| VARS2L | Valyl-tRNA synthetase 2-like | NM_020442 |
| VAV1 | Vav 1 oncogene | NM_005428 |
| VGLL4 | Vestigial like 4 (Drosophila) | BQ013066 |
| VN1R1 | Vomeronasal 1 receptor 1 | NM_020633 |
| VSIG4 | V-set and immunoglobulin domain containing 4 | NM_007268 |
| WDR22 | WD repeat domain 22 | NM_003861 |
| WIF1 | WNT inhibitory factor 1 | NM_007191 |
| WWOX | WW domain containing oxidoreductase | AK094336 |
| XG | Xg blood group (pseudoautosomal boundary- | NM_175569 |
| | divided on the X chromosome) | |
| XIST | X (inactive)-specific transcript | AK025198 |
| XYLT2 | Xylosyltransferase II | NM_022167 |
| YPEL5 | Yippee-like 5 (Drosophila) | NM_016061 |
| ZBTB7 | Zinc finger and BTB domain containing 7 | NM_015898 |
| ZFHX1B | Zinc finger homeobox 1b | NM_014795 |
| ZFYVE26 | Zinc finger, FYVE domain containing 26 | NM_015346 |
| ZNF516 | Zinc finger protein 516 | D86975 |
| ZNF552 | Zinc finger protein 552 | AK023769 |
| ZNF572 | Zinc finger protein 572 | NM_152412 |
| ZP3 | Zona pellucida glycoprotein 3 (sperm receptor) | NM_007155 |
| ZSCAN2 | Zinc finger and SCAN domain containing 2 | NM_017894 |
| | No Annotation | A_23_BS113762 |
| | No Annotation | A_24_BS784213 |
| | No Annotation | A_24_BS926155 |
| | No Annotation | A_24_BS927614 |
| | No Annotation | A_24_BS934268 |
| | No Annotation | A_32_BS169243 |
| | No Annotation | A_32_BS200773 |
| | No Annotation | A_32_BS53976 |
| | No Annotation | A_32_BS73184 |
| | No Annotation | A_32_BS74588 |
| | No Annotation | AB065507 |
| | No Annotation | AC007051 |
| | No Annotation | AC007066 |
| | No Annotation | AC008453 |
| | No Annotation | AC025463 |
| | No Annotation | AC060234 |
| | No Annotation | AC087071 |
| | No Annotation | AC096677 |
| | Full length insert cDNA clone ZB81F12 | AF086167 |
| | No Annotation | AF089746 |
| | Amyloid lambda 6 light chain variable region | AF121762 |
| | SAR | |
| | IMAGE Consortium ID 839832, mRNA | AF124368 |
| | sequence | |
| | Clone FLB4246 PRO1102 mRNA, complete cds | AF130105 |
| | HSPC101 | AF161364 |
| | LOC440135 | AF318337 |
| | No Annotation | AF372624 |
| | No Annotation | AF533936 |
| | MRNA (fetal brain cDNA g6_1g) | AI791206 |
| | Hypothetical protein (ORF1), clone 00275 | AJ276555 |
| | No Annotation | AK001565 |
| | Hypothetical LOC388796 | AK022745 |
| | Homo sapiens, clone IMAGE: 4401608, mRNA | AK022793 |
| | Homo sapiens, clone IMAGE: 4214313, mRNA | AK022893 |
| | Homo sapiens, clone IMAGE: 5277945, mRNA | AK022997 |
| | CDNA: FLJ22769 fis, clone KAIA1316 | AK026422 |
| | CDNA FLJ31059 fis, clone HSYRA2000832 | AK055621 |
| | CDNA FLJ32177 fis, clone PLACE6001294 | AK056856 |
| | Homo sapiens, clone IMAGE: 5575764, mRNA | AK090500 |
| | Homo sapiens, clone IMAGE: 5575764, mRNA | AK092921 |
| | CDNA FLJ36725 fis, clone UTERU2012230 | AK094044 |
| | CDNA FLJ38235 fis, clone FCBBF2005428 | AK095554 |
| | CDNA FLJ25794 fis, clone TST07014 | AK098660 |
| | No Annotation | AL009178 |
| | MRNA; cDNA DKFZp566L0824 (from clone | AL050042 |
| | DKFZp566L0824) | |
| | No Annotation | AL109935 |
| | No Annotation | AL132874 |
| | Full-length cDNA clone CS0DJ001YJ05 of T | AL137761 |
| | cells (Jurkat cell line) Cot 10-normalized of | |
| | Homo sapiens (human) | |
| | No Annotation | AL391244 |
| | No Annotation | AL445486 |
| | No Annotation | AL591806 |
| | No Annotation | AL731541 |
| | No Annotation | AL928970 |
| | No Annotation | AY062331 |
| | No Annotation | AY372690 |
| | No Annotation | BC009051 |
| | LOC441164 | BC009220 |
| | CDNA clone IMAGE: 3462401, partial cds | BC010544 |
| | No Annotation | BC011367 |
| | No Annotation | BC015531 |
| | LOC440441 | BC020847 |
| | Homo sapiens, clone IMAGE: 5295565, mRNA, | BC031278 |
| | partial cds | |
| | Similar to jumonji domain containing 1A; testis- | BC035102 |
| | specific protein A; zinc finger protein | |
| | Homo sapiens, clone IMAGE: 5575764, mRNA | BC035647 |
| | Hypothetical LOC197387 | BC038761 |
| | Hypothetical gene supported by BC039664 | BC039664 |
| | No Annotation | BC107852 |
| | No Annotation | BG252130 |
| | Full-length cDNA clone CS0DI009YA14 of | BG327427 |
| | Placenta Cot 25-normalized of Homo sapiens | |
| | (human) | |
| | Hypothetical LOC339352 | BG620990 |
| | Similar to PI-3-kinase-related kinase SMG-1 | BI014689 |
| | isoform 2; lambda/iota protein kinase C- | |
| | interacting protein; phosphatidylinositol 3-kinase- | |
| | related protein kinase | |
| | Similar to D(1B) dopamine receptor (D(5) | BM561346 |
| | dopamine receptor) (D1beta dopamine receptor) | |
| | No Annotation | BM839360 |
| | Transcribed locus | BM925639 |
| | No Annotation | BM928667 |
| | Transcribed locus | BQ049338 |
| | No Annotation | BQ346290 |
| | Homo sapiens, clone IMAGE: 4838137, mRNA | BU587941 |
| | LOC441139 | BX118328 |
| | No Annotation | D80006 |
| | No Annotation | DQ101103 |
| | No Annotation | DQ188807 |
| | No Annotation | ENST00000242479 |
| | No Annotation | ENST00000246627 |
| | No Annotation | ENST00000259219 |
| | No Annotation | ENST00000259550 |
| | No Annotation | ENST00000293569 |
| | No Annotation | ENST00000296448 |
| | No Annotation | ENST00000298643 |
| | No Annotation | ENST00000299756 |
| | No Annotation | ENST00000300068 |
| | No Annotation | ENST00000305402 |
| | No Annotation | ENST00000305824 |
| | No Annotation | ENST00000307901 |
| | No Annotation | ENST00000308307 |
| | No Annotation | ENST00000310210 |
| | No Annotation | ENST00000312401 |
| | No Annotation | ENST00000312412 |
| | No Annotation | ENST00000312966 |
| | No Annotation | ENST00000313904 |
| | No Annotation | ENST00000318669 |
| | No Annotation | ENST00000321112 |
| | No Annotation | ENST00000321656 |
| | No Annotation | ENST00000322114 |
| | No Annotation | ENST00000322404 |
| | No Annotation | ENST00000322803 |
| | No Annotation | ENST00000324770 |
| | No Annotation | ENST00000325204 |
| | No Annotation | ENST00000325773 |
| | No Annotation | ENST00000327591 |
| | No Annotation | ENST00000327870 |
| | No Annotation | ENST00000328059 |
| | No Annotation | ENST00000328708 |
| | No Annotation | ENST00000329246 |
| | No Annotation | ENST00000329358 |
| | No Annotation | ENST00000329491 |
| | No Annotation | ENST00000329660 |
| | No Annotation | ENST00000330875 |
| | No Annotation | ENST00000331096 |
| | No Annotation | ENST00000331577 |
| | No Annotation | ENST00000331640 |
| | No Annotation | ENST00000332271 |
| | No Annotation | ENST00000332944 |
| | No Annotation | ENST00000332989 |
| | No Annotation | ENST00000333517 |
| | No Annotation | ENST00000333784 |
| | Transcribed locus, weakly similar to | H16080 |
| | NP_808455.1 hypothetical protein 9830102E05 | |
| | [Mus musculus] | |
| | No Annotation | I_1000437 |
| | No Annotation | I_1100650 |
| | No Annotation | I_1221777 |
| | No Annotation | I_1861543 |
| | No Annotation | I_1879042 |
| | No Annotation | I_1882608 |
| | No Annotation | I_1891291 |
| | No Annotation | I_1893151 |
| | No Annotation | I_1980505 |
| | No Annotation | I_1985061 |
| | No Annotation | I_3335767 |
| | No Annotation | I_3344109 |
| | No Annotation | I_3551568 |
| | No Annotation | I_3575384 |
| | No Annotation | I_3576071 |
| | No Annotation | I_3580313 |
| | No Annotation | I_3588329 |
| | No Annotation | I_930906 |
| | No Annotation | I_932413 |
| | No Annotation | I_943866 |
| | No Annotation | I_944092 |
| | No Annotation | I_962800 |
| | No Annotation | I_964340 |
| | No Annotation | I_966091 |
| | No Annotation | I_966691 |
| | No Annotation | M15073 |
| | No Annotation | M64260 |
| | No Annotation | NG_001019 |
| | No Annotation | NM_001005360 |
| | No Annotation | NM_001008528 |
| | No Annotation | NM_001009555 |
| | No Annotation | NM_001009569 |
| | No Annotation | NM_001010919 |
| | No Annotation | NM_001011708 |
| | No Annotation | NM_001013632 |
| | No Annotation | NM_001013680 |
| | No Annotation | NM_001014975 |
| | No Annotation | NM_001018006 |
| | No Annotation | NM_001018011 |
| | No Annotation | NM_001018076 |
| | No Annotation | NM_001024227 |
| | No Annotation | NM_001024465 |
| | No Annotation | NM_001024808 |
| | No Annotation | NM_001025077 |
| | No Annotation | NM_001025201 |
| | No Annotation | NM_001031677 |
| | No Annotation | NM_001033044 |
| | No Annotation | NM_001033569 |
| | No Annotation | NM_003671 |
| | No Annotation | NM_014758 |
| | No Annotation | NM_015262 |
| | No Annotation | NM_018350 |
| | No Annotation | NM_018506 |
| | No Annotation | NM_080432 |
| | No Annotation | NM_138411 |
| | No Annotation | NM_153030 |
| | No Annotation | NM_153237 |
| | No Annotation | NM_172020 |
| | No Annotation | NM_173705 |
| | No Annotation | NM_173709 |
| | No Annotation | NM_178429 |
| | No Annotation | NM_178467 |
| | No Annotation | NM_213595 |
| | No Annotation | NR_001544 |
| | No Annotation | NR_002184 |
| | No Annotation | NR_002225 |
| | Anti-HIV-1 gp120 V3 loop antibody DO142-10 | S62210 |
| | light chain variable region | |
| | No Annotation | S80864 |
| | No Annotation | THC1409898 |
| | No Annotation | THC1419743 |
| | No Annotation | THC1429821 |
| | No Annotation | THC1434038 |
| | No Annotation | THC1438453 |
| | No Annotation | THC1441583 |
| | No Annotation | THC1448600 |
| | No Annotation | THC1457058 |
| | No Annotation | THC1457118 |
| | No Annotation | THC1459712 |
| | No Annotation | THC1461073 |
| | No Annotation | THC1469536 |
| | No Annotation | THC1475763 |
| | No Annotation | THC1477639 |
| | No Annotation | THC1484458 |
| | No Annotation | THC1490378 |
| | No Annotation | THC1493219 |
| | No Annotation | THC1504780 |
| | No Annotation | THC1505917 |
| | No Annotation | THC1506312 |
| | No Annotation | THC1511927 |
| | No Annotation | THC1515028 |
| | No Annotation | THC1525318 |
| | No Annotation | THC1531579 |
| | No Annotation | THC1537124 |
| | No Annotation | THC1543691 |
| | No Annotation | THC1544941 |
| | No Annotation | THC1551463 |
| | No Annotation | THC1555359 |
| | No Annotation | THC1559236 |
| | No Annotation | THC1560798 |
| | No Annotation | THC1562602 |
| | No Annotation | THC1563147 |
| | No Annotation | THC1564329 |
| | No Annotation | THC1572906 |
| | No Annotation | THC1572972 |
| | No Annotation | THC1574967 |
| | No Annotation | THC1578318 |
| | No Annotation | THC1581022 |
| | No Annotation | THC1584122 |
| | No Annotation | THC1589164 |
| | No Annotation | THC1591470 |
| | Hypothetical gene LOC133874 | U31733 |
| | No Annotation | U62539 |
| | No Annotation | X68990 |
| | No Annotation | XM_065006 |
| | No Annotation | XM_165930 |
| | No Annotation | XM_170211 |
| | Similar to ARHQ protein | XM_209429 |
| | No Annotation | XM_210579 |
| | No Annotation | XM_291496 |
| | No Annotation | XM_291718 |
| | No Annotation | XM_295760 |
| | No Annotation | XM_301448 |
| | No Annotation | XM_303638 |
| | No Annotation | XM_305652 |
| | Similar to Tubulin beta-4q chain | XM_371684 |
| | Similar to CXYorf1-related protein | XM_377073 |
| | Similar to immunoglobulin M chain | Y11328 |

### Example 3

### Use of Linear Discriminant Analysis (LDA) to Distinguish the Diffuse-Proliferation and Inflammatory Groups

Genes that distinguished samples in the Diffuse-Proliferation and Inflammatory groups were selected using Linear Discriminant Analysis (LDA), described in Example 2, and the initial skin biopsy gene expression datasets. Examination of the expression data for single genes shows that the expression any one single gene may not always clearly distinguish between the groups of proliferation and no proliferation. In contrast, the multivariable LDA analysis results in LDA scores that separated the two groups more than by using the gene expression of single genes alone. Particularly in the case of testing the results of the LDA equation for the Inflammatory group in a separate dataset, the multivariate analysis resulted in clear separation of the two groups. This analysis therefore provides potential biomarkers in the skin for identifying the intrinsic subsets in SSc in new skin biopsies.

For the Diffuse-Proliferation group, LDA Score=−1.902(NM_{—}004703)-1.908(NM_{—}020422)+1.475(AGI_HUM1_OLIGO_A_{—}24_P690235)+1.83(NM_{—}173511), where NM_{—}004703 corresponds to RABEP1, NM_{—}020422 corresponds to promethin, AGI_HUM l_OLIGO_A_{—}24_P690235 refers to novel gene transcript ENST00000312412, and NM_{—}173511 refers to ALS2CR13.

For the Inflammatory group, LDA score=4.365(NM_{—}002119)+2.926(NM_{—}006851)−2.620(NM_{—}017570)+6.601(NM_{—}022163)+2.033(NM_{—}012110), where NM_{—}002119 refers to HLA-DOA, NM_{—}006851 refers to GLIPR1, NM_{—}017570 refers to OPLAH, NM_{—}022163 refers to MRPL46, and NM_{—}012110 refers to CHIC2.

### Example 4

### IL-13 and IL-4 Gene Signatures Identify the Inflammatory Subset

In addition to TGFβ, gene expression signatures associated with pro-fibrotic cytokines IL-13 (NM_{—}002188) and IL-4 (NM_{—}000589) were determined in cultured adult human dermal fibroblasts. The 490 genes of the IL-13 gene signature are presented in Table 4. The genes of the IL-4 gene signature are presented in Table 5. This analysis indicated that IL-13 and IL-4 share an approximately 60% overlap of inducible genes. In contrast, the TGFβ inducible signature was composed of a distinct set of gene expression targets demonstrating a 5% overlap with the IL-13 and IL-4 signatures.

Gene expression signatures were used to determine the potential drivers of fibrosis in a large well-controlled gene expression dataset of SSc skin biopsies, which were demonstrated herein as molecular subsets in scleroderma skin. The TGFβ signature was largely expressed in a subset of diffuse patients and was more highly expressed in patients with more severe skin disease (p<0.01) and scleroderma lung disease (p<0.01). The IL-13 and IL-4 gene expression signatures showed increased expression in the Inflammatory subset of SSc patients biopsies, and represent the earliest disease stages.

It is contemplated that fibrosis in different SSc subsets is driven by different molecular mechanisms tied to either TGFβ or IL-13 and IL-4. These finding indicate that patient subsetting is necessary in order to target different anti-fibrotic treatments based on molecular subclassifications of SSc patients.

TABLE 4

| Gene Symbol | Gene Name | Accession No. |
|---|---|---|
| ABCA6 | ATP-binding cassette, sub-family A (ABC1), member 6 | NM_080284 |
| ACTA1 | Actin, alpha 1, skeletal muscle | NM_001100 |
| ADAMTS1 | A disintegrin-like and metalloprotease (reprolysin type) | NM_006988 |
| | with thrombospondin type 1 motif, 1 | |
| ADCY4 | Adenylate cyclase 4 | NM_139247 |
| ADH1A | Alcohol dehydrogenase 1A (class I), alpha polypeptide | NM_000667 |
| ADRA2C | Adrenergic, alpha-2C-, receptor | NM_000683 |
| AHR | Aryl hydrocarbon receptor | NM_001621 |
| AKAP12 | A kinase (PRKA) anchor protein (gravin) 12 | NM_144497 |
| AMPH | Amphiphysin (Stiff-Man syndrome with breast cancer | NM_001635 |
| | 128 kDa autoantigen) | |
| ANGPTL4 | Angiopoietin-like 4 | NM_139314 |
| ANK1 | Ankyrin 1, erythrocytic | NM_020478 |
| ANLN | Anillin, actin binding protein (scraps homolog, | NM_018685 |
| | Drosophila) | |
| ANXA3 | Annexin A3 | NM_005139 |
| APCDD1 | Adenomatosis polyposis coli down-regulated 1 | NM_153000 |
| APOD | Apolipoprotein D | NM_001647 |
| APOH | Apolipoprotein H (beta-2-glycoprotein I) | NM_000042 |
| ARHGAP18 | Rho GTPase activating protein 18 | NM_033515 |
| ARHGDIB | Rho GDP dissociation inhibitor (GDI) beta | NM_001175 |
| ARNT2 | Aryl-hydrocarbon receptor nuclear translocator 2 | NM_014862 |
| ARRDC4 | Arrestin domain containing 4 | NM_183376 |
| ASB9 | Ankyrin repeat and SOCS box-containing 9 | NM_024087 |
| ASCL2 | Achaete-scute complex-like 2 (Drosophila) | NM_005170 |
| ASPA | Aspartoacylase (aminoacylase 2, Canavan disease) | NM_000049 |
| ASPM | Asp (abnormal spindle)-like, microcephaly associated | NM_018136 |
| | (Drosophila) | |
| ASPM | Asp (abnormal spindle)-like, microcephaly associated | NM_018136 |
| | (Drosophila) | |
| ATF3 | Activating transcription factor 3 | NM_004024 |
| ATF7IP2 | Activating transcription factor 7 interacting protein 2 | CR626222 |
| BCL11A | B-cell CLL/lymphoma 11A (zinc finger protein) | BU540282 |
| BDKRB1 | Bradykinin receptor B1 | NM_000710 |
| BDKRB1 | Bradykinin receptor B1 | NM_000710 |
| BDKRB2 | Bradykinin receptor B2 | NM_000623 |
| BIRC5 | Baculoviral IAP repeat-containing 5 (survivin) | BC007606 |
| BNC1 | Basonuclin 1 | NM_001717 |
| BNC2 | Basonuclin 2 | BC020879 |
| BNC2 | Basonuclin 2 | NM_017637 |
| BNC2 | Basonuclin 2 | NM_017637 |
| BSPRY | B-box and SPRY domain containing | NM_017688 |
| BUB1 | BUB1 budding uninhibited by benzimidazoles 1 | NM_004336 |
| | homolog (yeast) | |
| C10orf10 | Chromosome 10 open reading frame 10 | NM_007021 |
| C10orf3 | Chromosome 10 open reading frame 3 | NM_018131 |
| C10orf72 | Chromosome 10 open reading frame 72 | AK001062 |
| C13orf3 | Chromosome 13 open reading frame 3 | BC013418 |
| C18orf11 | Chromosome 18 open reading frame 11 | NM_022751 |
| C18orf11 | Chromosome 18 open reading frame 11 | NM_022751 |
| C18orf4 | Chromosome 18 open reading frame 4 | NM_032160 |
| C20orf129 | Chromosome 20 open reading frame 129 | NM_030919 |
| C21orf81 | Chromosome 21 open reading frame 81 | NM_153750 |
| C4BPA | Complement component 4 binding protein, alpha | NM_000715 |
| C5orf13 | Chromosome 5 open reading frame 13 | NM_004772 |
| C5orf4 | Chromosome 5 open reading frame 4 | NM_032385 |
| C8orf22 | Chromosome 8 open reading frame 22 | NM_001007176 |
| C9orf58 | Chromosome 9 open reading frame 58 | NM_001002260 |
| C9orf58 | Chromosome 9 open reading frame 58 | NM_001002260 |
| CA8 | Carbonic anhydrase VIII | NM_004056 |
| CAV1 | Caveolin 1, caveolae protein, 22 kDa | NM_001753 |
| CAV1 | Caveolin 1, caveolae protein, 22 kDa | NM_001753 |
| CCL2 | Chemokine (C-C motif) ligand 2 | NM_002982 |
| CCL26 | Chemokine (C-C motif) ligand 26 | NM_006072 |
| CCNB1 | Cyclin B1 | NM_031966 |
| CCNB2 | Cyclin B2 | NM_004701 |
| CCR1 | Chemokine (C-C motif) receptor 1 | NM_001295 |
| CCRL1 | Chemokine (C-C motif) receptor-like 1 | NM_178445 |
| CD200 | CD200 antigen | NM_001004196 |
| CD33 | CD33 antigen (gp67) | NM_001772 |
| CD38 | CD38 antigen (p45) | NM_001775 |
| CD3G | CD3G antigen, gamma polypeptide (TiT3 complex) | NM_000073 |
| CDC2 | Cell division cycle 2, G1 to S and G2 to M | NM_001786 |
| CDC20 | CDC20 cell division cycle 20 homolog (S. cerevisiae) | NM_001255 |
| CDC25C | Cell division cycle 25C | NM_001790 |
| CDC37L1 | Cell division cycle 37 homolog (S. cerevisiae)-like 1 | NM_017913 |
| CDCA2 | Cell division cycle associated 2 | NM_152562 |
| CDCA5 | Cell division cycle associated 5 | NM_080668 |
| CDCA8 | Cell division cycle associated 8 | NM_018101 |
| CDH1 | Cadherin 1, type 1, E-cadherin (epithelial) | NM_004360 |
| CDH18 | Cadherin 18, type 2 | NM_004934 |
| CDKN3 | Cyclin-dependent kinase inhibitor 3 (CDK2-associated | NM_005192 |
| | dual specificity phosphatase) | |
| CEACAM1 | Carcinoembryonic antigen-related cell adhesion | NM_001712 |
| | molecule 1 (biliary glycoprotein) | |
| CENPF | Centromere protein F, 350/400ka (mitosin) | NM_016343 |
| CGA | Glycoprotein hormones, alpha polypeptide | NM_000735 |
| CH25H | Cholesterol 25-hydroxylase | NM_003956 |
| CHST6 | Carbohydrate (N-acetylglucosamine 6-O) | NM_021615 |
| | sulfotransferase 6 | |
| CISH | Cytokine inducible SH2-containing protein | NM_145071 |
| CITED4 | Cbp/p300-interacting transactivator, with Glu/Asp-rich | NM_133467 |
| | carboxy-terminal domain, 4 | |
| CKLFSF8 | Chemokine-like factor super family 8 | NM_178868 |
| CLDN11 | Claudin 11 (oligodendrocyte transmembrane protein) | AF085871 |
| CMKOR1 | Chemokine orphan receptor 1 | NM_020311 |
| CNIH3 | Cornichon homolog 3 (Drosophila) | NM_152495 |
| COL4A6 | Collagen, type IV, alpha 6 | NM_033641 |
| COL8A2 | Collagen, type VIII, alpha 2 | NM_005202 |
| CP | Ceruloplasmin (ferroxidase) | NM_000096 |
| CPB2 | Carboxypeptidase B2 (plasma, carboxypeptidase U) | NM_001872 |
| CPXM2 | Carboxypeptidase X (M14 family), member 2 | NM_198148 |
| CTGF | Connective tissue growth factor | NM_001901 |
| CTNNAL1 | Catenin (cadherin-associated protein), alpha-like 1 | NM_003798 |
| CX3CL1 | Chemokine (C—X3—C motif) ligand 1 | NM_002996 |
| CX3CR1 | Chemokine (C—X3—C motif) receptor 1 | NM_001337 |
| CXCL1 | Chemokine (C—X—C motif) ligand 1 (melanoma growth | NM_001511 |
| | stimulating activity, alpha) | |
| CXCL14 | Chemokine (C—X—C motif) ligand 14 | NM_004887 |
| CXCR4 | chemokine (C—X—C motif) receptor 4 | NM_001008540 |
| CYP2F1 | Cytochrome P450, family 2, subfamily F, polypeptide 1 | NM_000774 |
| DCAMKL1 | Doublecortin and CaM kinase-like 1 | NM_004734 |
| DCN | Decorin | BQ004014 |
| DKFZP434B061 | DKFZP434B061 protein | AL117481 |
| DKFZP434I216 | DKFZP434I216 protein | NM_015432 |
| DKFZp564I1922 | Adlican | NM_015419 |
| DKFZP586A0522 | DKFZP586A0522 protein | NM_014033 |
| DKFZP586A0522 | DKFZP586A0522 protein | NM_014033 |
| DKFZP586K1520 | DKFZP586K1520 protein | AL050153 |
| DLG7 | Discs, large homolog 7 (Drosophila) | NM_014750 |
| DMD | Dystrophin (muscular dystrophy, Duchenne and Becker | NM_004010 |
| | types) | |
| DOK1 | Docking protein 1, 62 kDa (downstream of tyrosine | NM_001381 |
| | kinase 1) | |
| DRCTNNB1A | Down-regulated by Ctnnb1, a | NM_032581 |
| DUSP6 | Dual specificity phosphatase 6 | NM_001946 |
| ECHDC3 | Enoyl Coenzyme A hydratase domain containing 3 | NM_024693 |
| ECM2 | Extracellular matrix protein 2, female organ and | NM_001393 |
| | adipocyte specific | |
| EDN1 | Endothelin 1 | NM_001955 |
| EFNB2 | Ephrin-B2 | NM_004093 |
| EGLN3 | Egl nine homolog 3 (C. elegans) | NM_022073 |
| EGR1 | Early growth response 1 | NM_001964 |
| EN1 | Engrailed homolog 1 | NM_001426 |
| ENC1 | Ectodermal-neural cortex (with BTB-like domain) | NM_003633 |
| ENC1 | Ectodermal-neural cortex (with BTB-like domain) | NM_003633 |
| EPHA4 | EPH receptor A4 | NM_004438 |
| EPHX2 | Epoxide hydrolase 2, cytoplasmic | NM_001979 |
| EXOSC8 | Exosome component 8 | NM_181503 |
| EXOSC8 | Exosome component 8 | NM_181503 |
| FABP1 | Fatty acid binding protein 1, liver | NM_001443 |
| FADS1 | Fatty acid desaturase 1 | NM_013402 |
| FBXO32 | F-box protein 32 | NM_058229 |
| FCGR2A | Fc fragment of IgG, low affinity IIa, receptor for | NM_021642 |
| | (CD32) | |
| FGF7 | Galactokinase 2 | NM_002009 |
| FGF7 | Galactokinase 2 | NM_002009 |
| FGF7 | Galactokinase 2 | NM_002009 |
| FHL2 | Four and a half LIM domains 2 | NM_201555 |
| FKSG14 | Leucine zipper protein FKSG14 | NM_022145 |
| FLJ10156 | Hypothetical protein FLJ10156 | NM_019013 |
| FLJ13391 | Hypothetical protein FLJ13391 | NM_032181 |
| FLJ14712 | Hypothetical protein FLJ14712 | AK027618 |
| FLJ20255 | Hypothetical protein FLJ20255 | AK000262 |
| FLJ31340 | Hypothetical protein FLJ31340 | NM_152748 |
| FLJ35767 | FLJ35767 protein | NM_207459 |
| FLJ36031 | Hypothetical protein FLJ36031 | AK098422 |
| FLJ36031 | Hypothetical protein FLJ36031 | NM_175884 |
| FLJ37478 | Hypothetical protein FLJ37478 | NM_178557 |
| FLJ40629 | Hypothetical protein FLJ40629 | NM_152515 |
| FMN | Formin (limb deformity) | BC029107 |
| FOXQ1 | Forkhead box Q1 | NM_033260 |
| FZD10 | Frizzled homolog 10 (Drosophila) | NM_007197 |
| FZD4 | Frizzled homolog 4 (Drosophila) | NM_012193 |
| G2 | G2 protein | U10991 |
| GAL | Galanin | NM_015973 |
| GAS1 | Growth arrest-specific 1 | NM_002048 |
| GATA6 | GATA binding protein 6 | NM_005257 |
| GDF3 | Growth differentiation factor 3 | NM_020634 |
| GEM | GTP binding protein overexpressed in skeletal muscle | NM_005261 |
| GLCCI1 | Glucocorticoid induced transcript 1 | NM_138426 |
| GNG11 | Guanine nucleotide binding protein (G protein), gamma | NM_004126 |
| | 11 | |
| GPR68 | G protein-coupled receptor 68 | NM_003485 |
| GREM1 | Gremlin 1 homolog, cysteine knot superfamily | NM_013372 |
| | (Xenopus laevis) | |
| GSG1 | Germ cell associated 1 | NM_031289 |
| GTSE1 | G-2 and S-phase expressed 1 | NM_016426 |
| HAS3 | Hyaluronan synthase 3 | NM_005329 |
| HCAP-G | Chromosome condensation protein G | NM_022346 |
| HES1 | Hairy and enhancer of split 1, (Drosophila) | NM_005524 |
| HIST1H4B | Histone 1, H4b | NM_003544 |
| HIST1H4C | Histone 1, H4c | NM_003542 |
| HIST1H4L | Histone 1, H4l | NM_003546 |
| HLF | Hepatic leukemia factor | NM_002126 |
| HMMR | Hyaluronan-mediated motility receptor (RHAMM) | NM_012484 |
| HRH1 | Histamine receptor H1 | NM_000861 |
| HT008 | Uncharacterized hypothalamus protein HT008 | NM_018469 |
| ICA1 | Islet cell autoantigen 1, 69 kDa | NM_004968 |
| ICAM5 | Intercellular adhesion molecule 5, telencephalin | NM_003259 |
| ID1 | Inhibitor of DNA binding 1, dominant negative helix- | NM_002165 |
| | loop-helix protein | |
| IFI44 | Interferon-induced protein 44 | NM_006417 |
| IL6 | Interleukin 6 (interferon, beta 2) | NM_000600 |
| INSIG2 | Insulin induced gene 2 | NM_016133 |
| INSIG2 | Insulin induced gene 2 | NM_016133 |
| IRF5 | Interferon regulatory factor 5 | NM_002200 |
| JAG1 | Jagged 1 (Alagille syndrome) | NM_000214 |
| KCNH2 | Potassium voltage-gated channel, subfamily H (eag- | NM_000238 |
| | related), member 2 | |
| KCNMB4 | Potassium large conductance calcium-activated | NM_014505 |
| | channel, subfamily M, beta member 4 | |
| KCTD12 | Potassium channel tetramerisation domain containing | NM_138444 |
| | 12 | |
| KIAA0101 | KIAA0101 | NM_014736 |
| KIAA1199 | KIAA1199 | NM_018689 |
| KIAA1199 | KIAA1199 | NM_018689 |
| KIAA1217 | KIAA1217 | AK022045 |
| KIAA1217 | KIAA1217 | NM_019590 |
| KIAA1509 | KIAA1509 | AB040942 |
| KIAA1644 | KIAA1644 protein | AB051431 |
| KIAA1666 | KIAA1666 protein | BC035246 |
| KIAA1913 | KIAA1913 | BC044246 |
| KIF18A | Kinesin family member 18A | NM_031217 |
| KIF20A | Kinesin family member 20A | NM_005733 |
| KIF2C | Kinesin family member 2C | NM_006845 |
| KIF4A | Kinesin family member 4A | NM_012310 |
| KLF2 | Kruppel-like factor 2 (lung) | NM_016270 |
| KLK8 | Kallikrein 8 (neuropsin/ovasin) | NM_144505 |
| KLRC1 | Killer cell lectin-like receptor subfamily C, member 1 | NM_002259 |
| KNTC2 | Kinetochore associated 2 | NM_006101 |
| KRT23 | Keratin 23 (histone deacetylase inducible) | NM_015515 |
| KRTAP1-5 | Keratin associated protein 1-5 | NM_031957 |
| LAD1 | Ladinin 1 | NM_005558 |
| LAMA2 | Laminin, alpha 2 (merosin, congenital muscular | NM_000426 |
| | dystrophy) | |
| LEF1 | Lymphoid enhancer-binding factor 1 | NM_016269 |
| LHX2 | LIM homeobox 2 | NM_004789 |
| LIPE | Lipase, hormone-sensitive | NM_005357 |
| LMNB1 | Lamin B1 | NM_005573 |
| LOC126755 | Hypothetical protein LOC126755 | CR622769 |
| LOC150166 | Hypothetical protein LOC150166 | AK056836 |
| LOC150271 | Hypothetical LOC388889 | AK098753 |
| LOC199964 | Hypothetical protein LOC199964 | NM_182532 |
| LOC222171 | Hypothetical protein LOC222171 | NM_175887 |
| LOC255480 | Hypothetical protein LOC255480 | AK091766 |
| LOC284018 | Hypothetical protein LOC284018 | NM_181655 |
| LOC285733 | Hypothetical protein LOC285733 | AK091900 |
| LOC286254 | Hypothetical protein LOC286254 | AK092751 |
| LOC51334 | Mesenchymal stem cell protein DSC54 | NM_016644 |
| LOXL3 | Lysyl oxidase-like 3 | NM_032603 |
| LOXL3 | Lysyl oxidase-like 3 | NM_032603 |
| LPXN | Leupaxin | NM_004811 |
| LRP8 | Low density lipoprotein receptor-related protein 8, | NM_033300 |
| | apolipoprotein e receptor | |
| LYZ | Lysozyme (renal amyloidosis) | NM_000239 |
| LZTS1 | Leucine zipper, putative tumor suppressor 1 | NM_021020 |
| MAD2L1 | MAD2 mitotic arrest deficient-like 1 (yeast) | NM_002358 |
| MAFB | V-maf musculoaponeurotic fibrosarcoma oncogene | NM_005461 |
| | homolog B (avian) | |
| MAGEA1 | Melanoma antigen, family A, 1 (directs expression of | NM_004988 |
| | antigen MZ2-E) | |
| MAL2 | Mal, T-cell differentiation protein 2 | NM_052886 |
| MAOB | Monoamine oxidase B | NM_000898 |
| MAP3K8 | Mitogen-activated protein kinase kinase kinase 8 | NM_005204 |
| MARLIN1 | Multiple coiled-coil GABABR1-binding protein | NM_144720 |
| MEST | Mesoderm specific transcript homolog (mouse) | NM_002402 |
| MGAT3 | Mannosyl (beta-1,4-)-glycoprotein beta-1,4-N- | AK125361 |
| | acetylglucosaminyltransferase | |
| MGC13040 | Hypothetical protein MGC13040 | NM_032930 |
| MGC22265 | Hypothetical protein MGC22265 | BC048193 |
| MGC2574 | Hypothetical protein MGC2574 | NM_024098 |
| MGC2574 | Hypothetical protein MGC2574 | NM_024098 |
| MGC33365 | Hypothetical protein MGC33365 | NM_173552 |
| MLANA | Melan-A | NM_005511 |
| MMP12 | Matrix metalloproteinase 12 (macrophage elastase) | NM_002426 |
| MSX1 | Msh homeo box homolog 1 (Drosophila) | NM_002448 |
| MT1B | Metallothionein 1B (functional) | NM_005947 |
| MT1E | Metallothionein 1E (functional) | NM_175617 |
| MT1G | Metallothionein 1G | NM_005950 |
| MT1K | Metallothionein 1K | NM_176870 |
| MT1L | Metallothionein 1L | X97261 |
| MT1X | Metallothionein 1X | NM_005952 |
| MT2A | Metallothionein 2A | NM_005953 |
| MT2A | Metallothionein 2A | NM_005953 |
| MTL5 | Metallothionein-like 5, testis-specific (tesmin) | NM_004923 |
| MYCN | V-myc myelocytomatosis viral related oncogene, | NM_005378 |
| | neuroblastoma derived (avian) | |
| MYO10 | Myosin X | NM_012334 |
| MYO10 | Myosin X | NM_012334 |
| MYO5B | Myosin VB | AK025336 |
| MYO5C | Myosin VC | NM_018728 |
| MYRIP | Myosin VIIA and Rab interacting protein | NM_015460 |
| NAV2 | Neuron navigator 2 | NM_182964 |
| NET1 | Neuroepithelial cell transforming gene 1 | NM_005863 |
| NETO2 | Neuropilin (NRP) and tolloid (TLL)-like 2 | NM_018092 |
| NFE2 | Nuclear factor (erythroid-derived 2), 45 kDa | NM_006163 |
| NFIL3 | Nuclear factor, interleukin 3 regulated | NM_005384 |
| NGEF | Neuronal guanine nucleotide exchange factor | NM_019850 |
| NID2 | Nidogen 2 (osteonidogen) | NM_007361 |
| NOSTRIN | Nitric oxide synthase trafficker | NM_052946 |
| NOV | Nephroblastoma overexpressed gene | NM_002514 |
| NR0B1 | Nuclear receptor subfamily 0, group B, member 1 | NM_000475 |
| NR0B2 | Nuclear receptor subfamily 0, group B, member 2 | NM_021969 |
| NSE1 | NSE1 | NM_145175 |
| NTN4 | Netrin 4 | NM_021229 |
| NTS | Neurotensin | NM_006183 |
| ODZ3 | Odz, odd Oz/ten-m homolog 3 (Drosophila) | AB040888 |
| ODZ3 | Odz, odd Oz/ten-m homolog 3 (Drosophila) | AB040888 |
| OIP5 | Opa-interacting protein 5 | NM_007280 |
| OLFML2A | Olfactomedin-like 2A | NM_182487 |
| OR7E140P | Olfactory receptor, family 7, subfamily E, member 140 | BC073935 |
| | pseudogene | |
| OVOS2 | Ovostatin 2 | BC039117 |
| PAG | Phosphoprotein associated with glycosphingolipid- | NM_018440 |
| | enriched microdomains | |
| PBEF1 | Pre-B-cell colony enhancing factor 1 | NM_005746 |
| PBEF1 | Pre-B-cell colony enhancing factor 1 | NM_182790 |
| PCANAP6 | Prostate cancer associated protein 6 | NM_033102 |
| PCSK5 | Proprotein convertase subtilisin/kexin type 5 | NM_006200 |
| PDGFA | Platelet-derived growth factor alpha polypeptide | NM_002607 |
| PDGFC | Platelet derived growth factor C | NM_016205 |
| PDGFD | DNA-damage inducible protein 1 | NM_025208 |
| PHACTR1 | Phosphatase and actin regulator 1 | NM_030948 |
| PHLDA1 | Pleckstrin homology-like domain, family A, member 1 | NM_007350 |
| PHLDA1 | Pleckstrin homology-like domain, family A, member 1 | NM_007350 |
| PHLDB2 | Pleckstrin homology-like domain, family B, member 2 | NM_145753 |
| PIK3R1 | Phosphoinositide-3-kinase, regulatory subunit 1 (p85 | NM_181523 |
| | alpha) | |
| PIM1 | Pim-1 oncogene | NM_002648 |
| PKD1L2 | Polycystic kidney disease 1-like 2 | NM_052892 |
| PKD2 | Polycystic kidney disease 2 (autosomal dominant) | NM_000297 |
| PLAC8 | Placenta-specific 8 | NM_016619 |
| PLAC8 | Placenta-specific 8 | NM_016619 |
| PLD1 | Phospholipase D1, phophatidylcholine-specific | NM_002662 |
| PLK2 | Polo-like kinase 2 (Drosophila) | NM_006622 |
| PLP1 | Proteolipid protein 1 (Pelizaeus-Merzbacher disease, | M54927 |
| | spastic paraplegia 2, uncomplicated) | |
| PMAIP1 | Phorbol-12-myristate-13-acetate-induced protein 1 | NM_021127 |
| PPP1R1A | Protein phosphatase 1, regulatory (inhibitor) subunit 1A | NM_006741 |
| PPP1R3B | Protein phosphatase 1, regulatory (inhibitor) subunit 3B | AK091994 |
| PPP2R3A | Protein phosphatase 2 (formerly 2A), regulatory subunit | NM_002718 |
| | B″, alpha | |
| PRC1 | Protein regulator of cytokinesis 1 | NM_003981 |
| PREX1 | Phosphatidylinositol 3,4,5-trisphosphate-dependent | NM_020820 |
| | RAC exchanger 1 | |
| PRKCB1 | Protein kinase C, beta 1 | NM_002738 |
| PRKCB1 | Protein kinase C, beta 1 | NM_002738 |
| PROC | Protein C (inactivator of coagulation factors Va and | NM_000312 |
| | VIIIa) | |
| PSCDBP | Pleckstrin homology, Sec7 and coiled-coil domains, | NM_004288 |
| | binding protein | |
| PSD3 | Pleckstrin and Sec7 domain containing 3 | NM_015310 |
| PSG11 | Pregnancy specific beta-1-glycoprotein 11 | NM_002785 |
| PSG3 | Pregnancy specific beta-1-glycoprotein 3 | NM_021016 |
| PTGER4 | Prostaglandin E receptor 4 (subtype EP4) | NM_000958 |
| PTGFR | Prostaglandin F receptor (FP) | NM_000959 |
| PTTG1 | Pituitary tumor-transforming 1 | NM_004219 |
| PTTG2 | Pituitary tumor-transforming 2 | NM_006607 |
| RAB11FIP2 | RAB11 family interacting protein 2 (class I) | NM_014904 |
| RACGAP1 | Rac GTPase activating protein 1 | NM_013277 |
| RAD52B | RAD52 homolog B (S. cerevisiae) | NM_145654 |
| RAMP1 | Receptor (calcitonin) activity modifying protein 1 | NM_005855 |
| RANBP9 | RAN binding protein 9 | NM_005493 |
| RANBP9 | RAN binding protein 9 | NM_005493 |
| RANBP9 | RAN binding protein 9 | NM_005493 |
| RASD1 | RAS, dexamethasone-induced 1 | NM_016084 |
| REV3L | REV3-like, catalytic subunit of DNA polymerase zeta | NM_002912 |
| | (yeast) | |
| RGS2 | Regulator of G-protein signalling 2, 24 kDa | NM_002923 |
| RIMS3 | Regulating synaptic membrane exocytosis 3 | NM_014747 |
| RIPK3 | Receptor-interacting serine-threonine kinase 3 | NM_006871 |
| RIPK4 | Receptor-interacting serine-threonine kinase 4 | NM_020639 |
| ROBO3 | Roundabout, axon guidance receptor, homolog 3 | NM_022370 |
| | (Drosophila) | |
| RPESP | RPE-spondin | NM_153225 |
| RRM2 | Ribonucleotide reductase M2 polypeptide | NM_001034 |
| RTN4R | Reticulon 4 receptor | NM_023004 |
| SALL2 | Sal-like 2 (Drosophila) | NM_005407 |
| SAMSN1 | SAM domain, SH3 domain and nuclear localisation | NM_022136 |
| | signals, 1 | |
| SATB1 | Special AT-rich sequence binding protein 1 (binds to | NM_002971 |
| | nuclear matrix/scaffold-associating DNA's) | |
| SCIN | Scinderin | NM_033128 |
| SECTM1 | Secreted and transmembrane 1 | NM_003004 |
| SEMA6A | Sema domain, transmembrane domain (TM), and | NM_020796 |
| | cytoplasmic domain, (semaphorin) 6A | |
| SEPP1 | Selenoprotein P, plasma, 1 | NM_005410 |
| SERPINA5 | Serine (or cysteine) proteinase inhibitor, clade A | NM_000624 |
| | (alpha-1 antiproteinase, antitrypsin), member 5 | |
| SERPINA7 | Serine (or cysteine) proteinase inhibitor, clade A | NM_000354 |
| | (alpha-1 antiproteinase, antitrypsin), member 7 | |
| SH2D1A | SH2 domain protein 1A, Duncan's disease | NM_002351 |
| | (lymphoproliferative syndrome) | |
| SLC16A6 | Solute carrier family 16 (monocarboxylic acid | NM_004694 |
| | transporters), member 6 | |
| SLC1A1 | Solute carrier family 1 (neuronal/epithelial high affinity | NM_004170 |
| | glutamate transporter, system Xag), member 1 | |
| SLC20A1 | Solute carrier family 20 (phosphate transporter), | NM_005415 |
| | member 1 | |
| SLC2A1 | Solute carrier family 2 (facilitated glucose transporter), | NM_006516 |
| | member 1 | |
| SLC39A8 | Solute carrier family 39 (zinc transporter), member 8 | NM_022154 |
| SLC40A1 | Solute carrier family 40 (iron-regulated transporter), | NM_014585 |
| | member 1 | |
| SLC7A5 | Solute carrier family 7 (cationic amino acid transporter, | NM_003486 |
| | y+ system), member 5 | |
| SLC9A9 | Solute carrier family 9 (sodium/hydrogen exchanger), | NM_173653 |
| | isoform 9 | |
| SLIT3 | Slit homolog 3 (Drosophila) | BC032027 |
| SLPI | Secretory leukocyte protease inhibitor | NM_003064 |
| | (antileukoproteinase) | |
| SMOC1 | SPARC related modular calcium binding 1 | NM_022137 |
| SMOC2 | SPARC related modular calcium binding 2 | NM_022138 |
| SNAI2 | Snail homolog 2 (Drosophila) | NM_003068 |
| SNFT | Jun dimerization protein p21SNFT | NM_018664 |
| SOCS1 | Suppressor of cytokine signaling 1 | NM_003745 |
| SORL1 | Sortilin-related receptor, L(DLR class) A repeats- | NM_003105 |
| | containing | |
| SOX4 | SRY (sex determining region Y)-box 4 | AW946823 |
| SOX4 | SRY (sex determining region Y)-box 4 | NM_003107 |
| SOX4 | SRY (sex determining region Y)-box 4 | NM_003107 |
| SP5 | Sp5 transcription factor | NM_001003845 |
| Spc25 | Kinetochore protein Spc25 | NM_020675 |
| SPHK1 | Sphingosine kinase 1 | NM_021972 |
| SPINT2 | Serine protease inhibitor, Kunitz type, 2 | NM_021102 |
| SRC | V-src sarcoma (Schmidt-Ruppin A-2) viral oncogene | NM_005417 |
| | homolog (avian) | |
| STAC | SH3 and cysteine rich domain | NM_003149 |
| STC2 | Stanniocalcin 2 | NM_003714 |
| STMN1 | Stathmin 1/oncoprotein 18 | NM_203401 |
| T3JAM | TRAF3-interacting Jun N-terminal kinase (JNK)- | NM_025228 |
| | activating modulator | |
| TCEAL7 | Transcription elongation factor A (SII)-like 7 | NM_152278 |
| TCF4 | Transcription factor 4 | AK021980 |
| TIGD2 | Tigger transposable element derived 2 | NM_145715 |
| TIMP3 | Tissue inhibitor of metalloproteinase 3 (Sorsby fundus | AA837799 |
| | dystrophy, pseudoinflammatory) | |
| TK1 | Thymidine kinase 1, soluble | NM_003258 |
| TM4SF1 | Transmembrane 4 superfamily member 1 | NM_014220 |
| TMPRSS4 | Transmembrane protease, serine 4 | NM_019894 |
| TMSNB | Thymosin, beta, identified in neuroblastoma cells | NM_021992 |
| TNC | Tenascin C (hexabrachion) | NM_002160 |
| TncRNA | Trophoblast-derived noncoding RNA | U60873 |
| TNFAIP6 | Tumor necrosis factor, alpha-induced protein 6 | NM_007115 |
| TNFRSF17 | Tumor necrosis factor receptor superfamily, member 17 | NM_001192 |
| TOP2A | Topoisomerase (DNA) II alpha 170 kDa | NM_001067 |
| TOPK | T-LAK cell-originated protein kinase | NM_018492 |
| TPD52 | Tumor protein D52 | NM_005079 |
| TPM1 | Tropomyosin 1 (alpha) | NM_000366 |
| TPX2 | TPX2, microtubule-associated protein homolog | NM_012112 |
| | (Xenopus laevis) | |
| TRIB1 | Tribbles homolog 1 (Drosophila) | NM_025195 |
| TRIB2 | Tribbles homolog 2 (Drosophila) | NM_021643 |
| TROAP | Trophinin associated protein (tastin) | NM_005480 |
| TRPS1 | Trichorhinophalangeal syndrome I | NM_014112 |
| TTK | TTK protein kinase | NM_003318 |
| TXNIP | Thioredoxin interacting protein | NM_006472 |
| TYRP1 | Tyrosinase-related protein 1 | NM_000550 |
| UAP1 | UDP-N-acteylglucosamine pyrophosphorylase 1 | NM_003115 |
| UBD | Ubiquitin D | NM_006398 |
| UBE2C | Ubiquitin-conjugating enzyme E2C | NM_181803 |
| UGT2B11 | UDP glycosyltransferase 2 family, polypeptide B11 | NM_001073 |
| UST | Uronyl-2-sulfotransferase | NM_005715 |
| UTS2 | Urotensin 2 | NM_021995 |
| UTS2 | Urotensin 2 | NM_021995 |
| VIL1 | Villin 1 | NM_007127 |
| YPEL4 | Yippee-like 4 (Drosophila) | NM_145008 |
| ZAP70 | Zeta-chain (TCR) associated protein kinase 70 kDa | NM_001079 |
| ZNF179 | Zinc finger protein 179 | NM_007148 |
| ZNF503 | Zinc finger protein 503 | NM_032772 |
| | | A_23_P15226 |
| | | A_23_P170719 |
| | | A_23_P43744 |
| | | A_24_P290087 |
| | | A_24_P686014 |
| | | A_24_P927205 |
| | | A_32_P182135 |
| | | A_32_P205792 |
| | | A_32_P225328 |
| | | A_32_P232647 |
| | | A_32_P55438 |
| | | AF256215 |
| | Hypothetical gene supported by AK026189 | AK022865 |
| | CDNA: FLJ22994 fis, clone KAT11918 | AK026647 |
| | CDNA: FLJ23131 fis, clone LNG08502 | AK026784 |
| | CDNA FLJ31059 fis, clone HSYRA2000832 | AK055621 |
| | Hypothetical LOC388397 | AK057167 |
| | Homo sapiens, clone IMAGE: 4214962, mRNA | AK091547 |
| | CDNA FLJ41489 fis, clone BRTHA2004582 | AK123483 |
| | MRNA full length insert cDNA clone EUROIMAGE | AK124841 |
| | 51148 | |
| | CDNA FLJ43172 fis, clone FCBBF3007242 | AK125162 |
| | CDNA FLJ26031 fis, clone PNC08078 | AK129542 |
| | Homo sapiens, clone IMAGE: 5285282, mRNA | AK129982 |
| | Similar to bA110H4.2 (similar to membrane protein) | AK130705 |
| | Transcribed locus | AW972815 |
| | Hypothetical gene supported by AY007155 | AY007155 |
| | Homo sapiens, clone IMAGE: 3869276, mRNA | BC018597 |
| | CDNA clone MGC: 65154 IMAGE: 5122136, complete | BC056907 |
| | cds | |
| | | BE893137 |
| | Transcribed locus, moderately similar to XP_497060.1 | BM989848 |
| | similar to FKSG60 [Homo sapiens] | |
| | Full-length cDNA clone CS0DJ001YJ05 of T cells | CR601458 |
| | (Jurkat cell line) Cot 10-normalized of Homo sapiens | |
| | (human) | |
| | Full-length cDNA clone CS0DC002YA18 of | CR624517 |
| | Neuroblastoma Cot 25-normalized of Homo sapiens | |
| | (human) | |
| | | CR936791 |
| | | CR936791 |
| | | CX788817 |
| | | ENST00000245185 |
| | | ENST00000261569 |
| | | ENST00000312275 |
| | | ENST00000314238 |
| | | ENST00000343505 |
| | | ENST00000371256 |
| | | ENST00000371655 |
| | | ENST00000375377 |
| | | ENST00000381889 |
| | | NM_001006641 |
| | | NM_001008708 |
| | | NM_001010911 |
| | | NM_001010915 |
| | | NM_001011543 |
| | | NM_001012271 |
| | | NM_001017420 |
| | | NM_001017424 |
| | | NM_001017535 |
| | | NM_001040100 |
| | | NM_001040167 |
| | | NM_001040457 |
| | | NM_002263 |
| | | NM_003621 |
| | | NM_014867 |
| | | NM_017577 |
| | | NM_020872 |
| | | NM_020872 |
| | | NM_020872 |
| | | NM_025135 |
| | | NM_032199 |
| | | NM_032532 |
| | | NR_001558 |
| | | THC2274524 |
| | | THC2308675 |
| | | THC2343246 |
| | | THC2347909 |
| | | THC2373845 |
| | | THC2376729 |
| | | THC2398598 |
| | | THC2405710 |
| | | THC2406576 |
| | | THC2407823 |
| | | THC2438492 |
| | | THC2438512 |
| | | THC2442210 |
| | | THC2442586 |
| | | THC2443654 |
| | | THC2455149 |
| | Similar to hypothetical protein LOC231503 | XM_496707 |
| | | XM_932314 |

TABLE 5

| Gene Symbol | Gene Name | Accession No. |
|---|---|---|
| ABCA6 | ATP-binding cassette, sub-family A (ABC1), member 6 | NM_080284 |
| ADAMTS1 | A disintegrin-like and metalloprotease (reprolysin type) | NM_006988 |
| | with thrombospondin type 1 motif, 1 | |
| ADAMTS1 | A disintegrin-like and metalloprotease (reprolysin type) | NM_006988 |
| | with thrombospondin type 1 motif, 1 | |
| ADCY4 | Adenylate cyclase 4 | NM_139247 |
| AFAP | Hypothetical protein LOC254848 | BC014113 |
| AGR2 | Anterior gradient 2 homolog (Xenopus laevis) | NM_006408 |
| ALOX5AP | Arachidonate 5-lipoxygenase-activating protein | NM_001629 |
| AMD1 | Adenosylmethionine decarboxylase 1 | NM_001634 |
| ANGPTL4 | Angiopoietin-like 4 | NM_139314 |
| ANK1 | Ankyrin 1, erythrocytic | NM_020478 |
| ANK3 | Ankyrin 3, node of Ranvier (ankyrin G) | NM_020987 |
| ANLN | Anillin, actin binding protein (scraps homolog, | NM_018685 |
| | Drosophila) | |
| ANXA3 | Annexin A3 | NM_005139 |
| APCDD1 | Adenomatosis polyposis coli down-regulated 1 | NM_153000 |
| APOBEC3B | Apolipoprotein B mRNA editing enzyme, catalytic | NM_004900 |
| | polypeptide-like 3B | |
| APOL6 | Apolipoprotein L, 6 | NM_030641 |
| AREG | Amphiregulin (schwannoma-derived growth factor) | NM_001657 |
| ARHGDIB | Rho GDP dissociation inhibitor (GDI) beta | NM_001175 |
| ARL4A | ADP-ribosylation factor-like 4A | NM_005738 |
| ARRDC4 | Arrestin domain containing 4 | NM_183376 |
| ASB9 | Ankyrin repeat and SOCS box-containing 9 | NM_024087 |
| ASPA | Aspartoacylase (aminoacylase 2, Canavan disease) | NM_000049 |
| ASPM | Asp (abnormal spindle)-like, microcephaly associated | NM_018136 |
| | (Drosophila) | |
| ASPM | Asp (abnormal spindle)-like, microcephaly associated | NM_018136 |
| | (Drosophila) | |
| ASRGL1 | Asparaginase like 1 | BC006267 |
| ASRGL1 | Asparaginase like 1 | NM_025080 |
| ATF3 | Activating transcription factor 3 | NM_004024 |
| BCL11A | B-cell CLL/lymphoma 11A (zinc finger protein) | BU540282 |
| BCL11A | B-cell CLL/lymphoma 11A (zinc finger protein) | NM_022893 |
| BDKRB1 | Bradykinin receptor B1 | NM_000710 |
| BDKRB2 | Bradykinin receptor B2 | NM_000623 |
| BIRC5 | Baculoviral IAP repeat-containing 5 (survivin) | BC007606 |
| BNC1 | Basonuclin 1 | NM_001717 |
| BNC2 | Basonuclin 2 | BC020879 |
| BNC2 | Basonuclin 2 | NM_017637 |
| BUB1 | BUB1 budding uninhibited by benzimidazoles 1 | NM_004336 |
| | homolog (yeast) | |
| C10orf3 | Chromosome 10 open reading frame 3 | NM_018131 |
| C13orf3 | Chromosome 13 open reading frame 3 | BC013418 |
| C18orf11 | Chromosome 18 open reading frame 11 | NM_022751 |
| C20orf103 | Chromosome 20 open reading frame 103 | NM_012261 |
| C5orf13 | Chromosome 5 open reading frame 13 | NM_004772 |
| C6orf176 | Chromosome 6 open reading frame 176 | CR618615 |
| C8orf22 | Chromosome 8 open reading frame 22 | NM_001007176 |
| CAV1 | Caveolin 1, caveolae protein, 22 kDa | NM_001753 |
| CAV1 | Caveolin 1, caveolae protein, 22 kDa | NM_001753 |
| CAV3 | Caveolin 3 | NM_001234 |
| CCL2 | Chemokine (C-C motif) ligand 2 | NM_002982 |
| CCNB1 | Cyclin B1 | NM_031966 |
| CCNB2 | Cyclin B2 | NM_004701 |
| CCR1 | Chemokine (C-C motif) receptor 1 | NM_001295 |
| CD1A | CD1A antigen, a polypeptide | BC031645 |
| CD200 | CD200 antigen | NM_001004196 |
| CD28 | CD28 antigen (Tp44) | NM_006139 |
| CD33 | CD33 antigen (gp67) | NM_001772 |
| CD38 | CD38 antigen (p45) | NM_001775 |
| CD3Z | CD3Z antigen, zeta polypeptide (TiT3 complex) | NM_198053 |
| CDC2 | Cell division cycle 2, G1 to S and G2 to M | NM_001786 |
| CDC20 | CDC20 cell division cycle 20 homolog (S. cerevisiae) | NM_001255 |
| CDC37L1 | Cell division cycle 37 homolog (S. cerevisiae)-like 1 | NM_017913 |
| CDCA1 | Cell division cycle associated 1 | NM_145697 |
| CDCA2 | Cell division cycle associated 2 | NM_152562 |
| CDCA7 | Cell division cycle associated 7 | NM_031942 |
| CDCA8 | Cell division cycle associated 8 | NM_018101 |
| CDH1 | Cadherin 1, type 1, E-cadherin (epithelial) | NM_004360 |
| CDH18 | Cadherin 18, type 2 | NM_004934 |
| CDKN3 | Cyclin-dependent kinase inhibitor 3 (CDK2-associated | NM_005192 |
| | dual specificity phosphatase) | |
| CENPA | Centromere protein A, 17 kDa | NM_001809 |
| CENPF | Centromere protein F, 350/400ka (mitosin) | NM_016343 |
| CGA | Glycoprotein hormones, alpha polypeptide | NM_000735 |
| CGA | Glycoprotein hormones, alpha polypeptide | NM_000735 |
| CH25H | Cholesterol 25-hydroxylase | NM_003956 |
| CHD7 | Chromodomain helicase DNA binding protein 7 | NM_017780 |
| CHSY1 | Carbohydrate (chondroitin) synthase 1 | NM_014918 |
| CISH | Cytokine inducible SH2-containing protein | NM_145071 |
| CITED4 | Cbp/p300-interacting transactivator, with Glu/Asp-rich | NM_133467 |
| | carboxy-terminal domain, 4 | |
| CLDN11 | Claudin 11 (oligodendrocyte transmembrane protein) | AF085871 |
| CLIC3 | Chloride intracellular channel 3 | NM_004669 |
| CMKOR1 | Chemokine orphan receptor 1 | NM_020311 |
| CMRF-35H | Leukocyte membrane antigen | NM_007261 |
| CNIH3 | Cornichon homolog 3 (Drosophila) | NM_152495 |
| COBLL1 | COBL-like 1 | NM_014900 |
| COCH | Coagulation factor C homolog, cochlin (Limulus | NM_004086 |
| | polyphemus) | |
| COL3A1 | Collagen, type III, alpha 1 (Ehlers-Danlos syndrome | NM_000090 |
| | type IV, autosomal dominant) | |
| COL4A6 | Collagen, type IV, alpha 6 | NM_033641 |
| COL8A1 | Collagen, type VIII, alpha 1 | AL359062 |
| CPB2 | Carboxypeptidase B2 (plasma, carboxypeptidase U) | NM_001872 |
| CTGF | Connective tissue growth factor | NM_001901 |
| CTNNAL1 | Catenin (cadherin-associated protein), alpha-like 1 | NM_003798 |
| CTNND2 | Catenin (cadherin-associated protein), delta 2 (neural | NM_001332 |
| | plakophilin-related arm-repeat protein) | |
| CX3CR1 | Chemokine (C—X3—C motif) receptor 1 | NM_001337 |
| CXCL1 | Chemokine (C—X—C motif) ligand 1 (melanoma growth | NM_001511 |
| | stimulating activity, alpha) | |
| CXCR4 | chemokine (C—X—C motif) receptor 4 | NM_001008540 |
| DDC | Dopa decarboxylase (aromatic L-amino acid | NM_000790 |
| | decarboxylase) | |
| DEPDC1 | DEP domain containing 1 | NM_017779 |
| DEPDC1B | DEP domain containing 1B | NM_018369 |
| DKFZP434B061 | DKFZP434B061 protein | AL117481 |
| DKFZP547L112 | Hypothetical protein DKFZp547L112 | AL512723 |
| DKFZP586A0522 | DKFZP586A0522 protein | NM_014033 |
| DKFZP586A0522 | DKFZP586A0522 protein | NM_014033 |
| DKK2 | Dickkopf homolog 2 (Xenopus laevis) | NM_014421 |
| DLG7 | Discs, large homolog 7 (Drosophila) | NM_014750 |
| DMD | Dystrophin (muscular dystrophy, Duchenne and Becker | NM_004010 |
| | types) | |
| DNAJC12 | DnaJ (Hsp40) homolog, subfamily C, member 12 | NM_021800 |
| DNM3 | Dynamin 3 | AK021543 |
| DOK1 | Docking protein 1, 62 kDa (downstream of tyrosine | NM_001381 |
| | kinase 1) | |
| DPPA4 | Developmental pluripotency associated 4 | NM_018189 |
| DUSP6 | Dual specificity phosphatase 6 | NM_001946 |
| ECM2 | Extracellular matrix protein 2, female organ and | NM_001393 |
| | adipocyte specific | |
| EDN1 | Endothelin 1 | NM_001955 |
| EFNB2 | Ephrin-B2 | NM_004093 |
| EGLN3 | Egl nine homolog 3 (C. elegans) | NM_022073 |
| EGR1 | Early growth response 1 | NM_001964 |
| ELF3 | E74-like factor 3 (ets domain transcription factor, | NM_004433 |
| | epithelial-specific) | |
| EN1 | Engrailed homolog 1 | NM_001426 |
| ENC1 | Ectodermal-neural cortex (with BTB-like domain) | NM_003633 |
| ENC1 | Ectodermal-neural cortex (with BTB-like domain) | NM_003633 |
| EPB41L4B | Erythrocyte membrane protein band 4.1 like 4B | NM_018424 |
| EPHA4 | EPH receptor A4 | NM_004438 |
| EPHX2 | Epoxide hydrolase 2, cytoplasmic | NM_001979 |
| EVA1 | Epithelial V-like antigen 1 | NM_144765 |
| EXOSC8 | Exosome component 8 | NM_181503 |
| EXOSC8 | Exosome component 8 | NM_181503 |
| F11 | Coagulation factor XI (plasma thromboplastin | NM_000128 |
| | antecedent) | |
| F3 | Coagulation factor III (thromboplastin, tissue factor) | NM_001993 |
| FA2H | Fatty acid 2-hydroxylase | NM_024306 |
| FADS1 | Fatty acid desaturase 1 | NM_013402 |
| FBXL16 | F-box and leucine-rich repeat protein 16 | NM_153350 |
| FBXO32 | F-box protein 32 | NM_058229 |
| FCGBP | Fc fragment of IgG binding protein | NM_003890 |
| FGA | Fibrinogen, A alpha polypeptide | NM_000508 |
| FGF7 | Galactokinase 2 | NM_002009 |
| FGF7 | Galactokinase 2 | NM_002009 |
| FHL2 | Four and a half LIM domains 2 | NM_201555 |
| FLJ10156 | Hypothetical protein FLJ10156 | NM_019013 |
| FLJ10901 | Hypothetical protein FLJ10901 | NM_018265 |
| FLJ13072 | Hypothetical gene FLJ13072 | AK023134 |
| FLJ13391 | Hypothetical protein FLJ13391 | NM_032181 |
| FLJ13840 | Hypothetical protein FLJ13840 | BC007638 |
| FLJ14712 | Hypothetical protein FLJ14712 | AK027618 |
| FLJ14834 | Hypothetical protein FLJ14834 | NM_032849 |
| FLJ30681 | KIAA1983 protein | NM_133459 |
| FLJ31340 | Hypothetical protein FLJ31340 | NM_152748 |
| FLJ31461 | Hypothetical protein FLJ31461 | NM_152454 |
| FLJ35767 | FLJ35767 protein | NM_207459 |
| FLJ36031 | Hypothetical protein FLJ36031 | AK098422 |
| FLJ37478 | Hypothetical protein FLJ37478 | NM_178557 |
| FLJ37970 | Hypothetical protein FLJ37970 | NM_032251 |
| FLJ39739 | FLJ39739 protein | AK026418 |
| FLJ45273 | FLJ45273 protein | NM_198461 |
| FLRT2 | Fibronectin leucine rich transmembrane protein 2 | NM_013231 |
| FOS | V-fos FBJ murine osteosarcoma viral oncogene homolog | NM_005252 |
| FOXA1 | Forkhead box A1 | NM_004496 |
| FOXA2 | Forkhead box A2 | NM_021784 |
| FOXM1 | Forkhead box M1 | NM_202002 |
| FOXQ1 | Forkhead box Q1 | NM_033260 |
| FRMD3 | FERM domain containing 3 | BG216229 |
| FZD10 | Frizzled homolog 10 (Drosophila) | NM_007197 |
| G2 | G2 protein | U10991 |
| GAJ | GAJ protein | NM_032117 |
| GAS1 | Growth arrest-specific 1 | NM_002048 |
| GATA6 | GATA binding protein 6 | NM_005257 |
| GDF15 | Growth differentiation factor 15 | NM_004864 |
| GDF3 | Growth differentiation factor 3 | NM_020634 |
| GEM | GTP binding protein overexpressed in skeletal muscle | NM_005261 |
| GPR68 | G protein-coupled receptor 68 | NM_003485 |
| GREM1 | Gremlin 1 homolog, cysteine knot superfamily | NM_013372 |
| | (Xenopus laevis) | |
| GSG1 | Germ cell associated 1 | NM_031289 |
| GTSE1 | G-2 and S-phase expressed 1 | NM_016426 |
| HCAP-G | Chromosome condensation protein G | NM_022346 |
| HLF | Hepatic leukemia factor | NM_002126 |
| HMMR | Hyaluronan-mediated motility receptor (RHAMM) | NM_012484 |
| HRH1 | Histamine receptor H1 | NM_000861 |
| HS6ST2 | Heparan sulfate 6-O-sulfotransferase 2 | NM_147175 |
| HSD11B2 | Hydroxysteroid (11-beta) dehydrogenase 2 | NM_000196 |
| HT008 | Uncharacterized hypothalamus protein HT008 | NM_018469 |
| ID1 | Inhibitor of DNA binding 1, dominant negative helix- | NM_002165 |
| | loop-helix protein | |
| IFI44 | Interferon-induced protein 44 | NM_006417 |
| IL10RA | Interleukin 10 receptor, alpha | NM_001558 |
| IL6 | Interleukin 6 (interferon, beta 2) | NM_000600 |
| INSIG2 | Insulin induced gene 2 | NM_016133 |
| INSIG2 | Insulin induced gene 2 | NM_016133 |
| IRF5 | Interferon regulatory factor 5 | NM_002200 |
| IRX4 | Iroquois homeobox protein 4 | NM_016358 |
| JAG1 | Jagged 1 (Alagille syndrome) | NM_000214 |
| KCNH2 | Potassium voltage-gated channel, subfamily H (eag- | NM_000238 |
| | related), member 2 | |
| KCNK6 | Potassium channel, subfamily K, member 6 | NM_004823 |
| KCNMB4 | Potassium large conductance calcium-activated channel, | NM_014505 |
| | subfamily M, beta member 4 | |
| KIAA0101 | KIAA0101 | NM_014736 |
| KIAA1199 | KIAA1199 | NM_018689 |
| KIAA1217 | KIAA1217 | AK022045 |
| KIAA1509 | KIAA1509 | AB040942 |
| KIAA1666 | KIAA1666 protein | BC035246 |
| KIAA1913 | KIAA1913 | BC044246 |
| KIF20A | Kinesin family member 20A | NM_005733 |
| KIF2C | Kinesin family member 2C | NM_006845 |
| KLF2 | Kruppel-like factor 2 (lung) | NM_016270 |
| KLRC3 | Killer cell lectin-like receptor subfamily C, member 2 | NM_002260 |
| KNSL7 | Kinesin-like 7 | NM_020242 |
| KNTC2 | Kinetochore associated 2 | NM_006101 |
| KRTAP1-5 | Keratin associated protein 1-5 | NM_031957 |
| KRTHB6 | Keratin, hair, basic, 6 (monilethrix) | NM_002284 |
| LAD1 | Ladinin 1 | NM_005558 |
| LAMA2 | Laminin, alpha 2 (merosin, congenital muscular | NM_000426 |
| | dystrophy) | |
| LAPTM5 | Lysosomal associated multispanning membrane protein 5 | NM_006762 |
| LASS5 | LAG1 longevity assurance homolog 5 (S. cerevisiae) | NM_147190 |
| LEF1 | Lymphoid enhancer-binding factor 1 | NM_016269 |
| LGALS2 | Lectin, galactoside-binding, soluble, 2 (galectin 2) | NM_006498 |
| LHX2 | LIM homeobox 2 | NM_004789 |
| LOC120224 | Hypothetical protein BC016153 | NM_138788 |
| LOC150166 | Hypothetical protein LOC150166 | AK056836 |
| LOC150271 | Hypothetical LOC388889 | AK098753 |
| LOC150759 | Hypothetical protein LOC150759 | AK057596 |
| LOC222171 | Hypothetical protein LOC222171 | NM_175887 |
| LOC284018 | Hypothetical protein LOC284018 | NM_181655 |
| LOC285733 | Hypothetical protein LOC285733 | AK091900 |
| LOC286254 | Hypothetical protein LOC286254 | AK092751 |
| LOC338773 | Hypothetical protein LOC338773 | NM_181724 |
| LOC92312 | Hypothetical protein LOC92312 | XM_044166 |
| LOXL3 | Lysyl oxidase-like 3 | NM_032603 |
| LPL | Lipoprotein lipase | NM_000237 |
| LRP12 | Low density lipoprotein-related protein 12 | NM_013437 |
| LRP12 | Low density lipoprotein-related protein 12 | NM_013437 |
| LRP8 | Low density lipoprotein receptor-related protein 8, | NM_033300 |
| | apolipoprotein e receptor | |
| LRRC5 | Leucine rich repeat containing 5 | NM_018103 |
| LTBP2 | Latent transforming growth factor beta binding protein 2 | NM_000428 |
| LYPDC1 | LY6/PLAUR domain containing 1 | NM_144586 |
| MAD2L1 | MAD2 mitotic arrest deficient-like 1 (yeast) | NM_002358 |
| MAFB | V-maf musculoaponeurotic fibrosarcoma oncogene | NM_005461 |
| | homolog B (avian) | |
| MAGEA1 | Melanoma antigen, family A, 1 (directs expression of | NM_004988 |
| | antigen MZ2-E) | |
| MAL2 | Mal, T-cell differentiation protein 2 | NM_052886 |
| MAOB | Monoamine oxidase B | NM_000898 |
| MAP7 | Microtubule-associated protein 7 | NM_003980 |
| MASP1 | Mannan-binding lectin serine protease 1 (C4/C2 | NM_139125 |
| | activating component of Ra-reactive factor) | |
| MCM10 | MCM10 minichromosome maintenance deficient 10 (S. cerevisiae) | NM_182751 |
| MEST | Mesoderm specific transcript homolog (mouse) | NM_002402 |
| MGAT3 | Mannosyl (beta-1,4-)-glycoprotein beta-1,4-N- | AK125361 |
| | acetylglucosaminyltransferase | |
| MGC16121 | Hypothetical protein MGC16121 | BC007360 |
| MGC22265 | Hypothetical protein MGC22265 | BC048193 |
| MGC2574 | Hypothetical protein MGC2574 | NM_024098 |
| MGC2610 | Hypothetical protein MGC2610 | NM_144711 |
| MGC27165 | Hypothetical protein MGC27165 | AF343666 |
| MGC33365 | Hypothetical protein MGC33365 | NM_173552 |
| MK2S4 | Protein kinase substrate MK2S4 | NM_052862 |
| MMP12 | Matrix metalloproteinase 12 (macrophage elastase) | NM_002426 |
| MSX1 | Msh homeo box homolog 1 (Drosophila) | NM_002448 |
| MSX1 | Msh homeo box homolog 1 (Drosophila) | NM_002448 |
| MT1B | Metallothionein 1B (functional) | NM_005947 |
| MT1E | Metallothionein 1E (functional) | NM_175617 |
| MT1G | Metallothionein 1G | NM_005950 |
| MT1H | Metallothionein 1H | NM_005951 |
| MT1H | Metallothionein 1H | NM_005951 |
| MT1K | Metallothionein 1K | NM_176870 |
| MT1L | Metallothionein 1L | X97261 |
| MT1X | Metallothionein 1X | NM_005952 |
| MT1X | Metallothionein 1X | NM_005952 |
| MT2A | Metallothionein 2A | NM_005953 |
| MT2A | Metallothionein 2A | NM_005953 |
| MYB | V-myb myeloblastosis viral oncogene homolog (avian) | NM_005375 |
| MYBL1 | V-myb myeloblastosis viral oncogene homolog (avian)- | X66087 |
| | like 1 | |
| MYLIP | Myosin regulatory light chain interacting protein | NM_013262 |
| MYO10 | Myosin X | NM_012334 |
| MYO1G | Myosin IG | NM_033054 |
| MYO5B | Myosin VB | AK025336 |
| MYO5C | Myosin VC | NM_018728 |
| MYRIP | Myosin VIIA and Rab interacting protein | NM_015460 |
| NAP1L1 | Nucleosome assembly protein 1-like 1 | NM_139207 |
| NAV2 | Neuron navigator 2 | NM_182964 |
| NEK2 | NIMA (never in mitosis gene a)-related kinase 2 | NM_002497 |
| NET1 | Neuroepithelial cell transforming gene 1 | NM_005863 |
| NFE2 | Nuclear factor (erythroid-derived 2), 45 kDa | NM_006163 |
| NFE2L3 | Nuclear factor (erythroid-derived 2)-like 3 | NM_004289 |
| NFIL3 | Nuclear factor, interleukin 3 regulated | NM_005384 |
| NGEF | Neuronal guanine nucleotide exchange factor | NM_019850 |
| NID2 | Nidogen 2 (osteonidogen) | NM_007361 |
| NOSTRIN | Nitric oxide synthase trafficker | NM_052946 |
| NOV | Nephroblastoma overexpressed gene | NM_002514 |
| NPTX1 | Neuronal pentraxin I | NM_002522 |
| NR0B1 | Nuclear receptor subfamily 0, group B, member 1 | NM_000475 |
| NR2F1 | Nuclear receptor subfamily 2, group F, member 1 | NM_005654 |
| NSE1 | NSE1 | NM_145175 |
| NSE2 | Breast cancer membrane protein 101 | NM_174911 |
| NTN4 | Netrin 4 | NM_021229 |
| NUP210 | Nucleoporin 210 kDa | NM_024923 |
| NUSAP1 | Nucleolar and spindle associated protein 1 | NM_016359 |
| ODZ3 | Odz, odd Oz/ten-m homolog 3 (Drosophila) | AB040888 |
| ODZ3 | Odz, odd Oz/ten-m homolog 3 (Drosophila) | AB040888 |
| OIP5 | Opa-interacting protein 5 | NM_007280 |
| OLIG1 | Oligodendrocyte transcription factor 1 | NM_138983 |
| OSAP | Ovary-specific acidic protein | NM_032623 |
| OVOS2 | Ovostatin 2 | BC039117 |
| P2RY8 | Purinergic receptor P2Y, G-protein coupled, 8 | NM_178129 |
| PAPPA | Pregnancy-associated plasma protein A, pappalysin 1 | NM_002581 |
| PAQR4 | Progestin and adipoQ receptor family member IV | NM_152341 |
| PASD1 | PAS domain containing 1 | NM_173493 |
| PBEF1 | Pre-B-cell colony enhancing factor 1 | NM_005746 |
| PBEF1 | Pre-B-cell colony enhancing factor 1 | NM_005746 |
| PBEF1 | Pre-B-cell colony enhancing factor 1 | NM_182790 |
| PCSK5 | Proprotein convertase subtilisin/kexin type 5 | NM_006200 |
| PDGFC | Platelet derived growth factor C | NM_016205 |
| PEPP-2 | PEPP subfamily gene 2 | NM_032498 |
| PHLDA1 | Pleckstrin homology-like domain, family A, member 1 | NM_007350 |
| PIK3R1 | Phosphoinositide-3-kinase, regulatory subunit 1 (p85 | NM_181523 |
| | alpha) | |
| PIM1 | Pim-1 oncogene | NM_002648 |
| PITX2 | Paired-like homeodomain transcription factor 2 | NM_153426 |
| PLAC8 | Placenta-specific 8 | NM_016619 |
| PLAC8 | Placenta-specific 8 | NM_016619 |
| PLD1 | Phospholipase D1, phophatidylcholine-specific | NM_002662 |
| PLK2 | Polo-like kinase 2 (Drosophila) | NM_006622 |
| PLOD2 | Procollagen-lysine, 2-oxoglutarate 5-dioxygenase (lysine | NM_182943 |
| | hydroxylase) 2 | |
| PLP1 | Proteolipid protein 1 (Pelizaeus-Merzbacher disease, | M54927 |
| | spastic paraplegia 2, uncomplicated) | |
| PMAIP1 | Phorbol-12-myristate-13-acetate-induced protein 1 | NM_021127 |
| PON3 | Paraoxonase 3 | NM_000940 |
| POSTN | Periostin, osteoblast specific factor | NM_006475 |
| PPP1R1A | Protein phosphatase 1, regulatory (inhibitor) subunit 1A | NM_006741 |
| PPP1R3B | Protein phosphatase 1, regulatory (inhibitor) subunit 3B | AK091994 |
| PRC1 | Protein regulator of cytokinesis 1 | NM_003981 |
| PREX1 | Phosphatidylinositol 3,4,5-trisphosphate-dependent RAC | NM_020820 |
| | exchanger 1 | |
| PSD3 | Pleckstrin and Sec7 domain containing 3 | NM_015310 |
| PSD3 | Pleckstrin and Sec7 domain containing 3 | NM_015310 |
| PSG1 | Pregnancy specific beta-1-glycoprotein 1 | NM_006905 |
| PSG3 | Pregnancy specific beta-1-glycoprotein 3 | NM_021016 |
| PTGFR | Prostaglandin F receptor (FP) | NM_000959 |
| PTGIR | Prostaglandin I2 (prostacyclin) receptor (IP) | NM_000960 |
| PTTG1 | Pituitary tumor-transforming 1 | NM_004219 |
| PTTG2 | Pituitary tumor-transforming 2 | NM_006607 |
| RACGAP1 | Rac GTPase activating protein 1 | NM_013277 |
| RAMP1 | Receptor (calcitonin) activity modifying protein 1 | NM_005855 |
| RANBP9 | RAN binding protein 9 | NM_005493 |
| RANBP9 | RAN binding protein 9 | NM_005493 |
| RASD1 | RAS, dexamethasone-induced 1 | NM_016084 |
| RASGRP1 | RAS guanyl releasing protein 1 (calcium and DAG- | NM_005739 |
| | regulated) | |
| RGS2 | Regulator of G-protein signalling 2, 24 kDa | NM_002923 |
| RIPK3 | Receptor-interacting serine-threonine kinase 3 | NM_006871 |
| RTN4R | Reticulon 4 receptor | NM_023004 |
| S100B | S100 calcium binding protein, beta (neural) | NM_006272 |
| SAMSN1 | SAM domain, SH3 domain and nuclear localisation | NM_022136 |
| | signals, 1 | |
| SECTM1 | Secreted and transmembrane 1 | NM_003004 |
| SEMA3C | Sema domain, immunoglobulin domain (Ig), short basic | NM_006379 |
| | domain, secreted, (semaphorin) 3C | |
| SEMA3D | Sema domain, immunoglobulin domain (Ig), short basic | NM_152754 |
| | domain, secreted, (semaphorin) 3D | |
| SERPINA5 | Serine (or cysteine) proteinase inhibitor, clade A (alpha- | NM_000624 |
| | 1 antiproteinase, antitrypsin), member 5 | |
| SGOL2 | Shugoshin-like 2 (S. pombe) | NM_152524 |
| SIAT7C | Sialyltransferase 7 ((alpha-N-acetylneuraminyl-2,3-beta- | NM_152996 |
| | galactosyl-1,3)-N-acetyl galactosaminide alpha-2,6- | |
| | sialyltransferase) C | |
| SLC24A3 | Solute carrier family 24 (sodium/potassium/calcium | NM_020689 |
| | exchanger), member 3 | |
| SLC27A2 | Solute carrier family 27 (fatty acid transporter), member 2 | NM_003645 |
| SLC2A1 | Solute carrier family 2 (facilitated glucose transporter), | NM_006516 |
| | member 1 | |
| SLC39A8 | Solute carrier family 39 (zinc transporter), member 8 | NM_022154 |
| SLC40A1 | Solute carrier family 40 (iron-regulated transporter), | NM_014585 |
| | member 1 | |
| SLC7A5 | Solute carrier family 7 (cationic amino acid transporter, | NM_003486 |
| | y+ system), member 5 | |
| SMARCA3 | SWI/SNF related, matrix associated, actin dependent | NM_139048 |
| | regulator of chromatin, subfamily a, member 3 | |
| SMOC1 | SPARC related modular calcium binding 1 | NM_022137 |
| SMOC2 | SPARC related modular calcium binding 2 | NM_022138 |
| SNAI2 | Snail homolog 2 (Drosophila) | NM_003068 |
| SNFT | Jun dimerization protein p21SNFT | NM_018664 |
| SNX10 | Sorting nexin 10 | NM_013322 |
| SOCS1 | Suppressor of cytokine signaling 1 | NM_003745 |
| SOCS3 | Suppressor of cytokine signaling 3 | NM_003955 |
| SOX2 | SRY (sex determining region Y)-box 2 | NM_003106 |
| SOX4 | SRY (sex determining region Y)-box 4 | AW946823 |
| SOX4 | SRY (sex determining region Y)-box 4 | NM_003107 |
| SOX4 | SRY (sex determining region Y)-box 4 | NM_003107 |
| SP5 | Sp5 transcription factor | NM_001003845 |
| SPAG5 | Sperm associated antigen 5 | NM_006461 |
| SPHK1 | Sphingosine kinase 1 | NM_021972 |
| SPINT2 | Serine protease inhibitor, Kunitz type, 2 | NM_021102 |
| SPTA1 | Spectrin, alpha, erythrocytic 1 (elliptocytosis 2) | NM_003126 |
| STAC | SH3 and cysteine rich domain | NM_003149 |
| STC2 | Stanniocalcin 2 | NM_003714 |
| STMN1 | Stathmin 1/oncoprotein 18 | NM_203401 |
| SYTL5 | Synaptotagmin-like 5 | BX647688 |
| T3JAM | TRAF3-interacting Jun N-terminal kinase (JNK)- | NM_025228 |
| | activating modulator | |
| TCEAL7 | Transcription elongation factor A (SII)-like 7 | NM_152278 |
| TFPI2 | Tissue factor pathway inhibitor 2 | NM_006528 |
| THSD2 | Thrombospondin, type I, domain containing 2 | NM_032784 |
| TIMP3 | Tissue inhibitor of metalloproteinase 3 (Sorsby fundus | AA837799 |
| | dystrophy, pseudoinflammatory) | |
| TK1 | Thymidine kinase 1, soluble | NM_003258 |
| TM4SF1 | Transmembrane 4 superfamily member 1 | NM_014220 |
| TMSNB | Thymosin, beta, identified in neuroblastoma cells | NM_021992 |
| TNC | Tenascin C (hexabrachion) | NM_002160 |
| TncRNA | Trophoblast-derived noncoding RNA | U60873 |
| TNFRSF17 | Tumor necrosis factor receptor superfamily, member 17 | NM_001192 |
| TOP2A | Topoisomerase (DNA) II alpha 170 kDa | NM_001067 |
| TOPK | T-LAK cell-originated protein kinase | NM_018492 |
| TPD52 | Tumor protein D52 | NM_005079 |
| TPX2 | TPX2, microtubule-associated protein homolog | NM_012112 |
| | (Xenopus laevis) | |
| TRIB1 | Tribbles homolog 1 (Drosophila) | NM_025195 |
| TRIM45 | Tripartite motif-containing 45 | NM_025188 |
| TROAP | Trophinin associated protein (tastin) | NM_005480 |
| TRPS1 | Trichorhinophalangeal syndrome I | NM_014112 |
| TWIST1 | Twist homolog 1 (acrocephalosyndactyly 3; Saethre- | NM_000474 |
| | Chotzen syndrome) (Drosophila) | |
| TYR | Tyrosinase (oculocutaneous albinism IA) | NM_000372 |
| TYRP1 | Tyrosinase-related protein 1 | NM_000550 |
| UAP1 | UDP-N-acteylglucosamine pyrophosphorylase 1 | NM_003115 |
| UBD | Ubiquitin D | NM_006398 |
| UBE2C | Ubiquitin-conjugating enzyme E2C | NM_181803 |
| UTS2 | Urotensin 2 | NM_021995 |
| UTS2 | Urotensin 2 | NM_021995 |
| VCX3 | Variable charge, X-linked | NM_016379 |
| XK | Kell blood group precursor (McLeod phenotype) | NM_021083 |
| YPEL4 | Yippee-like 4 (Drosophila) | NM_145008 |
| ZBTB20 | Zinc finger and BTB domain containing 20 | BC010934 |
| | | A_23_P170719 |
| | | A_23_P28927 |
| | | A_24_P112542 |
| | | A_24_P195454 |
| | | A_24_P290087 |
| | | A_24_P358131 |
| | | A_24_P927205 |
| | | A_32_P225328 |
| | | A_32_P75141 |
| | | AF256215 |
| | MRNA (fetal brain cDNA g6_1g) | AI791206 |
| | Hypothetical gene supported by AK026189 | AK022865 |
| | Hypothetical gene supported by AK026328 | AK026328 |
| | CDNA: FLJ23131 fis, clone LNG08502 | AK026784 |
| | CDNA FLJ31059 fis, clone HSYRA2000832 | AK055621 |
| | Homo sapiens, clone IMAGE: 4214962, mRNA | AK091547 |
| | | AK098506 |
| | Homo sapiens, clone IMAGE: 4512785, mRNA | AK124558 |
| | CDNA FLJ43172 fis, clone FCBBF3007242 | AK125162 |
| | CDNA FLJ26031 fis, clone PNC08078 | AK129542 |
| | Transcribed locus | AW972815 |
| | | BC005081 |
| | Similar to ankyrin repeat domain 20A | BC016022 |
| | Homo sapiens, clone IMAGE: 3869276, mRNA | BC018597 |
| | Homo sapiens, Similar to hect domain and RLD 2, clone | BC018626 |
| | IMAGE: 4581928, mRNA | |
| | Homo sapiens, clone IMAGE: 3357292, mRNA, partial | BC033117 |
| | cds | |
| | CDNA clone MGC: 65154 IMAGE: 5122136, complete | BC056907 |
| | cds | |
| | MRNA; cDNA DKFZp586O0724 (from clone | BF508144 |
| | DKFZp586O0724) | |
| | Transcribed locus | BQ717518 |
| | Transcribed locus, strongly similar to XP_355557.2 | CD048206 |
| | similar to multi sex combs CG12058-PA [Mus | |
| | musculus] | |
| | Full-length cDNA clone CS0DM001YA20 of Fetal liver | CR601260 |
| | of Homo sapiens (human) | |
| | Full-length cDNA clone CS0DJ001YJ05 of T cells | CR601458 |
| | (Jurkat cell line) Cot 10-normalized of Homo sapiens | |
| | (human) | |
| | Full-length cDNA clone CS0DC002YA18 of | CR624517 |
| | Neuroblastoma Cot 25-normalized of Homo sapiens | |
| | (human) | |
| | | CR936791 |
| | | CX788817 |
| | | ENST00000245185 |
| | | ENST00000261569 |
| | | ENST00000369158 |
| | | ENST00000371256 |
| | | ENST00000371327 |
| | | ENST00000371655 |
| | | ENST00000374541 |
| | | ENST00000375077 |
| | | ENST00000375855 |
| | | ENST00000376155 |
| | | ENST00000381889 |
| | | NM_001006641 |
| | | NM_001009954 |
| | | NM_001010911 |
| | | NM_001010915 |
| | | NM_001012271 |
| | | NM_001017424 |
| | | NM_001017535 |
| | | NM_001017915 |
| | | NM_001017978 |
| | | NM_001018115 |
| | | NM_001031716 |
| | | NM_001040100 |
| | | NM_001040167 |
| | | NM_002263 |
| | | NM_003621 |
| | | NM_012454 |
| | | NM_014867 |
| | | NM_020872 |
| | | NM_020872 |
| | | NM_025135 |
| | | NM_032199 |
| | | NM_032521 |
| | | NR_001564 |
| | | THC2270231 |
| | | THC2281706 |
| | | THC2281732 |
| | | THC2282958 |
| | | THC2309960 |
| | | THC2314600 |
| | | THC2317680 |
| | | THC2343936 |
| | | THC2347909 |
| | | THC2364621 |
| | | THC2373845 |
| | | THC2376729 |
| | | THC2381061 |
| | | THC2407823 |
| | | THC2411757 |
| | | THC2434166 |
| | | THC2438492 |
| | | THC2442210 |
| | | THC2446045 |
| | | W95609 |
| | Similar to hypothetical protein LOC231503 | XM_496707 |
| | | XM_934971 |

This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

Each of the foregoing patents, patent applications and references is hereby incorporated by reference, particularly for the teaching referenced herein.

Having thus described several aspects of at least one embodiment of this invention, it is to be appreciated various alterations, modifications, and improvements will readily occur to those skilled in the art. Such alterations, modifications, and improvements are intended to be part of this disclosure, and are intended to be within the spirit and scope of the invention. Accordingly, the foregoing description and drawings are by way of example only.

TABLE 1A
FDR for Change in Gene Expression Values Between Pre-and Post-Treatment Responders

| Agilent Probe ID | Gene Symbol | Gene Name | Accession | Entrez Gene ID | FDR |
|---|---|---|---|---|---|
| AGI_HUM1_OLIGO_A_24_P745883 | | | A_24_P745883 | | 0.032464275 |
| AGI_HUM1_OLIGO_A_24_P243396 | | | NM_001008657 | | 0.039492348 |
| AGI_HUM1_OLIGO_A_24_P53080 | ETFA | Electron-transfer-flavoprotein, alpha polypeptide (glutaric aciduria II) | NM_000126 | 2108 | 0.039492348 |
| AGI_HUM1_OLIGO_A_23_P117298 | F7 | Coagulation factor VII (serum prothrombin conversion accelerator) | NM_000131 | 2155 | 0.060597251 |
| AGI_HUM1_OLIGO_A_23_P14124 | RASL11A | RAS-like, family 11, member A | NM_206827 | 387496 | 0.060597251 |
| AGI_HUM1_OLIGO_A_23_P156708 | TNXB | Tenascin XB | NM_019105 | 7148 | 0.060597251 |
| AGI_HUM1_OLIGO_A_23_P205177 | F10 | Coagulation factor X | NM_000504 | 2159 | 0.060597251 |
| AGI_HUM1_OLIGO_A_23_P345650 | KIAA1280 | KIAA1280 protein | NM_015691 | 55841 | 0.060597251 |
| AGI_HUM1_OLIGO_A_23_P5281 | LYL1 | Lymphoblastic leukemia derived sequence 1 | NM_005583 | 4066 | 0.060597251 |
| AGI_HUM1_OLIGO_A_24_P169843 | | | A_24_P169843 | | 0.060597251 |
| AGI_HUM1_OLIGO_A_24_P180383 | NIPBL | Nipped-B homolog (Drosophila) | NM_015384 | 25836 | 0.060597251 |
| AGI_HUM1_OLIGO_A_24_P185604 | NDRG3 | NDRG family member 3 | NM_032013 | 57446 | 0.060597251 |
| AGI_HUM1_OLIGO_A_24_P29966 | TRIM32 | Tripartite motif-containing 32 | NM_012210 | 22954 | 0.060597251 |
| AGI_HUM1_OLIGO_A_24_P727884 | G2 | G2 protein | U10991 | 25758 | 0.060597251 |
| AGI_HUM1_OLIGO_A_24_P924040 | SSFA2 | Sperm specific antigen 2 | NM_006751 | 6744 | 0.060597251 |
| AGI_HUM1_OLIGO_A_23_P165783 | MLPH | Melanophilin | NM_024101 | 79083 | 0.068421106 |
| AGI_HUM1_OLIGO_A_23_P162866 | HSPCA | Heat shock 90 kDa protein 1, alpha | NM_005348 | 3320 | 0.070083415 |
| AGI_HUM1_OLIGO_A_23_P44363 | CASKIN2 | CASK interacting protein 2 | NM_020753 | 57513 | 0.070083415 |
| AGI_HUM1_OLIGO_A_23_P45324 | TMEM35 | Transmembrane protein 35 | NM_021637 | 59353 | 0.070083415 |
| AGI_HUM1_OLIGO_A_23_P130418 | NDUFV2 | NADH dehydrogenase (ubiquinone) flavoprotein 2, 24 kDa | NM_021074 | 4729 | 0.075584295 |
| AGI_HUM1_OLIGO_A_23_P203115 | TMEM25 | Transmembrane protein 25 | NM_032780 | 84866 | 0.075584295 |
| AGI_HUM1_OLIGO_A_23_P502363 | EYA1 | Eyes absent homolog 1 (Drosophila) | NM_000503 | 2138 | 0.075584295 |
| AGI_HUM1_OLIGO_A_23_P52227 | GDF10 | Growth differentiation factor 10 | NM_004962 | 2662 | 0.075584295 |
| AGI_HUM1_OLIGO_A_23_P94103 | MGC45780 | Hypothetical protein MGC45780 | NM_173833 | 286133 | 0.075584295 |
| AGI_HUM1_OLIGO_A_24_P607904 | ITGA9 | Integrin, alpha 9 | AK026826 | 3680 | 0.075584295 |
| AGI_HUM1_OLIGO_A_23_P106532 | D4ST1 | Dermatan 4 sulfotransferase 1 | NM_130468 | 113189 | 0.083158024 |
| AGI_HUM1_OLIGO_A_23_P111860 | FLJ10324 | Hypothetical protein FLJ10324 | NM_018059 | 55698 | 0.083158024 |
| AGI_HUM1_OLIGO_A_23_P11664 | SFRS11 | Splicing factor, arginine/serine-rich 11 | NM_004768 | 9295 | 0.083158024 |
| AGI_HUM1_OLIGO_A_23_P121533 | SPON2 | Spondin 2, extracellular matrix protein | NM_012445 | 10417 | 0.083158024 |
| AGI_HUM1_OLIGO_A_23_P133694 | SLC29A1 | Solute carrier family 29 (nucleoside transporters), member 1 | NM_004955 | 2030 | 0.083158024 |
| AGI_HUM1_OLIGO_A_23_P153640 | ANKRD27 | Ankyrin repeat domain 27 (VPS9 domain) | NM_032139 | 84079 | 0.083158024 |
| AGI_HUM1_OLIGO_A_23_P212263 | MASP1 | Mannan-binding lectin serine protease 1 (C4/C2 activating component of Ra- | NM_139125 | 5648 | 0.083158024 |
| | | reactive factor) | | | |
| AGI_HUM1_OLIGO_A_23_P212339 | FYCO1 | FYVE and coiled-coil domain containing 1 | NM_024513 | 79443 | 0.083158024 |
| AGI_HUM1_OLIGO_A_23_P21324 | TWIST2 | Twist homolog 2 (Drosophila) | NM_057179 | 117581 | 0.083158024 |
| AGI_HUM1_OLIGO_A_23_P253982 | HOXA4 | Homeo box A4 | NM_002141 | 3201 | 0.083158024 |
| AGI_HUM1_OLIGO_A_23_P28507 | MGC52110 | Hypothetical protein MGC52110 | NM_012214 | 493753 | 0.083158024 |
| AGI_HUM1_OLIGO_A_23_P30363 | P4HA2 | Procollagen-proline, 2-oxoglutarate 4-dioxygenase (proline 4-hydroxylase), alpha | NM_004199 | 8974 | 0.083158024 |
| | | polypeptide II | | | |
| AGI_HUM1_OLIGO_A_23_P345692 | IL17D | Interleukin 17D | NM_138284 | 53342 | 0.083158024 |
| AGI_HUM1_OLIGO_A_23_P3562 | FLJ13291 | Hypothetical protein FLJ13291 | NM_032178 | 84138 | 0.083158024 |
| AGI_HUM1_OLIGO_A_23_P40072 | MTIF2 | Mitochondrial translational initiation factor 2 | NM_001005369 | 4528 | 0.083158024 |
| AGI_HUM1_OLIGO_A_23_P41976 | LOC133957 | Similar to RIKEN cDNA 0610011N22 | NM_145265 | 133957 | 0.083158024 |
| AGI_HUM1_OLIGO_A_23_P422851 | CABLES1 | Cdk5 and Abl enzyme substrate 1 | NM_138375 | 91768 | 0.083158024 |
| AGI_HUM1_OLIGO_A_23_P430181 | ZBTB3 | Zinc finger and BTB domain containing 3 | NM_024784 | 79842 | 0.083158024 |
| AGI_HUM1_OLIGO_A_23_P432598 | Shax3 | Snf7 homologue associated with Alix 3 | NM_152284 | 92421 | 0.083158024 |
| AGI_HUM1_OLIGO_A_23_P433016 | FBLN1 | Fibulin 1 | NM_001996 | 2192 | 0.083158024 |
| AGI_HUM1_OLIGO_A_23_P500410 | ATP6V1G2 | ATPase, H+ transporting, lysosomal 13 kDa, V1 subunit G isoform 2 | NM_130463 | 534 | 0.083158024 |
| AGI_HUM1_OLIGO_A_23_P56578 | VIT | Vitrin | NM_053276 | 5212 | 0.083158024 |
| AGI_HUM1_OLIGO_A_23_P74229 | MGC4796 | Ser/Thr-like kinase | NM_032017 | 83931 | 0.083158024 |
| AGI_HUM1_OLIGO_A_23_P77568 | EIF3S8 | Eukaryotic translation initiation factor 3, subunit 8, 110 kDa | NM_003752 | 8663 | 0.083158024 |
| AGI_HUM1_OLIGO_A_23_P8240 | FAM50B | Family with sequence similarity 50, member B | NM_012135 | 26240 | 0.083158024 |
| AGI_HUM1_OLIGO_A_23_P87839 | SFRS2IP | Splicing factor, arginine/serine-rich 2, interacting protein | NM_004719 | 9169 | 0.083158024 |
| AGI_HUM1_OLIGO_A_24_P137501 | SFRP2 | Secreted frizzled-related protein 2 | NM_003013 | 6423 | 0.083158024 |
| AGI_HUM1_OLIGO_A_24_P184385 | LOC220074 | Hypothetical 55.1 kDa protein F09G8.5 in chromosome III | NM_145309 | 220074 | 0.083158024 |
| AGI_HUM1_OLIGO_A_24_P184388 | LOC220074 | Hypothetical 55.1 kDa protein F09G8.5 in chromosome III | NM_145309 | 220074 | 0.083158024 |
| AGI_HUM1_OLIGO_A_24_P365954 | THAP11 | THAP domain containing 11 | NM_020457 | 57215 | 0.083158024 |
| AGI_HUM1_OLIGO_A_24_P406514 | UBE2V2 | Ubiquitin-conjugating enzyme E2 variant 2 | NM_003350 | 7336 | 0.083158024 |
| AGI_HUM1_OLIGO_A_24_P48862 | SNAP29 | Synaptosomal-associated protein, 29 kDa | NM_004782 | 9342 | 0.083158024 |
| AGI_HUM1_OLIGO_A_24_P598406 | RBAK | RB-associated KRAB repressor | BC068459 | 57786 | 0.083158024 |
| AGI_HUM1_OLIGO_A_24_P71700 | KIAA1190 | Hypothetical protein KIAA1190 | NM_145166 | 92999 | 0.083158024 |
| AGI_HUM1_OLIGO_A_24_P7470 | | | A_24_P7470 | | 0.083158024 |
| AGI_HUM1_OLIGO_A_24_P916916 | CYP3A5 | Cytochrome P450, family 3, subfamily A, polypeptide 5 | AF355801 | 1577 | 0.083158024 |
| AGI_HUM1_OLIGO_A_24_P98109 | SNX10 | Sorting nexin 10 | NM_013322 | 29887 | 0.083158024 |
| AGI_HUM1_OLIGO_A_32_P120084 | HSPC128 | HSPC128 protein | NM_014167 | 29080 | 0.083158024 |
| AGI_HUM1_OLIGO_A_32_P35220 | CBWD1 | COBW domain containing 1 | NM_172003 | 55871 | 0.083158024 |
| AGI_HUM1_OLIGO_A_32_P58163 | C15orf29 | Chromosome 15 open reading frame 29 | NM_024713 | 79768 | 0.083158024 |
| AGI_HUM1_OLIGO_A_23_P100022 | SV2B | Synaptic vesicle glycoprotein 2B | NM_014848 | 9899 | 0.083256971 |
| AGI_HUM1_OLIGO_A_23_P114423 | RGN | Regucalcin (senescence marker protein-30) | NM_004683 | 9104 | 0.083256971 |
| AGI_HUM1_OLIGO_A_23_P119857 | | | NM_001008237 | | 0.083256971 |
| AGI_HUM1_OLIGO_A_23_P143981 | FBLN2 | Fibulin 2 | NM_001004019 | 2199 | 0.083256971 |
| AGI_HUM1_OLIGO_A_23_P151895 | CILP | Cartilage intermediate layer protein, nucleotide pyrophosphohydrolase | NM_003613 | 8483 | 0.083256971 |
| AGI_HUM1_OLIGO_A_23_P155835 | CYP2U1 | Cytochrome P450, family 2, subfamily U, polypeptide 1 | NM_183075 | 113612 | 0.083256971 |
| AGI_HUM1_OLIGO_A_23_P157736 | C9orf67 | Chromosome 9 open reading frame 67 | NM_032728 | 84814 | 0.083256971 |
| AGI_HUM1_OLIGO_A_23_P170649 | RPESP | RPE-spondin | NM_153225 | 157869 | 0.083256971 |
| AGI_HUM1_OLIGO_A_23_P200015 | AK5 | Adenylate kinase 5 | NM_174858 | 26289 | 0.083256971 |
| AGI_HUM1_OLIGO_A_23_P204286 | MGP | Matrix Gla protein | NM_000900 | 4256 | 0.083256971 |
| AGI_HUM1_OLIGO_A_23_P209987 | POLR1B | Polymerase (RNA) I polypeptide B, 128 kDa | NM_019014 | 84172 | 0.083256971 |
| AGI_HUM1_OLIGO_A_23_P214281 | C6orf33 | Chromosome 6 open reading frame 33 | NM_133367 | 85315 | 0.083256971 |
| AGI_HUM1_OLIGO_A_23_P22647 | TMEM28 | Transmembrane protein 28 | NM_015686 | 27112 | 0.083256971 |
| AGI_HUM1_OLIGO_A_23_P27810 | ZNF607 | Zinc finger protein 607 | NM_032689 | 84775 | 0.083256971 |
| AGI_HUM1_OLIGO_A_23_P367899 | EPOR | Erythropoietin receptor | NM_000121 | 2057 | 0.083256971 |
| AGI_HUM1_OLIGO_A_23_P391906 | KIAA1913 | KIAA1913 | BC044246 | 114801 | 0.083256971 |
| AGI_HUM1_OLIGO_A_23_P398044 | MGC20255 | Hypothetical protein MGC20255 | NM_052848 | 90324 | 0.083256971 |
| AGI_HUM1_OLIGO_A_23_P415006 | RAB11FIP5 | RAB11 family interacting protein 5 (class I) | NM_015470 | 26056 | 0.083256971 |
| AGI_HUM1_OLIGO_A_23_P421401 | PDGFRB | Platelet-derived growth factor receptor, beta polypeptide | NM_002609 | 5159 | 0.083256971 |
| AGI_HUM1_OLIGO_A_23_P69738 | RASL11B | RAS-like, family 11, member B | NM_023940 | 65997 | 0.083256971 |
| AGI_HUM1_OLIGO_A_23_P76136 | | Similar to Peptidylprolyl isomerase A, isoform 1 | AY358804 | 390299 | 0.083256971 |
| AGI_HUM1_OLIGO_A_23_P76749 | | | NM_020692 | | 0.083256971 |
| AGI_HUM1_OLIGO_A_23_P83579 | ARNT2 | Aryl-hydrocarbon receptor nuclear translocator 2 | NM_014862 | 9915 | 0.083256971 |
| AGI_HUM1_OLIGO_A_23_P99226 | SIRT4 | Sirtuin (silent mating type information regulation 2 homolog) 4 (S. cerevisiae) | NM_012240 | 23409 | 0.083256971 |
| AGI_HUM1_OLIGO_A_24_P156886 | DKFZP564B1023 | Hypothetical protein DKFZp564B1023 | NM_031306 | 83479 | 0.083256971 |
| AGI_HUM1_OLIGO_A_24_P217330 | FLJ14566 | Hypothetical protein FLJ14566 | NM_032806 | 84892 | 0.083256971 |
| AGI_HUM1_OLIGO_A_24_P217489 | GLRB | Glycine receptor, beta | NM_000824 | 2743 | 0.083256971 |
| AGI_HUM1_OLIGO_A_24_P245298 | TNFSF13 | Tumor necrosis factor (ligand) superfamily, member 12 | NM_003809 | 8741 | 0.083256971 |
| AGI_HUM1_OLIGO_A_24_P414719 | LOC221442 | Hypothetical protein LOC221442 | AK002098 | 221442 | 0.083256971 |
| AGI_HUM1_OLIGO_A_24_P475556 | LOC146346 | Hypothetical protein LOC146346 | AK057359 | 146346 | 0.083256971 |
| AGI_HUM1_OLIGO_A_24_P720185 | | | BC092421 | | 0.083256971 |
| AGI_HUM1_OLIGO_A_24_P83848 | | Similar to Gamma-glutamyltranspeptidase 1 precursor (Gamma- | M30474 | 440796 | 0.083256971 |
| | | glutamyltransferase 1) (CD224 antigen) | | | |
| AGI_HUM1_OLIGO_A_32_P113887 | | | THC2446900 | | 0.083256971 |
| AGI_HUM1_OLIGO_A_32_P42574 | FLJ14525 | Hypothetical protein FLJ14525 | NM_032800 | 84886 | 0.083256971 |
| AGI_HUM1_OLIGO_A_32_P5251 | RARA | Retinoic acid receptor, alpha | NM_000964 | 5914 | 0.083256971 |
| AGI_HUM1_OLIGO_A_32_P6408 | DKFZp547K054 | Hypothetical protein DKFZp547K054 | AL390175 | 56974 | 0.083256971 |
| AGI_HUM1_OLIGO_A_32_P64096 | | | NM_001012981 | | 0.083256971 |
| AGI_HUM1_OLIGO_A_23_P19322 | C6orf64 | Chromosome 6 open reading frame 64 | NM_018322 | 55776 | 0.083597114 |
| AGI_HUM1_OLIGO_A_23_P201672 | KIAA0859 | KIAA0859 | NM_015935 | 51603 | 0.083597114 |
| AGI_HUM1_OLIGO_A_23_P322704 | C14orf24 | Chromosome 14 open reading frame 24 | NM_173607 | 283635 | 0.083597114 |
| AGI_HUM1_OLIGO_A_23_P403445 | CGREF1 | Cell growth regulator with EF hand domain 1 | NM_006569 | 10669 | 0.083597114 |
| AGI_HUM1_OLIGO_A_23_P82929 | NOV | Nephroblastoma overexpressed gene | NM_002514 | 4856 | 0.083597114 |
| AGI_HUM1_OLIGO_A_23_P89410 | BECN1 | Beclin 1 (coiled-coil, myosin-like BCL2 interacting protein) | NM_003766 | 8678 | 0.083597114 |
| AGI_HUM1_OLIGO_A_24_P943613 | TBC1D1 | TBC1 (tre-2/USP6, BUB2, cdc16) domain family, member 1 | NM_015173 | 23216 | 0.083597114 |
| AGI_HUM1_OLIGO_A_32_P84009 | CKLFSF4 | Chemokine-like factor super family 4 | NM_178818 | 146223 | 0.083597114 |
| AGI_HUM1_OLIGO_A_24_P109652 | KIAA2002 | KIAA2002 protein | AB082533 | 79834 | 0.083793896 |
| AGI_HUM1_OLIGO_A_23_P50167 | SLC39A6 | Solute carrier family 39 (zinc transporter), member 6 | NM_012319 | 25800 | 0.083837055 |
| AGI_HUM1_OLIGO_A_23_P158231 | APBA2 | Amyloid beta (A4) precursor protein-binding, family A, member 2 (X11-like) | NM_005503 | 321 | 0.085898602 |
| AGI_HUM1_OLIGO_A_23_P201338 | Cab45 | Calcium binding protein Cab45 precursor | NM_016176 | 51150 | 0.085898602 |
| AGI_HUM1_OLIGO_A_23_P209499 | PAX3 | Paired box gene 3 (Waardenburg syndrome 1) | NM_181458 | 5077 | 0.085898602 |
| AGI_HUM1_OLIGO_A_23_P26534 | HCFC1R1 | Host cell factor C1 regulator 1 (XPO1 dependant) | NM_017885 | 54985 | 0.085898602 |
| AGI_HUM1_OLIGO_A_24_P931944 | | CDNA FLJ25106 fis, clone CBR01467 | AK128814 | | 0.086294408 |
| AGI_HUM1_OLIGO_A_23_P153489 | SCGF | Stem cell growth factor; lymphocyte secreted C-type lectin | NM_002975 | 6320 | 0.086334725 |
| AGI_HUM1_OLIGO_A_23_P103601 | MAN1C1 | Mannosidase, alpha, class 1C, member 1 | NM_020379 | 57134 | 0.08767326 |
| AGI_HUM1_OLIGO_A_23_P168951 | ZHX2 | Zinc fingers and homeoboxes 2 | NM_014943 | 22882 | 0.08767326 |
| AGI_HUM1_OLIGO_A_24_P316364 | DKFZP566E144 | Small fragment nuclease | NM_015523 | 25996 | 0.08767326 |
| AGI_HUM1_OLIGO_A_32_P170925 | | | ENST0000036020: | | 0.08767326 |
| AGI_HUM1_OLIGO_A_23_P112311 | TRIM32 | Tripartite motif-containing 32 | NM_012210 | 22954 | 0.087758013 |
| AGI_HUM1_OLIGO_A_23_P201002 | FLJ10597 | Hypothetical protein FLJ10597 | NM_018150 | 55182 | 0.087758013 |
| AGI_HUM1_OLIGO_A_23_P205355 | SERPINA5 | Serine (or cysteine) proteinase inhibitor, clade A (alpha-1 antiproteinase, | NM_000624 | 5104 | 0.087758013 |
| | | antitrypsin), member 5 | | | |
| AGI_HUM1_OLIGO_A_23_P26890 | MMP28 | Matrix metalloproteinase 28 | NM_024302 | 79148 | 0.087758013 |
| AGI_HUM1_OLIGO_A_23_P390596 | PSKH1 | Protein serine kinase H1 | NM_006742 | 5681 | 0.087758013 |
| AGI_HUM1_OLIGO_A_24_P375132 | | | A_24_P375132 | | 0.087758013 |
| AGI_HUM1_OLIGO_A_24_P382489 | SLC27A1 | Solute carrier family 27 (fatty acid transporter), member 1 | NM_198580 | 376497 | 0.087758013 |
| AGI_HUM1_OLIGO_A_24_P89872 | ZNF189 | Zinc finger protein 189 | NM_197977 | 7743 | 0.087758013 |
| AGI_HUM1_OLIGO_A_23_P12363 | ROR1 | Receptor tyrosine kinase-like orphan receptor 1 | NM_005012 | 4919 | 0.088567004 |
| AGI_HUM1_OLIGO_A_23_P131887 | | | U35612 | | 0.088567004 |
| AGI_HUM1_OLIGO_A_23_P149496 | FLJ10307 | Hypothetical protein FLJ10307 | NM_018053 | 55113 | 0.088567004 |
| AGI_HUM1_OLIGO_A_23_P162746 | CRYL1 | Crystallin, lambda 1 | NM_015974 | 51084 | 0.088567004 |
| AGI_HUM1_OLIGO_A_23_P165927 | STMN3 | Stathmin-like 3 | NM_015894 | 50861 | 0.088567004 |
| AGI_HUM1_OLIGO_A_23_P211680 | MLC1 | Megalencephalic leukoencephalopathy with subcortical cysts 1 | NM_015166 | 23209 | 0.088567004 |
| AGI_HUM1_OLIGO_A_23_P214908 | FTHFSDC1 | Formyltetrahydrofolate synthetase domain containing 1 | AY374131 | 25902 | 0.088567004 |
| AGI_HUM1_OLIGO_A_23_P2216 | HRB2 | HIV-1 rev binding protein 2 | NM_007043 | 11103 | 0.088567004 |
| AGI_HUM1_OLIGO_A_23_P311640 | HRBL | HIV-1 Rev binding protein-like | NM_006076 | 3268 | 0.088567004 |
| AGI_HUM1_OLIGO_A_23_P31399 | PON2 | Paraoxonase 2 | NM_000305 | 5445 | 0.088567004 |
| AGI_HUM1_OLIGO_A_23_P357185 | CHDH | Choline dehydrogenase | NM_018397 | 55349 | 0.088567004 |
| AGI_HUM1_OLIGO_A_23_P377819 | SFRS5 | Splicing factor, arginine/serine-rich 5 | NM_006925 | 6430 | 0.088567004 |
| AGI_HUM1_OLIGO_A_23_P500892 | TUB | Tubby homolog (mouse) | NM_003320 | 7275 | 0.088567004 |
| AGI_HUM1_OLIGO_A_23_P501134 | LIG3 | Ligase III, DNA, ATP-dependent | NM_002311 | 3980 | 0.088567004 |
| AGI_HUM1_OLIGO_A_23_P52017 | ASPM | Asp (abnormal spindle)-like, microcephaly associated (Drosophila) | NM_018136 | 259266 | 0.088567004 |
| AGI_HUM1_OLIGO_A_23_P54041 | THTPA | Thiamine triphosphatase | NM_024328 | 79178 | 0.088567004 |
| AGI_HUM1_OLIGO_A_23_P94889 | CGI-90 | CGI-90 protein | BX640636 | 51115 | 0.088567004 |
| AGI_HUM1_OLIGO_A_24_P230877 | | CDNA FLJ33330 fis, clone BRACE2000441 | BC009463 | | 0.088567004 |
| AGI_HUM1_OLIGO_A_24_P322635 | ELMO2 | Engulfment and cell motility 2 (ced-12 homolog, C. elegans) | NM_182764 | 63916 | 0.088567004 |
| AGI_HUM1_OLIGO_A_24_P336728 | | | NM_014873 | | 0.088567004 |
| AGI_HUM1_OLIGO_A_24_P370702 | GBP3 | Guanylate binding protein 3 | NM_018284 | 2635 | 0.088567004 |
| AGI_HUM1_OLIGO_A_24_P399065 | SDOS | Hypothetical protein MGC11275 | NM_032349 | 84309 | 0.088567004 |
| AGI_HUM1_OLIGO_A_24_P413988 | TGOLN2 | Trans-golgi network protein 2 | NM_006464 | 10618 | 0.088567004 |
| AGI_HUM1_OLIGO_A_24_P683905 | | Hypothetical gene supported by AK001829 | AK001829 | 440498 | 0.088567004 |
| AGI_HUM1_OLIGO_A_24_P73290 | ATP2A2 | ATPase, Ca++ transporting, cardiac muscle, slow twitch 2 | NM_001681 | 488 | 0.088567004 |
| AGI_HUM1_OLIGO_A_24_P76313 | FAM43B | Family with sequence similarity 43, member B | NM_207334 | 163933 | 0.088567004 |
| AGI_HUM1_OLIGO_A_24_P867201 | | Homo sapiens, clone IMAGE: 5277945, mRNA | CR613944 | | 0.088567004 |
| AGI_HUM1_OLIGO_A_24_P94722 | TMEM34 | Transmembrane protein 34 | NM_018241 | 55751 | 0.088567004 |
| AGI_HUM1_OLIGO_A_32_P108738 | DKFZp761B107 | Hypothetical protein DKFZp761B107 | AK023472 | 91050 | 0.088567004 |
| AGI_HUM1_OLIGO_A_32_P11372 | FLJ30435 | Hypothetical protein FLJ30435 | NM_174950 | 387628 | 0.088567004 |
| AGI_HUM1_OLIGO_A_32_P149967 | | Transcribed locus | BX105952 | | 0.088567004 |
| AGI_HUM1_OLIGO_A_32_P196142 | | | THC2400010 | | 0.088567004 |
| AGI_HUM1_OLIGO_A_23_P165778 | MLPH | Melanophilin | NM_024101 | 79083 | 0.088872647 |
| AGI_HUM1_OLIGO_A_23_P107552 | KIAA1012 | KIAA1012 | NM_014939 | 22878 | 0.089684374 |
| AGI_HUM1_OLIGO_A_23_P203389 | MGC13379 | HSPC244 | NM_016499 | 51259 | 0.089684374 |
| AGI_HUM1_OLIGO_A_23_P416965 | DKFZP564J102 | DKFZP564J102 protein | NM_015398 | 25854 | 0.089684374 |
| AGI_HUM1_OLIGO_A_24_P225679 | IRS1 | Insulin receptor substrate 1 | NM_005544 | 3667 | 0.089684374 |
| AGI_HUM1_OLIGO_A_24_P944054 | CXCL12 | Chemokine (C—X—C motif) ligand 12 (stromal cell-derived factor 1) | AK090482 | 6387 | 0.089684374 |
| AGI_HUM1_OLIGO_A_23_P11764 | EIF2C1 | Eukaryotic translation initiation factor 2C, 1 | NM_012199 | 26523 | 0.09025495 |
| AGI_HUM1_OLIGO_A_23_P138725 | MARVELD1 | MARVEL domain containing 1 | NM_031484 | 83742 | 0.09025495 |
| AGI_HUM1_OLIGO_A_23_P139965 | | | NM_001010897 | | 0.09025495 |
| AGI_HUM1_OLIGO_A_24_P133085 | MDS025 | Hypothetical protein MDS025 | NM_021825 | 60492 | 0.09025495 |
| AGI_HUM1_OLIGO_A_24_P56467 | GMPR2 | Guanosine monophosphate reductase 2 | NM_001002000 | 51292 | 0.09025495 |
| AGI_HUM1_OLIGO_A_32_P222362 | DRG1 | Developmentally regulated GTP binding protein 1 | AK127132 | 4733 | 0.09025495 |
| AGI_HUM1_OLIGO_A_23_P102462 | FLJ37440 | Hypothetical protein FLJ37440 | NM_153214 | 129804 | 0.090484176 |
| AGI_HUM1_OLIGO_A_23_P103398 | PSEN2 | Presenilin 2 (Alzheimer disease 4) | NM_000447 | 5664 | 0.090484176 |
| AGI_HUM1_OLIGO_A_23_P112673 | FLJ10036 | Zwilch | NM_017975 | 55055 | 0.090484176 |
| AGI_HUM1_OLIGO_A_23_P117190 | DNAJD1 | DnaJ (Hsp40) homolog, subfamily D, member 1 | NM_013238 | 29103 | 0.090484176 |
| AGI_HUM1_OLIGO_A_23_P120472 | TFAP2C | Transcription factor AP-2 gamma (activating enhancer binding protein 2 gamma) | NM_003222 | 7022 | 0.090484176 |
| AGI_HUM1_OLIGO_A_23_P120942 | G22P1 | Thyroid autoantigen 70 kDa (Ku antigen) | NM_001469 | 2547 | 0.090484176 |
| AGI_HUM1_OLIGO_A_23_P121686 | SCYE1 | Small inducible cytokine subfamily E, member 1 (endothelial monocyte-activating) | NM_004757 | 9255 | 0.090484176 |
| AGI_HUM1_OLIGO_A_23_P128613 | KDELC1 | KDEL (Lys-Asp-Glu-Leu) containing 1 | NM_024089 | 79070 | 0.090484176 |
| AGI_HUM1_OLIGO_A_23_P133506 | DOK3 | Docking protein 3 | NM_024872 | 79930 | 0.090484176 |
| AGI_HUM1_OLIGO_A_23_P139455 | FLJ14451 | Hypothetical protein FLJ14451 | NM_032786 | 84872 | 0.090484176 |
| AGI_HUM1_OLIGO_A_23_P139509 | PTX1 | PTX1 protein | NM_016570 | 51290 | 0.090484176 |
| AGI_HUM1_OLIGO_A_23_P147025 | RAB33A | RAB33A, member RAS oncogene family | NM_004794 | 9363 | 0.090484176 |
| AGI_HUM1_OLIGO_A_23_P147495 | BCORL1 | BCL6 co-repressor-like 1 | NM_021946 | 63035 | 0.090484176 |
| AGI_HUM1_OLIGO_A_23_P147874 | MGC3234 | Hypothetical protein MGC3234 | NM_023947 | 66005 | 0.090484176 |
| AGI_HUM1_OLIGO_A_23_P149042 | PYCR2 | Pyrroline-5-carboxylate reductase family, member 2 | NM_013328 | 29920 | 0.090484176 |
| AGI_HUM1_OLIGO_A_23_P150267 | BBS1 | Bardet-Biedl syndrome 1 | NM_024649 | 582 | 0.090484176 |
| AGI_HUM1_OLIGO_A_23_P151529 | C14orf132 | Chromosome 14 open reading frame 132 | NM_020215 | 56967 | 0.090484176 |
| AGI_HUM1_OLIGO_A_23_P154349 | HIBCH | 3-hydroxyisobutyryl-Coenzyme A hydrolase | NM_014362 | 26275 | 0.090484176 |
| AGI_HUM1_OLIGO_A_23_P155765 | HMGB2 | High-mobility group box 2 | NM_002129 | 3148 | 0.090484176 |
| AGI_HUM1_OLIGO_A_23_P156667 | PPP1R10 | Protein phosphatase 1, regulatory subunit 10 | NM_002714 | 5514 | 0.090484176 |
| AGI_HUM1_OLIGO_A_23_P156842 | EEF1E1 | Eukaryotic translation elongation factor 1 epsilon 1 | NM_004280 | 9521 | 0.090484176 |
| AGI_HUM1_OLIGO_A_23_P162142 | TSK | Likely ortholog of chicken tsukushi | NM_015516 | 25987 | 0.090484176 |
| AGI_HUM1_OLIGO_A_23_P162846 | LAMP1 | Lysosomal-associated membrane protein 1 | NM_005561 | 3916 | 0.090484176 |
| AGI_HUM1_OLIGO_A_23_P165952 | ACTR5 | ARP5 actin-related protein 5 homolog (yeast) | NM_024855 | 79913 | 0.090484176 |
| AGI_HUM1_OLIGO_A_23_P166087 | RASSF2 | Ras association (RalGDS/AF-6) domain family 2 | NM_014737 | 9770 | 0.090484176 |
| AGI_HUM1_OLIGO_A_23_P166566 | FLJ12057 | Hypothetical protein FLJ12057 | NM_024768 | 79825 | 0.090484176 |
| AGI_HUM1_OLIGO_A_23_P168479 | RALA | V-ral simian leukemia viral oncogene homolog A (ras related) | NM_005402 | 5898 | 0.090484176 |
| AGI_HUM1_OLIGO_A_23_P169117 | RRAGA | Ras-related GTP binding A | NM_006570 | 10670 | 0.090484176 |
| AGI_HUM1_OLIGO_A_23_P18082 | FLJ14566 | Hypothetical protein FLJ14566 | NM_032806 | 84892 | 0.090484176 |
| AGI_HUM1_OLIGO_A_23_P19142 | KCNMB1 | Potassium large conductance calcium-activated channel, subfamily M, beta | NM_004137 | 3779 | 0.090484176 |
| | | member 1 | | | |
| AGI_HUM1_OLIGO_A_23_P204511 | FLJ20489 | Hypothetical protein FLJ20489 | NM_017842 | 55652 | 0.090484176 |
| AGI_HUM1_OLIGO_A_23_P205370 | ASB2 | Ankyrin repeat and SOCS box-containing 2 | NM_016150 | 51676 | 0.090484176 |
| AGI_HUM1_OLIGO_A_23_P205435 | NOVA1 | Neuro-oncological ventral antigen 1 | NM_002515 | 4857 | 0.090484176 |
| AGI_HUM1_OLIGO_A_23_P206788 | AP1G1 | Adaptor-related protein complex 1, gamma 1 subunit | NM_001128 | 164 | 0.090484176 |
| AGI_HUM1_OLIGO_A_23_P207742 | THRA | Thyroid hormone receptor, alpha (erythroblastic leukemia viral (v-erb-a) oncogene | NM_003250 | 7067 | 0.090484176 |
| | | homolog, avian) | | | |
| AGI_HUM1_OLIGO_A_23_P207999 | PMAIP1 | Phorbol-12-myristate-13-acetate-induced protein 1 | NM_021127 | 5366 | 0.090484176 |
| AGI_HUM1_OLIGO_A_23_P211631 | FBLN1 | Fibulin 1 | NM_006486 | 2192 | 0.090484176 |
| AGI_HUM1_OLIGO_A_23_P212213 | THUMPD3 | THUMP domain containing 3 | NM_015453 | 25917 | 0.090484176 |
| AGI_HUM1_OLIGO_A_23_P24135 | TACR2 | Tachykinin receptor 2 | NM_001057 | 6865 | 0.090484176 |
| AGI_HUM1_OLIGO_A_23_P250102 | | | ENST0000029598 [symbol] | | 0.090484176 |
| AGI_HUM1_OLIGO_A_23_P25060 | FLJ13769 | Hypothetical protein FLJ13769 | AK023831 | 80079 | 0.090484176 |
| AGI_HUM1_OLIGO_A_23_P250930 | CRBN | Cereblon | NM_016302 | 51185 | 0.090484176 |
| AGI_HUM1_OLIGO_A_23_P254081 | LIAS | Lipoic acid synthetase | NM_006859 | 11019 | 0.090484176 |
| AGI_HUM1_OLIGO_A_23_P254702 | DEK | DEK oncogene (DNA binding) | NM_003472 | 7913 | 0.090484176 |
| AGI_HUM1_OLIGO_A_23_P26314 | ZNF319 | Zinc finger protein 319 | NM_020807 | 57567 | 0.090484176 |
| AGI_HUM1_OLIGO_A_23_P27035 | DKFZP564K1964 | DKFZP564K1964 protein | NM_015544 | 26022 | 0.090484176 |
| AGI_HUM1_OLIGO_A_23_P27584 | | | NM_001020818 | | 0.090484176 |
| AGI_HUM1_OLIGO_A_23_P28263 | CTDSP1 | CTD (carboxy-terminal domain, RNA polymerase II, polypeptide A) small | NM_021198 | 58190 | 0.090484176 |
| | | phosphatase 1 | | | |
| AGI_HUM1_OLIGO_A_23_P30020 | PLA2G12A | Phospholipase A2, group XIIA | NM_030821 | 81579 | 0.090484176 |
| AGI_HUM1_OLIGO_A_23_P303763 | ZNF79 | Zinc finger protein 79 (pT7) | NM_007135 | 7633 | 0.090484176 |
| AGI_HUM1_OLIGO_A_23_P303833 | SCN4B | Sodium channel, voltage-gated, type IV, beta | NM_174934 | 6330 | 0.090484176 |
| AGI_HUM1_OLIGO_A_23_P310956 | COL6A2 | Collagen, type VI, alpha 2 | NM_058175 | 1292 | 0.090484176 |
| AGI_HUM1_OLIGO_A_23_P327426 | MGC3794 | Putative MAPK activating protein | NM_152902 | 261726 | 0.090484176 |
| AGI_HUM1_OLIGO_A_23_P331908 | PRDM11 | PR domain containing 11 | AK097878 | 56981 | 0.090484176 |
| AGI_HUM1_OLIGO_A_23_P34402 | NCSTN | Nicastrin | NM_015331 | 23385 | 0.090484176 |
| AGI_HUM1_OLIGO_A_23_P345674 | ZNF71 | Zinc finger protein 71 (Cos26) | NM_021216 | 58491 | 0.090484176 |
| AGI_HUM1_OLIGO_A_23_P350824 | DPYSL2 | Dihydropyrimidinase-like 2 | NM_001386 | 1808 | 0.090484176 |
| AGI_HUM1_OLIGO_A_23_P352950 | PNMA5 | Paraneoplastic antigen like 5 | NM_052926 | 114824 | 0.090484176 |
| AGI_HUM1_OLIGO_A_23_P35617 | PLCE1 | Phospholipase C, epsilon 1 | NM_016341 | 51196 | 0.090484176 |
| AGI_HUM1_OLIGO_A_23_P357936 | ASTN2 | Astrotactin 2 | NM_014010 | 23245 | 0.090484176 |
| AGI_HUM1_OLIGO_A_23_P360964 | MGC15476 | Thymus expressed gene 3-like | NM_145056 | 147906 | 0.090484176 |
| AGI_HUM1_OLIGO_A_23_P360983 | LOC91942 | Hypothetical protein LOC91942 | NM_174889 | 91942 | 0.090484176 |
| AGI_HUM1_OLIGO_A_23_P364056 | | | NM_001011724 | | 0.090484176 |
| AGI_HUM1_OLIGO_A_23_P369047 | | | ENST0000032498 [symbol] | | 0.090484176 |
| AGI_HUM1_OLIGO_A_23_P369994 | DCAMKL1 | Doublecortin and CaM kinase-like 1 | NM_004734 | 9201 | 0.090484176 |
| AGI_HUM1_OLIGO_A_23_P374288 | CASC3 | Cancer susceptibility candidate 3 | NM_007359 | 22794 | 0.090484176 |
| AGI_HUM1_OLIGO_A_23_P37505 | DYX1C1 | Dyslexia susceptibility 1 candidate 1 | NM_130810 | 161582 | 0.090484176 |
| AGI_HUM1_OLIGO_A_23_P37514 | DKFZP434H132 | DKFZP434H132 protein | BC011905 | 56905 | 0.090484176 |
| AGI_HUM1_OLIGO_A_23_P387374 | HSD17B1 | Hydroxysteroid (17-beta) dehydrogenase 1 | BC033110 | 3292 | 0.090484176 |
| AGI_HUM1_OLIGO_A_23_P389907 | ATXN2 | Ataxin 2 | NM_002973 | 6311 | 0.090484176 |
| AGI_HUM1_OLIGO_A_23_P394216 | KIAA0329 | KIAA0329 | NM_014844 | 9895 | 0.090484176 |
| AGI_HUM1_OLIGO_A_23_P395566 | FBXO31 | F-box protein 31 | NM_024735 | 79791 | 0.090484176 |
| AGI_HUM1_OLIGO_A_23_P407142 | LUZP1 | Leucine zipper protein 1 | NM_033631 | 7798 | 0.090484176 |
| AGI_HUM1_OLIGO_A_23_P409945 | OAZ1 | Ornithine decarboxylase antizyme 1 | NM_004152 | 4946 | 0.090484176 |
| AGI_HUM1_OLIGO_A_23_P41314 | F11 | Coagulation factor XI (plasma thromboplastin antecedent) | NM_000128 | 2160 | 0.090484176 |
| AGI_HUM1_OLIGO_A_23_P416581 | GNAZ | Guanine nucleotide binding protein (G protein), alpha z polypeptide | NM_002073 | 2781 | 0.090484176 |
| AGI_HUM1_OLIGO_A_23_P42036 | DJ122O8.2 | Hypothetical protein dJ122O8.2 | NM_020466 | 57226 | 0.090484176 |
| AGI_HUM1_OLIGO_A_23_P42042 | DJ122O8.2 | Hypothetical protein dJ122O8.2 | NM_020466 | 57226 | 0.090484176 |
| AGI_HUM1_OLIGO_A_23_P421638 | KIAA1190 | Hypothetical protein KIAA1190 | NM_145166 | 92999 | 0.090484176 |
| AGI_HUM1_OLIGO_A_23_P42975 | PRKAR2B | Protein kinase, cAMP-dependent, regulatory, type II, beta | NM_002736 | 5577 | 0.090484176 |
| AGI_HUM1_OLIGO_A_23_P43276 | GPR124 | G protein-coupled receptor 124 | NM_032777 | 25960 | 0.090484176 |
| AGI_HUM1_OLIGO_A_23_P43566 | NDUFA8 | NADH dehydrogenase (ubiquinone) 1 alpha subcomplex, 8, 19 kDa | NM_014222 | 4702 | 0.090484176 |
| AGI_HUM1_OLIGO_A_23_P44291 | CRTAP | Cartilage associated protein | NM_006371 | 10491 | 0.090484176 |
| AGI_HUM1_OLIGO_A_23_P48717 | NPC2 | Niemann-Pick disease, type C2 | NM_006432 | 10577 | 0.090484176 |
| AGI_HUM1_OLIGO_A_23_P53588 | WNT5B | Wingless-type MMTV integration site family, member 5B | NM_030775 | 81029 | 0.090484176 |
| AGI_HUM1_OLIGO_A_23_P54576 | KIFC3 | Kinesin family member C3 | NM_005550 | 3801 | 0.090484176 |
| AGI_HUM1_OLIGO_A_23_P5568 | MGC5391 | Hypothetical protein MGC5391 | NM_032740 | 84826 | 0.090484176 |
| AGI_HUM1_OLIGO_A_23_P60146 | PDGFRL | Platelet-derived growth factor receptor-like | NM_006207 | 5157 | 0.090484176 |
| AGI_HUM1_OLIGO_A_23_P61406 | SHC3 | Chromosome 9 open reading frame 33 | NM_016848 | 53358 | 0.090484176 |
| AGI_HUM1_OLIGO_A_23_P64489 | RIC-8 | Likely ortholog of mouse synembryn | NM_021932 | 60626 | 0.090484176 |
| AGI_HUM1_OLIGO_A_23_P67360 | PLEKHA4 | Pleckstrin homology domain containing, family A (phosphoinositide binding | NM_020904 | 57664 | 0.090484176 |
| | | specific) member 4 | | | |
| AGI_HUM1_OLIGO_A_23_P69267 | DKFZP564O243 | DKFZP564O243 protein | NM_015407 | 25864 | 0.090484176 |
| AGI_HUM1_OLIGO_A_23_P69497 | TNA | Tetranectin (plasminogen binding protein) | NM_003278 | 7123 | 0.090484176 |
| AGI_HUM1_OLIGO_A_23_P71091 | GCC1 | GRIP and coiled-coil domain containing 1 | NM_024523 | 79571 | 0.090484176 |
| AGI_HUM1_OLIGO_A_23_P7144 | CXCL1 | Chemokine (C—X—C motif) ligand 1 (melanoma growth stimulating activity, alpha) | NM_001511 | 2919 | 0.090484176 |
| AGI_HUM1_OLIGO_A_23_P74349 | CDCA1 | Cell division cycle associated 1 | NM_145697 | 83540 | 0.090484176 |
| AGI_HUM1_OLIGO_A_23_P82068 | NOL7 | Nucleolar protein 7, 27 kDa | NM_016167 | 51406 | 0.090484176 |
| AGI_HUM1_OLIGO_A_23_P8281 | IFNGR1 | Interferon gamma receptor 1 | NM_000416 | 3459 | 0.090484176 |
| AGI_HUM1_OLIGO_A_23_P84018 | PPIC | Peptidylprolyl isomerase C (cyclophilin C) | NM_000943 | 5480 | 0.090484176 |
| AGI_HUM1_OLIGO_A_23_P84070 | HDCMA18P | HDCMA18P protein | NM_016648 | 51574 | 0.090484176 |
| AGI_HUM1_OLIGO_A_23_P88559 | LIPC | Lipase, hepatic | NM_000236 | 3990 | 0.090484176 |
| AGI_HUM1_OLIGO_A_23_P9232 | GCNT1 | Glucosaminyl (N-acetyl) transferase 1, core 2 (beta-1,6-N- | NM_001490 | 2650 | 0.090484176 |
| | | acetylglucosaminyltransferase) | | | |
| AGI_HUM1_OLIGO_A_23_P98350 | BIRC3 | Baculoviral IAP repeat-containing 3 | NM_001165 | 330 | 0.090484176 |
| AGI_HUM1_OLIGO_A_24_P106624 | MEOX2 | Mesenchyme homeo box 2 (growth arrest-specific homeo box) | NM_005924 | 4223 | 0.090484176 |
| AGI_HUM1_OLIGO_A_24_P115621 | | | ENST0000037321 [symbol] | | 0.090484176 |
| AGI_HUM1_OLIGO_A_24_P116535 | MMP15 | Matrix metalloproteinase 15 (membrane-inserted) | NM_002428 | 4324 | 0.090484176 |
| AGI_HUM1_OLIGO_A_24_P12435 | NCOA7 | Nuclear receptor coactivator 7 | NM_181782 | 135112 | 0.090484176 |
| AGI_HUM1_OLIGO_A_24_P131522 | ANTXR1 | Anthrax toxin receptor 1 | NM_032208 | 84168 | 0.090484176 |
| AGI_HUM1_OLIGO_A_24_P143843 | | | ENST0000034268 [symbol] | | 0.090484176 |
| AGI_HUM1_OLIGO_A_24_P144784 | HILS1 | Spermatid-specific linker histone H1-like protein | NM_194072 | 373861 | 0.090484176 |
| AGI_HUM1_OLIGO_A_24_P148373 | | | ENST0000037401 [symbol] | | 0.090484176 |
| AGI_HUM1_OLIGO_A_24_P149124 | C5orf13 | Chromosome 5 open reading frame 13 | NM_004772 | 9315 | 0.090484176 |
| AGI_HUM1_OLIGO_A_24_P163632 | OXR1 | Oxidation resistance 1 | NM_181354 | 55074 | 0.090484176 |
| AGI_HUM1_OLIGO_A_24_P167473 | ARPC3 | Actin related protein 2/3 complex, subunit 3, 21 kDa | NM_005719 | 10094 | 0.090484176 |
| AGI_HUM1_OLIGO_A_24_P17722 | KIAA0676 | KIAA0676 protein | NM_198868 | 23061 | 0.090484176 |
| AGI_HUM1_OLIGO_A_24_P178273 | PTK9 | PTK9 protein tyrosine kinase 9 | NM_198974 | 5756 | 0.090484176 |
| AGI_HUM1_OLIGO_A_24_P195205 | | | ENST0000036142 [symbol] | | 0.090484176 |
| AGI_HUM1_OLIGO_A_24_P196774 | NUDT1 | Nudix (nucleoside diphosphate linked moiety X)-type motif 1 | BC014395 | 4521 | 0.090484176 |
| AGI_HUM1_OLIGO_A_24_P205130 | FNBP1 | Formin binding protein 1 | NM_015033 | 23048 | 0.090484176 |
| AGI_HUM1_OLIGO_A_24_P218587 | CRSP6 | Cofactor required for Sp1 transcriptional activation, subunit 6, 77 kDa | NM_004268 | 9440 | 0.090484176 |
| AGI_HUM1_OLIGO_A_24_P22436 | MGC4606 | Hypothetical protein MGC4606 | NM_024516 | 79447 | 0.090484176 |
| AGI_HUM1_OLIGO_A_24_P234871 | | | A_24_P234871 | | 0.090484176 |
| AGI_HUM1_OLIGO_A_24_P251899 | CSNK1A1 | Casein kinase 1, alpha 1 | NM_001892 | 1452 | 0.090484176 |
| AGI_HUM1_OLIGO_A_24_P252846 | LOC119710 | Hypothetical protein BC009561 | NM_138787 | 119710 | 0.090484176 |
| AGI_HUM1_OLIGO_A_24_P264644 | | | A_24_P264644 | | 0.090484176 |
| AGI_HUM1_OLIGO_A_24_P281730 | | | A_24_P281730 | | 0.090484176 |
| AGI_HUM1_OLIGO_A_24_P298545 | MGC42415 | Hypothetical protein MGC42415 | NM_153363 | 168417 | 0.090484176 |
| AGI_HUM1_OLIGO_A_24_P29885 | NOV | Nephroblastoma overexpressed gene | NM_002514 | 4856 | 0.090484176 |
| AGI_HUM1_OLIGO_A_24_P30670 | WBP4 | WW domain binding protein 4 (formin binding protein 21) | NM_007187 | 11193 | 0.090484176 |
| AGI_HUM1_OLIGO_A_24_P320328 | PC4 | Activated RNA polymerase II transcription cofactor 4 | NM_006713 | 10923 | 0.090484176 |
| AGI_HUM1_OLIGO_A_24_P32836 | | | A_24_P32836 | | 0.090484176 |
| AGI_HUM1_OLIGO_A_24_P330668 | KIAA1970 | KIAA1970 protein | NM_019116 | 124454 | 0.090484176 |
| AGI_HUM1_OLIGO_A_24_P339416 | KIAA1001 | Arylsulfatase G | NM_014960 | 22901 | 0.090484176 |
| AGI_HUM1_OLIGO_A_24_P339611 | PDCD5 | Programmed cell death 5 | NM_004708 | 9141 | 0.090484176 |
| AGI_HUM1_OLIGO_A_24_P340066 | ELF4 | E74-like factor 4 (ets domain transcription factor) | NM_001421 | 2000 | 0.090484176 |
| AGI_HUM1_OLIGO_A_24_P345377 | GOLPH3L | Golgi phosphoprotein 3-like | NM_018178 | 55204 | 0.090484176 |
| AGI_HUM1_OLIGO_A_24_P346269 | | | THC2342624 | | 0.090484176 |
| AGI_HUM1_OLIGO_A_24_P348649 | NDUFA1 | NADH dehydrogenase (ubiquinone) 1 alpha subcomplex, 1, 7.5 kDa | NM_004541 | 4694 | 0.090484176 |
| AGI_HUM1_OLIGO_A_24_P351435 | CRBN | Cereblon | NM_016302 | 51185 | 0.090484176 |
| AGI_HUM1_OLIGO_A_24_P376391 | PLXND1 | Plexin D1 | NM_015103 | 23129 | 0.090484176 |
| AGI_HUM1_OLIGO_A_24_P378302 | | | NM_001040150 | | 0.090484176 |
| AGI_HUM1_OLIGO_A_24_P379820 | ITM2C | Integral membrane protein 2C | NM_030926 | 81618 | 0.090484176 |
| AGI_HUM1_OLIGO_A_24_P408047 | PLEKHA4 | Pleckstrin homology domain containing, family A (phosphoinositide binding | NM_020904 | 57664 | 0.090484176 |
| | | specific) member 4 | | | |
| AGI_HUM1_OLIGO_A_24_P41339 | | | XM_930460 | | 0.090484176 |
| AGI_HUM1_OLIGO_A_24_P43588 | MAD2L1BP | MAD2L1 binding protein | NM_001003690 | 9587 | 0.090484176 |
| AGI_HUM1_OLIGO_A_24_P478556 | | CDNA clone IMAGE: 4796690, partial cds | BC030102 | | 0.090484176 |
| AGI_HUM1_OLIGO_A_24_P49597 | | | ENST0000029975 [symbol] | | 0.090484176 |
| AGI_HUM1_OLIGO_A_24_P592591 | | | THC2270231 | | 0.090484176 |
| AGI_HUM1_OLIGO_A_24_P59607 | | | ENST0000037919 [symbol] | | 0.090484176 |
| AGI_HUM1_OLIGO_A_24_P605612 | THBS2 | Thrombospondin 2 | NM_003247 | 7058 | 0.090484176 |
| AGI_HUM1_OLIGO_A_24_P62521 | PSEN2 | Presenilin 2 (Alzheimer disease 4) | NM_000447 | 5664 | 0.090484176 |
| AGI_HUM1_OLIGO_A_24_P68381 | DCT | Dopachrome tautomerase (dopachrome delta-isomerase, tyrosine-related protein | NM_001922 | 1638 | 0.090484176 |
| | | 2) | | | |
| AGI_HUM1_OLIGO_A_24_P6850 | | | A_24_P6850 | | 0.090484176 |
| AGI_HUM1_OLIGO_A_24_P73599 | IL16 | Interleukin 16 (lymphocyte chemoattractant factor) | NM_172217 | 3603 | 0.090484176 |
| AGI_HUM1_OLIGO_A_24_P832156 | C9orf10OS | Chromosome 9 open reading frame 10 opposite strand | NM_198841 | 158293 | 0.090484176 |
| AGI_HUM1_OLIGO_A_24_P920447 | C14orf132 | Chromosome 14 open reading frame 132 | NM_020215 | 56967 | 0.090484176 |
| AGI_HUM1_OLIGO_A_24_P940006 | EFNB3 | Ephrin-B3 | NM_001406 | 1949 | 0.090484176 |
| AGI_HUM1_OLIGO_A_32_P101860 | | | XM_930086 | | 0.090484176 |
| AGI_HUM1_OLIGO_A_32_P108722 | | Full-length cDNA clone CS0DC003YA14 of Neuroblastoma Cot 25-normalized of | AK127572 | | 0.090484176 |
| | | Homo sapiens (human) | | | |
| AGI_HUM1_OLIGO_A_32_P113404 | | | A_32_P113404 | | 0.090484176 |
| AGI_HUM1_OLIGO_A_32_P125338 | FAM43B | Family with sequence similarity 43, member B | NM_207334 | 163933 | 0.090484176 |
| AGI_HUM1_OLIGO_A_32_P1445 | PTPN2 | Protein tyrosine phosphatase, non-receptor type 2 | NM_080423 | 5771 | 0.090484176 |
| AGI_HUM1_OLIGO_A_32_P150040 | | Full-length cDNA clone CS0DF003YF10 of Fetal brain of Homo sapiens (human) | CR606969 | | 0.090484176 |
| AGI_HUM1_OLIGO_A_32_P161432 | CBWD1 | COBW domain containing 1 | NM_172003 | 55871 | 0.090484176 |
| AGI_HUM1_OLIGO_A_32_P169131 | ALG1 | Asparagine-linked glycosylation 1 homolog (yeast, beta-1,4-mannosyltransferase) | NM_019109 | 56052 | 0.090484176 |
| AGI_HUM1_OLIGO_A_32_P172141 | CDON | Cell adhesion molecule-related/down-regulated by oncogenes | NM_016952 | 50937 | 0.090484176 |
| AGI_HUM1_OLIGO_A_32_P178099 | | | EB386378 | | 0.090484176 |
| AGI_HUM1_OLIGO_A_32_P179676 | TOB2 | Transducer of ERBB2, 2 | NM_016272 | 10766 | 0.090484176 |
| AGI_HUM1_OLIGO_A_32_P188860 | IL17RD | Interleukin 17 receptor D | AK125591 | 54756 | 0.090484176 |
| AGI_HUM1_OLIGO_A_32_P229065 | | | NM_001031800 | | 0.090484176 |
| AGI_HUM1_OLIGO_A_32_P53524 | | | BC092429 | | 0.090484176 |
| AGI_HUM1_OLIGO_A_32_P59549 | GFRA1 | GDNF family receptor alpha 1 | NM_145793 | 2674 | 0.090484176 |
| AGI_HUM1_OLIGO_A_32_P65395 | | | XM_209655 | | 0.090484176 |
| AGI_HUM1_OLIGO_A_32_P78285 | | | XM_938617 | | 0.090484176 |
| AGI_HUM1_OLIGO_A_32_P79396 | PBEF1 | Pre-B-cell colony enhancing factor 1 | NM_005746 | 10135 | 0.090484176 |
| AGI_HUM1_OLIGO_A_32_P80089 | | | ENST0000034606 [symbol] | | 0.090484176 |
| AGI_HUM1_OLIGO_A_32_P84707 | | | THC2308802 | | 0.090484176 |
| AGI_HUM1_OLIGO_A_32_P84846 | LOC163131 | Hypothetical BC331191_1 | NM_001005851 | 163131 | 0.090484176 |
| AGI_HUM1_OLIGO_A_32_P94087 | | | NM_001011538 | | 0.090484176 |
| AGI_HUM1_OLIGO_A_32_P99902 | MGC29937 | Hypothetical protein MGC29937 | NM_144597 | 123207 | 0.090484176 |
| AGI_HUM1_OLIGO_A_24_P110967 | LOC115294 | Similar to hypothetical protein FLJ10883 | NM_052937 | 115294 | 0.090566641 |
| AGI_HUM1_OLIGO_A_23_P208961 | MUM1 | Melanoma associated antigen (mutated) 1 | NM_032853 | 84939 | 0.090776283 |
| AGI_HUM1_OLIGO_A_23_P81103 | SFRP2 | Secreted frizzled-related protein 2 | NM_003013 | 6423 | 0.090875466 |
| AGI_HUM1_OLIGO_A_32_P62997 | TOPK | T-LAK cell-originated protein kinase | NM_018492 | 55872 | 0.090875466 |
| AGI_HUM1_OLIGO_A_23_P433820 | THAP8 | THAP domain containing 8 | NM_152658 | 199745 | 0.091250181 |
| AGI_HUM1_OLIGO_A_24_P311694 | MAML1 | Mastermind-like 1 (Drosophila) | NM_014757 | 9794 | 0.091250181 |
| AGI_HUM1_OLIGO_A_24_P50829 | TRPM7 | Transient receptor potential cation channel, subfamily M, member 7 | NM_017672 | 54822 | 0.091250181 |
| AGI_HUM1_OLIGO_A_32_P101301 | USP39 | Ubiquitin specific protease 39 | NM_006590 | 10713 | 0.091250181 |
| AGI_HUM1_OLIGO_A_23_P100711 | PMP22 | Peripheral myelin protein 22 | NM_000304 | 5376 | 0.091253805 |
| AGI_HUM1_OLIGO_A_23_P145463 | SLC35B3 | Solute carrier family 35, member B3 | NM_015948 | 51000 | 0.091522184 |
| AGI_HUM1_OLIGO_A_23_P419624 | BLCAP | Bladder cancer associated protein | NM_006698 | 10904 | 0.091522184 |
| AGI_HUM1_OLIGO_A_23_P400794 | | | ENST0000031163 [symbol] | | 0.092872595 |
| AGI_HUM1_OLIGO_A_23_P86884 | SART1 | Squamous cell carcinoma antigen recognised by T cells | NM_005146 | 9092 | 0.092872595 |
| AGI_HUM1_OLIGO_A_24_P419211 | MTMR6 | Myotubularin related protein 6 | NM_004685 | 9107 | 0.092872595 |
| AGI_HUM1_OLIGO_A_23_P12911 | C11orf24 | Chromosome 11 open reading frame 24 | NM_022338 | 53838 | 0.093105978 |
| AGI_HUM1_OLIGO_A_23_P32861 | CGI-07 | CGI-07 protein | NM_015938 | 51068 | 0.093105978 |
| AGI_HUM1_OLIGO_A_23_P47665 | HBE1 | Hemoglobin, epsilon 1 | NM_005330 | 3046 | 0.093105978 |
| AGI_HUM1_OLIGO_A_23_P51202 | ZNF436 | Zinc finger protein 436 | NM_030634 | 80818 | 0.093105978 |
| AGI_HUM1_OLIGO_A_23_P7229 | RPL34 | Ribosomal protein L34 | NM_033625 | 6164 | 0.093105978 |
| AGI_HUM1_OLIGO_A_23_P8513 | SNX10 | Sorting nexin 10 | NM_013322 | 29887 | 0.093105978 |
| AGI_HUM1_OLIGO_A_23_P157038 | MGC40499 | Hypothetical protein MGC40499 | NM_152755 | 245812 | 0.093210689 |
| AGI_HUM1_OLIGO_A_24_P246825 | | | THC2295607 | | 0.093210689 |
| AGI_HUM1_OLIGO_A_32_P32254 | COL6A1 | Collagen, type VI, alpha 1 | NM_001848 | 1291 | 0.093210689 |
| AGI_HUM1_OLIGO_A_23_P124003 | P2RX2 | Purinergic receptor P2X, ligand-gated ion channel, 2 | NM_170683 | 22953 | 0.093708677 |
| AGI_HUM1_OLIGO_A_23_P101871 | FBXO17 | F-box protein 17 | NM_024907 | 115290 | 0.093887665 |
| AGI_HUM1_OLIGO_A_23_P111132 | HSPA1A | Heat shock 70 kDa protein 1A | NM_005345 | 3303 | 0.093887665 |
| AGI_HUM1_OLIGO_A_23_P112289 | TMOD1 | Tropomodulin 1 | NM_003275 | 7111 | 0.093887665 |
| AGI_HUM1_OLIGO_A_23_P114952 | TMEM9 | Transmembrane protein 9 | NM_016456 | 252839 | 0.093887665 |
| AGI_HUM1_OLIGO_A_23_P115356 | AKR7A2 | Aldo-keto reductase family 7, member A2 (aflatoxin aldehyde reductase) | NM_003689 | 8574 | 0.093887665 |
| AGI_HUM1_OLIGO_A_23_P115460 | RPL22 | Ribosomal protein L22 | NM_000983 | 6146 | 0.093887665 |
| AGI_HUM1_OLIGO_A_23_P115922 | EIF4EBP2 | Eukaryotic translation initiation factor 4E binding protein 2 | NM_004096 | 1979 | 0.093887665 |
| AGI_HUM1_OLIGO_A_23_P117082 | HEBP1 | Heme binding protein 1 | NM_015987 | 50865 | 0.093887665 |
| AGI_HUM1_OLIGO_A_23_P117654 | MYO5A | Myosin VA (heavy polypeptide 12, myoxin) | NM_000259 | 4644 | 0.093887665 |
| AGI_HUM1_OLIGO_A_23_P118493 | TOM1L1 | Target of myb1-like 1 (chicken) | NM_005486 | 10040 | 0.093887665 |
| AGI_HUM1_OLIGO_A_23_P119562 | DF | D component of complement (adipsin) | NM_001928 | 1675 | 0.093887665 |
| AGI_HUM1_OLIGO_A_23_P120103 | KCNS3 | Potassium voltage-gated channel, delayed-rectifier, subfamily S, member 3 | NM_002252 | 3790 | 0.093887665 |
| AGI_HUM1_OLIGO_A_23_P128956 | ZFYVE1 | Zinc finger, FYVE domain containing 1 | NM_021260 | 53349 | 0.093887665 |
| AGI_HUM1_OLIGO_A_23_P1331 | COL13A1 | Collagen, type XIII, alpha 1 | NM_005203 | 1305 | 0.093887665 |
| AGI_HUM1_OLIGO_A_23_P139476 | CD63 | CD63 antigen (melanoma 1 antigen) | NM_001780 | 967 | 0.093887665 |
| AGI_HUM1_OLIGO_A_23_P147514 | DKFZp547E052 | Hypothetical protein DKFZp547E052 | BC062636 | 84236 | 0.093887665 |
| AGI_HUM1_OLIGO_A_23_P157051 | AP4M1 | Adaptor-related protein complex 4, mu 1 subunit | NM_004722 | 9179 | 0.093887665 |
| AGI_HUM1_OLIGO_A_23_P162795 | SUGT1 | SGT1, suppressor of G2 allele of SKP1 (S. cerevisiae) | NM_006704 | 10910 | 0.093887665 |
| AGI_HUM1_OLIGO_A_23_P165608 | SEMA4F | Sema domain, immunoglobulin domain (Ig), transmembrane domain (TM) and | NM_004263 | 10505 | 0.093887665 |
| | | short cytoplasmic domain, (semaphorin) 4F | | | |
| AGI_HUM1_OLIGO_A_23_P201963 | PARD3 | Par-3 partitioning defective 3 homolog (C. elegans) | AF196185 | 56288 | 0.093887665 |
| AGI_HUM1_OLIGO_A_23_P211015 | C21orf91 | Chromosome 21 open reading frame 91 | NM_017447 | 54149 | 0.093887665 |
| AGI_HUM1_OLIGO_A_23_P21382 | LAMB2 | Laminin, beta 2 (laminin S) | NM_002292 | 3913 | 0.093887665 |
| AGI_HUM1_OLIGO_A_23_P253052 | CD99L2 | CD99 antigen-like 2 | NM_031462 | 83692 | 0.093887665 |
| AGI_HUM1_OLIGO_A_23_P254165 | RAI2 | Retinoic acid induced 2 | NM_021785 | 10742 | 0.093887665 |
| AGI_HUM1_OLIGO_A_23_P254978 | TATDN1 | TatD DNase domain containing 1 | NM_032026 | 83940 | 0.093887665 |
| AGI_HUM1_OLIGO_A_23_P258666 | | MRNA, endogenous retrovirus | BC021996 | | 0.093887665 |
| AGI_HUM1_OLIGO_A_23_P26184 | DET1 | De-etiolated 1 | NM_017996 | 55070 | 0.093887665 |
| AGI_HUM1_OLIGO_A_23_P26954 | VAT1 | Vesicle amine transport protein 1 homolog (T californica) | NM_006373 | 10493 | 0.093887665 |
| AGI_HUM1_OLIGO_A_23_P28434 | VAMP8 | Vesicle-associated membrane protein 8 (endobrevin) | NM_003761 | 8673 | 0.093887665 |
| AGI_HUM1_OLIGO_A_23_P304171 | | | ENST0000027358 [symbol] | | 0.093887665 |
| AGI_HUM1_OLIGO_A_23_P313645 | MAP3K2 | Mitogen-activated protein kinase kinase kinase 2 | NM_006609 | 10746 | 0.093887665 |
| AGI_HUM1_OLIGO_A_23_P321377 | FLJ38984 | Hypothetical protein FLJ38984 | NM_152374 | 127703 | 0.093887665 |
| AGI_HUM1_OLIGO_A_23_P329890 | MGC17839 | Hypothetical protein MGC17839 | NM_174926 | 219902 | 0.093887665 |
| AGI_HUM1_OLIGO_A_23_P334709 | FKBP9 | FK506 binding protein 9, 63 kDa | NM_007270 | 11328 | 0.093887665 |
| AGI_HUM1_OLIGO_A_23_P339480 | HAT1 | Histone acetyltransferase 1 | NM_003642 | 8520 | 0.093887665 |
| AGI_HUM1_OLIGO_A_23_P34233 | QPRT | Quinolinate phosphoribosyltransferase (nicotinate-nucleotide pyrophosphorylase | NM_014298 | 23475 | 0.093887665 |
| | | (carboxylating)) | | | |
| AGI_HUM1_OLIGO_A_23_P356004 | CSEN | Calsenilin, presenilin binding protein, EF hand transcription factor | NM_013434 | 30818 | 0.093887665 |
| AGI_HUM1_OLIGO_A_23_P391689 | PCP2 | Purkinje cell protein 2 | CR602285 | 126006 | 0.093887665 |
| AGI_HUM1_OLIGO_A_23_P401 | CENPF | Centromere protein F, 350/400ka (mitosin) | NM_016346 | 1063 | 0.093887665 |
| AGI_HUM1_OLIGO_A_23_P402610 | PFAS | Phosphoribosylformylglycinamidine synthase (FGAR amidotransferase) | NM_012393 | 5198 | 0.093887665 |
| AGI_HUM1_OLIGO_A_23_P408996 | OACT1 | O-acyltransferase (membrane bound) domain containing 1 | AK131269 | 154141 | 0.093887665 |
| AGI_HUM1_OLIGO_A_23_P411953 | PYGO2 | Pygopus homolog 2 (Drosophila) | NM_138300 | 90780 | 0.093887665 |
| AGI_HUM1_OLIGO_A_23_P4212 | HOXB13 | Homeo box B13 | NM_006361 | 10481 | 0.093887665 |
| AGI_HUM1_OLIGO_A_23_P432573 | MRGPRF | MAS-related GPR, member F | NM_145015 | 219928 | 0.093887665 |
| AGI_HUM1_OLIGO_A_23_P44684 | ECT2 | Epithelial cell transforming sequence 2 oncogene | NM_018098 | 1894 | 0.093887665 |
| AGI_HUM1_OLIGO_A_23_P502174 | D2LIC | Dynein 2 light intermediate chain | NM_015522 | 51626 | 0.093887665 |
| AGI_HUM1_OLIGO_A_23_P51346 | | | ENST0000037657 [symbol] | | 0.093887665 |
| AGI_HUM1_OLIGO_A_23_P56328 | PLVAP | Plasmalemma vesicle associated protein | NM_031310 | 83483 | 0.093887665 |
| AGI_HUM1_OLIGO_A_23_P56938 | REL | V-rel reticuloendotheliosis viral oncogene homolog (avian) | NM_002908 | 5966 | 0.093887665 |
| AGI_HUM1_OLIGO_A_23_P59921 | PC4 | Activated RNA polymerase II transcription cofactor 4 | NM_006713 | 10923 | 0.093887665 |
| AGI_HUM1_OLIGO_A_23_P66766 | RIP | RPA interacting protein | NM_032308 | 84268 | 0.093887665 |
| AGI_HUM1_OLIGO_A_23_P69121 | SIAH2 | Seven in absentia homolog 2 (Drosophila) | NM_005067 | 6478 | 0.093887665 |
| AGI_HUM1_OLIGO_A_23_P84836 | NPEPPS | Aminopeptidase puromycin sensitive | NM_006310 | 9520 | 0.093887665 |
| AGI_HUM1_OLIGO_A_23_P98218 | HSPC148 | Hypothetical protein HSPC148 | NM_016403 | 51503 | 0.093887665 |
| AGI_HUM1_OLIGO_A_24_P121631 | MGC13138 | Hypothetical protein MGC13138 | NM_033410 | 92595 | 0.093887665 |
| AGI_HUM1_OLIGO_A_24_P123658 | | | ENST0000037287 [symbol] | | 0.093887665 |
| AGI_HUM1_OLIGO_A_24_P150428 | FLJ30707 | Hypothetical protein FLJ30707 | NM_145019 | 220108 | 0.093887665 |
| AGI_HUM1_OLIGO_A_24_P175989 | VPS29 | Vacuolar protein sorting 29 (yeast) | NM_057180 | 51699 | 0.093887665 |
| AGI_HUM1_OLIGO_A_24_P189458 | C10orf86 | Chromosome 10 open reading frame 86 | NM_017615 | 54780 | 0.093887665 |
| AGI_HUM1_OLIGO_A_24_P191067 | | | NM_001009566 | | 0.093887665 |
| AGI_HUM1_OLIGO_A_24_P200427 | PAICS | Phosphoribosylaminoimidazole carboxylase, phosphoribosylaminoimidazole | NM_006452 | 10606 | 0.093887665 |
| | | succinocarboxamide synthetase | | | |
| AGI_HUM1_OLIGO_A_24_P203479 | MSH3 | MutS homolog 3 (E. coli) | NM_002439 | 4437 | 0.093887665 |
| AGI_HUM1_OLIGO_A_24_P213487 | LAT1-3TM | LAT1-3TM protein | AF271775 | 81893 | 0.093887665 |
| AGI_HUM1_OLIGO_A_24_P229728 | | | A_24_P229728 | | 0.093887665 |
| AGI_HUM1_OLIGO_A_24_P271363 | CDS2 | CDP-diacylglycerol synthase (phosphatidate cytidylyltransferase) 2 | NM_003818 | 8760 | 0.093887665 |
| AGI_HUM1_OLIGO_A_24_P277657 | GMPR | Guanosine monophosphate reductase | NM_006877 | 2766 | 0.093887665 |
| AGI_HUM1_OLIGO_A_24_P281395 | | | ENST0000037228 [symbol] | | 0.093887665 |
| AGI_HUM1_OLIGO_A_24_P286013 | HIP2 | Huntingtin interacting protein 2 | NM_005339 | 3093 | 0.093887665 |
| AGI_HUM1_OLIGO_A_24_P296808 | FLJ10781 | Hypothetical protein FLJ10781 | NM_018215 | 55228 | 0.093887665 |
| AGI_HUM1_OLIGO_A_24_P296907 | THAP8 | THAP domain containing 8 | NM_152658 | 199745 | 0.093887665 |
| AGI_HUM1_OLIGO_A_24_P341426 | | | A_24_P341426 | | 0.093887665 |
| AGI_HUM1_OLIGO_A_24_P409420 | | | A_24_P409420 | | 0.093887665 |
| AGI_HUM1_OLIGO_A_24_P466374 | EYA3 | Eyes absent homolog 3 (Drosophila) | BX648945 | 2140 | 0.093887665 |
| AGI_HUM1_OLIGO_A_24_P48177 | SIAT4B | Sialyltransferase 4B (beta-galactoside alpha-2,3-sialyltransferase) | AK127322 | 6483 | 0.093887665 |
| AGI_HUM1_OLIGO_A_24_P511877 | | CDNA FLJ46881 fis, clone UTERU3015647, moderately similar to Embigin precursor | AK128714 | | 0.093887665 |
| AGI_HUM1_OLIGO_A_24_P782308 | NEDD4L | Neural precursor cell expressed, developmentally down-regulated 4-like | CR596491 | 23327 | 0.093887665 |
| AGI_HUM1_OLIGO_A_24_P844100 | | | A_24_P844100 | | 0.093887665 |
| AGI_HUM1_OLIGO_A_24_P944827 | APG7L | APG7 autophagy 7-like (S. cerevisiae) | AL122075 | 10533 | 0.093887665 |
| AGI_HUM1_OLIGO_A_24_P95029 | TAX1BP1 | Tax1 (human T-cell leukemia virus type I) binding protein 1 | NM_006024 | 8887 | 0.093887665 |
| AGI_HUM1_OLIGO_A_32_P112279 | | | NM_001039690 | | 0.093887665 |
| AGI_HUM1_OLIGO_A_32_P116058 | | | NR_002800 | | 0.093887665 |
| AGI_HUM1_OLIGO_A_32_P116203 | NCF1 | Neutrophil cytosolic factor 1 (47 kDa, chronic granulomatous disease, autosomal 1) | NM_000265 | 4687 | 0.093887665 |
| AGI_HUM1_OLIGO_A_32_P163469 | NFE2L1 | Nuclear factor (erythroid-derived 2)-like 1 | AL833530 | 4779 | 0.093887665 |
| AGI_HUM1_OLIGO_A_32_P171232 | | | A_32_P171232 | | 0.093887665 |
| AGI_HUM1_OLIGO_A_32_P184367 | RPL10 | Ribosomal protein L10 | NM_006013 | 6134 | 0.093887665 |
| AGI_HUM1_OLIGO_A_32_P225355 | CPEB2 | Cytoplasmic polyadenylation element binding protein 2 | NM_182485 | 132864 | 0.093887665 |
| AGI_HUM1_OLIGO_A_32_P230736 | | Hypothetical LOC389033 | XM_374010 | 389033 | 0.093887665 |
| AGI_HUM1_OLIGO_A_32_P2333 | PC4 | Activated RNA polymerase II transcription cofactor 4 | NM_006713 | 10923 | 0.093887665 |
| AGI_HUM1_OLIGO_A_32_P32179 | FLJ44670 | FLJ44670 protein | AK057056 | 375096 | 0.093887665 |
| AGI_HUM1_OLIGO_A_32_P32722 | LOC255458 | Hypothetical protein LOC255458 | CR603845 | 255458 | 0.093887665 |
| AGI_HUM1_OLIGO_A_32_P460399 | | MRNA full length insert cDNA clone EUROIMAGE 2362292 | AL365520 | | 0.093887665 |
| AGI_HUM1_OLIGO_A_32_P58215 | | | ENST0000036805 [symbol] | | 0.093887665 |
| AGI_HUM1_OLIGO_A_32_P6442 | | | THC2406192 | | 0.093887665 |
| AGI_HUM1_OLIGO_A_32_P83845 | HEY1 | Hairy/enhancer-of-split related with YRPW motif 1 | NM_012258 | 23462 | 0.093887665 |
| AGI_HUM1_OLIGO_A_23_P49279 | LOC388272 | Similar to RIKEN cDNA 4921524J17 | NM_001001436 | 388272 | 0.093920232 |
| AGI_HUM1_OLIGO_A_23_P78134 | | | NM_001014764 | | 0.093920232 |
| AGI_HUM1_OLIGO_A_23_P92441 | MAD2L1 | MAD2 mitotic arrest deficient-like 1 (yeast) | NM_002358 | 4085 | 0.093920232 |
| AGI_HUM1_OLIGO_A_24_P237778 | MAN1C1 | Mannosidase, alpha, class 1C, member 1 | NM_020379 | 57134 | 0.093920232 |
| AGI_HUM1_OLIGO_A_24_P358205 | | | A_24_P358205 | | 0.093920232 |
| AGI_HUM1_OLIGO_A_24_P47467 | MGC5508 | Hypothetical protein MGC5508 | NM_024092 | 79073 | 0.093920232 |
| AGI_HUM1_OLIGO_A_23_P216949 | PDCL | Phosducin-like | NM_005388 | 5082 | 0.094362947 |
| AGI_HUM1_OLIGO_A_23_P154306 | TANK | TRAF family member-associated NFKB activator | NM_004180 | 10010 | 0.095027623 |
| AGI_HUM1_OLIGO_A_23_P213385 | BASP1 | Brain abundant, membrane attached signal protein 1 | NM_006317 | 10409 | 0.095027623 |
| AGI_HUM1_OLIGO_A_23_P251387 | REPS2 | RALBP1 associated Eps domain containing 2 | NM_004726 | 9185 | 0.095027623 |
| AGI_HUM1_OLIGO_A_23_P254733 | MLF1IP | MLF1 interacting protein | NM_024629 | 79682 | 0.095027623 |
| AGI_HUM1_OLIGO_A_23_P29747 | RPL23A | Ribosomal protein L23a | NM_000984 | 6147 | 0.095027623 |
| AGI_HUM1_OLIGO_A_23_P371613 | CHCHD1 | Coiled-coil-helix-coiled-coil-helix domain containing 1 | NM_203298 | 118487 | 0.095027623 |
| AGI_HUM1_OLIGO_A_23_P434900 | C16orf34 | Chromosome 16 open reading frame 34 | NM_144570 | 90861 | 0.095027623 |
| AGI_HUM1_OLIGO_A_23_P65262 | PFAAP5 | Phosphonoformate immuno-associated protein 5 | NM_033111 | 10443 | 0.095027623 |
| AGI_HUM1_OLIGO_A_23_P71570 | OSR2 | Odd-skipped related 2 (Drosophila) | NM_053001 | 116039 | 0.095027623 |
| AGI_HUM1_OLIGO_A_24_P125311 | | | A_24_P125311 | | 0.095027623 |
| AGI_HUM1_OLIGO_A_24_P15765 | FMN2 | Formin 2 | AK098605 | 56776 | 0.095027623 |
| AGI_HUM1_OLIGO_A_24_P287503 | ZFYVE1 | Zinc finger, FYVE domain containing 1 | NM_021260 | 53349 | 0.095027623 |
| AGI_HUM1_OLIGO_A_24_P385119 | TAF2 | TAF2 RNA polymerase II, TATA box binding protein (TBP)-associated factor, | NM_003184 | 6873 | 0.095027623 |
| | | 150 kDa | | | |
| AGI_HUM1_OLIGO_A_24_P58899 | | | A_24_P58899 | | 0.095027623 |
| AGI_HUM1_OLIGO_A_24_P943843 | | CDNA FLJ30141 fis, clone BRACE2000148 | AK124515 | | 0.095027623 |
| AGI_HUM1_OLIGO_A_32_P113193 | ZNF592 | Zinc finger protein 592 | NM_014630 | 9640 | 0.095027623 |
| AGI_HUM1_OLIGO_A_32_P141445 | MGC34796 | SPR pseudogene | BC034822 | 414927 | 0.095027623 |
| AGI_HUM1_OLIGO_A_23_P140960 | NDUFAB1 | NADH dehydrogenase (ubiquinone) 1, alpha/beta subcomplex, 1, 8 kDa | NM_005003 | 4706 | 0.095262298 |
| AGI_HUM1_OLIGO_A_23_P214091 | LYPLA1 | Lysophospholipase I | NM_006330 | 10434 | 0.095262298 |
| AGI_HUM1_OLIGO_A_23_P315933 | WBSCR21 | Williams Beuren syndrome chromosome region 21 | NM_031295 | 83451 | 0.095262298 |
| AGI_HUM1_OLIGO_A_23_P50108 | KNTC2 | Kinetochore associated 2 | NM_006101 | 10403 | 0.095262298 |
| AGI_HUM1_OLIGO_A_23_P6674 | LXN | Latexin | NM_020169 | 56925 | 0.095262298 |
| AGI_HUM1_OLIGO_A_23_P88810 | MLYCD | Malonyl-CoA decarboxylase | NM_012213 | 23417 | 0.095262298 |
| AGI_HUM1_OLIGO_A_24_P124370 | PARVA | Parvin, alpha | NM_018222 | 55742 | 0.095262298 |
| AGI_HUM1_OLIGO_A_32_P96752 | SOX4 | SRY (sex determining region Y)-box 4 | AW946823 | 6659 | 0.095262298 |
| AGI_HUM1_OLIGO_A_23_P375165 | FLJ35767 | FLJ35767 protein | NM_207459 | 400629 | 0.095338015 |
| AGI_HUM1_OLIGO_A_24_P170887 | FLJ20989 | Hypothetical protein FLJ20989 | NM_023080 | 65265 | 0.095436493 |
| AGI_HUM1_OLIGO_A_23_P109001 | KIAA0406 | KIAA0406 gene product | NM_014657 | 9675 | 0.095538395 |
| AGI_HUM1_OLIGO_A_23_P202219 | FAM26B | Family with sequence similarity 26, member B | NM_015916 | 51063 | 0.095538395 |
| AGI_HUM1_OLIGO_A_23_P22460 | BIRC4 | Baculoviral IAP repeat-containing 4 | NM_001167 | 331 | 0.095538395 |
| AGI_HUM1_OLIGO_A_23_P401076 | SUSD3 | Sushi domain containing 3 | NM_145006 | 203328 | 0.095538395 |
| AGI_HUM1_OLIGO_A_23_P63026 | LGALS8 | Lectin, galactoside-binding, soluble, 8 (galectin 8) | NM_006499 | 3964 | 0.095538395 |
| AGI_HUM1_OLIGO_A_23_P96350 | PRAF2 | PRA1 domain family, member 2 | NM_007213 | 11230 | 0.095538395 |
| AGI_HUM1_OLIGO_A_24_P282013 | MEF2B | MADS box transcription enhancer factor 2, polypeptide B (myocyte enhancer factor | AK057161 | 4207 | 0.095538395 |
| | | 2B) | | | |
| AGI_HUM1_OLIGO_A_24_P943000 | POP1 | Processing of precursor 1, ribonuclease P/MRP subunit (S. cerevisiae) | D31765 | 10940 | 0.095538395 |
| AGI_HUM1_OLIGO_A_23_P154037 | AOX1 | Aldehyde oxidase 1 | NM_001159 | 316 | 0.09565787 |
| AGI_HUM1_OLIGO_A_23_P334282 | BMP2K | BMP2 inducible kinase | NM_017593 | 55589 | 0.095822416 |
| AGI_HUM1_OLIGO_A_23_P38864 | RABAC1 | Rab acceptor 1 (prenylated) | NM_006423 | 10567 | 0.095822416 |
| AGI_HUM1_OLIGO_A_23_P81898 | UBD | Ubiquitin D | NM_006398 | 10537 | 0.095822416 |
| AGI_HUM1_OLIGO_A_24_P196592 | MMP28 | Matrix metalloproteinase 28 | NM_024302 | 79148 | 0.095866261 |
| AGI_HUM1_OLIGO_A_32_P116113 | DIO2 | Deiodinase, iodothyronine, type II | CN479762 | 1734 | 0.095866261 |
| AGI_HUM1_OLIGO_A_24_P349547 | | | A_24_P349547 | | 0.096048224 |
| AGI_HUM1_OLIGO_A_23_P309619 | PLEKHM1 | Pleckstrin homology domain containing, family M (with RUN domain) member 1 | AK123704 | 9842 | 0.096145456 |
| AGI_HUM1_OLIGO_A_23_P396858 | FZD8 | Frizzled homolog 8 (Drosophila) | NM_031866 | 8325 | 0.096145456 |
| AGI_HUM1_OLIGO_A_23_P399169 | HIC1 | Hypermethylated in cancer 1 | NM_006497 | 3090 | 0.096145456 |
| AGI_HUM1_OLIGO_A_23_P58117 | ShrmL | Shroom-related protein | NM_020859 | 57619 | 0.096145456 |
| AGI_HUM1_OLIGO_A_23_P88470 | TRPM7 | Transient receptor potential cation channel, subfamily M, member 7 | NM_017672 | 54822 | 0.096145456 |
| AGI_HUM1_OLIGO_A_24_P344961 | AMOT | Angiomotin | NM_133265 | 154796 | 0.096145456 |
| AGI_HUM1_OLIGO_A_23_P140654 | | | THC2375798 | | 0.096332895 |
| AGI_HUM1_OLIGO_A_23_P148629 | EIF1AY | Eukaryotic translation Initiation factor 1A, Y-linked | NM_004681 | 9086 | 0.096332895 |
| AGI_HUM1_OLIGO_A_23_P163306 | CGNL1 | Cingulin-like 1 | NM_032866 | 84952 | 0.096332895 |
| AGI_HUM1_OLIGO_A_23_P168167 | C6orf168 | Chromosome 6 open reading frame 168 | NM_032511 | 84553 | 0.096332895 |
| AGI_HUM1_OLIGO_A_23_P18317 | SLC41A3 | Solute carrier family 41, member 3 | NM_017836 | 54946 | 0.096332895 |
| AGI_HUM1_OLIGO_A_23_P212196 | OGG1 | 8-oxoguanine DNA glycosylase | NM_016828 | 4968 | 0.096332895 |
| AGI_HUM1_OLIGO_A_23_P212535 | DKFZP564O123 | DKFZP564O123 protein | NM_014043 | 25978 | 0.096332895 |
| AGI_HUM1_OLIGO_A_23_P217049 | FREQ | Frequenin homolog (Drosophila) | NM_014286 | 23413 | 0.096332895 |
| AGI_HUM1_OLIGO_A_23_P217498 | GDPD2 | Glycerophosphodiester phosphodiesterase domain containing 2 | NM_017711 | 54857 | 0.096332895 |
| AGI_HUM1_OLIGO_A_23_P301855 | LSAMP | Limbic system-associated membrane protein | NM_002338 | 4045 | 0.096332895 |
| AGI_HUM1_OLIGO_A_23_P31315 | CBX3 | Chromobox homolog 3 (HP1 gamma homolog, Drosophila) | NM_016587 | 11335 | 0.096332895 |
| AGI_HUM1_OLIGO_A_23_P325924 | | | ENST0000028867 [symbol] | | 0.096332895 |
| AGI_HUM1_OLIGO_A_23_P36183 | GTF2H1 | General transcription factor IIH, polypeptide 1, 62 kDa | NM_005316 | 2965 | 0.096332895 |
| AGI_HUM1_OLIGO_A_23_P421011 | KAZALD1 | Kazal-type serine protease inhibitor domain 1 | NM_030929 | 81621 | 0.096332895 |
| AGI_HUM1_OLIGO_A_23_P500773 | MAP3K7 | Mitogen-activated protein kinase kinase kinase 7 | NM_003188 | 6885 | 0.096332895 |
| AGI_HUM1_OLIGO_A_23_P9192 | ROD1 | ROD1 regulator of differentiation 1 (S. pombe) | NM_005156 | 9991 | 0.096332895 |
| AGI_HUM1_OLIGO_A_24_P124992 | PSMA4 | Proteasome (prosome, macropain) subunit, alpha type, 4 | NM_002789 | 5685 | 0.096332895 |
| AGI_HUM1_OLIGO_A_24_P186746 | | | A_24_P186746 | | 0.096332895 |
| AGI_HUM1_OLIGO_A_24_P203827 | | | ENST0000031034 [symbol] | | 0.096332895 |
| AGI_HUM1_OLIGO_A_24_P307065 | | | XM_933001 | | 0.096332895 |
| AGI_HUM1_OLIGO_A_24_P313397 | | | ENST0000021916 [symbol] | | 0.096332895 |
| AGI_HUM1_OLIGO_A_24_P941166 | ZNF425 | Zinc finger protein 425 | NM_001001661 | 155054 | 0.096332895 |
| AGI_HUM1_OLIGO_A_23_P104876 | SPA17 | Sperm autoantigenic protein 17 | NM_017425 | 53340 | 0.096604616 |
| AGI_HUM1_OLIGO_A_23_P141946 | LTBP4 | Latent transforming growth factor beta binding protein 4 | NM_003573 | 8425 | 0.096604616 |
| AGI_HUM1_OLIGO_A_23_P252536 | MRPS26 | Mitochondrial ribosomal protein S26 | NM_030811 | 64949 | 0.096604616 |
| AGI_HUM1_OLIGO_A_23_P2705 | P2RY5 | Purinergic receptor P2Y, G-protein coupled, 5 | NM_005767 | 10161 | 0.096604616 |
| AGI_HUM1_OLIGO_A_23_P67228 | SNAPC2 | Small nuclear RNA activating complex, polypeptide 2, 45 kDa | NM_003083 | 6618 | 0.096604616 |
| AGI_HUM1_OLIGO_A_23_P91076 | FLJ14681 | Hypothetical protein FLJ14681 | CR621710 | 84910 | 0.096604616 |
| AGI_HUM1_OLIGO_A_24_P108242 | GOSR2 | Golgi SNAP receptor complex member 2 | NM_004287 | 9570 | 0.096604616 |
| AGI_HUM1_OLIGO_A_24_P168760 | GTPBP1 | GTP binding protein 1 | NM_004286 | 9567 | 0.096604616 |
| AGI_HUM1_OLIGO_A_24_P347480 | | | ENST0000023861 [symbol] | | 0.096604616 |
| AGI_HUM1_OLIGO_A_24_P912058 | | | S76980 | | 0.096604616 |
| AGI_HUM1_OLIGO_A_32_P31633 | SF3B1 | Splicing factor 3b, subunit 1, 155 kDa | NM_012433 | 23451 | 0.096604616 |
| AGI_HUM1_OLIGO_A_23_P30474 | FLJ10233 | Hypothetical protein FLJ10233 | NM_018034 | 55100 | 0.09712464 |
| AGI_HUM1_OLIGO_A_32_P178092 | AFAP | Hypothetical protein LOC254848 | BC014113 | 60312 | 0.09745375 |
| AGI_HUM1_OLIGO_A_23_P134714 | HRSP12 | Heat-responsive protein 12 | NM_005836 | 10247 | 0.09762118 |
| AGI_HUM1_OLIGO_A_32_P175098 | ASPH | Aspartate beta-hydroxylase | CR613736 | 444 | 0.09762118 |
| AGI_HUM1_OLIGO_A_23_P252145 | C1GALT1 | Core 1 UDP-galactose:N-acetylgalactosamine-alpha-R beta 1,3- | NM_020156 | 56913 | 0.097940226 |
| | | galactosyltransferase | | | |
| AGI_HUM1_OLIGO_A_23_P104692 | MGC35521 | Pellino 3 alpha | NM_145065 | 246330 | 0.098033415 |
| AGI_HUM1_OLIGO_A_23_P136238 | MGC14327 | Hypothetical protein MGC14327 | NM_053045 | 94107 | 0.098033415 |
| AGI_HUM1_OLIGO_A_23_P148879 | ATP1A2 | ATPase, Na+/K+ transporting, alpha 2 (+) polypeptide | NM_000702 | 477 | 0.098033415 |
| AGI_HUM1_OLIGO_A_23_P157147 | ZDHHC4 | Zinc finger, DHHC domain containing 4 | NM_018106 | 55146 | 0.098033415 |
| AGI_HUM1_OLIGO_A_23_P313512 | DCP1B | Decapping enzyme hDcp1b | NM_152640 | 196513 | 0.098033415 |
| AGI_HUM1_OLIGO_A_23_P336859 | LOC284542 | Hypothetical protein LOC284542 | AK090467 | 284542 | 0.098033415 |
| AGI_HUM1_OLIGO_A_23_P383009 | IGFBP5 | Insulin-like growth factor binding protein 5 | NM_000599 | 3488 | 0.098033415 |
| AGI_HUM1_OLIGO_A_23_P5051 | GNA11 | Guanine nucleotide binding protein (G protein), alpha 11 (Gq class) | BC063426 | 2767 | 0.098033415 |
| AGI_HUM1_OLIGO_A_24_P624439 | | | NM_001013672 | | 0.098033415 |
| AGI_HUM1_OLIGO_A_32_P125568 | | | THC2416690 | | 0.098033415 |
| AGI_HUM1_OLIGO_A_32_P161681 | | | NM_001008528 | | 0.098033415 |
| AGI_HUM1_OLIGO_A_32_P184746 | | | THC2378401 | | 0.098033415 |
| AGI_HUM1_OLIGO_A_32_P220127 | RPL34 | Ribosomal protein L34 | NM_033625 | 6164 | 0.098033415 |
| AGI_HUM1_OLIGO_A_23_P114144 | CXorf37 | Chromosome X open reading frame 37 | NM_014008 | 28952 | 0.09816416 |
| AGI_HUM1_OLIGO_A_23_P147185 | CANX | Calnexin | NM_001746 | 821 | 0.09816416 |
| AGI_HUM1_OLIGO_A_23_P213551 | CSNK1A1 | Casein kinase 1, alpha 1 | NM_001892 | 1452 | 0.09816416 |
| AGI_HUM1_OLIGO_A_24_P40165 | PIGT | Phosphatidylinositol glycan, class T | NM_015937 | 51604 | 0.09816416 |
| AGI_HUM1_OLIGO_A_24_P621023 | | | ENST0000037381 [symbol] | | 0.09816416 |
| AGI_HUM1_OLIGO_A_32_P10633 | C22orf4 | Chromosome 22 open reading frame 4 | BC022417 | 25771 | 0.09816416 |
| AGI_HUM1_OLIGO_A_23_P93722 | SDK1 | Sidekick homolog 1 (chicken) | AK074077 | 221935 | 0.098210685 |
| AGI_HUM1_OLIGO_A_23_P48835 | KIF23 | Kinesin family member 23 | NM_138555 | 9493 | 0.098273168 |
| AGI_HUM1_OLIGO_A_23_P129301 | C15orf25 | Chromosome 15 open reading frame 25 | NM_018097 | 55142 | 0.098500774 |
| AGI_HUM1_OLIGO_A_23_P162739 | TGFB1I4 | Transforming growth factor beta 1 induced transcript 4 | NM_183422 | 8848 | 0.098500774 |
| AGI_HUM1_OLIGO_A_32_P165713 | CIP29 | Cytokine induced protein 29 kDa | NM_033082 | 84324 | 0.098500774 |
| AGI_HUM1_OLIGO_A_32_P2738 | C10orf61 | Chromosome 10 open reading frame 61 | NM_015631 | 26123 | 0.098500774 |
| AGI_HUM1_OLIGO_A_23_P155477 | LOC51161 | G20 protein | NM_016210 | 51161 | 0.099065131 |
| AGI_HUM1_OLIGO_A_23_P164081 | MYH10 | Myosin, heavy polypeptide 10, non-muscle | NM_005964 | 4628 | 0.099065131 |
| AGI_HUM1_OLIGO_A_23_P354591 | | | NM_033446 | | 0.099065131 |
| AGI_HUM1_OLIGO_A_23_P43484 | CDKN2A | Cyclin-dependent kinase inhibitor 2A (melanoma, p16, inhibits CDK4) | NM_058197 | 1029 | 0.099065131 |
| AGI_HUM1_OLIGO_A_24_P195974 | | | A_24_P195974 | | 0.099131448 |
| AGI_HUM1_OLIGO_A_24_P269398 | CASP2 | Caspase 2, apoptosis-related cysteine protease (neural precursor cell expressed, | NM_032982 | 835 | 0.099131448 |
| | | developmentally down-regulated 2) | | | |
| AGI_HUM1_OLIGO_A_32_P73775 | KIAA0495 | KIAA0495 | NM_207306 | 57212 | 0.099131448 |
| AGI_HUM1_OLIGO_A_32_P168561 | | | THC2413666 | | 0.099301528 |
| AGI_HUM1_OLIGO_A_23_P140301 | PSMA3 | Proteasome (prosome, macropain) subunit, alpha type, 3 | NM_002788 | 5684 | 0.099409858 |
| AGI_HUM1_OLIGO_A_23_P140668 | IDH3A | Isocitrate dehydrogenase 3 (NAD+) alpha | NM_005530 | 3419 | 0.099409858 |
| AGI_HUM1_OLIGO_A_23_P163148 | C14orf133 | Chromosome 14 open reading frame 133 | NM_022067 | 63894 | 0.099409858 |
| AGI_HUM1_OLIGO_A_23_P219223 | | | NM_080701 | | 0.099409858 |
| AGI_HUM1_OLIGO_A_23_P23266 | | | ENST0000036780 [symbol] | | 0.099409858 |
| AGI_HUM1_OLIGO_A_23_P55045 | SLC25A19 | Solute carrier family 25 (mitochondrial deoxynucleotide carrier), member 19 | NM_021734 | 60386 | 0.099409858 |
| AGI_HUM1_OLIGO_A_23_P87752 | LEPREL2 | Leprecan-like 2 | NM_014262 | 10536 | 0.099409858 |
| AGI_HUM1_OLIGO_A_24_P175612 | SFXN2 | Sideroflexin 2 | NM_178858 | 118980 | 0.099409858 |
| AGI_HUM1_OLIGO_A_23_P20864 | ANGPTL2 | Angiopoietin-like 2 | NM_012098 | 23452 | 0.099636713 |
| AGI_HUM1_OLIGO_A_24_P220485 | OLFML2A | Olfactomedin-like 2A | NM_182487 | 169611 | 0.099636713 |
| AGI_HUM1_OLIGO_A_23_P125423 | C1R | Complement component 1, r subcomponent | NM_001733 | 715 | 0.099983113 |
| AGI_HUM1_OLIGO_A_23_P83493 | CGI-90 | CGI-90 protein | NM_016033 | 51115 | 0.099983113 |

[symbol]

indicates data missing or illegible when filed

TABLE 2
FDR for Comparison of Baseline Arm and Back Samples Between Responders and Non-Responders

| Agilent Probe ID | Gene Symbol | Gene Name | Accession | Entrez Gene ID | FDR |
|---|---|---|---|---|---|
| AGI_HUM1_OLIGO_A_23_P500410 | ATP6V1G2 | ATPase, H+ transporting, lysosomal 13 kDa, V1 subunit G isoform 2 | NM_130463 | 534 | 0.00556686 |
| AGI_HUM1_OLIGO_A_23_P250313 | KIAA1524 | KIAA1524 Protein | AB040957 | 57650 | 0.00592933 |
| AGI_HUM1_OLIGO_A_23_P18267 | TUSC4 | Tumor suppressor candidate 4 | NM_006545 | 10641 | 0.00611253 |
| AGI_HUM1_OLIGO_A_24_P93741 | LOC285398 | Hypothetical protein LOC285398 | AK098811 | 285398 | 0.00611253 |
| AGI_HUM1_OLIGO_A_23_P352684 | WDR22 | WD repeat domain 22 | NM_003861 | 8816 | 0.00611253 |
| AGI_HUM1_OLIGO_A_23_P329740 | UBC | Ubiquitin C | NM_021009 | 7316 | 0.00884742 |
| AGI_HUM1_OLIGO_A_32_P65473 | | | ENST00000330640 | | 0.00884742 |
| AGI_HUM1_OLIGO_A_23_P57227 | SNTA1 | Syntrophin, alpha 1 (dystrophin-associated protein A1, 59 kDa, acidic component) | NM_003098 | 6640 | 0.00884742 |
| AGI_HUM1_OLIGO_A_23_P360964 | MGC15476 | Thymus expressed gene 3-like | NM_145056 | 147906 | 0.00961462 |
| AGI_HUM1_OLIGO_A_23_P162466 | PKP2 | Plakophilin 2 | NM_004572 | 5318 | 0.01072345 |
| AGI_HUM1_OLIGO_A_24_P344961 | AMOT | Angiomotin | NM_133265 | 154796 | 0.01072345 |
| AGI_HUM1_OLIGO_A_24_P93462 | | | A_24_P93462 | | 0.01072345 |
| AGI_HUM1_OLIGO_A_24_P943613 | TBC1D1 | TBC1 (tre-2/USP6, BUB2, cdc16) domain family, member 1 | NM_015173 | 23216 | 0.01072345 |
| AGI_HUM1_OLIGO_A_23_P77310 | SH3BP3 | SH3-domain binding protein 3 | NM_022473 | 64397 | 0.01072345 |
| AGI_HUM1_OLIGO_A_23_P55926 | NR1H2 | Nuclear receptor subfamily 1, group H, member 2 | NM_007121 | 7376 | 0.01072345 |
| AGI_HUM1_OLIGO_A_23_P368484 | FLJ35696 | FLJ35696 protein | NM_207387 | 388341 | 0.01072345 |
| AGI_HUM1_OLIGO_A_24_P399680 | C20orf108 | Chromosome 20 open reading frame 108 | NM_080821 | 116151 | 0.01128431 |
| AGI_HUM1_OLIGO_A_23_P169738 | SOX7 | SRY (sex determining region Y)-box 7 | NM_031439 | 83595 | 0.01216998 |
| AGI_HUM1_OLIGO_A_24_P137434 | DCBLD2 | Discoidin, CUB and LCCL domain containing 2 | NM_080927 | 131566 | 0.01216998 |
| AGI_HUM1_OLIGO_A_23_P19134 | MASS1 | Monogenic, audiogenic seizure susceptibility 1 homolog (mouse) | NM_032119 | 84059 | 0.01231733 |
| AGI_HUM1_OLIGO_A_24_P200942 | THG-1 | TSC-22-like | NM_030935 | 81628 | 0.01331317 |
| AGI_HUM1_OLIGO_A_24_P337000 | TTC7A | Tetratricopeptide repeat domain 7A | NM_020458 | 57217 | 0.01331317 |
| AGI_HUM1_OLIGO_A_23_P333683 | IGSF10 | Immunoglobulin superfamily, member 10 | NM_178822 | 285313 | 0.01454206 |
| AGI_HUM1_OLIGO_A_24_P115621 | | | ENST00000373218 | | 0.01801736 |
| AGI_HUM1_OLIGO_A_23_P158885 | LOC114977 | Hypothetical protein BC014148 | AK096342 | 114977 | 0.01836372 |
| AGI_HUM1_OLIGO_A_24_P931944 | | CDNA FLJ25106 fis, clone CBR01467 | AK128814 | | 0.01836372 |
| AGI_HUM1_OLIGO_A_23_P146654 | BAG1 | BCL2-associated athanogene | NM_004323 | 573 | 0.01945735 |
| AGI_HUM1_OLIGO_A_24_P378987 | DHRSX | Zinc finger, BED domain containing 1 | NM_145177 | 207063 | 0.02031249 |
| AGI_HUM1_OLIGO_A_32_P135336 | | | NR_002556 | | 0.02031249 |
| AGI_HUM1_OLIGO_A_32_P196837 | DLK1 | Delta-like 1 homolog (Drosophila) | AA449494 | 8788 | 0.02031249 |
| AGI_HUM1_OLIGO_A_24_P314688 | USP40 | Ubiquitin specific protease 40 | NM_018218 | 55230 | 0.02031249 |
| AGI_HUM1_OLIGO_A_24_P289188 | | | ENST00000287144 | | 0.02066335 |
| AGI_HUM1_OLIGO_A_23_P24318 | RPS6KB2 | Ribosomal protein S6 kinase, 70 kDa, polypeptide 2 | NM_001007071 | 6199 | 0.0210813 |
| AGI_HUM1_OLIGO_A_23_P23630 | CENTB5 | Centaurin, beta 5 | NM_030649 | 116983 | 0.0210813 |
| AGI_HUM1_OLIGO_A_24_P287613 | MAP2K1 | Mitogen-activated protein kinase kinase 1 | BC066984 | 5604 | 0.0210813 |
| AGI_HUM1_OLIGO_A_23_P23194 | PINK1 | PTEN induced putative kinase 1 | NM_032409 | 65018 | 0.0210813 |
| AGI_HUM1_OLIGO_A_23_P94319 | | | NM_014867 | | 0.0210813 |
| AGI_HUM1_OLIGO_A_23_P151791 | LTB4R | Leukotriene B4 receptor | NM_181657 | 1241 | 0.0210813 |
| AGI_HUM1_OLIGO_A_23_P146354 | POMT1 | Protein-O-mannosyltransferase 1 | NM_007171 | 10585 | 0.02121958 |
| AGI_HUM1_OLIGO_A_23_P153461 | LPPR2 | Lipid phosphate phosphatase-related protein type 2 | NM_022737 | 64748 | 0.02121958 |
| AGI_HUM1_OLIGO_A_24_P820302 | | | THC2411433 | | 0.02121958 |
| AGI_HUM1_OLIGO_A_23_P308150 | FLJ39827 | Hypothetical protein FLJ39827 | NM_152424 | 139285 | 0.02121958 |
| AGI_HUM1_OLIGO_A_24_P873764 | BCR | Breakpoint cluster region | NM_004327 | 613 | 0.02121958 |
| AGI_HUM1_OLIGO_A_24_P280903 | | | ENST00000311218 | | 0.02121958 |
| AGI_HUM1_OLIGO_A_23_P395524 | PWP1 | Nuclear phosphoprotein similar to S. cerevisiae PWP1 | NM_007062 | 11137 | 0.02121958 |
| AGI_HUM1_OLIGO_A_32_P175935 | | CDNA FLJ43172 fis, clone FCBBF3007242 | AK125162 | | 0.02121958 |
| AGI_HUM1_OLIGO_A_23_P62387 | NUDT11 | Nudix (nucleoside diphosphate linked moiety X)-type motif 11 | NM_018159 | 55190 | 0.02121958 |
| AGI_HUM1_OLIGO_A_23_P393080 | CALML3 | Calmodulin-like 3 | NM_005185 | 810 | 0.02121958 |
| AGI_HUM1_OLIGO_A_24_P418019 | | | A_24_P418019 | | 0.02121958 |
| AGI_HUM1_OLIGO_A_23_P134693 | | | NM_001009552 | | 0.02121958 |
| AGI_HUM1_OLIGO_A_23_P207650 | ACADVL | Acyl-Coenzyme A dehydrogenase, very long chain | NM_000018 | 37 | 0.02121958 |
| AGI_HUM1_OLIGO_A_32_P116058 | | | NR_002800 | | 0.02121958 |
| AGI_HUM1_OLIGO_A_24_P737553 | | CDNA FLJ13712 fis, clone PLACE2000394 | AK023774 | | 0.02121958 |
| AGI_HUM1_OLIGO_A_32_P224586 | | | THC2342537 | | 0.02121958 |
| AGI_HUM1_OLIGO_A_23_P104295 | SHFM3 | Split hand/foot malformation (ectrodactyly) type 3 | NM_022039 | 6468 | 0.02121958 |
| AGI_HUM1_OLIGO_A_32_P108592 | | | THC2312637 | | 0.02121958 |
| AGI_HUM1_OLIGO_A_32_P100439 | Ells1 | Hypothetical protein Ells1 | NM_152793 | 222166 | 0.02121958 |
| AGI_HUM1_OLIGO_A_23_P218807 | RoXaN | Rotavirus X protein associated with NSP3 | NM_017590 | 23264 | 0.02121958 |
| AGI_HUM1_OLIGO_A_23_P50799 | OR10H2 | Olfactory receptor, family 10, subfamily H, member 2 | NM_013939 | 26538 | 0.02121958 |
| AGI_HUM1_OLIGO_A_24_P98411 | HSPA5 | Heat shock 70 kDa protein 5 (glucose-regulated protein, 78 kDa) | NM_005347 | 3309 | 0.02121958 |
| AGI_HUM1_OLIGO_A_24_P380348 | SMAP1 | Stromal membrane-associated protein 1 | NM_021940 | 60682 | 0.02121958 |
| AGI_HUM1_OLIGO_A_23_P356021 | FANCB | Fanconi anemia, complementation group B | NM_152633 | 2187 | 0.02121958 |
| AGI_HUM1_OLIGO_A_32_P220798 | CD34 | CD34 antigen | NM_001773 | 947 | 0.02121958 |
| AGI_HUM1_OLIGO_A_24_P814444 | | | THC2407545 | | 0.02172937 |
| AGI_HUM1_OLIGO_A_23_P159650 | COX7B | Cytochrome c oxidase subunit VIIb | NM_001866 | 1349 | 0.02172937 |
| AGI_HUM1_OLIGO_A_23_P97265 | GPATC4 | G patch domain containing 4 | NM_182679 | 54865 | 0.02172937 |
| AGI_HUM1_OLIGO_A_24_P612020 | SQSTM1 | Sequestosome 1 | AK096241 | 8878 | 0.02172937 |
| AGI_HUM1_OLIGO_A_24_P73577 | ALDH1A2 | Aldehyde dehydrogenase 1 family, member A2 | NM_170697 | 8854 | 0.02172937 |
| AGI_HUM1_OLIGO_A_24_P257201 | MRPL30 | Mitochondrial ribosomal protein L30 | NM_145213 | 51263 | 0.02172937 |
| AGI_HUM1_OLIGO_A_23_P374284 | CASC3 | Cancer susceptibility candidate 3 | NM_007359 | 22794 | 0.02172937 |
| AGI_HUM1_OLIGO_A_32_P142407 | | | A_32_P142407 | | 0.02172937 |
| AGI_HUM1_OLIGO_A_24_P924957 | KRT18 | Keratin 18 | L32537 | 3875 | 0.02244126 |
| AGI_HUM1_OLIGO_A_23_P152199 | CTRL | Chymotrypsin-like | NM_001907 | 1506 | 0.02244126 |
| AGI_HUM1_OLIGO_A_24_P67699 | RPL23A | Ribosomal protein L23a | NM_000984 | 6147 | 0.02274152 |
| AGI_HUM1_OLIGO_A_23_P48585 | SALL2 | Sal-like 2 (Drosophila) | NM_005407 | 6297 | 0.02274152 |
| AGI_HUM1_OLIGO_A_24_P42330 | | LOC440523 | AK090827 | 440523 | 0.02274152 |
| AGI_HUM1_OLIGO_A_23_P31833 | EEF1D | Eukaryotic translation elongation factor 1 delta (guanine nucleotide exchange | NM_032378 | 1936 | 0.02301926 |
| | | protein) | | | |
| AGI_HUM1_OLIGO_A_24_P389415 | | | NM_007257 | | 0.02309658 |
| AGI_HUM1_OLIGO_A_23_P119794 | | Full-length cDNA clone CS0DM002YA18 of Fetal liver of Homo sapiens (human) | CR608907 | | 0.02333418 |
| AGI_HUM1_OLIGO_A_23_P102420 | CCT4 | Chaperonin containing TCP1, subunit 4 (delta) | NM_006430 | 10575 | 0.02343542 |
| AGI_HUM1_OLIGO_A_24_P29966 | TRIM32 | Tripartite motif-containing 32 | NM_012210 | 22954 | 0.02352042 |
| AGI_HUM1_OLIGO_A_23_P66924 | FLJ20071 | Dymeclin | NM_017653 | 54808 | 0.02352042 |
| AGI_HUM1_OLIGO_A_23_P139950 | KPNA3 | Karyopherin alpha 3 (importin alpha 4) | NM_002267 | 3839 | 0.02352042 |
| AGI_HUM1_OLIGO_A_23_P156708 | TNXB | Tenascin XB | NM_019105 | 7148 | 0.02423024 |
| AGI_HUM1_OLIGO_A_24_P306558 | KIAA1049 | KIAA1049 protein | NM_014972 | 22980 | 0.02423024 |
| AGI_HUM1_OLIGO_A_23_P89824 | MGC11386 | Hypothetical protein MGC11386 | NM_032933 | 85019 | 0.02423447 |
| AGI_HUM1_OLIGO_A_32_P23525 | | CDNA FLJ42287 fis, clone TLIVE2005866 | AK124281 | | 0.02423447 |
| AGI_HUM1_OLIGO_A_24_P336577 | LOC55924 | Hypothetical protein LOC55924 | NM_019099 | 55924 | 0.02423447 |
| AGI_HUM1_OLIGO_A_23_P168847 | FBXO16 | F-box protein 16 | NM_172366 | 157574 | 0.02423447 |
| AGI_HUM1_OLIGO_A_24_P510377 | | Hypothetical gene supported by BC031250 | BC031250 | 400751 | 0.02439724 |
| AGI_HUM1_OLIGO_A_23_P202156 | NFKB2 | Nuclear factor of kappa light polypeptide gene enhancer in B-cells 2 (p49/p100) | NM_002502 | 4791 | 0.02439724 |
| AGI_HUM1_OLIGO_A_23_P57961 | PLXNB1 | Plexin B1 | NM_002673 | 5364 | 0.02445234 |
| AGI_HUM1_OLIGO_A_23_P208310 | ASE-1 | CD3-epsilon-associated protein; antisense to ERCC-1 | NM_012099 | 10849 | 0.02445234 |
| AGI_HUM1_OLIGO_A_23_P94660 | TBC1D13 | TBC1 domain family, member 13 | NM_018201 | 54662 | 0.02445234 |
| AGI_HUM1_OLIGO_A_24_P930796 | C10orf72 | Chromosome 10 open reading frame 72 | AK001062 | 196740 | 0.02445234 |
| AGI_HUM1_OLIGO_A_32_P112881 | | | ENST00000374274 | | 0.02452672 |
| AGI_HUM1_OLIGO_A_24_P22892 | KCTD15 | Potassium channel tetramerisation domain containing 15 | NM_024076 | 79047 | 0.02495697 |
| AGI_HUM1_OLIGO_A_32_P117760 | | Transcribed locus | BG208131 | | 0.02495697 |
| AGI_HUM1_OLIGO_A_23_P59022 | TRERF1 | Transcriptional regulating factor 1 | NM_033502 | 55809 | 0.02495697 |
| AGI_HUM1_OLIGO_A_23_P168449 | RASA4 | RAS p21 protein activator 4 | NM_006989 | 10156 | 0.02495697 |
| AGI_HUM1_OLIGO_A_23_P160159 | SLC2A5 | Solute carrier family 2 (facilitated glucose/fructose transporter), member 5 | NM_003039 | 6518 | 0.02495697 |
| AGI_HUM1_OLIGO_A_23_P127793 | FLJ35827 | Hypothetical protein FLJ35827 | NM_153265 | 256364 | 0.02495697 |
| AGI_HUM1_OLIGO_A_23_P205228 | ATP7B | ATPase, Cu++ transporting, beta polypeptide (Wilson disease) | NM_000053 | 540 | 0.02495697 |
| AGI_HUM1_OLIGO_A_24_P247820 | | | AK074633 | | 0.02505001 |
| AGI_HUM1_OLIGO_A_23_P351734 | NPHP4 | Nephronophthisis 4 | NM_015102 | 261734 | 0.0259092 |
| AGI_HUM1_OLIGO_A_32_P109755 | | Unknown mRNA | AF462446 | | 0.02600757 |
| AGI_HUM1_OLIGO_A_32_P142700 | C22orf15 | Chromosome 22 open reading frame 15 | NM_182520 | 150248 | 0.02600757 |
| AGI_HUM1_OLIGO_A_23_P362183 | SAMD6 | Sterile alpha motif domain containing 6 | NM_173551 | 203286 | 0.02600757 |
| AGI_HUM1_OLIGO_A_24_P475556 | LOC146346 | Hypothetical protein LOC146346 | AK057359 | 146346 | 0.02600757 |
| AGI_HUM1_OLIGO_A_23_P258972 | GOLGA1 | Golgi autoantigen, golgin subfamily a, 1 | NM_002077 | 2800 | 0.02600757 |
| AGI_HUM1_OLIGO_A_23_P2990 | CEBPE | CCAAT/enhancer binding protein (C/EBP), epsilon | NM_001805 | 1053 | 0.02600757 |
| AGI_HUM1_OLIGO_A_24_P177964 | KIAA1602 | KIAA1602 protein | BC033253 | 57701 | 0.02600757 |
| AGI_HUM1_OLIGO_A_24_P821168 | EEF1D | Eukaryotic translation elongation factor 1 delta (guanine nucleotide exchange | NM_032378 | 1936 | 0.02600757 |
| | | protein) | | | |
| AGI_HUM1_OLIGO_A_23_P160523 | TRIM45 | Tripartite motif-containing 45 | NM_025188 | 80263 | 0.02600757 |
| AGI_HUM1_OLIGO_A_23_P135826 | LOC114977 | Hypothetical protein BC014148 | AK096342 | 114977 | 0.02600757 |
| AGI_HUM1_OLIGO_A_24_P478940 | | | A_24_P478940 | | 0.02600757 |
| AGI_HUM1_OLIGO_A_32_P206989 | | CDNA FLJ13350 fis, clone OVARC1002143 | AK023412 | | 0.02600757 |
| AGI_HUM1_OLIGO_A_32_P524014 | | | ENST00000367545 | | 0.02600757 |
| AGI_HUM1_OLIGO_A_23_P117125 | RARG | Retinoic acid receptor, gamma | NM_000966 | 5916 | 0.02600757 |
| AGI_HUM1_OLIGO_A_23_P422144 | FAM43A | Family with sequence similarity 43, member A | NM_153690 | 131583 | 0.02600757 |
| AGI_HUM1_OLIGO_A_24_P417935 | CENTG1 | Centaurin, gamma 1 | CR590071 | 116986 | 0.02600757 |
| AGI_HUM1_OLIGO_A_32_P75888 | COMMD10 | COMM domain containing 10 | CD243286 | 51397 | 0.02600757 |
| AGI_HUM1_OLIGO_A_24_P902653 | MGC2747 | Hypothetical protein MGC2747 | CR616041 | 79086 | 0.02600757 |
| AGI_HUM1_OLIGO_A_23_P156284 | DBN1 | Drebrin 1 | NM_080881 | 1627 | 0.02600757 |
| AGI_HUM1_OLIGO_A_32_P224327 | | CDNA FLJ31839 fis, clone NT2RP7000086 | AK056401 | | 0.02600757 |
| AGI_HUM1_OLIGO_A_32_P144220 | | | ENST00000374534 | | 0.02600757 |
| AGI_HUM1_OLIGO_A_32_P192474 | C6orf31 | Chromosome 6 open reading frame 31 | NM_030651 | 80863 | 0.02600757 |
| AGI_HUM1_OLIGO_A_24_P399630 | PRKACA | Protein kinase, cAMP-dependent, catalytic, alpha | NM_002730 | 5566 | 0.02600757 |
| AGI_HUM1_OLIGO_A_24_P203072 | PHF16 | PHD finger protein 16 | NM_014735 | 9767 | 0.02600757 |
| AGI_HUM1_OLIGO_A_23_P50368 | OSCAR | Osteoclast-associated receptor | NM_206818 | 126014 | 0.02600757 |
| AGI_HUM1_OLIGO_A_23_P421401 | PDGFRB | Platelet-derived growth factor receptor, beta polypeptide | NM_002609 | 5159 | 0.02600757 |
| AGI_HUM1_OLIGO_A_23_P373598 | MAFK | V-maf musculoaponeurotic fibrosarcoma oncogene homolog K (avian) | NM_002360 | 7975 | 0.02600757 |
| AGI_HUM1_OLIGO_A_23_P103084 | TOM1 | Target of myb1 (chicken) | NM_005488 | 10043 | 0.02600757 |
| AGI_HUM1_OLIGO_A_23_P149427 | CAPN9 | Calpain 9 | NM_006615 | 10753 | 0.02600779 |
| AGI_HUM1_OLIGO_A_23_P254863 | MGC42105 | Hypothetical protein MGC42105 | NM_153361 | 167359 | 0.02610687 |
| AGI_HUM1_OLIGO_A_24_P344416 | DSC3 | Desmocollin 3 | NM_024423 | 1825 | 0.02615967 |
| AGI_HUM1_OLIGO_A_23_P216282 | ARHGEF10 | Rho guanine nucleotide exchange factor (GEF) 10 | NM_014629 | 9639 | 0.02626067 |
| AGI_HUM1_OLIGO_A_23_P14273 | ZFYVE21 | Zinc finger, FYVE domain containing 21 | NM_024071 | 79038 | 0.02691455 |
| AGI_HUM1_OLIGO_A_24_P713729 | | | THC2373625 | | 0.02706542 |
| AGI_HUM1_OLIGO_A_23_P397055 | CSGlcA-T | Chondroitin sulfate glucuronyltransferase | NM_019015 | 54480 | 0.02708649 |
| AGI_HUM1_OLIGO_A_24_P11045 | | | THC2271174 | | 0.02708649 |
| AGI_HUM1_OLIGO_A_32_P171232 | | | A_32_P171232 | | 0.02708649 |
| AGI_HUM1_OLIGO_A_23_P142125 | HRC | Histidine rich calcium binding protein | NM_002152 | 3270 | 0.02708649 |
| AGI_HUM1_OLIGO_A_23_P254165 | RAI2 | Retinoic acid induced 2 | NM_021785 | 10742 | 0.02871497 |
| AGI_HUM1_OLIGO_A_23_P208991 | PALM | Paralemmin | NM_002579 | 5064 | 0.02871497 |
| AGI_HUM1_OLIGO_A_24_P343289 | CDC2L6 | Cell division cycle 2-like 6 (CDK8-like) | NM_015076 | 23097 | 0.02871497 |
| AGI_HUM1_OLIGO_A_23_P114105 | LDOC1 | Leucine zipper, down-regulated in cancer 1 | NM_012317 | 23641 | 0.02934208 |
| AGI_HUM1_OLIGO_A_32_P460399 | | MRNA full length insert cDNA clone EUROIMAGE 2362292 | AL365520 | | 0.02949116 |
| AGI_HUM1_OLIGO_A_23_P4604 | ZNF600 | Zinc finger protein 600 | NM_198457 | 162966 | 0.02977394 |
| AGI_HUM1_OLIGO_A_23_P253723 | RRH | Retinal pigment epithelium-derived rhodopsin homolog | NM_006583 | 10692 | 0.02977394 |
| AGI_HUM1_OLIGO_A_24_P84279 | EBPL | Emopamil binding protein-like | NM_032565 | 84650 | 0.03082569 |
| AGI_HUM1_OLIGO_A_23_P502371 | PHF2 | PHD finger protein 2 | NM_024517 | 5253 | 0.03120828 |
| AGI_HUM1_OLIGO_A_32_P84342 | | | THC2315140 | | 0.03192985 |
| AGI_HUM1_OLIGO_A_23_P155477 | LOC51161 | G20 protein | NM_016210 | 51161 | 0.03192985 |
| AGI_HUM1_OLIGO_A_24_P923704 | TOLLIP | Toll interacting protein | AF321778 | 54472 | 0.03192985 |
| AGI_HUM1_OLIGO_A_23_P80048 | | | NR_001442 | | 0.03192985 |
| AGI_HUM1_OLIGO_A_23_P154338 | EFHD1 | EF hand domain containing 1 | NM_025202 | 80303 | 0.03192985 |
| AGI_HUM1_OLIGO_A_23_P408996 | OACT1 | O-acyltransferase (membrane bound) domain containing 1 | AK131269 | 154141 | 0.03192985 |
| AGI_HUM1_OLIGO_A_23_P211141 | DSCAM | Down syndrome cell adhesion molecule | NM_001389 | 1826 | 0.03192985 |
| AGI_HUM1_OLIGO_A_32_P16315 | | | ENST00000272035 | | 0.03192985 |
| AGI_HUM1_OLIGO_A_24_P289818 | | | ENST00000370659 | | 0.03192985 |
| AGI_HUM1_OLIGO_A_23_P375165 | FLJ35767 | FLJ35767 protein | NM_207459 | 400629 | 0.03192985 |
| AGI_HUM1_OLIGO_A_32_P45780 | | | THC2282384 | | 0.03192985 |
| AGI_HUM1_OLIGO_A_24_P921823 | | | ENST00000369397 | | 0.03192985 |
| AGI_HUM1_OLIGO_A_23_P144458 | CAMK2D | Calcium/calmodulin-dependent protein kinase (CaM kinase) II delta | NM_001221 | 817 | 0.03192985 |
| AGI_HUM1_OLIGO_A_23_P68759 | TFF1 | Trefoil factor 1 (breast cancer, estrogen-inducible sequence expressed in) | NM_003225 | 7031 | 0.03192985 |
| AGI_HUM1_OLIGO_A_32_P221799 | HIST1H2AM | Histone 1, H2am | NM_003514 | 8336 | 0.03192985 |
| AGI_HUM1_OLIGO_A_32_P110433 | | | THC2273687 | | 0.03192985 |
| AGI_HUM1_OLIGO_A_23_P165778 | MLPH | Melanophilin | NM_024101 | 79083 | 0.03192985 |
| AGI_HUM1_OLIGO_A_32_P156851 | DSCR1L1 | Down syndrome critical region gene 1-like 1 | NM_005822 | 10231 | 0.03192985 |
| AGI_HUM1_OLIGO_A_23_P315933 | WBSCR21 | Williams Beuren syndrome chromosome region 21 | NM_031295 | 83451 | 0.03192985 |
| AGI_HUM1_OLIGO_A_24_P64344 | BLNK | B-cell linker | NM_013314 | 29760 | 0.03192985 |
| AGI_HUM1_OLIGO_A_23_P32903 | OCRL | Oculocerebrorenal syndrome of Lowe | NM_000276 | 4952 | 0.03192985 |
| AGI_HUM1_OLIGO_A_23_P320553 | PPFIA3 | Protein tyrosine phosphatase, receptor type, f polypeptide (PTPRF), interacting | NM_003660 | 8541 | 0.0321749 |
| | | protein (liprin), alpha 3 | | | |
| AGI_HUM1_OLIGO_A_32_P55106 | | | THC2374814 | | 0.0321749 |
| AGI_HUM1_OLIGO_A_24_P263543 | | | ENST00000277860 | | 0.03231425 |
| AGI_HUM1_OLIGO_A_23_P65674 | TMOD3 | Tropomodulin 3 (ubiquitous) | NM_014547 | 29766 | 0.03231425 |
| AGI_HUM1_OLIGO_A_23_P257609 | RPL29 | Ribosomal protein L29 | NM_000992 | 6159 | 0.03231425 |
| AGI_HUM1_OLIGO_A_24_P276864 | FLJ20297 | Hypothetical protein FLJ20297 | NM_017951 | 55627 | 0.0327972 |
| AGI_HUM1_OLIGO_A_24_P118341 | KENAE | Kenae | BF869497 | 202243 | 0.03286109 |
| AGI_HUM1_OLIGO_A_23_P431268 | PLEKHA6 | Pleckstrin homology domain containing, family A member 6 | NM_014935 | 22874 | 0.03286109 |
| AGI_HUM1_OLIGO_A_24_P367804 | | | BC024651 | | 0.03286109 |
| AGI_HUM1_OLIGO_A_23_P24433 | CTSF | Cathepsin F | NM_003793 | 8722 | 0.03286109 |
| AGI_HUM1_OLIGO_A_32_P96692 | | | THC2340838 | | 0.03286109 |
| AGI_HUM1_OLIGO_A_23_P48570 | DHRS2 | Dehydrogenase/reductase (SDR family) member 2 | NM_182908 | 10202 | 0.03286109 |
| AGI_HUM1_OLIGO_A_24_P302898 | BAP1 | BRCA1 associated protein-1 (ubiquitin carboxy-terminal hydrolase) | NM_004656 | 8314 | 0.03286109 |
| AGI_HUM1_OLIGO_A_23_P96369 | FLJ10178 | Hypothetical protein FLJ10178 | NM_018015 | 55086 | 0.03286109 |
| AGI_HUM1_OLIGO_A_24_P322444 | NUP62 | Nucleoporin 62 kDa | NM_153719 | 23636 | 0.03286109 |
| AGI_HUM1_OLIGO_A_24_P936692 | | Homo sapiens, clone IMAGE: 4822684, mRNA | BC033528 | | 0.03286109 |
| AGI_HUM1_OLIGO_A_23_P164011 | SOX15 | SRY (sex determining region Y)-box 15 | NM_006942 | 6665 | 0.03286109 |
| AGI_HUM1_OLIGO_A_24_P289383 | CHD7 | Chromodomain helicase DNA binding protein 7 | NM_017780 | 55636 | 0.03300997 |
| AGI_HUM1_OLIGO_A_24_P160874 | DUT | DUTP pyrophosphatase | NM_001948 | 1854 | 0.03307712 |
| AGI_HUM1_OLIGO_A_24_P788227 | | LOC440156 | BC041856 | 440156 | 0.03307712 |
| AGI_HUM1_OLIGO_A_23_P338919 | | | AK055387 | | 0.03307712 |
| AGI_HUM1_OLIGO_A_24_P302374 | CLCN6 | Chloride channel 6 | NM_001286 | 1185 | 0.03307712 |
| AGI_HUM1_OLIGO_A_32_P222362 | DRG1 | Developmentally regulated GTP binding protein 1 | AK127132 | 4733 | 0.03326542 |
| AGI_HUM1_OLIGO_A_23_P85585 | LCN7 | Lipocalin 7 | NM_022164 | 64129 | 0.03326542 |
| AGI_HUM1_OLIGO_A_24_P185986 | MGC23937 | Hypothetical protein MGC23937 similar to CG4798 | NM_145052 | 139596 | 0.03340835 |
| AGI_HUM1_OLIGO_A_32_P27327 | ITM1 | Integral membrane protein 1 | W27166 | 3703 | 0.03340835 |
| AGI_HUM1_OLIGO_A_23_P253052 | CD99L2 | CD99 antigen-like 2 | NM_031462 | 83692 | 0.03377243 |
| AGI_HUM1_OLIGO_A_24_P409471 | | | ENST00000316861 | | 0.03449789 |
| AGI_HUM1_OLIGO_A_24_P275873 | TBCD | Tubulin-specific chaperone d | NM_005993 | 6904 | 0.03459929 |
| AGI_HUM1_OLIGO_A_23_P356004 | CSEN | Calsenilin, presenilin binding protein, EF hand transcription factor | NM_013434 | 30818 | 0.03471589 |
| AGI_HUM1_OLIGO_A_24_P226248 | ZMAT2 | Zinc finger, matrin type 2 | NM_144723 | 153527 | 0.03471589 |
| AGI_HUM1_OLIGO_A_24_P374652 | NUCKS | Nuclear ubiquitous casein kinase and cyclin-dependent kinase substrate | NM_022731 | 64710 | 0.03471589 |
| AGI_HUM1_OLIGO_A_23_P104224 | ACF | Apobec-1 complementation factor | NM_138933 | 29974 | 0.03471589 |
| AGI_HUM1_OLIGO_A_24_P270769 | VPS35 | Vacuolar protein sorting 35 (yeast) | NM_018206 | 55737 | 0.03471589 |
| AGI_HUM1_OLIGO_A_23_P89113 | FLJ14154 | Hypothetical protein FLJ14154 | NM_024845 | 79903 | 0.03471589 |
| AGI_HUM1_OLIGO_A_24_P942112 | POLR1B | Polymerase (RNA) I polypeptide B, 128 kDa | NM_019014 | 84172 | 0.0348849 |
| AGI_HUM1_OLIGO_A_23_P111452 | CENTG3 | Centaurin, gamma 3 | NM_031946 | 116988 | 0.03501603 |
| AGI_HUM1_OLIGO_A_23_P72127 | KIAA1446 | Brain-enriched guanylate kinase-associated protein | NM_020836 | 57596 | 0.03519474 |
| AGI_HUM1_OLIGO_A_32_P176018 | LOC81569 | Actin like protein | NM_030812 | 81569 | 0.03532587 |
| AGI_HUM1_OLIGO_A_32_P319200 | GGTL4 | Gamma-glutamyltransferase-like 4 | NM_199127 | 91227 | 0.03544083 |
| AGI_HUM1_OLIGO_A_24_P97849 | DBN1 | Drebrin 1 | NM_080881 | 1627 | 0.03544083 |
| AGI_HUM1_OLIGO_A_24_P75708 | | | A_24_P75708 | | 0.03544083 |
| AGI_HUM1_OLIGO_A_32_P82293 | | | A_32_P82293 | | 0.03544083 |
| AGI_HUM1_OLIGO_A_24_P358279 | | | A_24_P358279 | | 0.03544083 |
| AGI_HUM1_OLIGO_A_23_P79587 | ALPP | Alkaline phosphatase, placental (Regan isozyme) | NM_001632 | 250 | 0.03544083 |
| AGI_HUM1_OLIGO_A_24_P283928 | MGC2731 | Hypothetical protein MGC2731 | NM_024068 | 79035 | 0.03544083 |
| AGI_HUM1_OLIGO_A_24_P101271 | | | A_24_P101271 | | 0.03546775 |
| AGI_HUM1_OLIGO_A_32_P226186 | | | THC2266474 | | 0.03546775 |
| AGI_HUM1_OLIGO_A_23_P88580 | ARID3B | AT rich interactive domain 3B (BRIGHT-like) | NM_006465 | 10620 | 0.03562191 |
| AGI_HUM1_OLIGO_A_23_P146855 | C22orf1 | Chromosome 22 open reading frame 1 | NM_001585 | 758 | 0.03562191 |
| AGI_HUM1_OLIGO_A_23_P115743 | C10orf84 | Chromosome 10 open reading frame 84 | NM_022063 | 63877 | 0.03579344 |
| AGI_HUM1_OLIGO_A_23_P408768 | DOT1L | DOT1-like, histone H3 methyltransferase (S. cerevisiae) | NM_032482 | 84444 | 0.03579344 |
| AGI_HUM1_OLIGO_A_24_P58331 | FLJ22955 | Hypothetical protein FLJ22955 | NM_024819 | 79877 | 0.03579344 |
| AGI_HUM1_OLIGO_A_32_P101623 | | | THC2337763 | | 0.03579344 |
| AGI_HUM1_OLIGO_A_23_P141680 | BCAS3 | Breast carcinoma amplified sequence 3 | NM_017679 | 54828 | 0.03579344 |
| AGI_HUM1_OLIGO_A_23_P81880 | CTDSP2 | CTD (carboxy-terminal domain, RNA polymerase II, polypeptide A) small | NM_005730 | 10106 | 0.03579344 |
| | | phosphatase 2 | | | |
| AGI_HUM1_OLIGO_A_32_P26017 | | | DB380193 | | 0.03579344 |
| AGI_HUM1_OLIGO_A_23_P26713 | RPL23 | Ribosomal protein L23 | NM_000978 | 9349 | 0.03579344 |
| AGI_HUM1_OLIGO_A_23_P123256 | PDAP1 | PDGFA associated protein 1 | NM_014891 | 11333 | 0.03579344 |
| AGI_HUM1_OLIGO_A_24_P125311 | | | A_24_P125311 | | 0.03579344 |
| AGI_HUM1_OLIGO_A_23_P104252 | ITIH5 | Inter-alpha (globulin) inhibitor H5 | NM_030569 | 80760 | 0.03579344 |
| AGI_HUM1_OLIGO_A_32_P134657 | KIAA0240 | KIAA0240 | NM_015349 | 23506 | 0.03579344 |
| AGI_HUM1_OLIGO_A_24_P113295 | AP2S1 | Adaptor-related protein complex 2, sigma 1 subunit | NM_004069 | 1175 | 0.03579344 |
| AGI_HUM1_OLIGO_A_23_P17811 | SEC14L2 | SEC14-like 2 (S. cerevisiae) | NM_012429 | 23541 | 0.03579344 |
| AGI_HUM1_OLIGO_A_23_P13057 | PRDM11 | PR domain containing 11 | NM_020229 | 56981 | 0.03579344 |
| AGI_HUM1_OLIGO_A_23_P70827 | KIAA1549 | KIAA1549 protein | AL136736 | 57670 | 0.03579344 |
| AGI_HUM1_OLIGO_A_23_P84118 | CDH18 | Cadherin 18, type 2 | NM_004934 | 1016 | 0.03581628 |
| AGI_HUM1_OLIGO_A_32_P32653 | SENP5 | SUMO1/sentrin specific protease 5 | BC036626 | 205564 | 0.03588595 |
| AGI_HUM1_OLIGO_A_32_P183765 | ERBB4 | V-erb-a erythroblastic leukemia viral oncogene homolog 4 (avian) | AK126298 | 2066 | 0.03588595 |
| AGI_HUM1_OLIGO_A_23_P35820 | CFL1 | Cofilin 1 (non-muscle) | NM_005507 | 1072 | 0.03588595 |
| AGI_HUM1_OLIGO_A_24_P941268 | CA5B | Inactivation escape 2 | NM_007220 | 11238 | 0.03588595 |
| AGI_HUM1_OLIGO_A_23_P57199 | GGTLA4 | Gamma-glutamyltransferase-like activity 4 | NM_080920 | 92086 | 0.03588595 |
| AGI_HUM1_OLIGO_A_23_P101532 | RPS11 | Ribosomal protein S11 | NM_001015 | 6205 | 0.03590805 |
| AGI_HUM1_OLIGO_A_23_P143981 | FBLN2 | Fibulin 2 | NM_001004019 | 2199 | 0.03619918 |
| AGI_HUM1_OLIGO_A_24_P68008 | | | ENST00000294485 | | 0.03668376 |
| AGI_HUM1_OLIGO_A_32_P177477 | | | THC2344152 | | 0.03673976 |
| AGI_HUM1_OLIGO_A_23_P117380 | C14orf152 | Chromosome 14 open reading frame 152 | NM_138344 | 90050 | 0.0369129 |
| AGI_HUM1_OLIGO_A_23_P421011 | KAZALD1 | Kazal-type serine protease inhibitor domain 1 | NM_030929 | 81621 | 0.0370985 |
| AGI_HUM1_OLIGO_A_23_P80759 | PVRL3 | Poliovirus receptor-related 3 | BC017572 | 25945 | 0.0370985 |
| AGI_HUM1_OLIGO_A_23_P18292 | RPL14 | Ribosomal protein L14 | NM_003973 | 9045 | 0.03825962 |
| AGI_HUM1_OLIGO_A_24_P108779 | FLJ12242 | Hypothetical protein FLJ12242 | NM_024681 | 79734 | 0.03825962 |
| AGI_HUM1_OLIGO_A_24_P753592 | | | BC018589 | | 0.03825962 |
| AGI_HUM1_OLIGO_A_23_P250164 | HGD | Homogentisate 1,2-dioxygenase (homogentisate oxidase) | NM_000187 | 3081 | 0.03825962 |
| AGI_HUM1_OLIGO_A_23_P154849 | OLIG1 | Oligodendrocyte transcription factor 1 | NM_138983 | 116448 | 0.03825962 |
| AGI_HUM1_OLIGO_A_24_P296568 | CBX1 | Chromobox homolog 1 (HP1 beta homolog Drosophila) | NM_006807 | 10951 | 0.03825962 |
| AGI_HUM1_OLIGO_A_23_P315106 | | | ENST00000380410 | | 0.03892307 |
| AGI_HUM1_OLIGO_A_32_P38637 | KIAA1862 | KIAA1862 protein | NM_032534 | 84626 | 0.03892307 |
| AGI_HUM1_OLIGO_A_23_P22382 | DKFZP434P1750 | DKFZP434P1750 protein | NM_015527 | 26000 | 0.03956369 |
| AGI_HUM1_OLIGO_A_32_P194563 | | | THC2281660 | | 0.03973329 |
| AGI_HUM1_OLIGO_A_23_P78517 | SFRS16 | Splicing factor, arginine/serine-rich 16 (suppressor-of-white-apricot homolog, | NM_007056 | 11129 | 0.03973329 |
| | | Drosophila) | | | |
| AGI_HUM1_OLIGO_A_23_P168868 | PTDSS1 | Phosphatidylserine synthase 1 | NM_014754 | 9791 | 0.03973329 |
| AGI_HUM1_OLIGO_A_24_P7330 | | | A_24_P7330 | | 0.03973329 |
| AGI_HUM1_OLIGO_A_23_P316850 | C19orf19 | Chromosome 19 open reading frame 19 | NM_182577 | 284451 | 0.03973329 |
| AGI_HUM1_OLIGO_A_23_P428992 | | | ENST00000382579 | | 0.03973329 |
| AGI_HUM1_OLIGO_A_23_P69141 | DNCLI1 | Dynein, cytoplasmic, light intermediate polypeptide 1 | NM_016141 | 51143 | 0.03973329 |
| AGI_HUM1_OLIGO_A_23_P112429 | RPL35 | Ribosomal protein L35 | NM_007209 | 11224 | 0.03973329 |
| AGI_HUM1_OLIGO_A_23_P18082 | FLJ14566 | Hypothetical protein FLJ14566 | NM_032806 | 84892 | 0.03977095 |
| AGI_HUM1_OLIGO_A_23_P27424 | ZNF418 | Zinc finger protein 418 | NM_133460 | 147686 | 0.03977095 |
| AGI_HUM1_OLIGO_A_24_P392082 | | | ENST00000361800 | | 0.03977095 |
| AGI_HUM1_OLIGO_A_32_P104023 | | | THC2346121 | | 0.03977095 |
| AGI_HUM1_OLIGO_A_23_P159974 | KLHL13 | Kelch-like 13 (Drosophila) | NM_033495 | 90293 | 0.03978727 |
| AGI_HUM1_OLIGO_A_24_P659113 | FLJ40432 | Hypothetical protein FLJ40432 | NM_152523 | 151195 | 0.03998933 |
| AGI_HUM1_OLIGO_A_23_P203819 | GOLGA3 | Golgi autoantigen, golgin subfamily a, 3 | NM_005895 | 2802 | 0.04003625 |
| AGI_HUM1_OLIGO_A_32_P234996 | | | THC2311218 | | 0.04003625 |
| AGI_HUM1_OLIGO_A_23_P149375 | CTMP | C-terminal modulator protein | NM_053055 | 117145 | 0.04003625 |
| AGI_HUM1_OLIGO_A_23_P404059 | PASD1 | PAS domain containing 1 | NM_173493 | 139135 | 0.04003625 |
| AGI_HUM1_OLIGO_A_23_P201368 | CTBS | Chitobiase, di-N-acetyl- | NM_004388 | 1486 | 0.04008632 |
| AGI_HUM1_OLIGO_A_23_P117298 | F7 | Coagulation factor VII (serum prothrombin conversion accelerator) | NM_000131 | 2155 | 0.04008632 |
| AGI_HUM1_OLIGO_A_32_P49350 | | | CR609307 | | 0.040121 |
| AGI_HUM1_OLIGO_A_32_P26443 | | | THC2258710 | | 0.040121 |
| AGI_HUM1_OLIGO_A_24_P384119 | | | XM_942451 | | 0.04032726 |
| AGI_HUM1_OLIGO_A_23_P147388 | KIF13B | Kinesin family member 13B | NM_015254 | 23303 | 0.04032726 |
| AGI_HUM1_OLIGO_A_23_P393697 | MGC27345 | Hypothetical protein MGC27345 | BC024231 | 157247 | 0.0405513 |
| AGI_HUM1_OLIGO_A_24_P290263 | | | A_24_P290263 | | 0.04072885 |
| AGI_HUM1_OLIGO_A_23_P168556 | STX1A | Syntaxin 1A (brain) | NM_004603 | 6804 | 0.04072885 |
| AGI_HUM1_OLIGO_A_23_P218108 | WDFY2 | WD repeat and FYVE domain containing 2 | NM_052950 | 115825 | 0.04114115 |
| AGI_HUM1_OLIGO_A_24_P278853 | FLJ14624 | Hypothetical protein FLJ14624 | NM_032813 | 84899 | 0.04114115 |
| AGI_HUM1_OLIGO_A_23_P161041 | KLHL12 | Kelch-like 12 (Drosophila) | NM_021633 | 59349 | 0.04114115 |
| AGI_HUM1_OLIGO_A_32_P214758 | | | THC2383313 | | 0.04114115 |
| AGI_HUM1_OLIGO_A_23_P6398 | AP1B1 | Adaptor-related protein complex 1, beta 1 subunit | NM_001127 | 162 | 0.04114115 |
| AGI_HUM1_OLIGO_A_24_P40928 | | | ENST00000281228 | | 0.04285574 |
| AGI_HUM1_OLIGO_A_24_P3804 | | | NM_001039651 | | 0.04285574 |
| AGI_HUM1_OLIGO_A_24_P403403 | RPL35 | Ribosomal protein L35 | NM_007209 | 11224 | 0.04285574 |
| AGI_HUM1_OLIGO_A_23_P16866 | VIL1 | Villin 1 | NM_007127 | 7429 | 0.0429901 |
| AGI_HUM1_OLIGO_A_23_P202672 | RNH | Ribonuclease/angiogenin inhibitor | NM_002939 | 6050 | 0.04300262 |
| AGI_HUM1_OLIGO_A_24_P30314 | SCYL1 | SCY1-like 1 (S. cerevisiae) | NM_020680 | 57410 | 0.04341005 |
| AGI_HUM1_OLIGO_A_23_P168928 | CYP11B1 | Cytochrome P450, family 11, subfamily B, polypeptide 1 | NM_000497 | 1584 | 0.04343043 |
| AGI_HUM1_OLIGO_A_24_P567298 | CSAG2 | CSAG family, member 2 | NM_004909 | 9598 | 0.04350798 |
| AGI_HUM1_OLIGO_A_32_P164593 | FLJ13842 | Hypothetical protein FLJ13842 | NM_024645 | 79698 | 0.04353467 |
| AGI_HUM1_OLIGO_A_32_P118532 | | CDNA clone IMAGE: 5288145, partial cds | CR625518 | | 0.0436705 |
| AGI_HUM1_OLIGO_A_23_P45304 | XK | Kell blood group precursor (McLeod phenotype) | NM_021083 | 7504 | 0.04373896 |
| AGI_HUM1_OLIGO_A_23_P155360 | HDAC11 | Histone deacetylase 11 | NM_024827 | 79885 | 0.04377807 |
| AGI_HUM1_OLIGO_A_32_P107833 | | | THC2404842 | | 0.04377807 |
| AGI_HUM1_OLIGO_A_23_P76059 | | | ENST00000354743 | | 0.04382447 |
| AGI_HUM1_OLIGO_A_23_P50399 | | | NM_138353 | | 0.04382447 |
| AGI_HUM1_OLIGO_A_24_P269398 | CASP2 | Caspase 2, apoptosis-related cysteine protease (neural precursor cell expressed, | NM_032982 | 835 | 0.04426263 |
| | | developmentally down-regulated 2) | | | |
| AGI_HUM1_OLIGO_A_23_P96990 | NVL | Nuclear VCP-like | NM_002533 | 4931 | 0.04449602 |
| AGI_HUM1_OLIGO_A_23_P142671 | ZNF513 | Zinc finger protein 513 | NM_144631 | 130557 | 0.04484926 |
| AGI_HUM1_OLIGO_A_32_P192922 | LOC157562 | Hypothetical protein LOC157562 | AL050061 | 157562 | 0.04506291 |
| AGI_HUM1_OLIGO_A_24_P333357 | RAB40C | RAB40C, member RAS oncogene family | AK090463 | 57799 | 0.04506291 |
| AGI_HUM1_OLIGO_A_23_P210619 | C20orf43 | Chromosome 20 open reading frame 43 | NM_016407 | 51507 | 0.04506291 |
| AGI_HUM1_OLIGO_A_23_P143559 | CLTCL1 | Clathrin, heavy polypeptide-like 1 | NM_007098 | 8218 | 0.04506291 |
| AGI_HUM1_OLIGO_A_32_P134209 | | | THC2403913 | | 0.04506291 |
| AGI_HUM1_OLIGO_A_23_P205370 | ASB2 | Ankyrin repeat and SOCS box-containing 2 | NM_016150 | 51676 | 0.04509918 |
| AGI_HUM1_OLIGO_A_23_P67299 | DOCK6 | Dedicator of cytokinesis 6 | NM_020812 | 57572 | 0.04509918 |
| AGI_HUM1_OLIGO_A_23_P153651 | FSD1 | Fibronectin type 3 and SPRY domain containing 1 | NM_024333 | 79187 | 0.04509918 |
| AGI_HUM1_OLIGO_A_23_P22744 | PAGE-5 | PAGE-5 protein | NM_130467 | 90737 | 0.04509918 |
| AGI_HUM1_OLIGO_A_23_P208158 | DTNA | Dystrobrevin, alpha | NM_001392 | 1837 | 0.04509918 |
| AGI_HUM1_OLIGO_A_32_P133926 | | | A_32_P133926 | | 0.04509918 |
| AGI_HUM1_OLIGO_A_24_P298946 | | | A_24_P298946 | | 0.04542651 |
| AGI_HUM1_OLIGO_A_32_P19840 | | Hypothetical LOC402617 | CK300181 | 402617 | 0.04542651 |
| AGI_HUM1_OLIGO_A_23_P389897 | NGFR | Nerve growth factor receptor (TNFR superfamily, member 16) | NM_002507 | 4804 | 0.04574742 |
| AGI_HUM1_OLIGO_A_24_P383440 | KIAA0033 | KIAA0033 protein | BC035034 | 23027 | 0.04673016 |
| AGI_HUM1_OLIGO_A_32_P42780 | | | THC2338292 | | 0.04673016 |
| AGI_HUM1_OLIGO_A_23_P206454 | MGC16385 | Hypothetical protein MGC16385 | NM_145039 | 92806 | 0.04673016 |
| AGI_HUM1_OLIGO_A_24_P332971 | RPL7L1 | Ribosomal protein L7-like 1 | NM_198486 | 285855 | 0.04673016 |
| AGI_HUM1_OLIGO_A_24_P576445 | | | AK098124 | | 0.04676639 |
| AGI_HUM1_OLIGO_A_24_P125096 | MT1X | Metallothionein 1X | NM_005952 | 4501 | 0.04676639 |
| AGI_HUM1_OLIGO_A_23_P118135 | | | THC2308359 | | 0.04676639 |
| AGI_HUM1_OLIGO_A_23_P16615 | HNRPM | Heterogeneous nuclear ribonucleoprotein M | NM_005968 | 4670 | 0.04676639 |
| AGI_HUM1_OLIGO_A_24_P416961 | ARVCF | Armadillo repeat gene deletes in velocardiofacial syndrome | NM_001670 | 421 | 0.04676639 |
| AGI_HUM1_OLIGO_A_23_P107432 | FLJ35934 | FLJ35934 protein | NM_207453 | 400579 | 0.04676639 |
| AGI_HUM1_OLIGO_A_24_P270424 | DPF3 | D4, zinc and double PHD fingers, family 3 | AK124946 | 8110 | 0.04676639 |
| AGI_HUM1_OLIGO_A_23_P12603 | RNF153 | Ring finger protein 153 | NM_017824 | 54708 | 0.04676639 |
| AGI_HUM1_OLIGO_A_23_P333218 | | | NM_001031711 | | 0.04676639 |
| AGI_HUM1_OLIGO_A_32_P47616 | | | AW994273 | | 0.04681701 |
| AGI_HUM1_OLIGO_A_23_P7144 | CXCL1 | Chemokine (C—X—C motif) ligand 1 (melanoma growth stimulating activity, alpha) | NM_001511 | 2919 | 0.04688504 |
| AGI_HUM1_OLIGO_A_23_P500244 | TRPV1 | Transient receptor potential cation channel, subfamily V, member 1 | NM_080706 | 7442 | 0.04688504 |
| AGI_HUM1_OLIGO_A_23_P96302 | SPANXA2 | SPANX family, member A2 | NM_013453 | 64650 | 0.04688504 |
| AGI_HUM1_OLIGO_A_23_P350005 | FLJ25801 | Hypothetical protein FLJ25801 | NM_173553 | 205860 | 0.04688504 |
| AGI_HUM1_OLIGO_A_23_P309739 | ESR1 | Estrogen receptor 1 | NM_000125 | 2099 | 0.04688504 |
| AGI_HUM1_OLIGO_A_24_P945283 | DLG3 | Discs, large homolog 3 (neuroendocrine-dlg, Drosophila) | AB033058 | 1741 | 0.04688504 |
| AGI_HUM1_OLIGO_A_23_P218237 | LCAT | Lecithin-cholesterol acyltransferase | NM_000229 | 3931 | 0.04688504 |
| AGI_HUM1_OLIGO_A_23_P19036 | TNIP1 | TNFAIP3 interacting protein 1 | NM_006058 | 10318 | 0.04688504 |
| AGI_HUM1_OLIGO_A_23_P409168 | KIAA0540 | KIAA0540 protein | AK131104 | 23218 | 0.04688504 |
| AGI_HUM1_OLIGO_A_23_P141946 | LTBP4 | Latent transforming growth factor beta binding protein 4 | NM_003573 | 8425 | 0.0472703 |
| AGI_HUM1_OLIGO_A_23_P404005 | MGC29729 | Hypothetical protein MGC29729 | NM_152274 | 92002 | 0.0472703 |
| AGI_HUM1_OLIGO_A_32_P99690 | | | ENST00000380985 | | 0.0472703 |
| AGI_HUM1_OLIGO_A_32_P46313 | | | CF529502 | | 0.04754191 |
| AGI_HUM1_OLIGO_A_24_P7085 | | | NR_002778 | | 0.04774837 |
| AGI_HUM1_OLIGO_A_32_P220591 | | | THC2415124 | | 0.04774837 |
| AGI_HUM1_OLIGO_A_24_P931845 | HCG8 | HLA complex group 8 | AY358246 | 80869 | 0.04815713 |
| AGI_HUM1_OLIGO_A_23_P500892 | TUB | Tubby homolog (mouse) | NM_003320 | 7275 | 0.04823615 |
| AGI_HUM1_OLIGO_A_23_P24960 | FLJ23441 | Hypothetical protein FLJ23441 | NM_024678 | 79731 | 0.04823615 |
| AGI_HUM1_OLIGO_A_23_P133596 | RNASE3L | Nuclear RNase III Drosha | NM_013235 | 29102 | 0.04823615 |
| AGI_HUM1_OLIGO_A_23_P201672 | KIAA0859 | KIAA0859 | NM_015935 | 51603 | 0.04823615 |
| AGI_HUM1_OLIGO_A_24_P562242 | | | A_24_P562242 | | 0.04866814 |
| AGI_HUM1_OLIGO_A_32_P132276 | | | BE091362 | | 0.04869837 |
| AGI_HUM1_OLIGO_A_32_P212654 | | | BU616488 | | 0.04869837 |
| AGI_HUM1_OLIGO_A_24_P525749 | | Homo sapiens, clone IMAGE: 4214962, mRNA | AK091547 | | 0.04872217 |
| AGI_HUM1_OLIGO_A_23_P503182 | ABR | Active BCR-related gene | NM_021962 | 29 | 0.04875589 |
| AGI_HUM1_OLIGO_A_32_P155026 | | | ENST00000371189 | | 0.04875589 |
| AGI_HUM1_OLIGO_A_23_P167256 | PHF17 | PHD finger protein 17 | AK127326 | 79960 | 0.04894323 |
| AGI_HUM1_OLIGO_A_24_P392958 | C20orf140 | Chromosome 20 open reading frame 140 | BX647370 | 128637 | 0.04894323 |
| AGI_HUM1_OLIGO_A_32_P96752 | SOX4 | SRY (sex determining region Y)-box 4 | AW946823 | 6659 | 0.04894323 |
| AGI_HUM1_OLIGO_A_23_P215832 | ATP5J2 | ATP synthase, H+ transporting, mitochondrial F0 complex, subunit f, isoform 2 | NM_004889 | 9551 | 0.04894323 |
| AGI_HUM1_OLIGO_A_32_P1587 | ACYP2 | Acylphosphatase 2, muscle type | BQ066852 | 98 | 0.04894323 |
| AGI_HUM1_OLIGO_A_24_P99216 | LRP10 | Low density lipoprotein receptor-related protein 10 | NM_014045 | 26020 | 0.04894323 |
| AGI_HUM1_OLIGO_A_23_P72627 | HGS | Hepatocyte growth factor-regulated tyrosine kinase substrate | NM_004712 | 9146 | 0.04897374 |
| AGI_HUM1_OLIGO_A_32_P132827 | | | ENST00000355520 | | 0.04902203 |
| AGI_HUM1_OLIGO_A_32_P192692 | | | NM_001014797 | | 0.04902203 |
| AGI_HUM1_OLIGO_A_23_P68059 | NSPC1 | Likely ortholog of mouse nervous system polycomb 1 | NM_032673 | 84759 | 0.04902203 |
| AGI_HUMI_OLIGO_A_23_P130731 | TBC1D17 | TBC1 domain family, member 17 | NM_024682 | 79735 | 0.04902203 |
| AGI_HUM1_OLIGO_A_23_P217178 | MAGEC1 | Melanoma antigen, family C, 1 | NM_005462 | 9947 | 0.04902203 |
| AGI_HUM1_OLIGO_A_23_P204511 | FLJ20489 | Hypothetical protein FLJ20489 | NM_017842 | 55652 | 0.04902203 |
| AGI_HUM1_OLIGO_A_23_P85140 | TCEAL2 | Transcription elongation factor A (SII)-like 2 | NM_080390 | 140597 | 0.04902203 |
| AGI_HUM1_OLIGO_A_24_P136691 | MGC11257 | Hypothetical protein MGC11257 | BC025971 | 84310 | 0.04902203 |
| AGI_HUM1_OLIGO_A_32_P182186 | | | BC041955 | | 0.04902203 |
| AGI_HUM1_OLIGO_A_32_P81674 | LOC157627 | Hypothetical protein LOC157627 | AK091593 | 157627 | 0.04902203 |
| AGI_HUM1_OLIGO_A_23_P167537 | CPLX2 | Complexin 2 | NM_006650 | 10814 | 0.04902203 |
| AGI_HUM1_OLIGO_A_23_P202881 | FEZ1 | Fasciculation and elongation protein zeta 1 (zygin I) | NM_005103 | 9638 | 0.04902203 |
| AGI_HUM1_OLIGO_A_24_P412486 | | Similar to RIKEN cDNA 8030451K01 | CR601315 | 387921 | 0.04902203 |
| AGI_HUM1_OLIGO_A_32_P77742 | | | THC2338229 | | 0.04902203 |
| AGI_HUM1_OLIGO_A_23_P74887 | SDC3 | Syndecan 3 (N-syndecan) | AB007937 | 9672 | 0.04902203 |
| AGI_HUM1_OLIGO_A_23_P95764 | PRPS1 | Phosphoribosyl pyrophosphate synthetase 1 | NM_002764 | 5631 | 0.04902203 |
| AGI_HUM1_OLIGO_A_23_P107166 | ACBD4 | Acyl-Coenzyme A binding domain containing 4 | NM_024722 | 79777 | 0.04915923 |
| AGI_HUM1_OLIGO_A_23_P42435 | DTNBP1 | Dystrobrevin binding protein 1 | NM_183041 | 84062 | 0.04925664 |
| AGI_HUM1_OLIGO_A_32_P194915 | | | THC2449703 | | 0.04965406 |
| AGI_HUM1_OLIGO_A_32_P154079 | TGFB1I4 | Transforming growth factor beta 1 induced transcript 4 | AL533377 | 8848 | 0.0499513 |
| AGI_HUM1_OLIGO_A_24_P47467 | MGC5508 | Hypothetical protein MGC5508 | NM_024092 | 79073 | 0.0499513 |

1-12. (canceled)

13: A method for determining if a patient having Systemic Sclerosis (SSc) or scleroderma is a responder to mycophenolate mofetil (MMF) treatment comprising
(a) obtaining a gene expression signature from a skin sample of a patient having SSc or scleroderma;
(b) comparing the gene expression signature of (a) with a reference gene expression signature for a responder to MMF treatment;
(c) administering MMF to the patient when the comparison of (b) characterizes the patient as a responder to MMF treatment.

14: The method of claim 13, wherein the skin sample is obtained from an arm of the patient or back of the patient.

15: The method of claim 13, further comprising comparing the gene expression signature of (a) with a reference gene expression signature for a non-responder to MMF treatment.

16: The method of claim 13, wherein the gene expression signatures of (a) and (b) comprise the expression of at least two genes.
